(19) United States
(12) Patent Application Publication
(10) Pub. No.: US 2024/0092770 A1
(43) Pub. Date: Mar. 21, 2024

(54) Heterocycle RMB39 Modulators

(71) Applicant: Recursion Pharmaceuticals, Inc., Salt Lake City, UT (US)

(72) Inventors: Ashraf Saeed, Salt Lake City, UT (US); Chris Bailey, Salt Lake City, UT (US); Carl Brooks, Salt Lake City, UT (US); Kevin Fales, Layton, UT (US); Janet Paulsen, Salt Lake City, UT (US)

(21) Appl. No.: 18/234,341

(22) Filed: Aug. 15, 2023

Related U.S. Application Data
(60) Provisional application No. 63/398,050, filed on Aug. 15, 2022.

(51) Int. Cl. C07D 417/12; A61P 35/00; C07D 403/12; C07D 405/12; C07D 409/12; C07D 413/12; C07D 417/14

(52) U.S. Cl. CPC C07D 417/12; A61P 35/00; C07D 403/12; C07D 405/12; C07D 409/12; C07D 413/12; C07D 417/14

(57) ABSTRACT

Provided herein are compounds that modulate RMB39 and methods of using the compounds in RMB39-associated disorders, such as cancer (e.g., renal cell carcinoma).

### BACKGROUND

The present disclosure relates to thiazole derivatives and their use as pharmaceutical agents. Specifically, the present invention relates to the use of these compounds to degrade the activity of RNA-binding motif protein 39, also known as splicing factor HCC1, CAPERα, FSAP59, RNPC2, CAPER alpha containing 2, and commonly referred to as RBM39 (Xu, et al., Cell Death Discov. 7, 214 (2021)).

RBM39 (a 59.4 kDa protein) is an essential serine/arginine-rich RNA binding protein found in the nucleus of all living organisms and has been implicated in pre-mRNA splicing, transcriptional co-regulation, and translation (Xu, et al., Cell Death Discov. 7, 214 (2021)).

Several observations have led to the conclusion that RBM39 acts as a transcriptional co-activator of activating protein-1 (AP-1) and estrogen receptor alpha (ERα) with genes containing RBM39 regulated alternative exons linked to a wide variety of biological processes such as G2/M transition, cellular response to DNA damage, adherens junctions, and endocytosis (Mai, et al., Biochim Biophys Acta., 1859(8), 1014-1024 (2016)). RBM39 has also been associated with malignant progression in a number of solid and hematological cancers (Xu, et al., Cell Death Discov. 7, 214 (2021)).

A number of aryl sulfonamides (indisulam, tasisulam, CQS, and E7820) have been shown to act as molecular glue degraders of RBM39 by forming a ternary complex with RBM39 and the E3 ubiquitin ligase receptor DCAF15, with no detectable affinity for either species alone. These molecular glues promote the interaction of the RBM39 splicing factor and the CUL4-DCAF15 E3 ubiquitin ligase, leading to polyubiquitination and proteasomal degradation of RBM39. In human cancer cell lines treated with aryl sulfonamides, the degradation of RBM39 led to significant anti-proliferative effects. Additionally, using CRISPR-Cas9 to silence DCAF15 in cancer cells resisted RBM39 degradation by aryl sulfonamides, highlighting RBM39 degradation as the primary mechanism of anticancer effects seen with these compounds (Han, et al., Science., 356(6336), (2017)); Du, et al., Structure., 27, 1625-1633 (2019)). Furthermore, genetic knockout experiments of RBM39 deficient human cancer cells injected into mice slowed the growth of leukemia progression and improved overall survival (Wang, et al., Cancer Cell., 35(3), 369-384 (2019)).

Aryl sulfonamides have previously shown to exhibit an acceptable safety profile in clinical trials, with some anti-tumor efficacy seen across various cancers. However, overall response rates remained low, potentially due to a lack of understanding around the mechanism of action and potential biomarkers of response. Therefore, for specific patient populations, RBM39 degraders have the potential to effectively treat certain types of human cancers warranting further exploration (Wang, et al., Cancer Cell., 35(3), 369-384 (2019).

### SUMMARY

Provided herein are compounds of Formula (I), and pharmaceutically acceptable salts thereof:

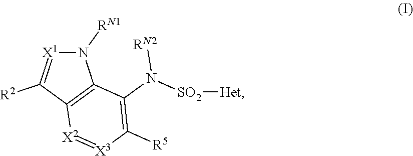

wherein R^{N1} is H or C₁₋₆alkyl optionally substituted with 1, 2, or 3 R⁷; R^{N2} is H or C₁₋₆alkyl optionally substituted with 1, 2, or 3 R⁷; X¹ is CR¹ or N; X² is CR³ or N; X³ is CR⁴ or N; R¹ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R² is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R³ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R⁴ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R⁵ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; each R^{N} is independently H, C₁₋₆alkyl optionally substituted with 1, 2, or 3 R⁷, or C₃₋₁₀ cycloalkyl; Het is a 5-, 6-, 7-, 8-, 9-, 10-, 11-, or 12-membered heteroaryl comprising 1, 2, or 3 ring heteroatoms selected from O, S, and N and optionally substituted with 1, 2, or 3 R⁶; each R⁶ is independently halo, CN, C₁₋₆alkyl, C₁₋₆haloalkyl, C₁₋₆alkoxy, NR^{N}R^{N}, COOH, C(O)NR^{N}R^{N}, C₁₋₆alkylene-C(O)OR^{N}, C₁₋₆alkylene-C(O)NR^{N}R^{N}, SO₂NR^{N}R^{N}, P(O)(R^{N})(R^{N}), C(O)-5- or 6-membered heterocycloalkyl comprising 1, 2, or 3 ring heteroatoms selected from O, S, and N, C₁₋₆alkylene-C₃₋₁₀cycloalkyl, C₃₋₁₀cycloalkyl, 4-6-membered heterocycloalkyl comprising 1, 2, or 3 ring heteroatoms selected from O, S, and N, C₃₋₁₀aryl, or 5- or 6-membered heteroaryl comprising 1, 2, or 3 ring heteroatoms selected from O, S, and N, wherein the C₃₋₁₀cycloalkyl, 4-6-membered heterocycloalkyl, C₃₋₁₀aryl, or 5- or 6-membered heteroaryl can optionally be substituted with 1, 2, or 3 R⁷ and each C₁₋₆alkyl, C₁₋₆alkylene, or C₁₋₆alkoxy can be optionally substituted with 1 or 2 substituents independently selected from C₁₋₆alkoxy, OH, CN, CO₂H, NR^{N}R^{N}, and CO₂C₁₋₆alkyl; and; each R⁷ is independently OH, halo, CN, C₁₋₆alkyl, C₁₋₆haloalkyl, C₁₋₆alkoxy, NH₂, NH(C₁₋₆alkyl), or N(C₁₋₆alkyl)₂. In some cases, R^{N1} is H or C₁₋₆alkyl optionally substituted with 1, 2, or 3 R⁷; R^{N2} is H or C₁₋₆alkyl optionally substituted with 1, 2, or 3 R⁷; X¹ is CR¹ or N; X² is CR³ or N; X³ is CR⁴ or N; R¹ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R² is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R³ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R⁴ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; R⁵ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl; each R^{N} is independently H or C₁₋₆alkyl optionally substituted with 1, 2, or 3 R⁷; Het is a 5-, 6-, 7-, 8-, 9-, 10-, 11-, or 12-membered heteroaryl comprising 1, 2, or 3 ring heteroatoms selected from O, S, and N and optionally substituted with 1, 2, or 3 R⁶; each R⁶ is independently halo, CN, C₁₋₆alkyl, C₁₋₆haloalkyl, C₁₋₆alkoxy, COOH, C(O)NR^{N}R^{N}, C₁₋₆alkylene-C(O)OR^{N}, P(O)(R^{N})(R^{N}), C(O)-5- or 6-membered heterocycloalkyl comprising 1, 2, or 3 ring heteroatoms selected from O, S, and N, C₃₋₁₀cycloalkyl, 5- or 6-membered heterocycloalkyl comprising 1, 2, or 3 ring heteroatoms selected from O, S, and N, C₃₋₁₀aryl, or 5- or 6-membered heteroaryl comprising 1, 2, or 3 ring heteroatoms selected from O, S, and N, wherein the C₃₋₁₀cycloalkyl, 5- or 6-membered heterocycloalkyl, C₃₋₁₀aryl, or 5- or 6-membered heteroaryl can optionally be substituted with 1, 2, or 3 R⁷ and each C₁₋₆alkyl or C₁₋₆alkylene can be optionally substituted with 1 or 2 substituents independently selected from C₁₋₆alkoxy, OH, CN, CO₂H, NR^{N}R^{N}, and CO₂C₁₋₆alkyl; and each R⁷ is independently OH, halo, CN, C₁₋₆alkyl, C₁₋₆haloalkyl, C₁₋₆alkoxy, NH₂, NH(C₁₋₆alkyl), or N(C₁₋₆alkyl) 2.

Also provided are compositions comprising compounds as disclosed herein and a pharmaceutically acceptable excipient. Further provided are methods of modulating RBM39 protein by contacting RBM30 protein with a compound as disclosed herein. Also provided are methods of treating a disease associated with aberrant RBM39 activity (e.g., cancer such as renal cell carcinoma) in a subject by administering a therapeutically effective amount of a compound as disclosed herein to the subject.

### DETAILED DESCRIPTION

Provided herein are compounds, and their use in treating or preventing diseases and disorders associated with aberrant RBM39 activity, e.g., cancer. Also provided are uses of the compounds described herein, or pharmaceutically acceptable salts thereof, or pharmaceutically acceptable compositions comprising such a compound or a pharmaceutically acceptable salt thereof, for the treatment or prevention of diseases and disorders associated with aberrant RBM39 activity, e.g., cancer.

### Compounds

Provided herein are compounds of Formula (I), and pharmaceutically acceptable salts thereof:

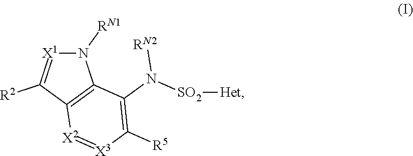

wherein
R^{N1} is H or C₁₋₆alkyl optionally substituted with 1, 2, or 3 R⁷;
R^{N2} is H or C₁₋₆alkyl optionally substituted with 1, 2, or 3 R⁷;
X¹ is CR¹ or N;
X² is CR³ or N;
X³ is CR⁴ or N;
R¹ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R² is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R³ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁴ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁵ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
each R^{N} is independently H, C₁₋₆alkyl optionally substituted with 1, 2, or 3 R⁷, or C₃₋₁₀cycloalkyl; Het is a 5-, 6-, 7-, 8-, 9-, 10-, 11-, or 12-membered heteroaryl comprising 1, 2, or 3 ring heteroatoms selected from O, S, and N and optionally substituted with 1, 2, or 3 R⁶;
each R⁶ is independently halo, CN, C₁₋₆alkyl, C₁₋₆haloalkyl, C₁₋₆alkoxy, NR^{N}R^{N}, COOH, C(O)NR^{N}R^{N}, C₁₋₆alkylene-C(O)OR^{N}, C₁₋₆alkylene-C(O)NR^{N}R^{N}, SO₂NR^{N}R^{N}, P(O)(R^{N})(R^{N}), C(O)-5- or 6-membered heterocycloalkyl comprising 1, 2, or 3 ring heteroatoms selected from O, S, and N, C₁₋₆alkylene-C₃₋₁₀ cycloalkyl, C₃₋₁₀ cycloalkyl, 4-6-membered heterocycloalkyl comprising 1, 2, or 3 ring heteroatoms selected from O, S, and N, C₆₋₁₀aryl, or 5- or 6-membered heteroaryl comprising 1, 2, or 3 ring heteroatoms selected from O, S, and N, wherein the C₃₋₁₀cycloalkyl, 4-6-membered heterocycloalkyl, C₃₋₁₀aryl, or 5- or 6-membered heteroaryl can optionally be substituted with 1, 2, or 3 R⁷ and each C₁₋₆alkyl, C₁₋₆alkylene, or C₁₋₆alkoxy can be optionally substituted with 1 or 2 substituents independently selected from C₁₋₆alkoxy, OH, CN, CO₂H, NR^{N}R^{N}, and CO₂C₁₋₆alkyl; and;
each R⁷ is independently OH, halo, CN, C₁₋₆alkyl, C₁₋₆haloalkyl, C₁₋₆alkoxy, NH₂, NH(C₁₋₆alkyl), or N(C₁₋₆alkyl)₂.

In some cases, R^{N1} is H or C₁₋₆alkyl optionally substituted with 1, 2, or 3 R⁷;
R^{N2} is H or C₁₋₆alkyl optionally substituted with 1, 2, or 3 R⁷;
X¹ is CR¹ or N;
X² is CR³ or N;
X³ is CR⁴ or N;
R¹ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R² is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R³ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁴ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁵ is H, C₁₋₆alkyl, C₁₋₆haloalkyl, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
each R^{N} is independently H or C₁₋₆alkyl optionally substituted with 1, 2, or 3 R⁷;
Het is a 5-, 6-, 7-, 8-, 9-, 10-, 11-, or 12-membered heteroaryl comprising 1, 2, or 3 ring heteroatoms selected from O, S, and N and optionally substituted with 1, 2, or 3 R⁶;
each R⁶ is independently halo, CN, C₁₋₆alkyl, C₁₋₆haloalkyl, C₁₋₆alkoxy, COOH, C(O)NR^{N}R^{N}, C₁₋₆alkylene-C(O)OR^{N}, P(O)(R^{N})(R^{N}), C(O)-5- or 6-membered heterocycloalkyl comprising 1, 2, or 3 ring heteroatoms selected from O, S, and N, C₃₋₁₀cycloalkyl, 5- or 6-membered heterocycloalkyl comprising 1, 2, or 3 ring heteroatoms selected from O, S, and N, C₃₋₁₀aryl, or 5- or 6-membered heteroaryl comprising 1, 2, or 3 ring heteroatoms selected from O, S, and N, wherein the C₃₋₁₀cycloalkyl, 5- or 6-membered heterocycloalkyl, C₃₋₁₀aryl, or 5- or 6-membered heteroaryl can optionally be substituted with 1, 2, or 3 R⁷ and each C₁₋₆alkyl or C₁₋₆alkylene can be optionally substituted with 1 or 2 substituents independently selected from C₁₋₆alkoxy, OH, CN, CO₂H, NR^{N}R^{N}, and CO₂C₁₋₆alkyl; and;
each R⁷ is independently OH, halo, CN, C₁₋₆alkyl, C₁₋₆haloalkyl, C₁₋₆alkoxy, NH₂, NH(C₁₋₆alkyl), or N(C₁₋₆alkyl)₂.

In some cases, Het is a 5- or 6-membered heteroaryl comprising 1, 2, or 3 ring heteroatoms selected from O, S, and N and optionally substituted with 1, 2, or 3 R⁶. In some cases, Het is a 5-membered heteroaryl comprising 1, 2, or 3 ring heteroatoms selected from O, S, and N and optionally substituted with 1, 2, or 3 R⁶. In some cases, Het is thiazolyl optionally substituted with 1, 2, or 3 R⁶. In some cases, Het is

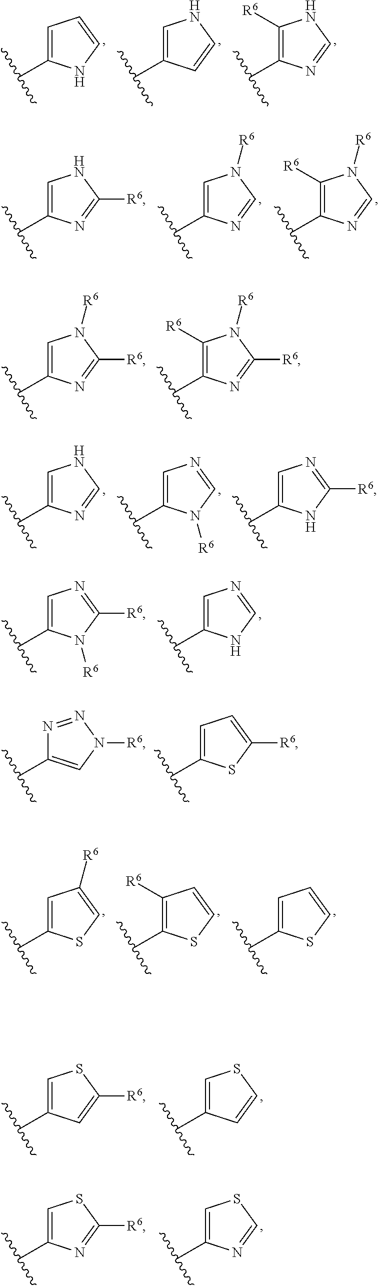

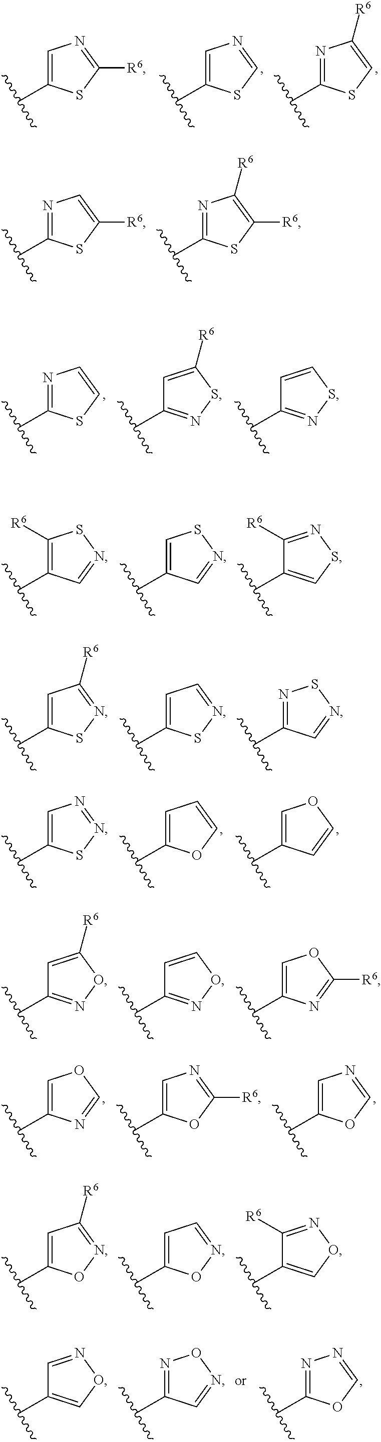

and R⁶ is C₁₋₆alkyl, C₁₋₆haloalkyl, C₁₋₆alkoxy, C₃₋₁₀cycloalkyl, P(O)(Me)₂, or C(O)-morpholinyl. In some cases, Het is

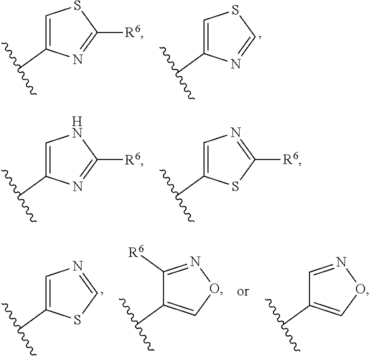

and R⁶ is C₁₋₆alkyl, C₁₋₆haloalkyl, C₁₋₆alkoxy, C₃₋₁₀cycloalkyl, P(O)(Me)₂, or C(O)-morpholinyl. In some cases, Het is thiazolyl optionally substituted with 1, 2, or 3 R⁶. In some cases, Het is

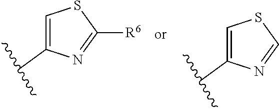

and R⁶ is C₁₋₆alkyl, C₁₋₆haloalkyl, C₁₋₆alkoxy, C₃₋₁₀cycloalkyl, P(O)(Me)₂, or C(O)-morpholinyl. In some cases, Het is

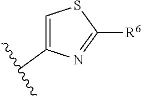

and R⁶ is C₁₋₆alkyl, C₁₋₆haloalkyl, C₁₋₆alkoxy, C₃₋₁₀cycloalkyl, P(O)(Me)₂, or C(O)-morpholinyl.

In some cases, X¹ is CR¹. In some cases, X² is CR³. In some cases, X³ is CR⁴. In some cases, X¹ is CR¹, X² is CR³, and X³ is CR⁴. In some cases, at least one of X¹, X², and X³ is N. In some cases, one of X¹, X², and X³ is N. In some cases, two of X¹, X², and X³ are N. In some cases, X¹ is N. In some cases, X² is N. In some cases, X³ is N.

In some cases, the compound has the structure of Formula (Ia):

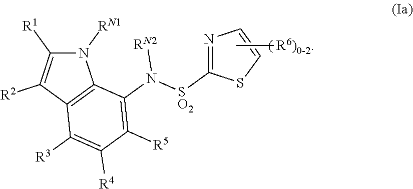

In some cases, one of R^{N1} and R^{N2} is H. In some cases, R^{N1} is H. In some cases, R^{N2} is H. In some cases, R^{N1} and R^{N2} are each H. In some cases, R^{N1} is C₁₋₆alkyl optionally substituted with 1, 2, or 3 R⁷. In some cases, R^{N2} is C₁₋₆alkyl optionally substituted with 1, 2, or 3 R⁷.

In some cases, R¹ is H or C₁₋₆alkyl, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl. In some cases, R¹ is H.

In some cases, R² is H, C₁₋₆alkyl, halo, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl. In some cases, R² is H, halo, or CN. In some cases, R² is Cl. In some cases, R² is CN.

In some cases, R³ is H, C₁₋₆alkyl, or halo, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl. In some cases, R³ is H. In some cases, R³ is C₁₋₆alkyl, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl. In some cases, R³ is methyl. In some cases, R³ is halo. In some cases, R³ is fluoro.

In some cases, R⁴ is H, C₁₋₆alkyl, or halo, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl. In some cases R⁴ is H.

In some cases, R⁵ is H, C₁₋₆alkyl, or halo, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl. In some cases, R⁵ is H.

In some cases, each R⁶ is independently halo, CN, C₁₋₆alkyl, C₁₋₆haloalkyl, C₁₋₆alkoxy, COOH, C(O)NR^{N}R^{N}, C₁₋₆alkylene-C(O)OR^{N}, P(O)(R^{N})(R^{N}), C(O)-5- or 6-membered heterocycloalkyl comprising 1, 2, or 3 ring heteroatoms selected from O, S, and N, C₃₋₅ cycloalkyl, 5- or 6-membered heterocycloalkyl comprising 1, 2, or 3 ring heteroatoms selected from O, S, and N, C₃₋₁₀aryl, or 5- or 6-membered heteroaryl comprising 1, 2, or 3 ring heteroatoms selected from O, S, and N, wherein the C₃₋₅ cycloalkyl, 5- or 6-membered heterocycloalkyl, C₆₋₁₀aryl, or 5- or 6-membered heteroaryl can optionally be substituted with 1, 2, or 3 R⁷ and each C₁₋₆alkyl or C₁₋₆alkylene can be optionally substituted with 1 or 2 substituents independently selected from C₁₋₆alkoxy, OH, CN, CO₂H, NR^{N}R^{N}, and CO₂C₁₋₆alkyl. In some cases, each R⁶ is independently halo, CN, C₁₋₆alkyl, C₁₋₆haloalkyl, C₁₋₆alkoxy, C₁₋₆alkylene-C(O)O—C₁₋₆alkyl, 5- or 6-membered heterocycloalkyl comprising 1, 2, or 3 ring heteroatoms selected from O, S, and N, C₃₋₁₀aryl, or 5- or 6-membered heteroaryl comprising 1, 2, or 3 ring heteroatoms selected from O, S, and N, wherein the 5- or 6-membered heterocycloalkyl, C₃₋₁₀aryl, or 5- or 6-membered heteroaryl can optionally be substituted with 1, 2, or 3 R⁷ and each C₁₋₆alkyl or C₁₋₆alkylene can be optionally substituted with 1 or 2 substituents independently selected from C₁₋₆alkoxy, OH, CN, CO₂H, NR^{N}R^{N}, and CO₂C₁₋₆alkyl. In some cases, each R⁶ is independently halo, C₁₋₆alkyl, or 5- or 6-membered heteroaryl comprising 1, 2, or 3 ring heteroatoms selected from O, S, and N, wherein the 5- or 6-membered heteroaryl can optionally be substituted with 1, 2, or 3 R⁷ and each C₁₋₆alkyl can be optionally substituted with 1 or 2 substituents independently selected from C₁₋₆alkoxy, OH, CN, CO₂H, NR^{N}R^{N}, and CO₂C₁₋₆alkyl. In some cases, at least one R⁶ is halo. In some cases, one R⁶ is halo. In some cases, at least one R⁶ is chloro or bromo. In some cases, one R⁶ is chloro or bromo. In some cases, R⁶ is chloro. In some cases, R⁶ is bromo. In some cases, at least one R⁶ is C₁₋₆alkyl, and each C₁₋₆alkyl can be optionally substituted with 1 or 2 substituents independently selected from C₁₋₆alkoxy, OH, CN, CO₂H, NR^{N}R^{N}, and CO₂C₁₋₆alkyl. In some cases, at least one R⁶ is methyl, ethyl, or isopropyl. In some cases, one R⁶ is methyl, ethyl, or isopropyl. In some cases, R⁶ is methyl. In some cases, R⁶ is ethyl. In some cases, R⁶ is isopropyl. In some cases, each R⁶ is independently 5- or 6-membered heteroaryl comprising 1, 2, or 3 ring heteroatoms selected from O, S, and N, wherein the 5- or 6-membered heteroaryl can optionally be substituted with 1, 2, or 3 R⁷. In some cases, at least one R⁶ is pyrazolyl, pyridinyl, pyridazinyl, or pyrimidinyl, each of which can optionally be substituted with 1, 2, or 3 R⁷. In some cases, one R⁶ is pyrazolyl, pyridinyl, pyridazinyl, or pyrimidinyl, each of which can optionally be substituted with 1, 2, or 3 R⁷. In some cases R⁶ is pyrazolyl optionally substituted with 1, 2, or 3 R⁷. In some cases R⁶ is pyridinyl optionally substituted with 1, 2, or 3 R⁷. In some cases R⁶ is pyridazinyl, or optionally substituted with 1, 2, or 3 R⁷. In some cases R⁶ is pyrimidinyl optionally substituted with 1, 2, or 3 R⁷.

In some cases, each R⁷ is independently halo, C₁₋₆alkyl, or C₁₋₆haloalkyl. In some cases, at least one R⁷ is C₁₋₆alkyl. In some cases, at least one R⁷ is methyl.

Specific compounds contemplated include compounds in the following Table A, or a pharmaceutically acceptable salt thereof. Compounds having a chiral center without indication of a particular stereoisomerism indicate a mixture of stereocenters at that chiral center.

TABLE A

| Com- | |
|---|---|
| pound | |
| No. | Structure |
| 1 | 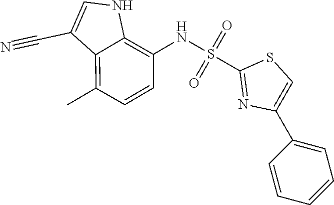 [SMILES: Cc1ccc(NS(=O)(=O)c2nc(-c3ccccc3)cs2)c2[nH]cc(C#N)c12] |
| 2 | 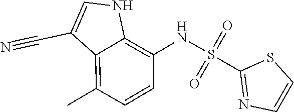 [SMILES: Cc1ccc(NS(=O)(=O)c2nccs2)c2[nH]cc(C#N)c12] |
| 3 | 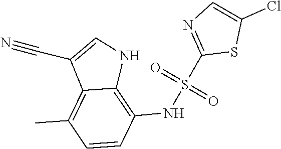 [SMILES: Cc1ccc(NS(=O)(=O)c2ncc(Cl)s2)c2[nH]cc(C#N)c12] |
| 4 | 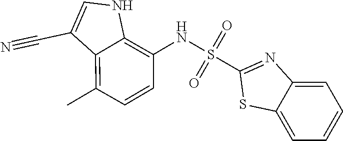 [SMILES: Cc1ccc(NS(=O)(=O)c2nc3ccccc3s2)c2[nH]cc(C#N)c12] |
| 5 | 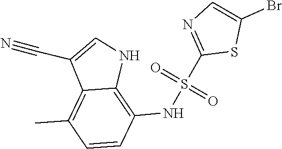 [SMILES: Cc1ccc(NS(=O)(=O)c2ncc(Br)s2)c2[nH]cc(C#N)c12] |
| 6 | 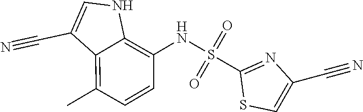 [SMILES: Cc1ccc(NS(=O)(=O)c2nc(C#N)cs2)c2[nH]cc(C#N)c12] |
| 7 | 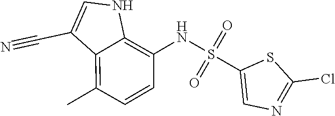 [SMILES: Cc1ccc(NS(=O)(=O)c2cnc(Cl)s2)c2[nH]cc(C#N)c12] |
| 8 | 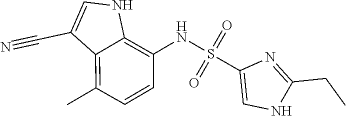 [SMILES: CCc1nc(S(=O)(=O)Nc2ccc(C)c3c(C#N)c[nH]c23)c[nH]1] |
| 9 | 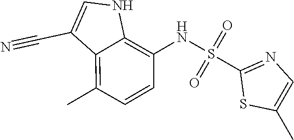 [SMILES: Cc1cnc(S(=O)(=O)Nc2ccc(C)c3c(C#N)c[nH]c23)s1] |
| 10 | 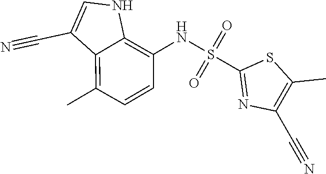 [SMILES: Cc1sc(S(=O)(=O)Nc2ccc(C)c3c(C#N)c[nH]c23)nc1C#N] |
| 11 | 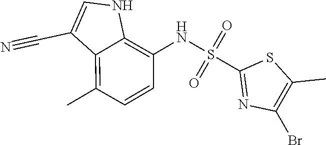 [SMILES: Cc1sc(S(=O)(=O)Nc2ccc(C)c3c(C#N)c[nH]c23)nc1Br] |
| 12 | 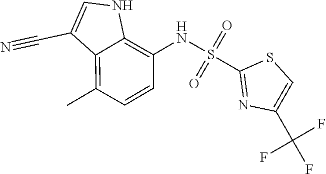 [SMILES: Cc1ccc(NS(=O)(=O)c2nc(C(F)(F)F)cs2)c2[nH]cc(C#N)c12] |
| 13 | 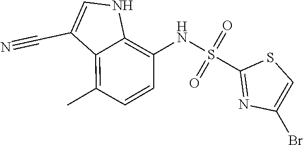 [SMILES: Cc1ccc(NS(=O)(=O)c2nc(Br)cs2)c2[nH]cc(C#N)c12] |
| 14 | 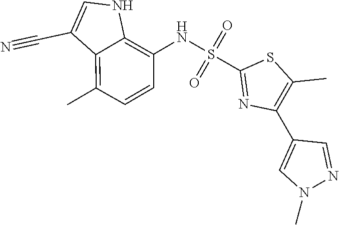 [SMILES: Cc1sc(S(=O)(=O)Nc2ccc(C)c3c(C#N)c[nH]c23)nc1-c1cnn(C)c1] |
| 15 | 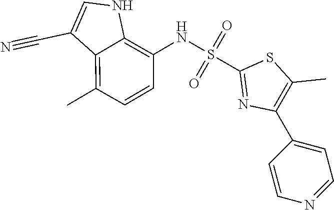 [SMILES: Cc1sc(S(=O)(=O)Nc2ccc(C)c3c(C#N)c[nH]c23)nc1-c1ccncc1] |
| 16 | 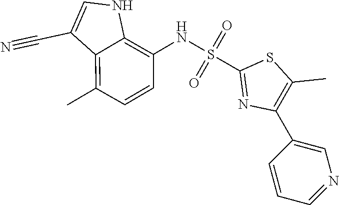 [SMILES: Cc1sc(S(=O)(=O)Nc2ccc(C)c3c(C#N)c[nH]c23)nc1-c1cccnc1] |
| 17 | 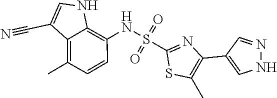 [SMILES: Cc1sc(S(=O)(=O)Nc2ccc(C)c3c(C#N)c[nH]c23)nc1-c1cn[nH]c1] |
| 18 | 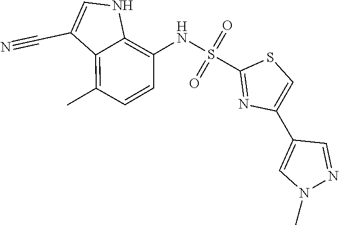 [SMILES: Cc1ccc(NS(=O)(=O)c2nc(-c3cnn(C)c3)cs2)c2[nH]cc(C#N)c12] |
| 19 | 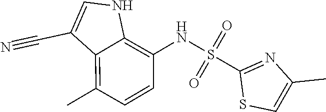 [SMILES: Cc1csc(S(=O)(=O)Nc2ccc(C)c3c(C#N)c[nH]c23)n1] |
| 20 | 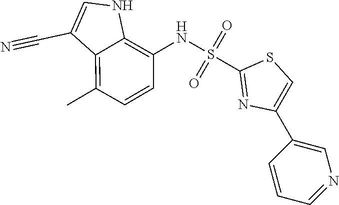 [SMILES: Cc1ccc(NS(=O)(=O)c2nc(-c3cccnc3)cs2)c2[nH]cc(C#N)c12] |
| 21 | 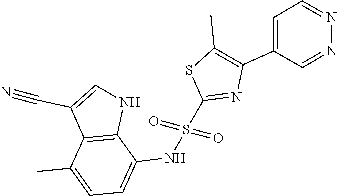 [SMILES: Cc1sc(S(=O)(=O)Nc2ccc(C)c3c(C#N)c[nH]c23)nc1-c1ccnnc1] |
| 22 | 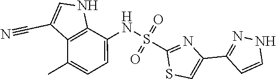 [SMILES: Cc1ccc(NS(=O)(=O)c2nc(-c3cc[nH]n3)cs2)c2[nH]cc(C#N)c12] |
| 23 | 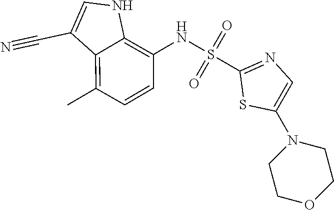 [SMILES: Cc1ccc(NS(=O)(=O)c2ncc(N3CCOCC3)s2)c2[nH]cc(C#N)c12] |
| 24 | 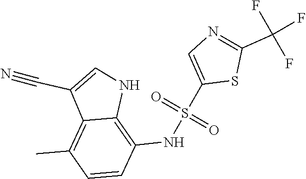 [SMILES: Cc1ccc(NS(=O)(=O)c2cnc(C(F)(F)F)s2)c2[nH]cc(C#N)c12] |
| 25 | 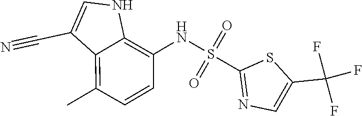 [SMILES: Cc1ccc(NS(=O)(=O)c2ncc(C(F)(F)F)s2)c2[nH]cc(C#N)c12] |
| 26 | 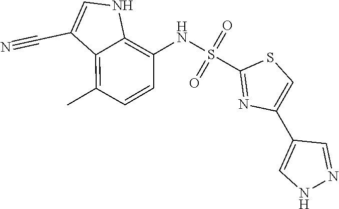 [SMILES: Cc1ccc(NS(=O)(=O)c2nc(-c3cn[nH]c3)cs2)c2[nH]cc(C#N)c12] |
| 27 | 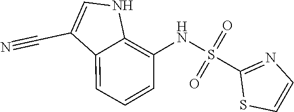 [SMILES: N#Cc1c[nH]c2c(NS(=O)(=O)c3nccs3)cccc12] |
| 28 | 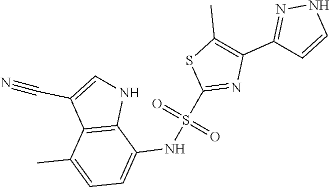 [SMILES: Cc1sc(S(=O)(=O)Nc2ccc(C)c3c(C#N)c[nH]c23)nc1-c1cc[nH]n1] |
| 29 | 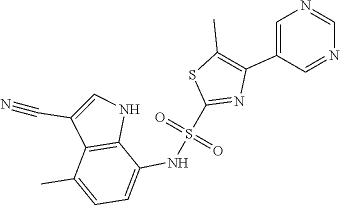 [SMILES: Cc1sc(S(=O)(=O)Nc2ccc(C)c3c(C#N)c[nH]c23)nc1-c1cncnc1] |
| 30 | 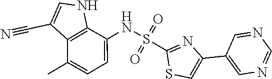 [SMILES: Cc1ccc(NS(=O)(=O)c2nc(-c3cncnc3)cs2)c2[nH]cc(C#N)c12] |
| 31 | 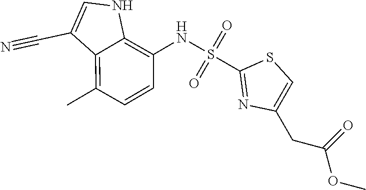 [SMILES: COC(=O)Cc1csc(S(=O)(=O)Nc2ccc(C)c3c(C#N)c[nH]c23)n1] |
| 32 | 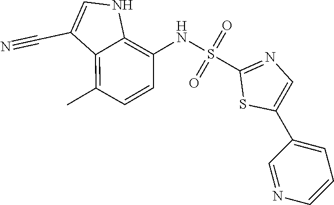 [SMILES: Cc1ccc(NS(=O)(=O)c2ncc(-c3cccnc3)s2)c2[nH]cc(C#N)c12] |
| 33 | 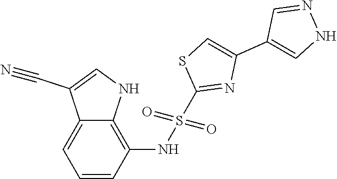 [SMILES: N#Cc1c[nH]c2c(NS(=O)(=O)c3nc(-c4cn[nH]c4)cs3)cccc12] |
| 34 | 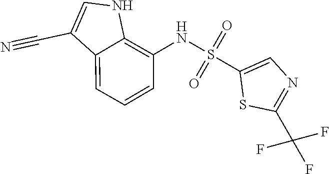 [SMILES: N#Cc1c[nH]c2c(NS(=O)(=O)c3cnc(C(F)(F)F)s3)cccc12] |
| 35 | 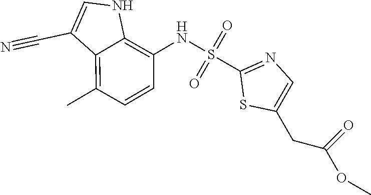 [SMILES: COC(=O)Cc1cnc(S(=O)(=O)Nc2ccc(C)c3c(C#N)c[nH]c23)s1] |
| 36 | 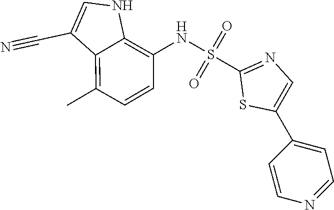 [SMILES: Cc1ccc(NS(=O)(=O)c2ncc(-c3ccncc3)s2)c2[nH]cc(C#N)c12] |
| 37 | 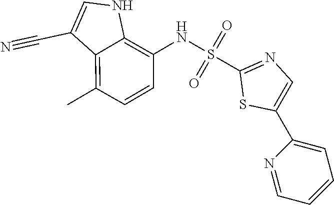 [SMILES: Cc1ccc(NS(=O)(=O)c2ncc(-c3ccccn3)s2)c2[nH]cc(C#N)c12] |
| 38 | 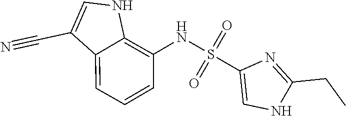 [SMILES: CCc1nc(S(=O)(=O)Nc2cccc3c(C#N)c[nH]c23)c[nH]1] |
| 39 | 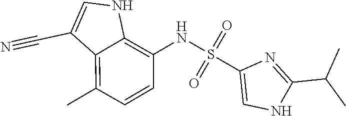 [SMILES: Cc1ccc(NS(=O)(=O)c2c[nH]c(C(C)C)n2)c2[nH]cc(C#N)c12] |
| 40 | 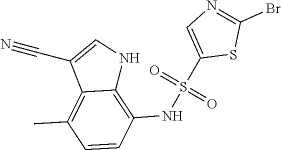 [SMILES: Cc1ccc(NS(=O)(=O)c2cnc(Br)s2)c2[nH]cc(C#N)c12] |
| 41 | 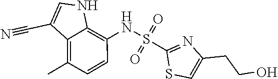 [SMILES: Cc1ccc(NS(=O)(=O)c2nc(CCO)cs2)c2[nH]cc(C#N)c12] |
| 42 | 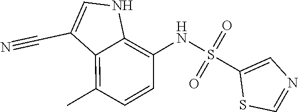 [SMILES: Cc1ccc(NS(=O)(=O)c2cncs2)c2[nH]cc(C#N)c12] |
| 43 | 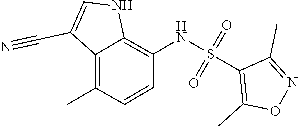 [SMILES: Cc1noc(C)c1S(=O)(=O)Nc1ccc(C)c2c(C#N)c[nH]c12] |
| 44 | 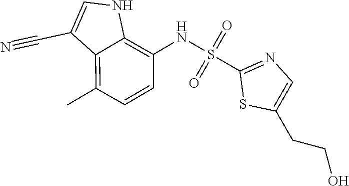 [SMILES: Cc1ccc(NS(=O)(=O)c2ncc(CCO)s2)c2[nH]cc(C#N)c12] |
| 45 | 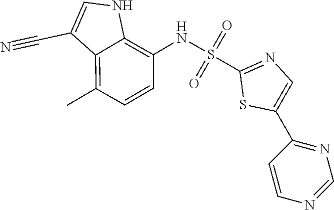 [SMILES: Cc1ccc(NS(=O)(=O)c2ncc(-c3ccncn3)s2)c2[nH]cc(C#N)c12] |
| 46 | 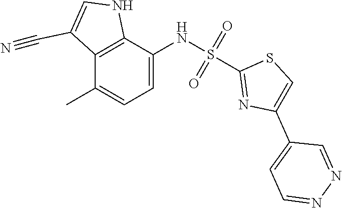 [SMILES: Cc1ccc(NS(=O)(=O)c2nc(-c3ccnnc3)cs2)c2[nH]cc(C#N)c12] |
| 47 | 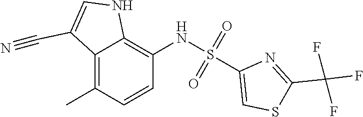 [SMILES: Cc1ccc(NS(=O)(=O)c2csc(C(F)(F)F)n2)c2[nH]cc(C#N)c12] |
| 48 | 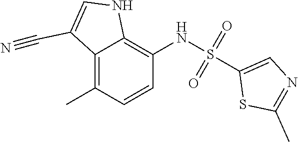 [SMILES: Cc1ncc(S(=O)(=O)Nc2ccc(C)c3c(C#N)c[nH]c23)s1] |
| 49 | 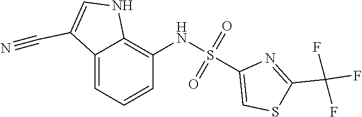 [SMILES: N#Cc1c[nH]c2c(NS(=O)(=O)c3csc(C(F)(F)F)n3)cccc12] |
| 50 | 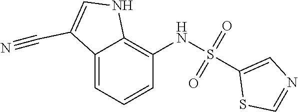 [SMILES: N#Cc1c[nH]c2c(NS(=O)(=O)c3cncs3)cccc12] |
| 51 | 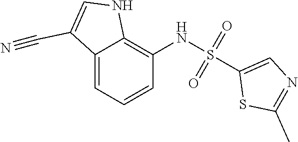 [SMILES: Cc1ncc(S(=O)(=O)Nc2cccc3c(C#N)c[nH]c23)s1] |
| 52 | 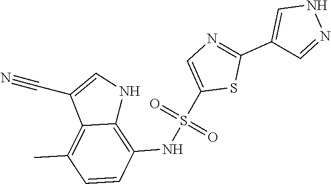 [SMILES: Cc1ccc(NS(=O)(=O)c2cnc(-c3cn[nH]c3)s2)c2[nH]cc(C#N)c12] |
| 53 | 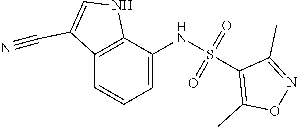 [SMILES: Cc1noc(C)c1S(=O)(=O)Nc1cccc2c(C#N)c[nH]c12] |
| 54 | 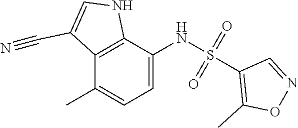 [SMILES: Cc1oncc1S(=O)(=O)Nc1ccc(C)c2c(C#N)c[nH]c12] |
| 55 | 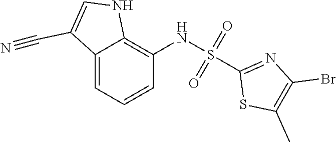 [SMILES: Cc1sc(S(=O)(=O)Nc2cccc3c(C#N)c[nH]c23)nc1Br] |
| 56 | 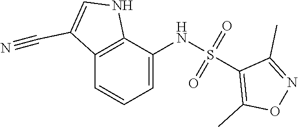 [SMILES: Cc1noc(C)c1S(=O)(=O)Nc1cccc2c(C#N)c[nH]c12] |
| 57 | 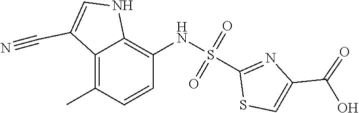 [SMILES: Cc1ccc(NS(=O)(=O)c2nc(C(=O)O)cs2)c2[nH]cc(C#N)c12] |
| 58 | 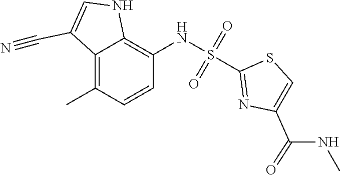 [SMILES: CNC(=O)c1csc(S(=O)(=O)Nc2ccc(C)c3c(C#N)c[nH]c23)n1] |
| 59 | 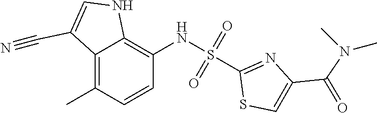 [SMILES: Cc1ccc(NS(=O)(=O)c2nc(C(=O)N(C)C)cs2)c2[nH]cc(C#N)c12] |
| 60 | 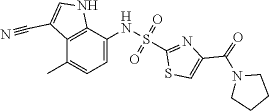 [SMILES: Cc1ccc(NS(=O)(=O)c2nc(C(=O)N3CCCC3)cs2)c2[nH]cc(C#N)c12] |
| 61 | 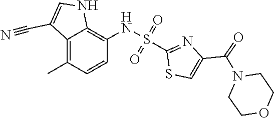 [SMILES: Cc1ccc(NS(=O)(=O)c2nc(C(=O)N3CCOCC3)cs2)c2[nH]cc(C#N)c12] |
| 62 | 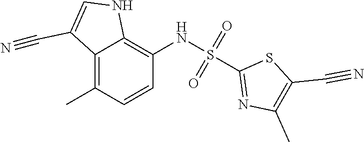 [SMILES: Cc1nc(S(=O)(=O)Nc2ccc(C)c3c(C#N)c[nH]c23)sc1C#N] |
| 63 | 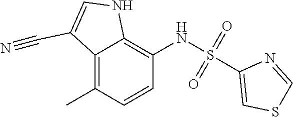 [SMILES: Cc1ccc(NS(=O)(=O)c2cscn2)c2[nH]cc(C#N)c12] |
| 64 | 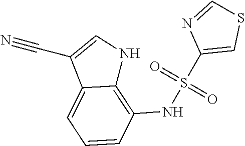 [SMILES: N#Cc1c[nH]c2c(NS(=O)(=O)c3cscn3)cccc12] |
| 65 | 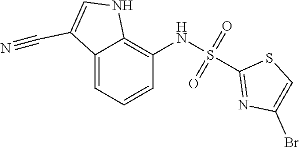 [SMILES: N#Cc1c[nH]c2c(NS(=O)(=O)c3nc(Br)cs3)cccc12] |
| 66 | 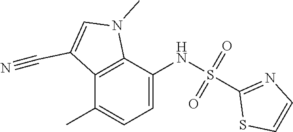 [SMILES: Cc1ccc(NS(=O)(=O)c2nccs2)c2c1c(C#N)cn2C] |
| 67 | 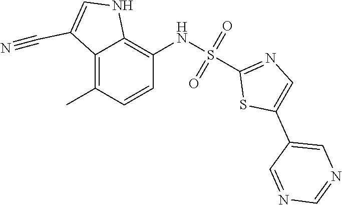 [SMILES: Cc1ccc(NS(=O)(=O)c2ncc(-c3cncnc3)s2)c2[nH]cc(C#N)c12] |
| 68 | 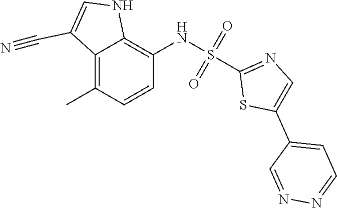 [SMILES: Cc1ccc(NS(=O)(=O)c2ncc(-c3ccnnc3)s2)c2[nH]cc(C#N)c12] |
| 69 | 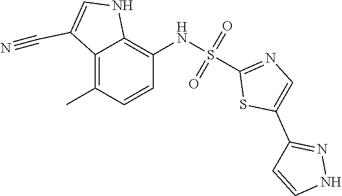 [SMILES: Cc1ccc(NS(=O)(=O)c2ncc(-c3cc[nH]n3)s2)c2[nH]cc(C#N)c12] |
| 70 | 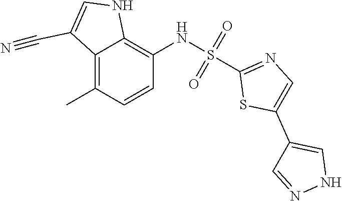 [SMILES: Cc1ccc(NS(=O)(=O)c2ncc(-c3cn[nH]c3)s2)c2[nH]cc(C#N)c12] |
| 71 | 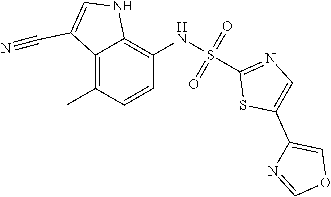 [SMILES: Cc1ccc(NS(=O)(=O)c2ncc(-c3cocn3)s2)c2[nH]cc(C#N)c12] |
| 72 | 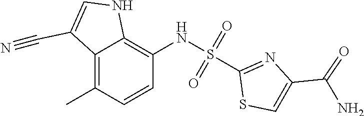 [SMILES: Cc1ccc(NS(=O)(=O)c2nc(C(N)=O)cs2)c2[nH]cc(C#N)c12] |
| 73 | 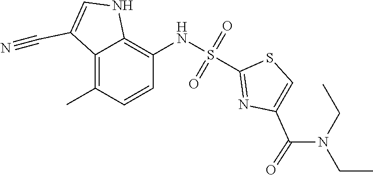 [SMILES: CCN(CC)C(=O)c1csc(S(=O)(=O)Nc2ccc(C)c3c(C#N)c[nH]c23)n1] |
| 74 | 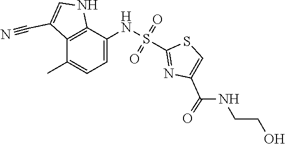 [SMILES: Cc1ccc(NS(=O)(=O)c2nc(C(=O)NCCO)cs2)c2[nH]cc(C#N)c12] |
| 75 | 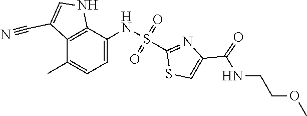 [SMILES: COCCNC(=O)c1csc(S(=O)(=O)Nc2ccc(C)c3c(C#N)c[nH]c23)n1] |
| 76 | 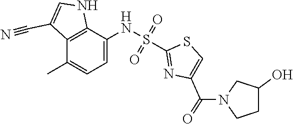 [SMILES: Cc1ccc(NS(=O)(=O)c2nc(C(=O)N3CCC(O)C3)cs2)c2[nH]cc(C#N)c12] |
| 77 | 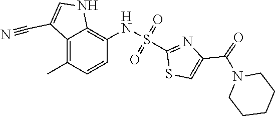 [SMILES: Cc1ccc(NS(=O)(=O)c2nc(C(=O)N3CCCCC3)cs2)c2[nH]cc(C#N)c12] |
| 78 | 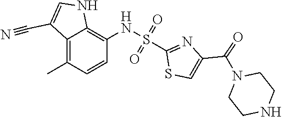 [SMILES: Cc1ccc(NS(=O)(=O)c2nc(C(=O)N3CCNCC3)cs2)c2[nH]cc(C#N)c12] |
| 79 | 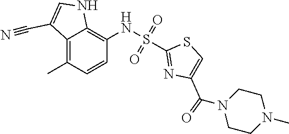 [SMILES: Cc1ccc(NS(=O)(=O)c2nc(C(=O)N3CCN(C)CC3)cs2)c2[nH]cc(C#N)c12] |
| 80 | 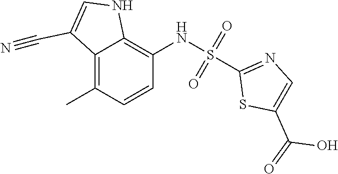 [SMILES: Cc1ccc(NS(=O)(=O)c2ncc(C(=O)O)s2)c2[nH]cc(C#N)c12] |
| 81 | 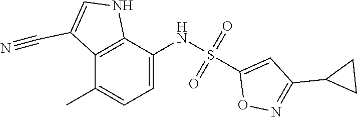 [SMILES: Cc1ccc(NS(=O)(=O)c2cc(C3CC3)no2)c2[nH]cc(C#N)c12] |
| 82 | 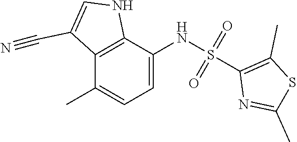 [SMILES: Cc1nc(S(=O)(=O)Nc2ccc(C)c3c(C#N)c[nH]c23)c(C)s1] |
| 83 | 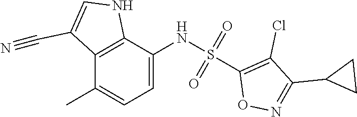 [SMILES: Cc1ccc(NS(=O)(=O)c2onc(C3CC3)c2Cl)c2[nH]cc(C#N)c12] |
| 84 | 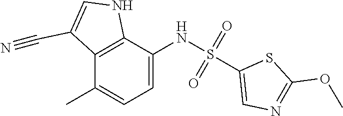 [SMILES: COc1ncc(S(=O)(=O)Nc2ccc(C)c3c(C#N)c[nH]c23)s1] |
| 85 | 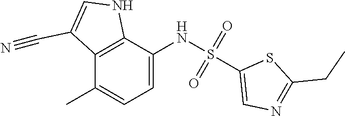 [SMILES: CCc1ncc(S(=O)(=O)Nc2ccc(C)c3c(C#N)c[nH]c23)s1] |
| 86 | 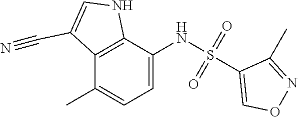 [SMILES: Cc1nocc1S(=O)(=O)Nc1ccc(C)c2c(C#N)c[nH]c12] |
| 87 | 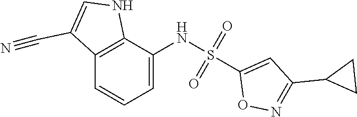 [SMILES: N#Cc1c[nH]c2c(NS(=O)(=O)c3cc(C4CC4)no3)cccc12] |
| 88 | 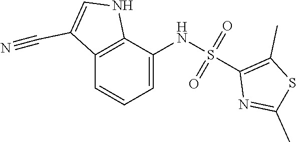 [SMILES: Cc1nc(S(=O)(=O)Nc2cccc3c(C#N)c[nH]c23)c(C)s1] |
| 89 | 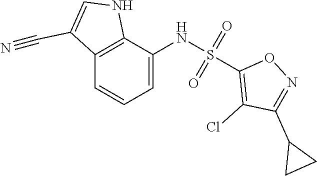 [SMILES: N#Cc1c[nH]c2c(NS(=O)(=O)c3onc(C4CC4)c3Cl)cccc12] |
| 90 | 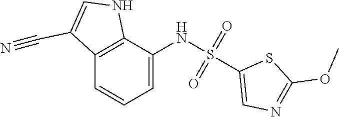 [SMILES: COc1ncc(S(=O)(=O)Nc2cccc3c(C#N)c[nH]c23)s1] |
| 91 | 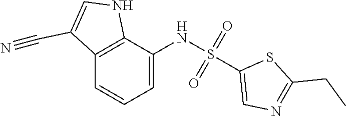 [SMILES: CCc1ncc(S(=O)(=O)Nc2cccc3c(C#N)c[nH]c23)s1] |
| 92 | 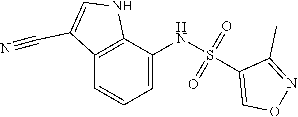 [SMILES: Cc1nocc1S(=O)(=O)Nc1cccc2c(C#N)c[nH]c12] |
| 93 | 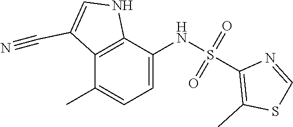 [SMILES: Cc1scnc1S(=O)(=O)Nc1ccc(C)c2c(C#N)c[nH]c12] |
| 94 | 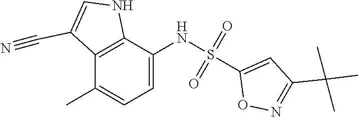 [SMILES: Cc1ccc(NS(=O)(=O)c2cc(C(C)(C)C)no2)c2[nH]cc(C#N)c12] |
| 95 | 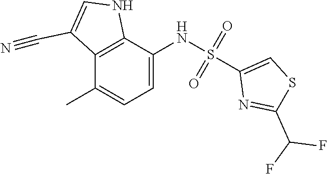 [SMILES: Cc1ccc(NS(=O)(=O)c2csc(C(F)F)n2)c2[nH]cc(C#N)c12] |
| 96 | 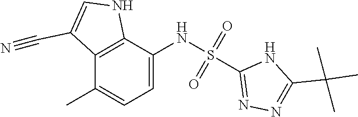 [SMILES: Cc1ccc(NS(=O)(=O)c2nnc(C(C)(C)C)[nH]2)c2[nH]cc(C#N)c12] |
| 97 | 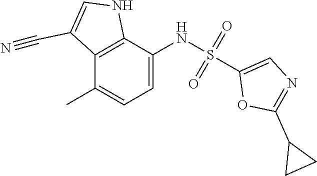 [SMILES: Cc1ccc(NS(=O)(=O)c2cnc(C3CC3)o2)c2[nH]cc(C#N)c12] |
| 98 | 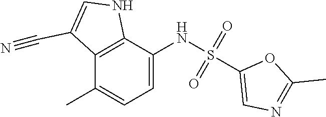 [SMILES: Cc1ncc(S(=O)(=O)Nc2ccc(C)c3c(C#N)c[nH]c23)o1] |
| 99 | 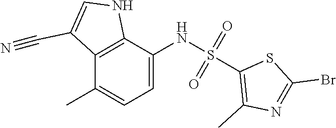 [SMILES: Cc1nc(Br)sc1S(=O)(=O)Nc1ccc(C)c2c(C#N)c[nH]c12] |
| 100 | 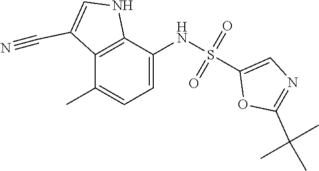 [SMILES: Cc1ccc(NS(=O)(=O)c2cnc(C(C)(C)C)o2)c2[nH]cc(C#N)c12] |
| 101 | 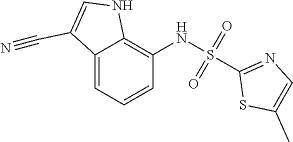 [SMILES: Cc1cnc(S(=O)(=O)Nc2cccc3c(C#N)c[nH]c23)s1] |
| 102 | 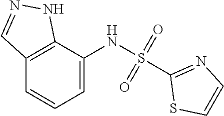 [SMILES: O=S(=O)(Nc1cccc2cn[nH]c12)c1nccs1] |
| 103 | 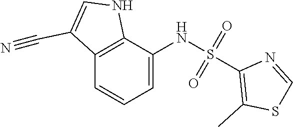 [SMILES: Cc1scnc1S(=O)(=O)Nc1cccc2c(C#N)c[nH]c12] |
| 104 | 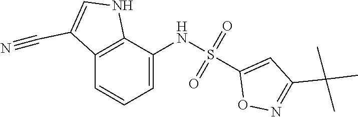 [SMILES: CC(C)(C)c1cc(S(=O)(=O)Nc2cccc3c(C#N)c[nH]c23)on1] |
| 105 | 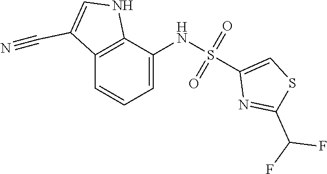 [SMILES: N#Cc1c[nH]c2c(NS(=O)(=O)c3csc(C(F)F)n3)cccc12] |
| 106 | 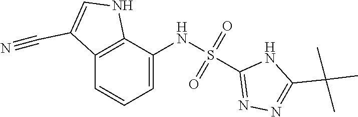 [SMILES: CC(C)(C)c1nnc(S(=O)(=O)Nc2cccc3c(C#N)c[nH]c23)[nH]1] |
| 107 | 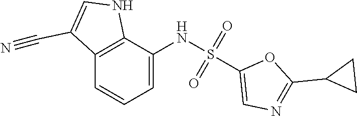 [SMILES: N#Cc1c[nH]c2c(NS(=O)(=O)c3cnc(C4CC4)o3)cccc12] |
| 108 | 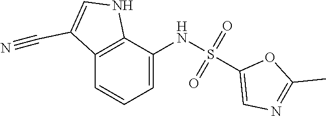 [SMILES: Cc1ncc(S(=O)(=O)Nc2cccc3c(C#N)c[nH]c23)o1] |
| 109 | 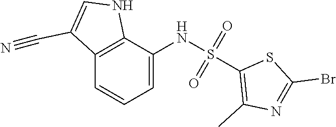 [SMILES: Cc1nc(Br)sc1S(=O)(=O)Nc1cccc2c(C#N)c[nH]c12] |
| 110 | 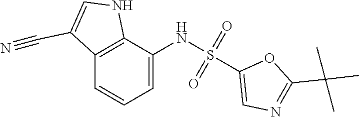 [SMILES: CC(C)(C)c1ncc(S(=O)(=O)Nc2cccc3c(C#N)c[nH]c23)o1] |
| 111 | 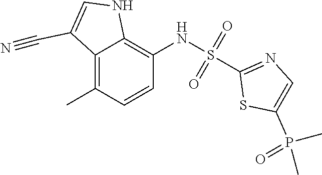 [SMILES: Cc1ccc(NS(=O)(=O)c2ncc(P(C)(C)=O)s2)c2[nH]cc(C#N)c12] |
| 112 | 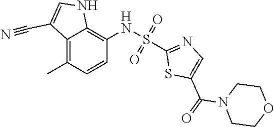 [SMILES: Cc1ccc(NS(=O)(=O)c2ncc(C(=O)N3CCOCC3)s2)c2[nH]cc(C#N)c12] |
| 113 | 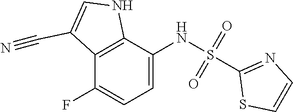 [SMILES: N#Cc1c[nH]c2c(NS(=O)(=O)c3nccs3)ccc(F)c12] |
| 114 | 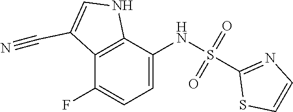 [SMILES: N#Cc1c[nH]c2c(NS(=O)(=O)c3nccs3)ccc(F)c12] |
| 115 | 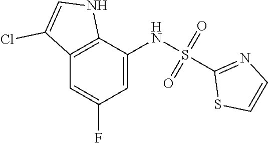 [SMILES: O=S(=O)(Nc1cc(F)cc2c(Cl)c[nH]c12)c1nccs1] |
| 116 | 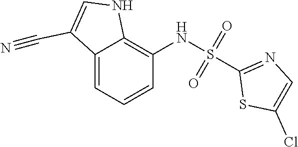 [SMILES: N#Cc1c[nH]c2c(NS(=O)(=O)c3ncc(Cl)s3)cccc12] |
| 117 | 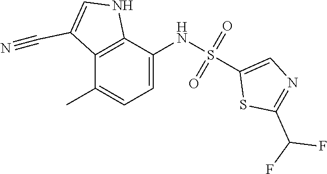 [SMILES: Cc1ccc(NS(=O)(=O)c2cnc(C(F)F)s2)c2[nH]cc(C#N)c12] |
| 118 | 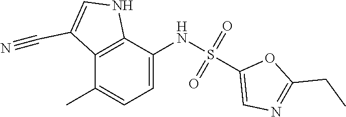 [SMILES: CCc1ncc(S(=O)(=O)Nc2ccc(C)c3c(C#N)c[nH]c23)o1] |
| 119 | 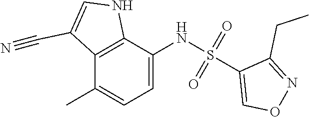 [SMILES: CCc1nocc1S(=O)(=O)Nc1ccc(C)c2c(C#N)c[nH]c12] |
| 120 | 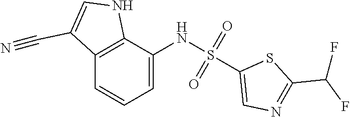 [SMILES: N#Cc1c[nH]c2c(NS(=O)(=O)c3cnc(C(F)F)s3)cccc12] |
| 121 | 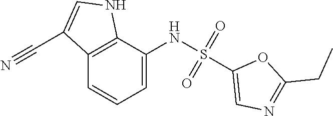 [SMILES: CCc1ncc(S(=O)(=O)Nc2cccc3c(C#N)c[nH]c23)o1] |
| 122 | 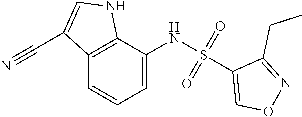 [SMILES: CCc1nocc1S(=O)(=O)Nc1cccc2c(C#N)c[nH]c12] |
| 123 | 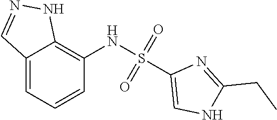 [SMILES: CCc1nc(S(=O)(=O)Nc2cccc3cn[nH]c23)c[nH]1] |
| 124 | 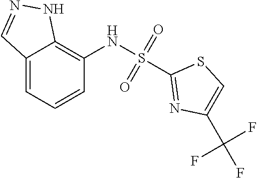 [SMILES: O=S(=O)(Nc1cccc2cn[nH]c12)c1nc(C(F)(F)F)cs1] |
| 125 | 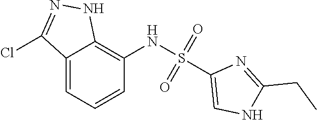 [SMILES: CCc1nc(S(=O)(=O)Nc2cccc3c(Cl)n[nH]c23)c[nH]1] |
| 126 | 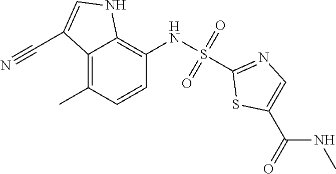 [SMILES: CNC(=O)c1cnc(S(=O)(=O)Nc2ccc(C)c3c(C#N)c[nH]c23)s1] |
| 127 | 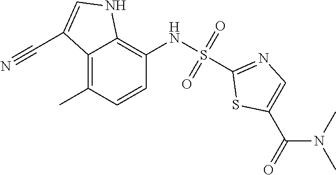 [SMILES: Cc1ccc(NS(=O)(=O)c2ncc(C(=O)N(C)C)s2)c2[nH]cc(C#N)c12] |
| 128 | 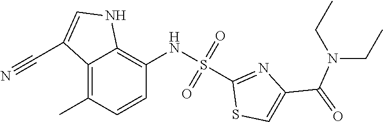 [SMILES: CCN(CC)C(=O)c1csc(S(=O)(=O)Nc2ccc(C)c3c(C#N)c[nH]c23)n1] |
| 129 | 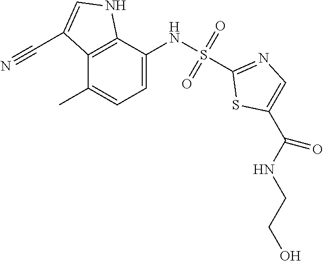 [SMILES: Cc1ccc(NS(=O)(=O)c2ncc(C(=O)NCCO)s2)c2[nH]cc(C#N)c12] |
| 130 | 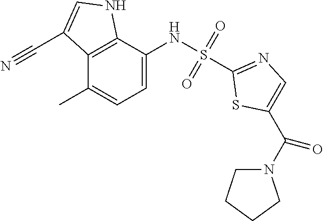 [SMILES: Cc1ccc(NS(=O)(=O)c2ncc(C(=O)N3CCCC3)s2)c2[nH]cc(C#N)c12] |
| 131 | 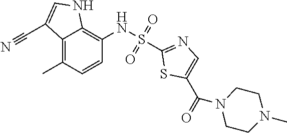 [SMILES: Cc1ccc(NS(=O)(=O)c2ncc(C(=O)N3CCN(C)CC3)s2)c2[nH]cc(C#N)c12] |
| 132 | 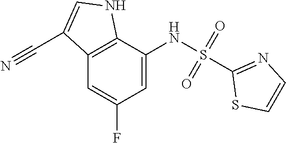 [SMILES: N#Cc1c[nH]c2c(NS(=O)(=O)c3nccs3)cc(F)cc12] |
| 133 | 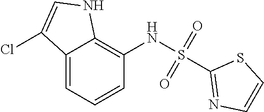 [SMILES: O=S(=O)(Nc1cccc2c(Cl)c[nH]c12)c1nccs1] |
| 134 | 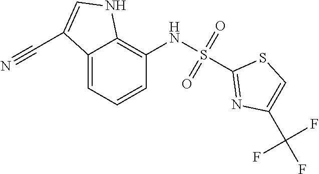 [SMILES: N#Cc1c[nH]c2c(NS(=O)(=O)c3nc(C(F)(F)F)cs3)cccc12] |
| 135 | 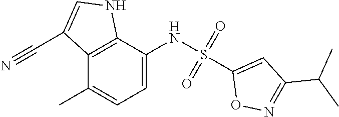 [SMILES: Cc1ccc(NS(=O)(=O)c2cc(C(C)C)no2)c2[nH]cc(C#N)c12] |
| 136 | 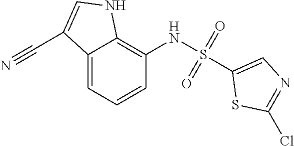 [SMILES: N#Cc1c[nH]c2c(NS(=O)(=O)c3cnc(Cl)s3)cccc12] |
| 137 | 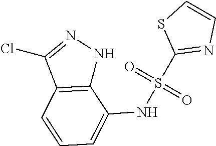 [SMILES: O=S(=O)(Nc1cccc2c(Cl)n[nH]c12)c1nccs1] |
| 138 | 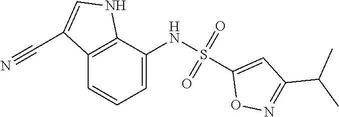 [SMILES: CC(C)c1cc(S(=O)(=O)Nc2cccc3c(C#N)c[nH]c23)on1] |
| 139 | 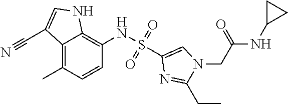 [SMILES: CCc1nc(S(=O)(=O)Nc2ccc(C)c3c(C#N)c[nH]c23)cn1CC(=O)NC1CC1] |
| 140 | 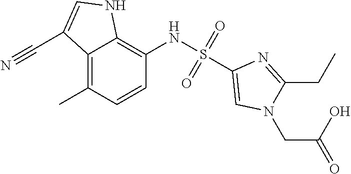 [SMILES: CCc1nc(S(=O)(=O)Nc2ccc(C)c3c(C#N)c[nH]c23)cn1CC(=O)O] |
| 141 | 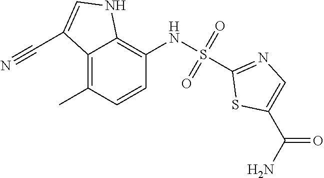 [SMILES: Cc1ccc(NS(=O)(=O)c2ncc(C(N)=O)s2)c2[nH]cc(C#N)c12] |
| 142 | 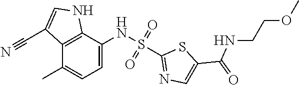 [SMILES: COCCNC(=O)c1cnc(S(=O)(=O)Nc2ccc(C)c3c(C#N)c[nH]c23)s1] |
| 143 | 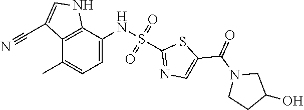 [SMILES: Cc1ccc(NS(=O)(=O)c2ncc(C(=O)N3CCC(O)C3)s2)c2[nH]cc(C#N)c12] |
| 144 | 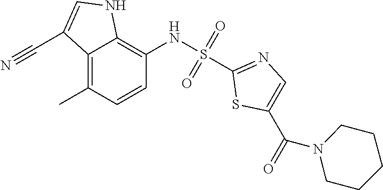 [SMILES: Cc1ccc(NS(=O)(=O)c2ncc(C(=O)N3CCCCC3)s2)c2[nH]cc(C#N)c12] |
| 145 | 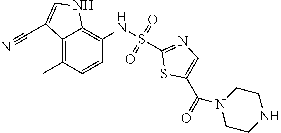 [SMILES: Cc1ccc(NS(=O)(=O)c2ncc(C(=O)N3CCNCC3)s2)c2[nH]cc(C#N)c12] |
| 146 | 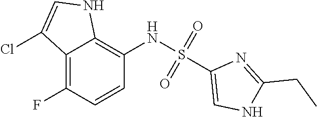 [SMILES: CCc1nc(S(=O)(=O)Nc2ccc(F)c3c(Cl)c[nH]c23)c[nH]1] |
| 147 | 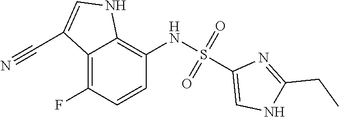 [SMILES: CCc1nc(S(=O)(=O)Nc2ccc(F)c3c(C#N)c[nH]c23)c[nH]1] |
| 148 | 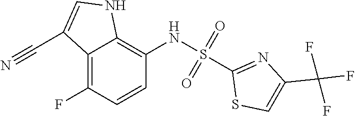 [SMILES: N#Cc1c[nH]c2c(NS(=O)(=O)c3nc(C(F)(F)F)cs3)ccc(F)c12] |
| 149 | 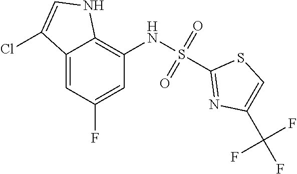 [SMILES: O=S(=O)(Nc1cc(F)cc2c(Cl)c[nH]c12)c1nc(C(F)(F)F)cs1] |
| 150 | 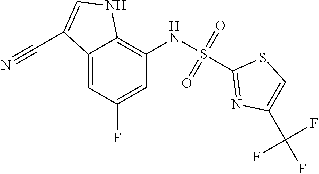 [SMILES: N#Cc1c[nH]c2c(NS(=O)(=O)c3nc(C(F)(F)F)cs3)cc(F)cc12] |
| 151 | 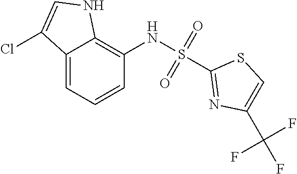 [SMILES: O=S(=O)(Nc1cccc2c(Cl)c[nH]c12)c1nc(C(F)(F)F)cs1] |
| 152 | 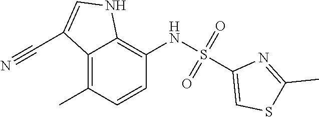 [SMILES: Cc1nc(S(=O)(=O)Nc2ccc(C)c3c(C#N)c[nH]c23)cs1] |
| 153 | 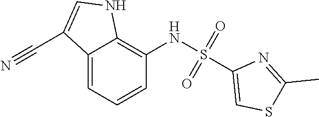 [SMILES: Cc1nc(S(=O)(=O)Nc2cccc3c(C#N)c[nH]c23)cs1] |
| 154 | 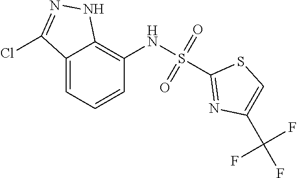 [SMILES: O=S(=O)(Nc1cccc2c(Cl)n[nH]c12)c1nc(C(F)(F)F)cs1] |
| 155 | 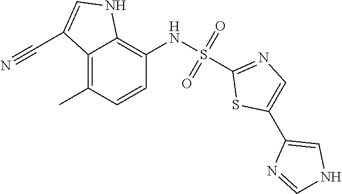 [SMILES: Cc1ccc(NS(=O)(=O)c2ncc(-c3c[nH]cn3)s2)c2[nH]cc(C#N)c12] |
| 156 | 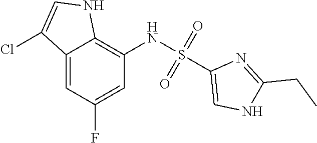 [SMILES: CCc1nc(S(=O)(=O)Nc2cc(F)cc3c(Cl)c[nH]c23)c[nH]1] |
| 157 | 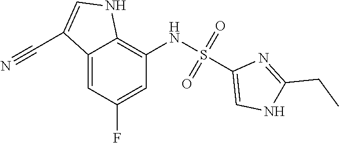 [SMILES: CCc1nc(S(=O)(=O)Nc2cc(F)cc3c(C#N)c[nH]c23)c[nH]1] |
| 158 | 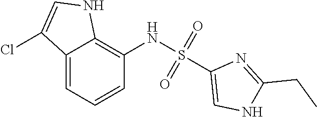 [SMILES: CCc1nc(S(=O)(=O)Nc2cccc3c(Cl)c[nH]c23)c[nH]1] |
| 159 | 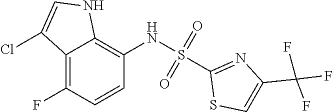 [SMILES: O=S(=O)(Nc1ccc(F)c2c(Cl)c[nH]c12)c1nc(C(F)(F)F)cs1] |
| 160 | 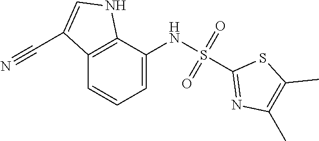 [SMILES: Cc1nc(S(=O)(=O)Nc2cccc3c(C#N)c[nH]c23)sc1C] |
| 161 | 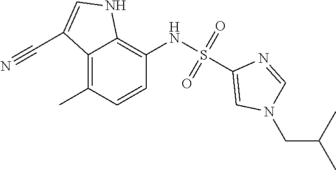 [SMILES: Cc1ccc(NS(=O)(=O)c2cn(CC(C)C)cn2)c2[nH]cc(C#N)c12] |
| 162 | 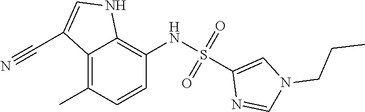 [SMILES: CCCn1cnc(S(=O)(=O)Nc2ccc(C)c3c(C#N)c[nH]c23)c1] |
| 163 | 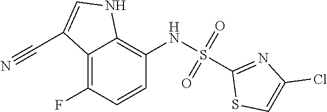 [SMILES: N#Cc1c[nH]c2c(NS(=O)(=O)c3nc(Cl)cs3)ccc(F)c12] |
| 164 | 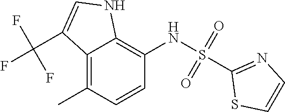 [SMILES: Cc1ccc(NS(=O)(=O)c2nccs2)c2[nH]cc(C(F)(F)F)c12] |
| 165 | 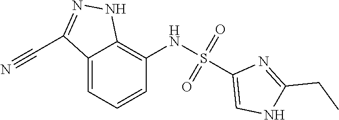 [SMILES: CCc1nc(S(=O)(=O)Nc2cccc3c(C#N)n[nH]c23)c[nH]1] |
| 166 | 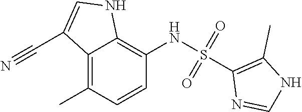 [SMILES: Cc1[nH]cnc1S(=O)(=O)Nc1ccc(C)c2c(C#N)c[nH]c12] |
| 167 | 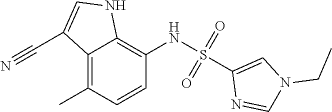 [SMILES: CCn1cnc(S(=O)(=O)Nc2ccc(C)c3c(C#N)c[nH]c23)c1] |
| 168 | 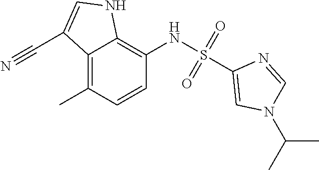 [SMILES: Cc1ccc(NS(=O)(=O)c2cn(C(C)C)cn2)c2[nH]cc(C#N)c12] |
| 169 | 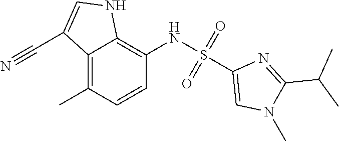 [SMILES: Cc1ccc(NS(=O)(=O)c2cn(C)c(C(C)C)n2)c2[nH]cc(C#N)c12] |
| 170 | 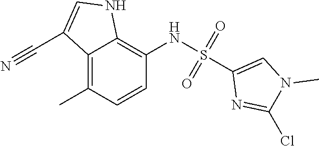 [SMILES: Cc1ccc(NS(=O)(=O)c2cn(C)c(Cl)n2)c2[nH]cc(C#N)c12] |
| 171 | 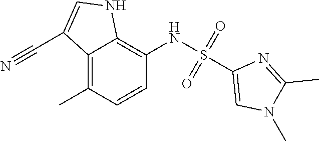 [SMILES: Cc1ccc(NS(=O)(=O)c2cn(C)c(C)n2)c2[nH]cc(C#N)c12] |
| 172 | 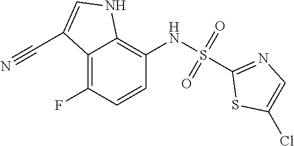 [SMILES: N#Cc1c[nH]c2c(NS(=O)(=O)c3ncc(Cl)s3)ccc(F)c12] |
| 173 | 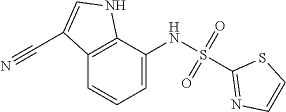 [SMILES: N#Cc1c[nH]c2c(NS(=O)(=O)c3nccs3)cccc12] |
| 174 | 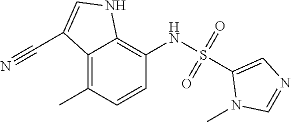 [SMILES: Cc1ccc(NS(=O)(=O)c2cncn2C)c2[nH]cc(C#N)c12] |
| 175 | 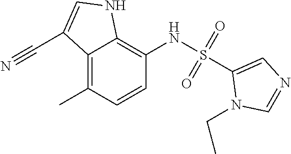 [SMILES: CCn1cncc1S(=O)(=O)Nc1ccc(C)c2c(C#N)c[nH]c12] |
| 176 | 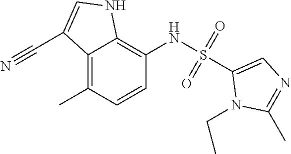 [SMILES: CCn1c(S(=O)(=O)Nc2ccc(C)c3c(C#N)c[nH]c23)cnc1C] |
| 177 | 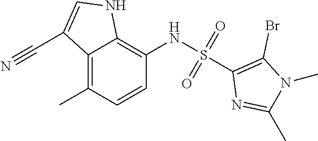 [SMILES: Cc1ccc(NS(=O)(=O)c2nc(C)n(C)c2Br)c2[nH]cc(C#N)c12] |
| 178 | 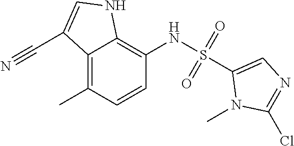 [SMILES: Cc1ccc(NS(=O)(=O)c2cnc(Cl)n2C)c2[nH]cc(C#N)c12] |
| 179 | 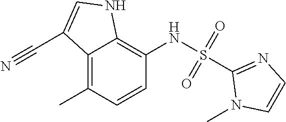 [SMILES: Cc1ccc(NS(=O)(=O)c2nccn2C)c2[nH]cc(C#N)c12] |
| 180 | 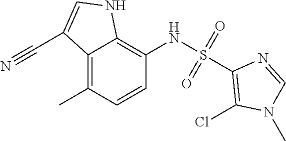 [SMILES: Cc1ccc(NS(=O)(=O)c2ncn(C)c2Cl)c2[nH]cc(C#N)c12] |
| 181 | 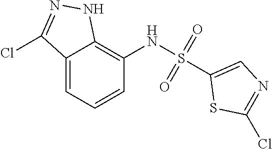 [SMILES: O=S(=O)(Nc1cccc2c(Cl)n[nH]c12)c1cnc(Cl)s1] |
| 182 | 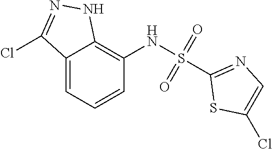 [SMILES: O=S(=O)(Nc1cccc2c(Cl)n[nH]c12)c1ncc(Cl)s1] |
| 183 | 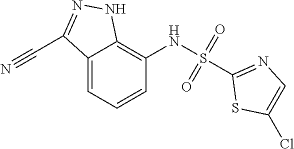 [SMILES: N#Cc1n[nH]c2c(NS(=O)(=O)c3ncc(Cl)s3)cccc12] |
| 184 | 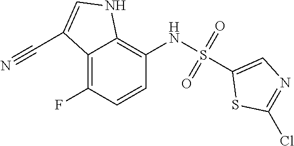 [SMILES: N#Cc1c[nH]c2c(NS(=O)(=O)c3cnc(Cl)s3)ccc(F)c12] |
| 185 | 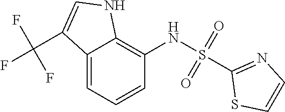 [SMILES: O=S(=O)(Nc1cccc2c(C(F)(F)F)c[nH]c12)c1nccs1] |
| 186 | 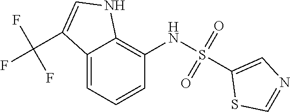 [SMILES: O=S(=O)(Nc1cccc2c(C(F)(F)F)c[nH]c12)c1cncs1] |
| 187 | 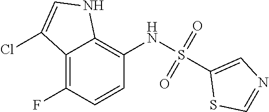 [SMILES: O=S(=O)(Nc1ccc(F)c2c(Cl)c[nH]c12)c1cncs1] |
| 188 | 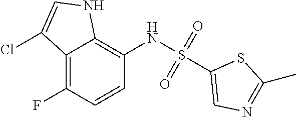 [SMILES: Cc1ncc(S(=O)(=O)Nc2ccc(F)c3c(Cl)c[nH]c23)s1] |
| 189 | 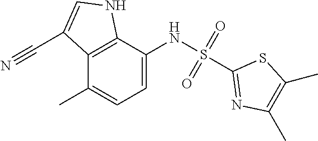 [SMILES: Cc1nc(S(=O)(=O)Nc2ccc(C)c3c(C#N)c[nH]c23)sc1C] |
| 190 | 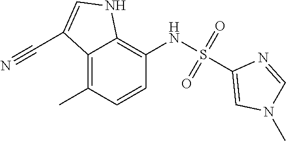 [SMILES: Cc1ccc(NS(=O)(=O)c2cn(C)cn2)c2[nH]cc(C#N)c12] |
| 191 | 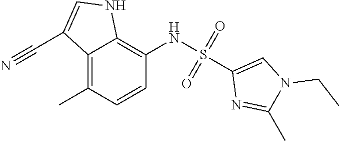 [SMILES: CCn1cc(S(=O)(=O)Nc2ccc(C)c3c(C#N)c[nH]c23)nc1C] |
| 192 | 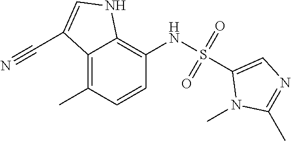 [SMILES: Cc1ccc(NS(=O)(=O)c2cnc(C)n2C)c2[nH]cc(C#N)c12] |
| 193 | 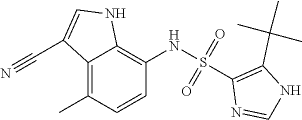 [SMILES: Cc1ccc(NS(=O)(=O)c2nc[nH]c2C(C)(C)C)c2[nH]cc(C#N)c12] |
| 194 | 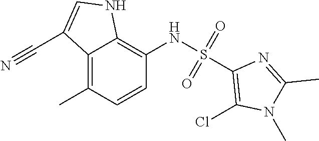 [SMILES: Cc1ccc(NS(=O)(=O)c2nc(C)n(C)c2Cl)c2[nH]cc(C#N)c12] |
| 195 | 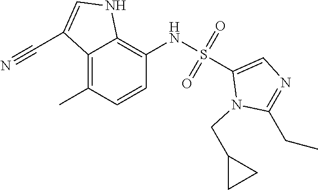 [SMILES: CCc1ncc(S(=O)(=O)Nc2ccc(C)c3c(C#N)c[nH]c23)n1CC1CC1] |
| 196 | 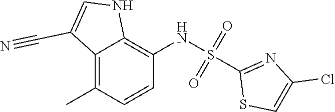 [SMILES: Cc1ccc(NS(=O)(=O)c2nc(Cl)cs2)c2[nH]cc(C#N)c12] |
| 197 | 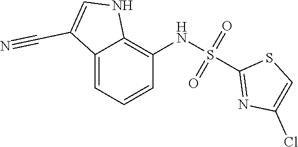 [SMILES: N#Cc1c[nH]c2c(NS(=O)(=O)c3nc(Cl)cs3)cccc12] |
| 198 | 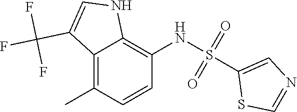 [SMILES: Cc1ccc(NS(=O)(=O)c2cncs2)c2[nH]cc(C(F)(F)F)c12] |
| 199 | 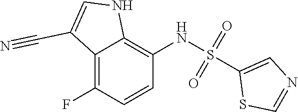 [SMILES: N#Cc1c[nH]c2c(NS(=O)(=O)c3cncs3)ccc(F)c12] |
| 200 | 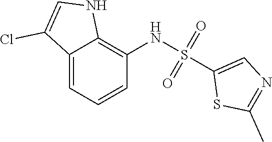 [SMILES: Cc1ncc(S(=O)(=O)Nc2cccc3c(Cl)c[nH]c23)s1] |
| 201 | 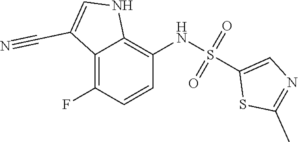 [SMILES: Cc1ncc(S(=O)(=O)Nc2ccc(F)c3c(C#N)c[nH]c23)s1] |
| 202 | 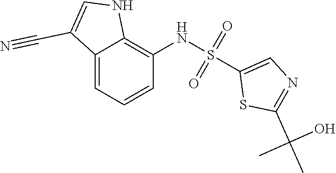 [SMILES: CC(C)(O)c1ncc(S(=O)(=O)Nc2cccc3c(C#N)c[nH]c23)s1] |
| 203 | 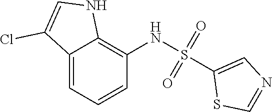 [SMILES: O=S(=O)(Nc1cccc2c(Cl)c[nH]c12)c1cncs1] |
| 204 | 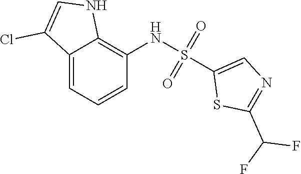 [SMILES: O=S(=O)(Nc1cccc2c(Cl)c[nH]c12)c1cnc(C(F)F)s1] |
| 205 | 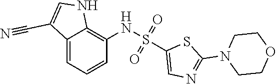 [SMILES: N#Cc1c[nH]c2c(NS(=O)(=O)c3cnc(N4CCOCC4)s3)cccc12] |
| 206 | 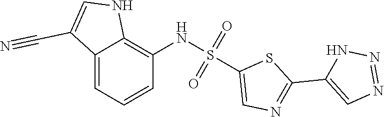 [SMILES: N#Cc1c[nH]c2c(NS(=O)(=O)c3cnc(-c4cnn[nH]4)s3)cccc12] |
| 207 | 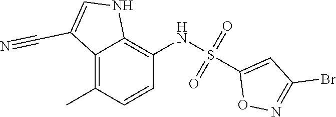 [SMILES: Cc1ccc(NS(=O)(=O)c2cc(Br)no2)c2[nH]cc(C#N)c12] |
| 208 | 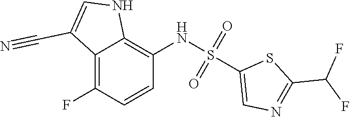 [SMILES: N#Cc1c[nH]c2c(NS(=O)(=O)c3cnc(C(F)F)s3)ccc(F)c12] |
| 209 | 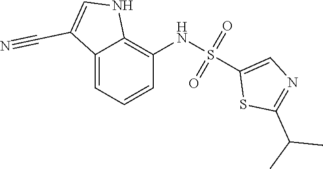 [SMILES: CC(C)c1ncc(S(=O)(=O)Nc2cccc3c(C#N)c[nH]c23)s1] |
| 210 | 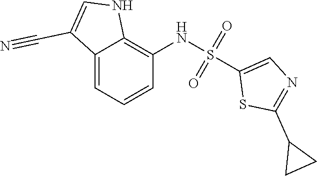 [SMILES: N#Cc1c[nH]c2c(NS(=O)(=O)c3cnc(C4CC4)s3)cccc12] |
| 211 | 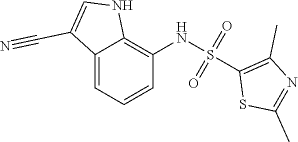 [SMILES: Cc1nc(C)c(S(=O)(=O)Nc2cccc3c(C#N)c[nH]c23)s1] |
| 212 | 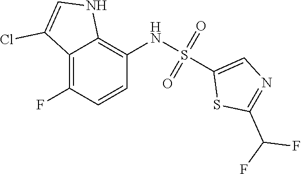 [SMILES: O=S(=O)(Nc1ccc(F)c2c(Cl)c[nH]c12)c1cnc(C(F)F)s1] |
| 213 | 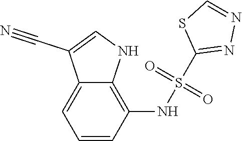 [SMILES: N#Cc1c[nH]c2c(NS(=O)(=O)c3nncs3)cccc12] |
| 214 | 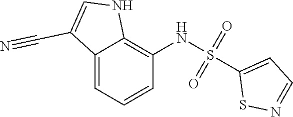 [SMILES: N#Cc1c[nH]c2c(NS(=O)(=O)c3ccns3)cccc12] |
| 215 | 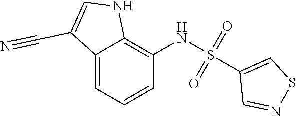 [SMILES: N#Cc1c[nH]c2c(NS(=O)(=O)c3cnsc3)cccc12] |
| 216 | 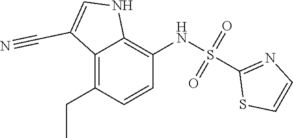 [SMILES: CCc1ccc(NS(=O)(=O)c2nccs2)c2[nH]cc(C#N)c12] |
| 217 | 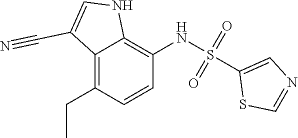 [SMILES: CCc1ccc(NS(=O)(=O)c2cncs2)c2[nH]cc(C#N)c12] |
| 218 | 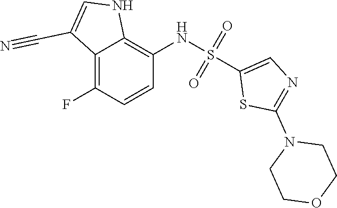 [SMILES: N#Cc1c[nH]c2c(NS(=O)(=O)c3cnc(N4CCOCC4)s3)ccc(F)c12] |
| 219 | 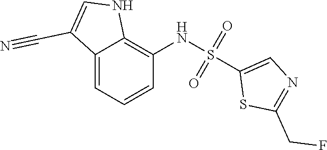 [SMILES: N#Cc1c[nH]c2c(NS(=O)(=O)c3cnc(CF)s3)cccc12] |
| 220 | 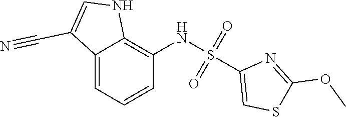 [SMILES: COc1nc(S(=O)(=O)Nc2cccc3c(C#N)c[nH]c23)cs1] |
| 221 | 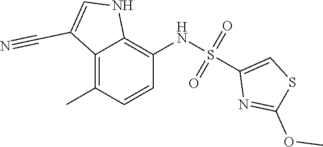 [SMILES: COc1nc(S(=O)(=O)Nc2ccc(C)c3c(C#N)c[nH]c23)cs1] |
| 222 | 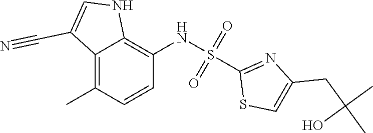 [SMILES: Cc1ccc(NS(=O)(=O)c2nc(CC(C)(C)O)cs2)c2[nH]cc(C#N)c12] |
| 223 | 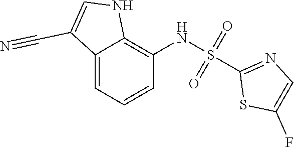 [SMILES: N#Cc1c[nH]c2c(NS(=O)(=O)c3ncc(F)s3)cccc12] |
| 224 | 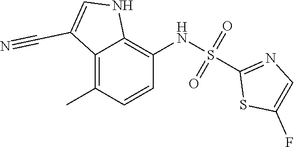 [SMILES: Cc1ccc(NS(=O)(=O)c2ncc(F)s2)c2[nH]cc(C#N)c12] |
| 225 | 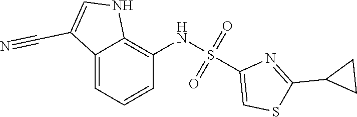 [SMILES: N#Cc1c[nH]c2c(NS(=O)(=O)c3csc(C4CC4)n3)cccc12] |
| 226 | 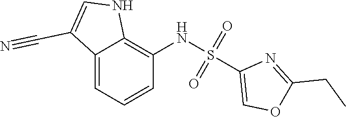 [SMILES: CCc1nc(S(=O)(=O)Nc2cccc3c(C#N)c[nH]c23)co1] |
| 227 | 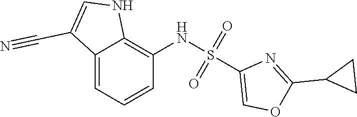 [SMILES: N#Cc1c[nH]c2c(NS(=O)(=O)c3coc(C4CC4)n3)cccc12] |
| 228 | 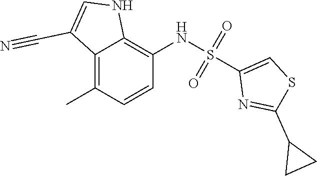 [SMILES: Cc1ccc(NS(=O)(=O)c2csc(C3CC3)n2)c2[nH]cc(C#N)c12] |
| 229 | 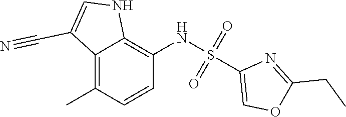 [SMILES: CCc1nc(S(=O)(=O)Nc2ccc(C)c3c(C#N)c[nH]c23)co1] |
| 230 | 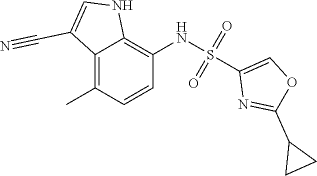 [SMILES: Cc1ccc(NS(=O)(=O)c2coc(C3CC3)n2)c2[nH]cc(C#N)c12] |
| 231 | 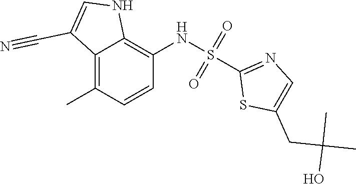 [SMILES: Cc1ccc(NS(=O)(=O)c2ncc(CC(C)(C)O)s2)c2[nH]cc(C#N)c12] |
| 232 | 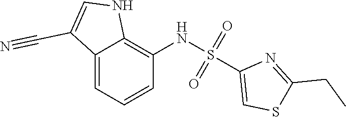 [SMILES: CCc1nc(S(=O)(=O)Nc2cccc3c(C#N)c[nH]c23)cs1] |
| 233 | 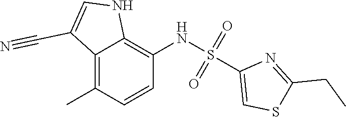 [SMILES: CCc1nc(S(=O)(=O)Nc2ccc(C)c3c(C#N)c[nH]c23)cs1] |
| 234 | 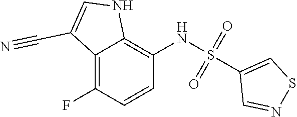 [SMILES: N#Cc1c[nH]c2c(NS(=O)(=O)c3cnsc3)ccc(F)c12] |
| 235 | 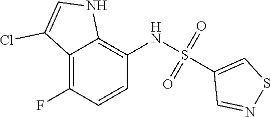 [SMILES: O=S(=O)(Nc1ccc(F)c2c(Cl)c[nH]c12)c1cnsc1] |
| 236 | 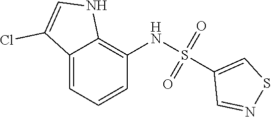 [SMILES: O=S(=O)(Nc1cccc2c(Cl)c[nH]c12)c1cnsc1] |
| 237 | 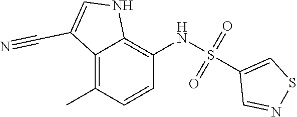 [SMILES: Cc1ccc(NS(=O)(=O)c2cnsc2)c2[nH]cc(C#N)c12] |
| 238 | 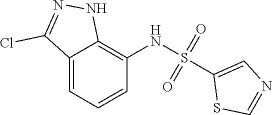 [SMILES: O=S(=O)(Nc1cccc2c(Cl)n[nH]c12)c1cncs1] |
| 239 | 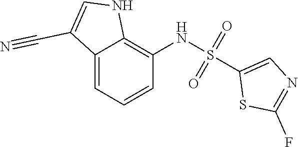 [SMILES: N#Cc1c[nH]c2c(NS(=O)(=O)c3cnc(F)s3)cccc12] |
| 240 | 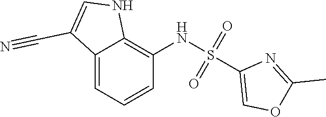 [SMILES: Cc1nc(S(=O)(=O)Nc2cccc3c(C#N)c[nH]c23)co1] |
| 241 | 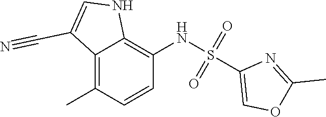 [SMILES: Cc1nc(S(=O)(=O)Nc2ccc(C)c3c(C#N)c[nH]c23)co1] |
| 242 | 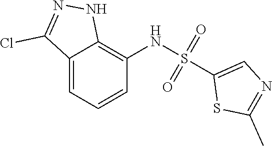 [SMILES: Cc1ncc(S(=O)(=O)Nc2cccc3c(Cl)n[nH]c23)s1] |
| 243 | 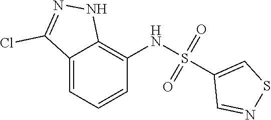 [SMILES: O=S(=O)(Nc1cccc2c(Cl)n[nH]c12)c1cnsc1] |
| 244 | 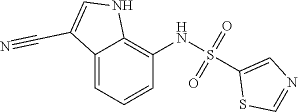 [SMILES: N#Cc1c[nH]c2c(NS(=O)(=O)c3cncs3)cccc12] |
| 245 | 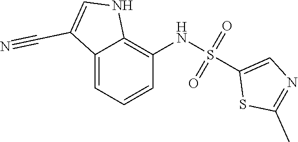 [SMILES: Cc1ncc(S(=O)(=O)Nc2cccc3c(C#N)c[nH]c23)s1] |
| 246 | 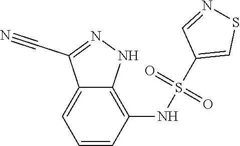 [SMILES: N#Cc1n[nH]c2c(NS(=O)(=O)c3cnsc3)cccc12] |
| 247 | 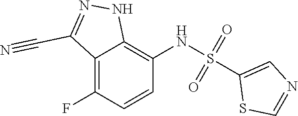 [SMILES: N#Cc1n[nH]c2c(NS(=O)(=O)c3cncs3)ccc(F)c12] |
| 248 | 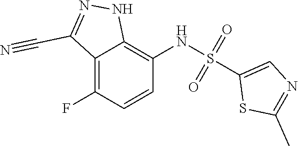 [SMILES: Cc1ncc(S(=O)(=O)Nc2ccc(F)c3c(C#N)n[nH]c23)s1] |
| 249 | 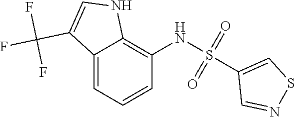 [SMILES: O=S(=O)(Nc1cccc2c(C(F)(F)F)c[nH]c12)c1cnsc1] |
| 250 | 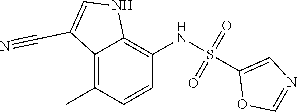 [SMILES: Cc1ccc(NS(=O)(=O)c2cnco2)c2[nH]cc(C#N)c12] |
| 251 | 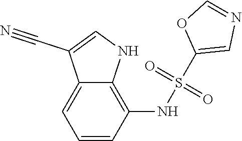 [SMILES: N#Cc1c[nH]c2c(NS(=O)(=O)c3cnco3)cccc12] |
| 252 | 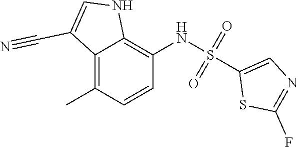 [SMILES: Cc1ccc(NS(=O)(=O)c2cnc(F)s2)c2[nH]cc(C#N)c12] |
| 253 | 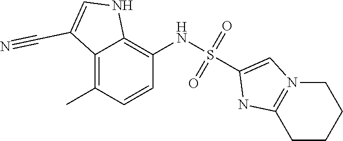 |
| 254 | 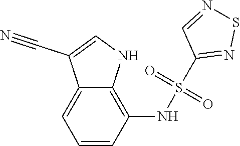 [SMILES: N#Cc1c[nH]c2c(NS(=O)(=O)c3cnsn3)cccc12] |
| 255 | 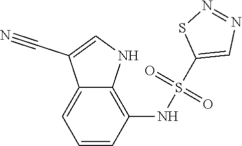 [SMILES: N#Cc1c[nH]c2c(NS(=O)(=O)c3cnns3)cccc12] |
| 256 | 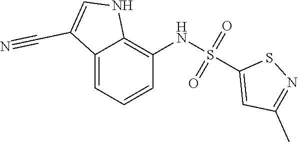 [SMILES: Cc1cc(S(=O)(=O)Nc2cccc3c(C#N)c[nH]c23)sn1] |
| 257 | 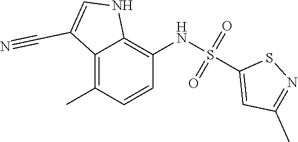 [SMILES: Cc1cc(S(=O)(=O)Nc2ccc(C)c3c(C#N)c[nH]c23)sn1] |
| 258 | 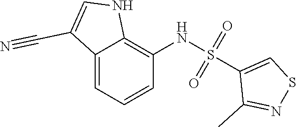 [SMILES: Cc1nscc1S(=O)(=O)Nc1cccc2c(C#N)c[nH]c12] |
| 259 | 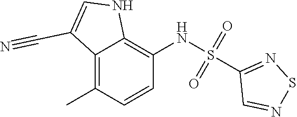 [SMILES: Cc1ccc(NS(=O)(=O)c2cnsn2)c2[nH]cc(C#N)c12] |
| 260 | 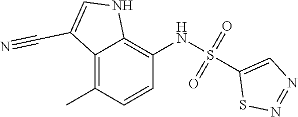 [SMILES: Cc1ccc(NS(=O)(=O)c2cnns2)c2[nH]cc(C#N)c12] |
| 261 | 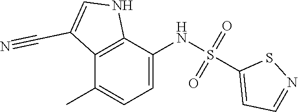 [SMILES: Cc1ccc(NS(=O)(=O)c2ccns2)c2[nH]cc(C#N)c12] |
| 262 | 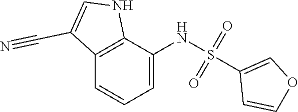 [SMILES: N#Cc1c[nH]c2c(NS(=O)(=O)c3ccoc3)cccc12] |
| 263 | 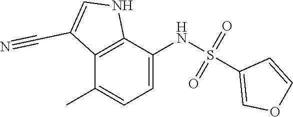 [SMILES: Cc1ccc(NS(=O)(=O)c2ccoc2)c2[nH]cc(C#N)c12] |
| 264 | 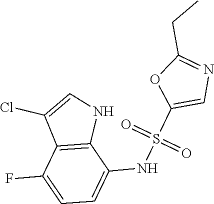 [SMILES: CCc1ncc(S(=O)(=O)Nc2ccc(F)c3c(Cl)c[nH]c23)o1] |
| 265 | 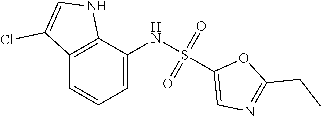 [SMILES: CCc1ncc(S(=O)(=O)Nc2cccc3c(Cl)c[nH]c23)o1] |
| 266 | 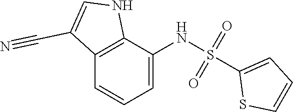 [SMILES: N#Cc1c[nH]c2c(NS(=O)(=O)c3cccs3)cccc12] |
| 267 | 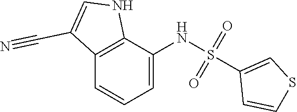 [SMILES: N#Cc1c[nH]c2c(NS(=O)(=O)c3ccsc3)cccc12] |
| 268 | 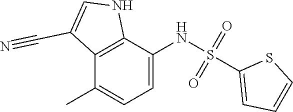 [SMILES: Cc1ccc(NS(=O)(=O)c2cccs2)c2[nH]cc(C#N)c12] |
| 269 | 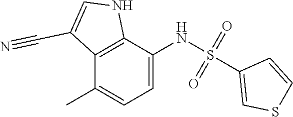 [SMILES: Cc1ccc(NS(=O)(=O)c2ccsc2)c2[nH]cc(C#N)c12] |
| 270 | 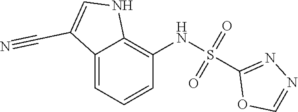 [SMILES: N#Cc1c[nH]c2c(NS(=O)(=O)c3nnco3)cccc12] |
| 271 | 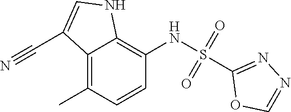 [SMILES: Cc1ccc(NS(=O)(=O)c2nnco2)c2[nH]cc(C#N)c12] |
| 272 | 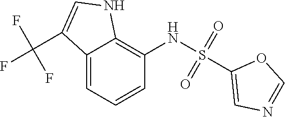 [SMILES: O=S(=O)(Nc1cccc2c(C(F)(F)F)c[nH]c12)c1cnco1] |
| 273 | 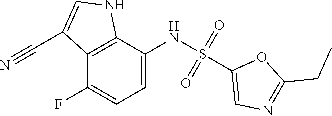 [SMILES: CCc1ncc(S(=O)(=O)Nc2ccc(F)c3c(C#N)c[nH]c23)o1] |
| 274 | 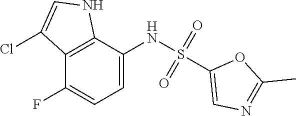 [SMILES: Cc1ncc(S(=O)(=O)Nc2ccc(F)c3c(Cl)c[nH]c23)o1] |
| 275 | 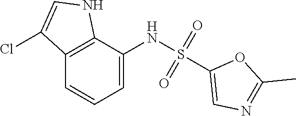 [SMILES: Cc1ncc(S(=O)(=O)Nc2cccc3c(Cl)c[nH]c23)o1] |
| 276 | 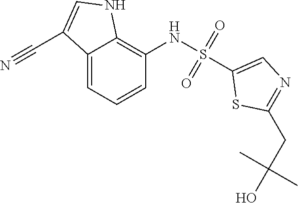 [SMILES: CC(C)(O)Cc1ncc(S(=O)(=O)Nc2cccc3c(C#N)c[nH]c23)s1] |
| 277 | 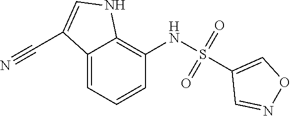 [SMILES: N#Cc1c[nH]c2c(NS(=O)(=O)c3cnoc3)cccc12] |
| 278 | 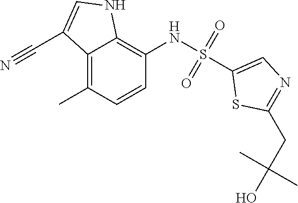 [SMILES: Cc1ccc(NS(=O)(=O)c2cnc(CC(C)(C)O)s2)c2[nH]cc(C#N)c12] |
| 279 | 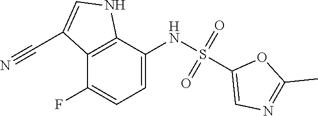 [SMILES: Cc1ncc(S(=O)(=O)Nc2ccc(F)c3c(C#N)c[nH]c23)o1] |
| 280 | 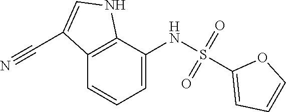 [SMILES: N#Cc1c[nH]c2c(NS(=O)(=O)c3ccco3)cccc12] |
| 281 | 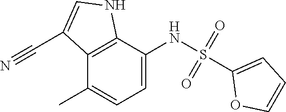 [SMILES: Cc1ccc(NS(=O)(=O)c2ccco2)c2[nH]cc(C#N)c12] |
| 282 | 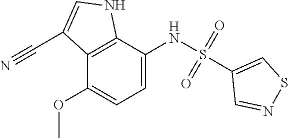 [SMILES: COc1ccc(NS(=O)(=O)c2cnsc2)c2[nH]cc(C#N)c12] |
| 283 | 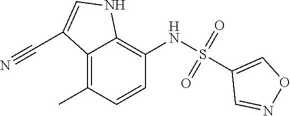 [SMILES: Cc1ccc(NS(=O)(=O)c2cnoc2)c2[nH]cc(C#N)c12] |
| 284 | 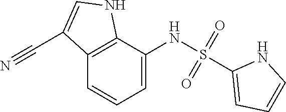 [SMILES: N#Cc1c[nH]c2c(NS(=O)(=O)c3ccc[nH]3)cccc12] |
| 285 | 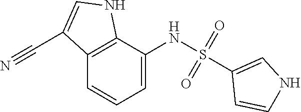 [SMILES: N#Cc1c[nH]c2c(NS(=O)(=O)c3cc[nH]c3)cccc12] |
| 286 | 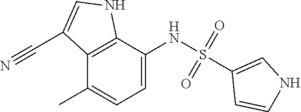 [SMILES: Cc1ccc(NS(=O)(=O)c2cc[nH]c2)c2[nH]cc(C#N)c12] |
| 287 | 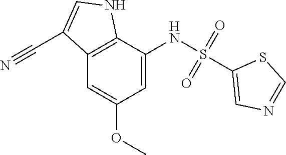 [SMILES: COc1cc(NS(=O)(=O)c2cncs2)c2[nH]cc(C#N)c2c1] |
| 288 | 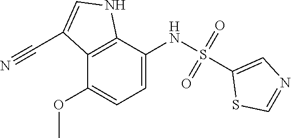 [SMILES: COc1ccc(NS(=O)(=O)c2cncs2)c2[nH]cc(C#N)c12] |
| 289 | 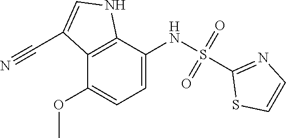 [SMILES: COc1ccc(NS(=O)(=O)c2nccs2)c2[nH]cc(C#N)c12] |
| 290 | 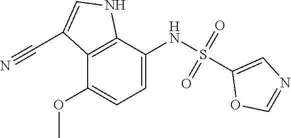 [SMILES: COc1ccc(NS(=O)(=O)c2cnco2)c2[nH]cc(C#N)c12] |
| 291 | 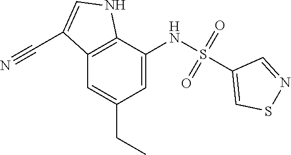 [SMILES: CCc1cc(NS(=O)(=O)c2cnsc2)c2[nH]cc(C#N)c2c1] |
| 292 | 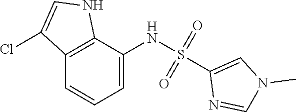 [SMILES: Cn1cnc(S(=O)(=O)Nc2cccc3c(Cl)c[nH]c23)c1] |
| 293 | 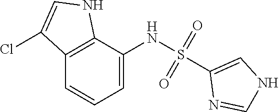 [SMILES: O=S(=O)(Nc1cccc2c(Cl)c[nH]c12)c1c[nH]cn1] |
| 294 | 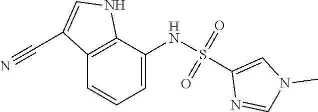 [SMILES: Cn1cnc(S(=O)(=O)Nc2cccc3c(C#N)c[nH]c23)c1] |
| 295 | 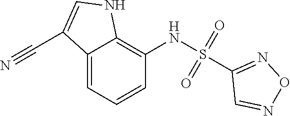 [SMILES: N#Cc1c[nH]c2c(NS(=O)(=O)c3cnon3)cccc12] |
| 296 | 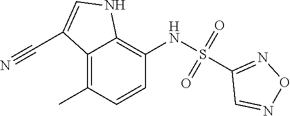 [SMILES: Cc1ccc(NS(=O)(=O)c2cnon2)c2[nH]cc(C#N)c12] |
| 297 | 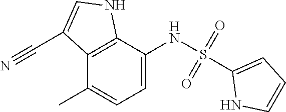 [SMILES: Cc1ccc(NS(=O)(=O)c2ccc[nH]2)c2[nH]cc(C#N)c12] |
| 298 | 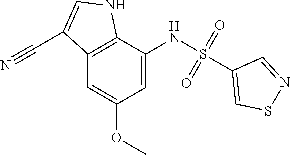 [SMILES: COc1cc(NS(=O)(=O)c2cnsc2)c2[nH]cc(C#N)c2c1] |
| 299 | 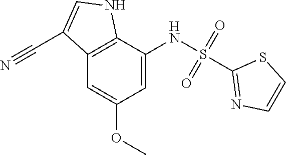 [SMILES: COc1cc(NS(=O)(=O)c2nccs2)c2[nH]cc(C#N)c2c1] |
| 300 | 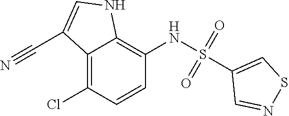 [SMILES: N#Cc1c[nH]c2c(NS(=O)(=O)c3cnsc3)ccc(Cl)c12] |
| 301 | 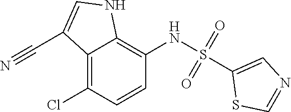 [SMILES: N#Cc1c[nH]c2c(NS(=O)(=O)c3cncs3)ccc(Cl)c12] |
| 302 | 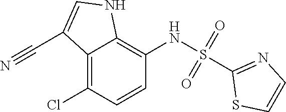 [SMILES: N#Cc1c[nH]c2c(NS(=O)(=O)c3nccs3)ccc(Cl)c12] |
| 303 | 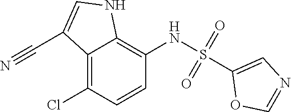 [SMILES: N#Cc1c[nH]c2c(NS(=O)(=O)c3cnco3)ccc(Cl)c12] |
| 304 | 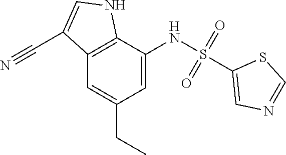 [SMILES: CCc1cc(NS(=O)(=O)c2cncs2)c2[nH]cc(C#N)c2c1] |
| 305 | 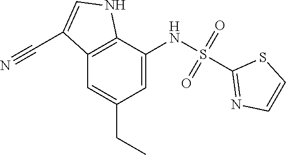 [SMILES: CCc1cc(NS(=O)(=O)c2nccs2)c2[nH]cc(C#N)c2c1] |
| 306 | 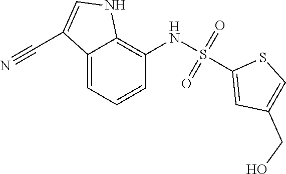 [SMILES: N#Cc1c[nH]c2c(NS(=O)(=O)c3cc(CO)cs3)cccc12] |
| 307 | 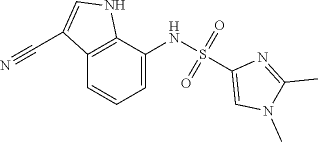 [SMILES: Cc1nc(S(=O)(=O)Nc2cccc3c(C#N)c[nH]c23)cn1C] |
| 308 | 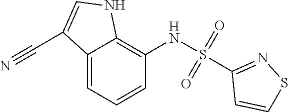 [SMILES: N#Cc1c[nH]c2c(NS(=O)(=O)c3ccsn3)cccc12] |
| 309 | 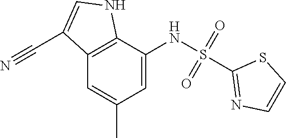 [SMILES: Cc1cc(NS(=O)(=O)c2nccs2)c2[nH]cc(C#N)c2c1] |
| 310 | 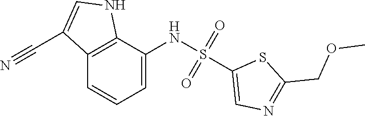 [SMILES: COCc1ncc(S(=O)(=O)Nc2cccc3c(C#N)c[nH]c23)s1] |
| 311 | 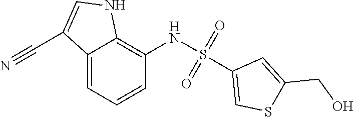 [SMILES: N#Cc1c[nH]c2c(NS(=O)(=O)c3csc(CO)c3)cccc12] |
| 312 | 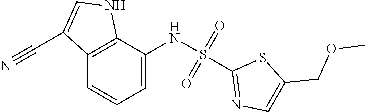 [SMILES: COCc1cnc(S(=O)(=O)Nc2cccc3c(C#N)c[nH]c23)s1] |
| 313 | 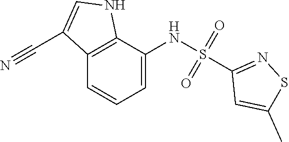 [SMILES: Cc1cc(S(=O)(=O)Nc2cccc3c(C#N)c[nH]c23)ns1] |
| 314 | 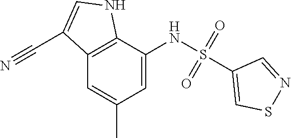 [SMILES: Cc1cc(NS(=O)(=O)c2cnsc2)c2[nH]cc(C#N)c2c1] |
| 315 | 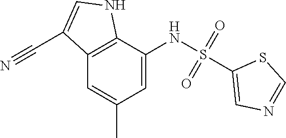 [SMILES: Cc1cc(NS(=O)(=O)c2cncs2)c2[nH]cc(C#N)c2c1] |
| 316 | 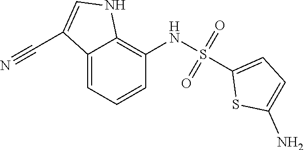 [SMILES: N#Cc1c[nH]c2c(NS(=O)(=O)c3ccc(N)s3)cccc12] |
| 317 | 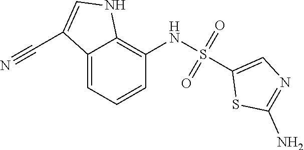 [SMILES: N#Cc1c[nH]c2c(NS(=O)(=O)c3cnc(N)s3)cccc12] |
| 318 | 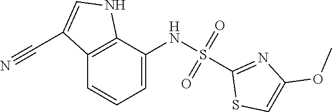 [SMILES: COc1csc(S(=O)(=O)Nc2cccc3c(C#N)c[nH]c23)n1] |
| 319 | 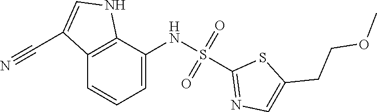 [SMILES: COCCc1cnc(S(=O)(=O)Nc2cccc3c(C#N)c[nH]c23)s1] |
| 320 | 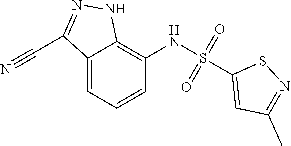 [SMILES: Cc1cc(S(=O)(=O)Nc2cccc3c(C#N)n[nH]c23)sn1] |
| 321 | 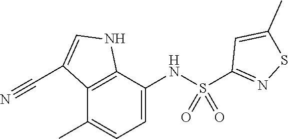 [SMILES: Cc1cc(S(=O)(=O)Nc2ccc(C)c3c(C#N)c[nH]c23)ns1] |
| 322 | 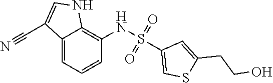 [SMILES: N#Cc1c[nH]c2c(NS(=O)(=O)c3csc(CCO)c3)cccc12] |
| 323 | 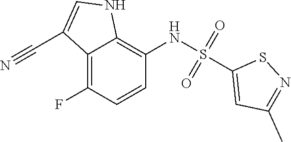 [SMILES: Cc1cc(S(=O)(=O)Nc2ccc(F)c3c(C#N)c[nH]c23)sn1] |
| 324 | 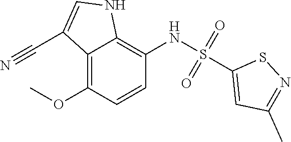 [SMILES: COc1ccc(NS(=O)(=O)c2cc(C)ns2)c2[nH]cc(C#N)c12] |
| 325 | 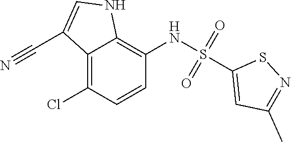 [SMILES: Cc1cc(S(=O)(=O)Nc2ccc(Cl)c3c(C#N)c[nH]c23)sn1] |
| 326 | 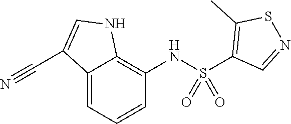 [SMILES: Cc1sncc1S(=O)(=O)Nc1cccc2c(C#N)c[nH]c12] |
| 327 | 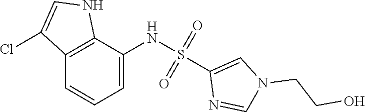 [SMILES: O=S(=O)(Nc1cccc2c(Cl)c[nH]c12)c1cn(CCO)cn1] |
| 328 | 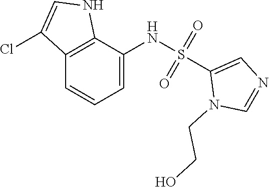 [SMILES: O=S(=O)(Nc1cccc2c(Cl)c[nH]c12)c1cncn1CCO] |
| 329 | 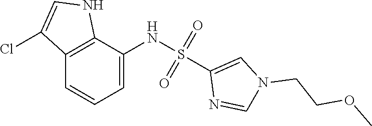 [SMILES: COCCn1cnc(S(=O)(=O)Nc2cccc3c(Cl)c[nH]c23)c1] |
| 330 | 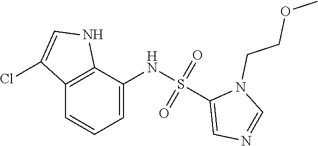 [SMILES: COCCn1cncc1S(=O)(=O)Nc1cccc2c(Cl)c[nH]c12] |
| 331 | 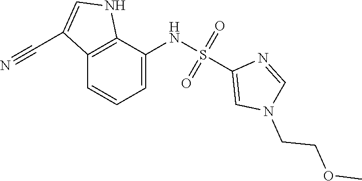 [SMILES: COCCn1cnc(S(=O)(=O)Nc2cccc3c(C#N)c[nH]c23)c1] |
| 332 | 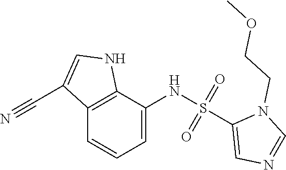 [SMILES: COCCn1cncc1S(=O)(=O)Nc1cccc2c(C#N)c[nH]c12] |
| 333 | 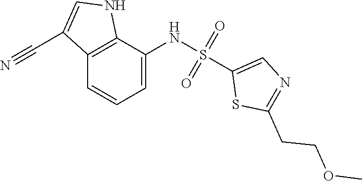 [SMILES: COCCc1ncc(S(=O)(=O)Nc2cccc3c(C#N)c[nH]c23)s1] |
| 334 | 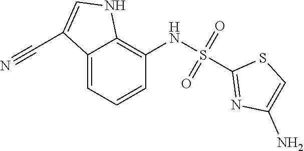 [SMILES: N#Cc1c[nH]c2c(NS(=O)(=O)c3nc(N)cs3)cccc12] |
| 335 | 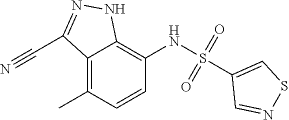 [SMILES: Cc1ccc(NS(=O)(=O)c2cnsc2)c2[nH]nc(C#N)c12] |
| 336 | 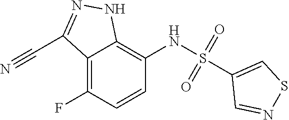 [SMILES: N#Cc1n[nH]c2c(NS(=O)(=O)c3cnsc3)ccc(F)c12] |
| 337 | 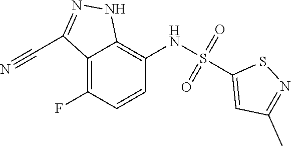 [SMILES: Cc1cc(S(=O)(=O)Nc2ccc(F)c3c(C#N)n[nH]c23)sn1] |
| 338 | 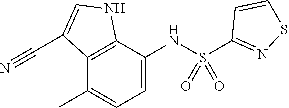 [SMILES: Cc1ccc(NS(=O)(=O)c2ccsn2)c2[nH]cc(C#N)c12] |
| 339 | 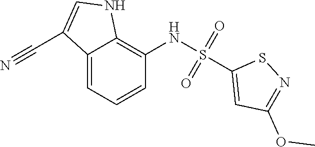 [SMILES: COc1cc(S(=O)(=O)Nc2cccc3c(C#N)c[nH]c23)sn1] |
| 340 | 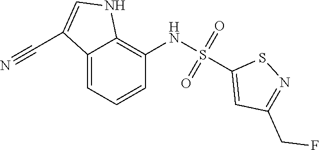 [SMILES: N#Cc1c[nH]c2c(NS(=O)(=O)c3cc(CF)ns3)cccc12] |
| 341 | 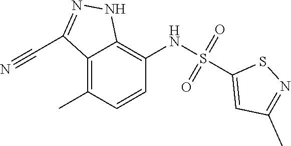 [SMILES: Cc1cc(S(=O)(=O)Nc2ccc(C)c3c(C#N)n[nH]c23)sn1] |
| 342 | 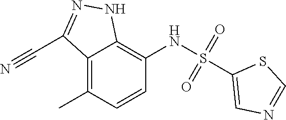 [SMILES: Cc1ccc(NS(=O)(=O)c2cncs2)c2[nH]nc(C#N)c12] |
| 343 | 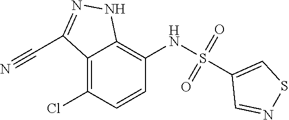 [SMILES: N#Cc1n[nH]c2c(NS(=O)(=O)c3cnsc3)ccc(Cl)c12] |
| 344 | 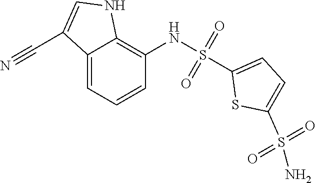 [SMILES: N#Cc1c[nH]c2c(NS(=O)(=O)c3ccc(S(N)(=O)=O)s3)cccc12] |
| 345 | 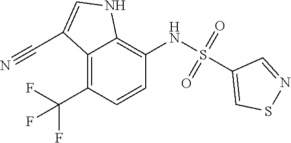 [SMILES: N#Cc1c[nH]c2c(NS(=O)(=O)c3cnsc3)ccc(C(F)(F)F)c12] |
| 346 | 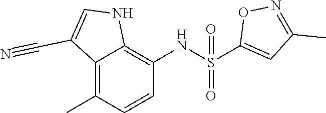 [SMILES: Cc1cc(S(=O)(=O)Nc2ccc(C)c3c(C#N)c[nH]c23)on1] |
| 347 | 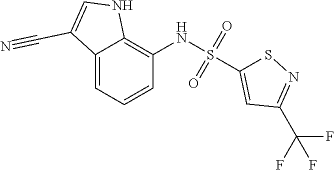 [SMILES: N#Cc1c[nH]c2c(NS(=O)(=O)c3cc(C(F)(F)F)ns3)cccc12] |
| 348 | 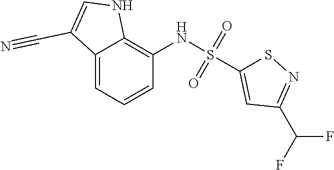 [SMILES: N#Cc1c[nH]c2c(NS(=O)(=O)c3cc(C(F)F)ns3)cccc12] |
| 349 | 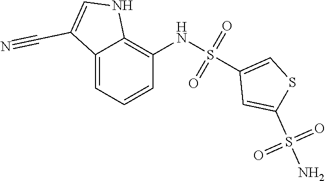 [SMILES: N#Cc1c[nH]c2c(NS(=O)(=O)c3csc(S(N)(=O)=O)c3)cccc12] |
| 350 | 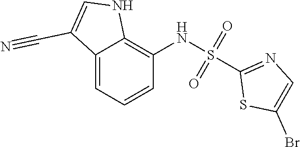 [SMILES: N#Cc1c[nH]c2c(NS(=O)(=O)c3ncc(Br)s3)cccc12] |
| 351 | 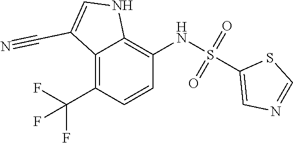 [SMILES: N#Cc1c[nH]c2c(NS(=O)(=O)c3cncs3)ccc(C(F)(F)F)c12] |
| 352 | 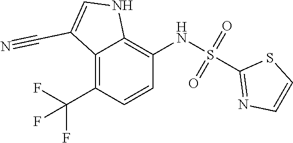 [SMILES: N#Cc1c[nH]c2c(NS(=O)(=O)c3nccs3)ccc(C(F)(F)F)c12] |
| 353 | 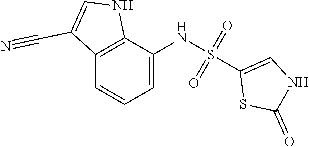 [SMILES: N#Cc1c[nH]c2c(NS(=O)(=O)c3c[nH]c(=O)s3)cccc12] |
| 354 | 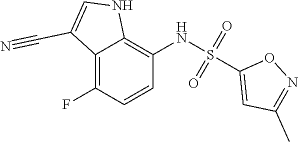 [SMILES: Cc1cc(S(=O)(=O)Nc2ccc(F)c3c(C#N)c[nH]c23)on1] |
| 355 | 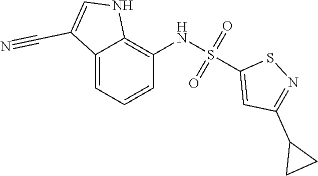 [SMILES: N#Cc1c[nH]c2c(NS(=O)(=O)c3cc(C4CC4)ns3)cccc12] |
| 356 | 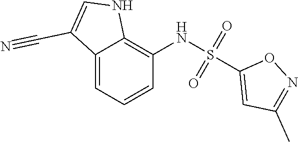 [SMILES: Cc1cc(S(=O)(=O)Nc2cccc3c(C#N)c[nH]c23)on1] |
| 357 | 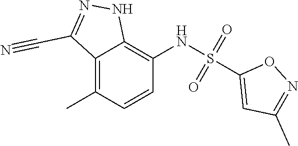 [SMILES: Cc1cc(S(=O)(=O)Nc2ccc(C)c3c(C#N)n[nH]c23)on1] |
| 358 | 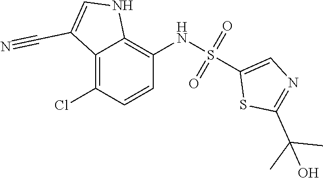 [SMILES: CC(C)(O)c1ncc(S(=O)(=O)Nc2ccc(Cl)c3c(C#N)c[nH]c23)s1] |
| 359 | 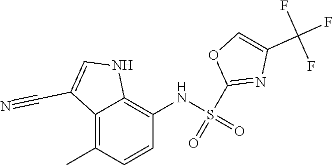 [SMILES: Cc1ccc(NS(=O)(=O)c2nc(C(F)(F)F)co2)c2[nH]cc(C#N)c12] |
| 360 | 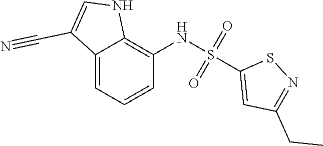 [SMILES: CCc1cc(S(=O)(=O)Nc2cccc3c(C#N)c[nH]c23)sn1] |
| 361 | 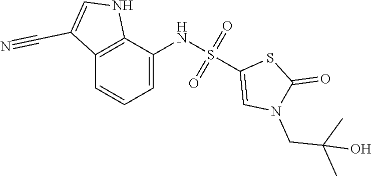 [SMILES: CC(C)(O)Cn1cc(S(=O)(=O)Nc2cccc3c(C#N)c[nH]c23)sc1=O] |
| 362 | 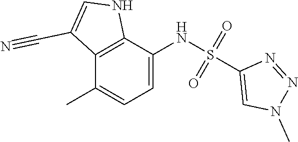 [SMILES: Cc1ccc(NS(=O)(=O)c2cn(C)nn2)c2[nH]cc(C#N)c12] |
| 363 | 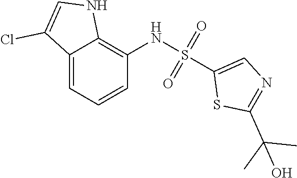 [SMILES: CC(C)(O)c1ncc(S(=O)(=O)Nc2cccc3c(Cl)c[nH]c23)s1] |
| 364 | 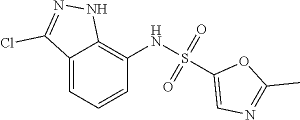 [SMILES: Cc1ncc(S(=O)(=O)Nc2cccc3c(Cl)n[nH]c23)o1] |
| 365 | 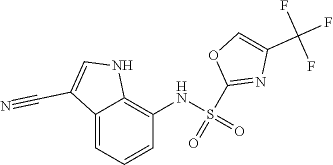 [SMILES: N#Cc1c[nH]c2c(NS(=O)(=O)c3nc(C(F)(F)F)co3)cccc12] |
| 366 | 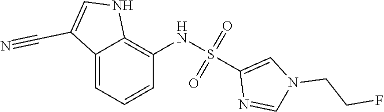 [SMILES: N#Cc1c[nH]c2c(NS(=O)(=O)c3cn(CCF)cn3)cccc12] |
| 367 | 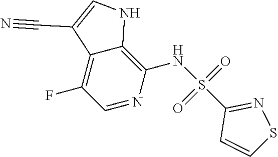 [SMILES: N#Cc1c[nH]c2c(NS(=O)(=O)c3ccsn3)ncc(F)c12] |
| 368 | 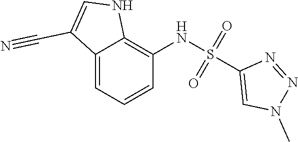 [SMILES: Cn1cc(S(=O)(=O)Nc2cccc3c(C#N)c[nH]c23)nn1] |
| 369 | 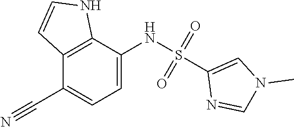 [SMILES: Cn1cnc(S(=O)(=O)Nc2ccc(C#N)c3cc[nH]c23)c1] |
| 370 | 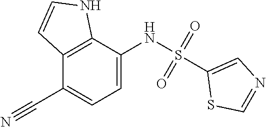 [SMILES: N#Cc1ccc(NS(=O)(=O)c2cncs2)c2[nH]ccc12] |
| 371 | 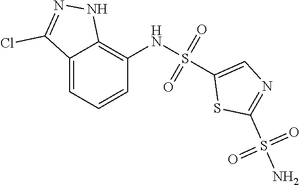 [SMILES: NS(=O)(=O)c1ncc(S(=O)(=O)Nc2cccc3c(Cl)n[nH]c23)s1] |
| 372 | 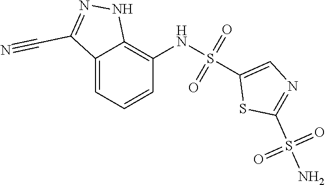 [SMILES: N#Cc1n[nH]c2c(NS(=O)(=O)c3cnc(S(N)(=O)=O)s3)cccc12] |
| 373 | 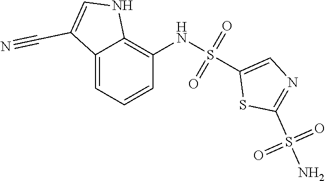 [SMILES: N#Cc1c[nH]c2c(NS(=O)(=O)c3cnc(S(N)(=O)=O)s3)cccc12] |
| 374 | 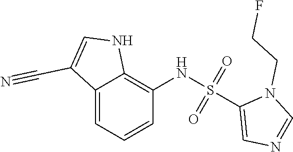 [SMILES: N#Cc1c[nH]c2c(NS(=O)(=O)c3cncn3CCF)cccc12] |
| 375 | 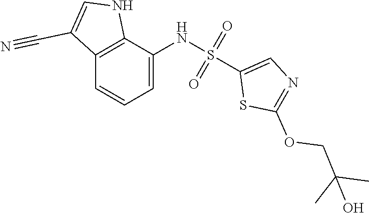 [SMILES: CC(C)(O)COc1ncc(S(=O)(=O)Nc2cccc3c(C#N)c[nH]c23)s1] |
| 376 | 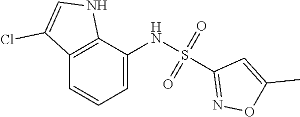 [SMILES: Cc1cc(S(=O)(=O)Nc2cccc3c(Cl)c[nH]c23)no1] |
| 377 | 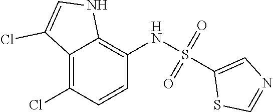 [SMILES: O=S(=O)(Nc1ccc(Cl)c2c(Cl)c[nH]c12)c1cncs1] |
| 378 | 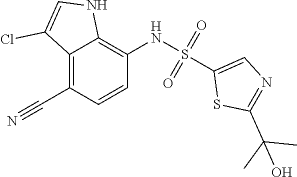 [SMILES: CC(C)(O)c1ncc(S(=O)(=O)Nc2ccc(C#N)c3c(Cl)c[nH]c23)s1] |
| 379 | 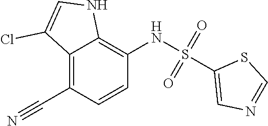 [SMILES: N#Cc1ccc(NS(=O)(=O)c2cncs2)c2[nH]cc(Cl)c12] |
| 380 | 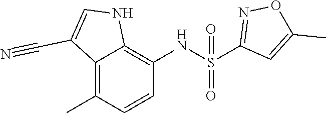 [SMILES: Cc1cc(S(=O)(=O)Nc2ccc(C)c3c(C#N)c[nH]c23)no1] |
| 381 | 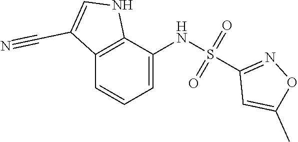 [SMILES: Cc1cc(S(=O)(=O)Nc2cccc3c(C#N)c[nH]c23)no1] |
| 382 | 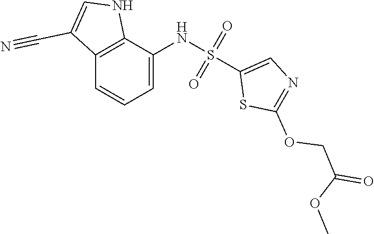 [SMILES: COC(=O)COc1ncc(S(=O)(=O)Nc2cccc3c(C#N)c[nH]c23)s1] |
| 383 | 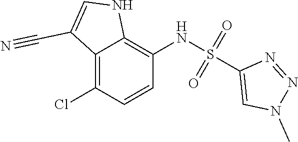 [SMILES: Cn1cc(S(=O)(=O)Nc2ccc(Cl)c3c(C#N)c[nH]c23)nn1] |
| 384 | 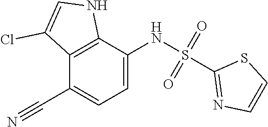 [SMILES: N#Cc1ccc(NS(=O)(=O)c2nccs2)c2[nH]cc(Cl)c12] |
| 385 | 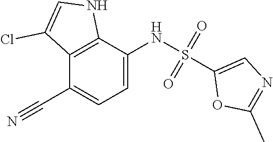 [SMILES: Cc1ncc(S(=O)(=O)Nc2ccc(C#N)c3c(Cl)c[nH]c23)o1] |
| 386 | 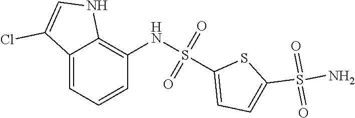 [SMILES: NS(=O)(=O)c1ccc(S(=O)(=O)Nc2cccc3c(Cl)c[nH]c23)s1] |
| 387 | 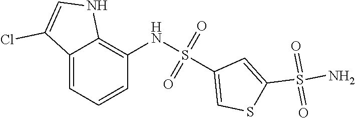 [SMILES: NS(=O)(=O)c1cc(S(=O)(=O)Nc2cccc3c(Cl)c[nH]c23)cs1] |
| 388 | 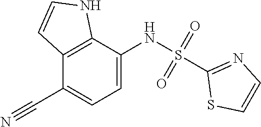 [SMILES: N#Cc1ccc(NS(=O)(=O)c2nccs2)c2[nH]ccc12] |
| 389 | 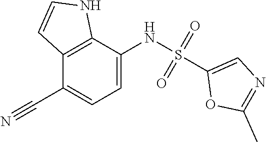 [SMILES: Cc1ncc(S(=O)(=O)Nc2ccc(C#N)c3cc[nH]c23)o1] |
| 390 | 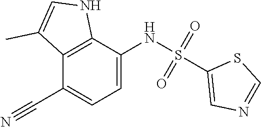 [SMILES: Cc1c[nH]c2c(NS(=O)(=O)c3cncs3)ccc(C#N)c12] |
| 391 | 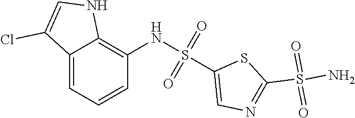 [SMILES: NS(=O)(=O)c1ncc(S(=O)(=O)Nc2cccc3c(Cl)c[nH]c23)s1] |
| 392 | 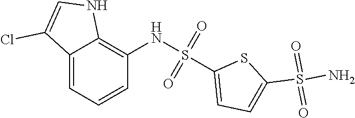 [SMILES: NS(=O)(=O)c1ccc(S(=O)(=O)Nc2cccc3c(Cl)c[nH]c23)s1] |
| 393 | 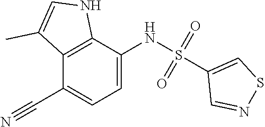 [SMILES: Cc1c[nH]c2c(NS(=O)(=O)c3cnsc3)ccc(C#N)c12] |
| 394 | 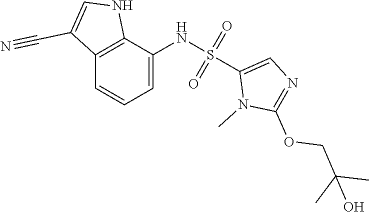 [SMILES: Cn1c(S(=O)(=O)Nc2cccc3c(C#N)c[nH]c23)cnc1OCC(C)(C)O] |
| 395 | 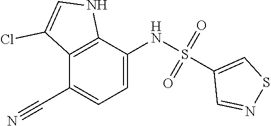 [SMILES: N#Cc1ccc(NS(=O)(=O)c2cnsc2)c2[nH]cc(Cl)c12] |
| 396 | 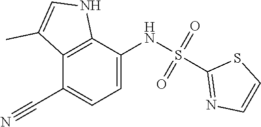 [SMILES: Cc1c[nH]c2c(NS(=O)(=O)c3nccs3)ccc(C#N)c12] |
| 397 | 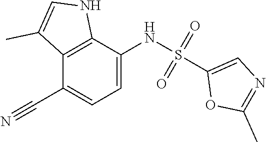 [SMILES: Cc1ncc(S(=O)(=O)Nc2ccc(C#N)c3c(C)c[nH]c23)o1] |
| 398 | 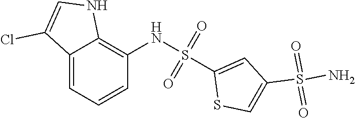 [SMILES: NS(=O)(=O)c1csc(S(=O)(=O)Nc2cccc3c(Cl)c[nH]c23)c1] |
| 399 | 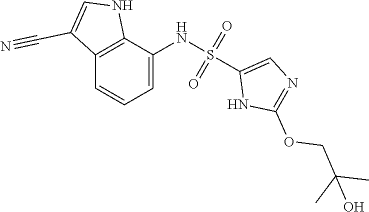 [SMILES: CC(C)(O)COc1ncc(S(=O)(=O)Nc2cccc3c(C#N)c[nH]c23)[nH]1] |
| 400 | 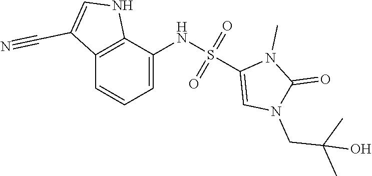 [SMILES: Cn1c(S(=O)(=O)Nc2cccc3c(C#N)c[nH]c23)cn(CC(C)(C)O)c1=O] |
| 401 | 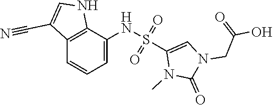 [SMILES: Cn1c(S(=O)(=O)Nc2cccc3c(C#N)c[nH]c23)cn(CC(=O)O)c1=O] |
| 402 | 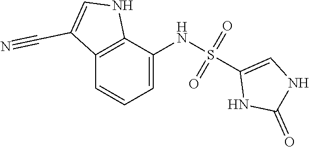 [SMILES: N#Cc1c[nH]c2c(NS(=O)(=O)c3c[nH]c(=O)[nH]3)cccc12] |
| 403 | 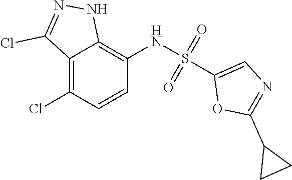 [SMILES: O=S(=O)(Nc1ccc(Cl)c2c(Cl)n[nH]c12)c1cnc(C2CC2)o1] |
| 404 | 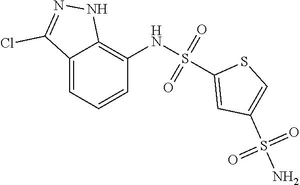 [SMILES: NS(=O)(=O)c1csc(S(=O)(=O)Nc2cccc3c(Cl)n[nH]c23)c1] |
| 405 | 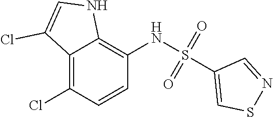 [SMILES: O=S(=O)(Nc1ccc(Cl)c2c(Cl)c[nH]c12)c1cnsc1] |
| 406 | 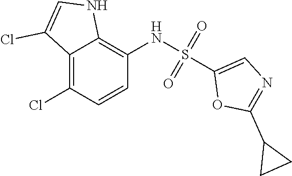 [SMILES: O=S(=O)(Nc1ccc(Cl)c2c(Cl)c[nH]c12)c1cnc(C2CC2)o1] |
| 407 | 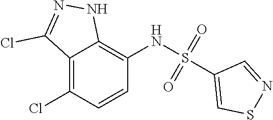 [SMILES: O=S(=O)(Nc1ccc(Cl)c2c(Cl)n[nH]c12)c1cnsc1] |
| 408 | 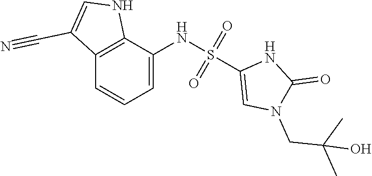 [SMILES: CC(C)(O)Cn1cc(S(=O)(=O)Nc2cccc3c(C#N)c[nH]c23)[nH]c1=O] |
| 409 | 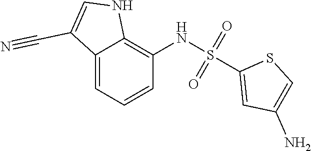 [SMILES: N#Cc1c[nH]c2c(NS(=O)(=O)c3cc(N)cs3)cccc12] |
| 410 | 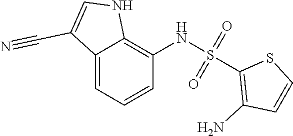 [SMILES: N#Cc1c[nH]c2c(NS(=O)(=O)c3sccc3N)cccc12] |
| 411 | 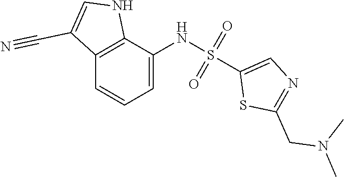 [SMILES: CN(C)Cc1ncc(S(=O)(=O)Nc2cccc3c(C#N)c[nH]c23)s1] |
| 412 | 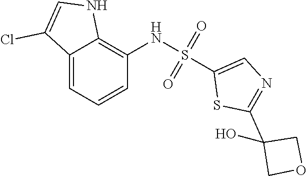 [SMILES: O=S(=O)(Nc1cccc2c(Cl)c[nH]c12)c1cnc(C2(O)COC2)s1] |
| 413 | 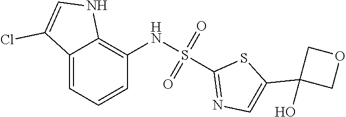 [SMILES: O=S(=O)(Nc1cccc2c(Cl)c[nH]c12)c1ncc(C2(O)COC2)s1] |
| 414 | 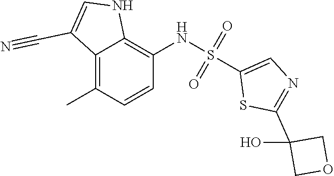 [SMILES: Cc1ccc(NS(=O)(=O)c2cnc(C3(O)COC3)s2)c2[nH]cc(C#N)c12] |
| 415 | 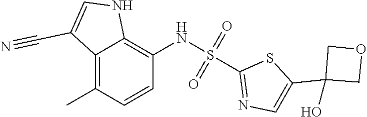 [SMILES: Cc1ccc(NS(=O)(=O)c2ncc(C3(O)COC3)s2)c2[nH]cc(C#N)c12] |
| 416 | 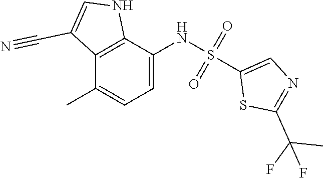 [SMILES: Cc1ccc(NS(=O)(=O)c2cnc(C(C)(F)F)s2)c2[nH]cc(C#N)c12] |
| 417 | 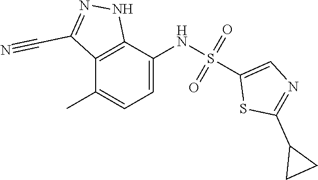 [SMILES: Cc1ccc(NS(=O)(=O)c2cnc(C3CC3)s2)c2[nH]nc(C#N)c12] |
| 418 | 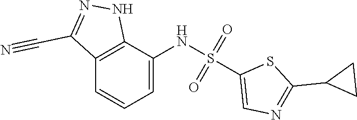 [SMILES: N#Cc1n[nH]c2c(NS(=O)(=O)c3cnc(C4CC4)s3)cccc12] |
| 419 | 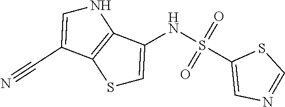 [SMILES: N#Cc1c[nH]c2c(NS(=O)(=O)c3cncs3)csc12] |
| 420 | 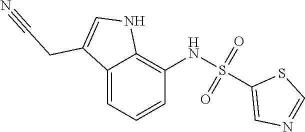 [SMILES: N#CCc1c[nH]c2c(NS(=O)(=O)c3cncs3)cccc12] |

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicates a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as analytical tools or probes in biological assays. Such compounds, especially deuterium analogs, can also be therapeutically useful.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

The compounds disclosed herein can be useful as modulators of RBM39, such as inhibitors of RBM39. These compounds can also be useful in the treatment or prevention of diseases and disorders associated with aberrant RBM39 activity, e.g., cancer, in a patient.

### Definitions

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₆ alkyl refers to an alkyl group that has 6 carbon atoms. C₁-C₆ alkyl refers to an alkyl group having a number of carbon atoms encompassing the entire range (e.g., 1 to 6 carbon atoms), as well as all subgroups (e.g., 1-6, 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1,1-dimethylethyl. Unless otherwise indicated, an alkyl group can be an unsubstituted alkyl group or a substituted alkyl group.

The term “haloalkyl” used herein refers to an alkyl group defined herein which is substituted with one or more halogen atoms.

The term “alkylene” used herein refers to an alkyl group having a substituent. For example, the term “alkylenehalo” refers to an alkyl group substituted with a halo group. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group.

As used herein, the term “cycloalkyl” refers to an aliphatic cyclic hydrocarbon group containing three to ten carbon atoms (e.g., 3, 4, 5, 6, 7, 8, 9, or 10 carbon atoms). The term Cₙ means the cycloalkyl group has “n” carbon atoms. For example, 05 cycloalkyl refers to a cycloalkyl group that has 5 carbon atoms in the ring. C₃-C₁₀ cycloalkyl refers to cycloalkyl groups having a number of carbon atoms encompassing the entire range (e.g., 3 to 10 carbon atoms), as well as all subgroups (e.g., 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2- 6, 3-6, 4-6, 5-6, 1-5, 2-5, 3-5, 4-5, 1-4, 2-4, 3-4, 1-3, 2-3, 1-2, 1, 2, 3, 4, 5, 6, 7, 8, 9, and 10 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycloalkyl group, an aryl group and/or a heteroaryl group. When a cycloalkyl group is fused to another cycloalkyl group, then each of the cycloalkyl groups can contain three to eight carbon atoms unless specified otherwise. Unless otherwise indicated, a cycloalkyl group can be unsubstituted or substituted.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of 5 or 6 atoms, of which 1, 2, or 3 of the ring atoms are heteroatoms independently selected from the group consisting of oxygen, nitrogen, and sulfur, and the remaining atoms in the ring are carbon atoms. Nonlimiting examples of heterocycloalkyl groups include piperdine, pyrazolidine, tetrahydrofuran, tetrahydropyran, dihydrofuran, morpholine, and the like.

Cycloalkyl and heterocycloalkyl groups can be saturated or partially unsaturated ring systems optionally substituted with, for example, one to three groups, independently selected alkyl, alkyleneOH, C(O)NH₂, NH₂, oxo (═O), aryl, alkylenehalo, halo, and OH. Heterocycloalkyl groups optionally can be further N-substituted with alkyl (e.g., methyl or ethyl), alkylene-OH, alkylenearyl, and alkyleneheteroaryl. The heterocycloalkyl groups described herein can be isolated or fused to another heterocycloalkyl group, a cycloalkyl group, an aryl group, and/or a heteroaryl group. When a heterocycloalkyl group is fused to another heterocycloalkyl group, then each of the heterocycloalkyl groups can contain three to twelve total ring atoms, and one to three heteroatoms. Unless otherwise indicated, a heterocycloalkyl group can be unsubstituted or substituted.

As used herein, the term “heteroaryl” refers to a monocyclic or bicyclic aromatic ring having 5 to 12 total ring atoms, and containing one to four heteroatoms selected from nitrogen, oxygen, and sulfur atoms in the aromatic ring. In particular, heteroaryls described herein contain 5 or 6 total ring atoms, and containing 1, 2, or 3 heteroatoms selected from nitrogen, oxygen, and sulfur in the aromatic ring. Unless otherwise indicated, a heteroaryl group can be unsubstituted or substituted with one or more, and in particular one to three, substituents as described herein. Examples of heteroaryl groups include, but are not limited to, thienyl, furyl, pyridyl, pyrrolyl, oxazolyl, triazinyl, triazolyl, thiazolyl, isothiazolyl, isoxazolyl, imidazolyl, pyrazinyl, pyrimidinyl, thiazolyl, thiadiazolyl, 1,4-dihydropyrrolo[3,2-b]pyrrolyl, 1,6-dihydropyrrolo[2,3-b]pyrrolyl, 6H-furo[2,3-b]pyrrolyl, 4H-furo[3,2-b]pyrrolyl, 6H-thieno[2,3-b]pyrrolyl, 4H-thieno[3,2-b]pyrrolyl, 1H-indolyl, 2H-isoindolyl, indolizyl, 1H-indazolyl, benzimidazolyl, 7-azaindolyl, 5-azaindolyl, 6-azaindolyl, 1,2-benzisoxazolyl, 1,2-benzisothiazolyl, 2,1-benzisothiazolyl, benzoxazolyl, benzthiazolyl, benzo[c][1,2,5]thiadiazolyl, 1,2-benzisothiazole-3(2H)-onyl, adenyl, guanyl, quinolyl, isoquinolyl, quinoxalyl, phthalazyl, quinazolyl, cinnolyl, 1,8-naphthyridyl, pyrido[3,2-d]pyrimidyl, pyrido[4,3-d]pyrimidyl, pyrido[3,4-b]pyrazyl, pyrido[2,3-b]pyrazyl, pteridyl, 2H-chromen-2-onyl, 2H-benzo[e][1,2]oxazyl, quinolin-2(1H)-onyl, and isoquinolin-1(2H)-onyl.

As used herein, the term “alkoxy” or “alkoxyl” as used herein refers to a “—O-alkyl” group. The alkoxy or alkoxyl group can be unsubstituted or substituted.

As used herein, the term “therapeutically effective amount” means an amount of a compound or combination of therapeutically active compounds that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans).

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

As used herein, the term “excipient” means any pharmaceutically acceptable additive, carrier, diluent, adjuvant, or other ingredient, other than the active pharmaceutical ingredient (API).

### Pharmaceutically Acceptable Salts

The compounds described herein can exist in free form, or, where appropriate, as salts. Those salts that are pharmaceutically acceptable are of particular interest since they are useful in administering the compounds described below for medical purposes. Salts that are not pharmaceutically acceptable are useful in manufacturing processes, for isolation and purification purposes, and in some instances, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄ alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations, Dosing, and Routes of Administration

Further provided are pharmaceutical formulations (alternatively referred to as compositions throughout herein) comprising a compound as described herein or pharmaceutically acceptable salt thereof, and a pharmaceutically acceptable excipient.

The compounds described herein can be administered to a subject in a therapeutically effective amount, alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to affect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, for example, from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by physico-chemical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, PA, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus, the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds described herein can modulate RBM39. In some embodiments, the compounds inhibit RBM39. In various embodiments, the compounds induce RBM39 degradation, i.e., the compounds are RBM39 degraders.

The term “RBM39 degrader” as used herein refers to a compound having the ability to induce the degradation of RBM39 protein formation of a complex between RBM39 protein and any portion of an E3 ubiquitin ligase complex.

Although RBM39 has been identified as being associated with malignant progression in a number of solid and hematological cancers, there is still a great need and opportunity for an improved approach to modulate the activity of this protein. For example, RBM39 is vital for colorectal cancer cell survival in vitro and in vivo (Owa, et al., Journal of Medicinal Chemistry., 42(19), 3789-3799 (1999); Han, et al., Science., 356(6336), (2017); Ozawa, et al., Eur J Cancer, 37(17), 2275-2282 (2001); Sillars-Hardebol, et al., Gut., (61), 1568-1575 (2012); Uehara, et al., Nat Chem Biol., 13, 675-680 (2017)), has been implicated in breast cancer progression where it mediates VEGF alternative splicing (Mercier, et al., Am J Pathol. 174(4), 1172-1190 (2009)), and is also upregulated in human non-small cell lung cancer (NSCLC) tissues in comparison with normal lung tissues, facilitating proliferation and migration (Chai, et al., Tumor Biol., 35, 6311-6317 (2014)). RBM39 protein is required for acute myeloid leukemia (AML) maintenance through mis-splicing of HOXA9 target genes, and is required for neuroblastoma cell survival in vitro and in vivo (Wang, et al., Cancer Cell., 35(3), 369-384 (2019); Singh, et al., Sci Adv., 7(47), (2021)). RBM39 is an emerging cancer target (Yuewei et al, 2021). Other cancers that showed promising therapeutic potential are Neuroblastoma with MYC-N amplification and tumors with KRAS mutations as highlighted herein.

The compounds disclosed herein are particularly advantageous for the treatment or prevention of diseases or disorders caused by aberrant RBM39 activity.

As used herein, “aberrant RBM39 activity” refers to RBM39 activity associated with malignant progression in cancers. Such RBM39-linked malignant progression is associated with a variety of cancers (Xu, et al., Cell Death Discov. 7, 214 (2021)). One example of aberrant RBM39 activity is the RBM39-induced splicing of proteins encoded by KRAS oncogenes, such as KRAS4A.

Given the importance of the biological roles of RBM39, the compounds of the present disclosures are useful for a number of applications in a variety of settings. For example and most simplistically, the active agents of the present disclosures are useful for inducing the degradation of RBM39 in a cell. In this regard, the present disclosures provide methods of inducing the degradation of RBM39 in a cell. The method comprises contacting the cell with a compound of the present disclosures, or a pharmaceutically acceptable salt thereof, in an amount effective to induce the degradation. In some aspects, the cell is part of an in vitro or ex vivo cell culture or in vitro or ex vivo tissue sample. In some aspects, the cell is an in vivo cell. In certain embodiments, the method is intended for research purposes, and, in other embodiments, the method is intended for therapeutic purposes.

As shown herein, a compound that induces the degradation of RBM39 increases tumor cell death. Thus, the present disclosures provides a method of increasing tumor cell death in a subject. The method comprises administering to the subject a compound of the present disclosure, or a pharmaceutically acceptable salt thereof, in an amount effective to increase tumor cell death.

In accordance with the foregoing, the present disclosures further provides methods of treating a cancer in a subject. The methods comprise administering to the subject a compound of the present disclosures, or a pharmaceutically acceptable salt thereof, in an amount effective to treat the cancer in the subject.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosures can provide any amount or any level of treatment of cancer. Furthermore, the treatment provided by the method of the present disclosures may include treatment of one or more conditions or symptoms of the cancer, being treated. Also, the treatment provided by the methods of the present disclosures may encompass slowing the progression of the cancer. For example, the methods can treat cancer by virtue of reducing tumor or cancer growth, reducing metastasis of tumor cells, increasing cell death of tumor or cancer cells, and the like.

The cancer treatable by the methods disclosed herein may be any cancer, e.g., any malignant growth or tumor caused by abnormal and uncontrolled cell division that may spread to other parts of the body through the lymphatic system or the blood stream. In some embodiments, the cancer is a cancer in which an RBM39 is expressed by the cells of the cancer. In some aspects, the cancer is a cancer in which an RBM39 protein is over-expressed, the gene encoding RBM39 is amplified, and/or an RBM39 mutant protein (e.g., truncated RBM39, point-mutated RBM39) is expressed.

Neuroblastoma is the most common pediatric solid tumor with poor prognosis for high-risk cases despite the use of multimodal treatment. Neuroblastoma, a MYC-driven cancer characterized by splicing dysregulation and spliceosomal dependency, requires the splicing factor RBM39 for survival. Shivendra et al, (See “Targeting the spliceosome through RBM39 degradation results in exceptional responses in high-risk neuroblastoma models”2021) showed that aberrant alternative pre-mRNA splicing plays a critical role in MYC-driven cancers and targeting the dysregulated spliceosome may represent a valid therapeutic strategy in these cancers. Genetic depletion or indisulam-mediated degradation of RBM39 induced significant genome-wide splicing anomalies and cell death in neuroblastoma, leading to significant responses in multiple high-risk disease models, without overt toxicity. Anke Nijhuis et al, (2022, See “Indisulam targets RNA splicing and metabolism to serve as a therapeutic strategy for high-risk neuroblastoma”) also confirmed that neuroblastoma lines are very sensitive to indisulam. RNAseq and proteomic analysis highlighted distinct disruption to cell cycle, metabolome & mitochondrial function both in vitro and in vivo. Their work also confirmed complete tumor regressions without relapse in both xenograft and the Th-MYCN transgenic model of neuroblastoma with indisulam treatments.

The KRAS oncogene that is mutated in many cancers encodes two distinct KRAS4A and KRAS4B proteins generated by differential splicing. Wei-Ching Chen et al, recently (2021) demonstrated that coordinated regulation of both KRAS4A and KRAS4B isoforms through control of splicing is essential for development of Kras mutant tumors. The minor KRAS4A isoform is enriched in cancer stem-like cells and responds to hypoxia, while the major KRAS4B is induced by ER stress. Splicing of KRAS4A is controlled by the DCAF15/RBM39 pathway. They experimentally illustrated that deletion of KRAS4A or pharmacological inhibition of RBM39 using indisulam leads to inhibition of cancer stem cells. Therefore sulfonamides that target KRAS4A splicing could have the potential to inhibit in human tumors that express minor KRAS4A isoform. Minor KRAS4A expression could be used as a biomarker of sensitivity these drugs (See Wei-Ching Chen et al, “Targeting KRAS4A splicing through the RBM39/DCAF15 pathway inhibits cancer stem cells”, Nature Communications 12:4288, (2021)).

Puvvula et al, (2021, “Inhibiting an RBM39/MLL1 epigenomic regulatory complex with dominant-negative peptides disrupts cancer cell transcription and proliferation”) demonstrated a pathologic complex between RBM39 and MLL1 regulates tumor formation, H3K4me3, and tumor suppressor and oncogene expression in breast cancer cells. They demonstrated the therapeutic potential of RBM39 RRM3-derived peptides that disrupt the RBM39/MLL1 complex, reduce H3K4me3 and cancer hallmarks in multiple breast cancer subtypes, and yet are nontoxic to normal cells.

The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, leukemia (e.g., chronic lymphocytic leukemia), chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and esophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In particular aspects, the cancer is an osimertinib-resistant cancer. In some cases, the cancer is pancreatic cancer, head and neck cancer, melanoma, colon cancer, renal cancer, leukemia, or breast cancer. In some cases, the cancer is melanoma, colon cancer, renal cancer, leukemia, or breast cancer. In some cases, the cancer is renal cancer. In some cases, the cancer is renal cell carcinoma.

Uses of the compounds disclosed herein in the preparation of a medicament for modulating RBM39, or for treating or preventing a disease or disorder associated with aberrant RBM39 activity also are provided herein.

The disclosure herein will be understood more readily by reference to the following examples, below.

In view of the many possible embodiments to which the principles of the disclosure may be applied, it should be recognized that the illustrated embodiments are only examples and should not be taken as limiting the scope of the invention.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

### Synthesis of Compounds of the Disclosure

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the synthetic schemes as described in the Examples section, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; and Greene, T. W., Wuts, P.G.M., Protective Groups in Organic Synthesis, 3 rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### EMBODIMENTS

The disclosure may be further understood in view of the following non-limiting embodiments.

Embodiment 1. A compound of Formula (I), or a pharmaceutically acceptable salt thereof:

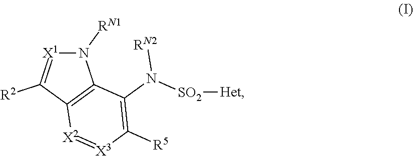

wherein
R^{N1} is H or C₁₋₆alkyl optionally substituted with 1, 2, or 3 R⁷;
R^{N2} is H or C₁₋₆alkyl optionally substituted with 1, 2, or 3 R⁷;
X¹ is CR¹ or N;
X² is CR³ or N;
X³ is CR⁴ or N;
R¹ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R² is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R³ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁴ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁵ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
each R^{N} is independently H, C₁₋₆alkyl optionally substituted with 1, 2, or 3 R⁷, or C₃₋₁₀cycloalkyl;
Het is a 5-, 6-, 7-, 8-, 9-, 10-, 11-, or 12-membered heteroaryl comprising 1, 2, or 3 ring heteroatoms selected from O, S, and N and optionally substituted with 1, 2, or 3 R⁶;
each R⁶ is independently halo, CN, C₁₋₆alkyl, C₁₋₆haloalkyl, C₁₋₆alkoxy, NR^{N}R^{N}, COOH, C(O)NR^{N}R^{N}, C₁₋₆alkylene-C(O)OR^{N}, C₁₋₆alkylene-C(O)NR^{N}R^{N}, SO₂NR^{N}R^{N}, P(O)(R^{N})(R^{N}), C(O)-5- or 6-membered heterocycloalkyl comprising 1, 2, or 3 ring heteroatoms selected from O, S, and N, C₁₋₆alkylene-C₃₋₁₀ cycloalkyl, C₃₋₁₀cycloalkyl, 4-6-membered heterocycloalkyl comprising 1, 2, or 3 ring heteroatoms selected from O, S, and N, C₆₋₁₀aryl, or 5- or 6-membered heteroaryl comprising 1, 2, or 3 ring heteroatoms selected from O, S, and N, wherein the C₃₋₁₀cycloalkyl, 4-6-membered heterocycloalkyl, C₃₋₁₀aryl, or 5- or 6-membered heteroaryl can optionally be substituted with 1, 2, or 3 R⁷ and each C₁₋₆alkyl, C₁₋₆alkylene, or C₁₋₆alkoxy can be optionally substituted with 1 or 2 substituents independently selected from C₁₋₆alkoxy, OH, CN, CO₂H, NR^{N}R^{N}, and CO₂C₁₋₆alkyl; and
each R⁷ is independently OH, halo, CN, C₁₋₆alkyl, C₁₋₆haloalkyl, C₁₋₆alkoxy, NH₂, NH(C₁₋₆alkyl), or N(C₁₋₆alkyl)₂.

Embodiment 2. The compound or salt of embodiment 1, wherein Het is a 5-membered heteroaryl comprising 1, 2, or 3 ring heteroatoms selected from O, S, and N and optionally substituted with 1, 2, or 3 R⁶.

Embodiment 3. The compound or salt of embodiment 2, wherein Het is thiazolyl optionally substituted with 1, 2, or 3 R⁶.

Embodiment 4. The compound or salt of embodiment 1 or 2, wherein Het is

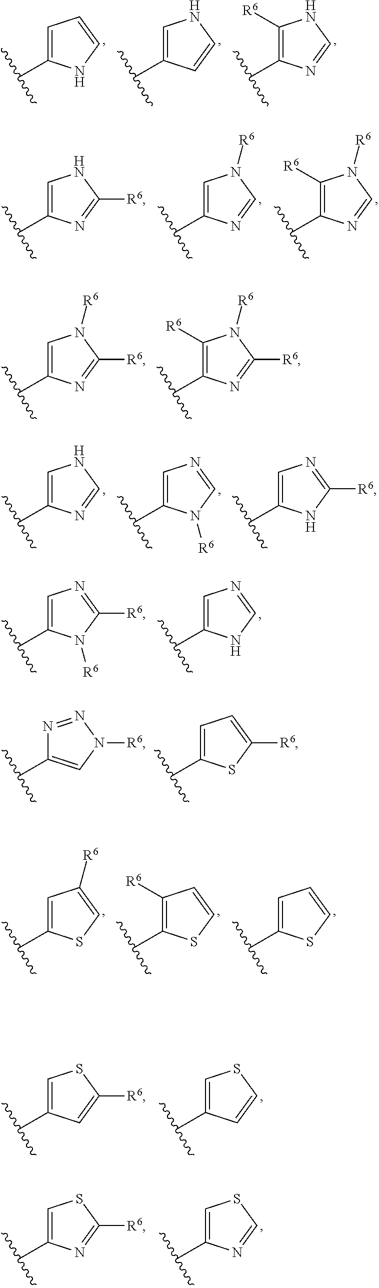

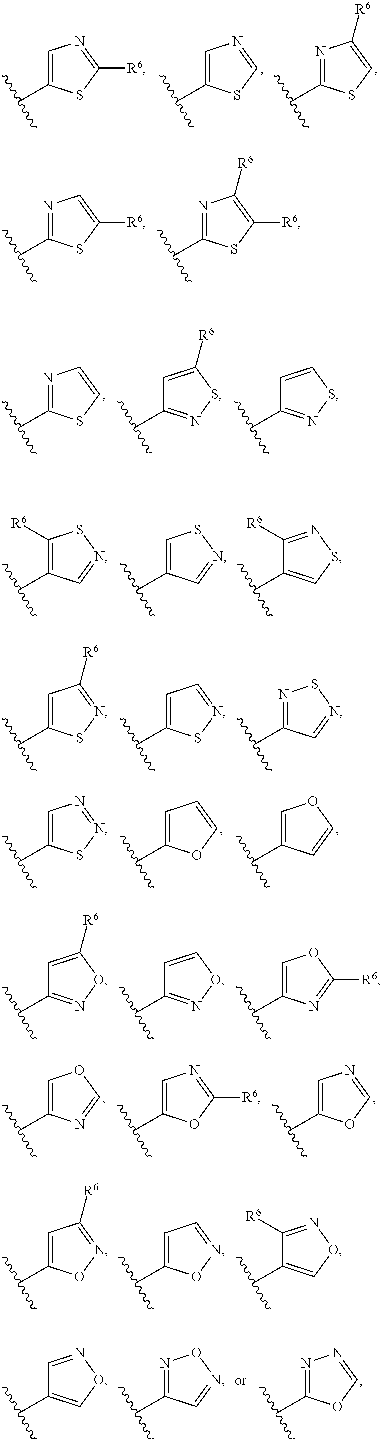

and R⁶ is C₁₋₆alkyl, C₁₋₆haloalkyl, C₁₋₆alkoxy, C₃₋₁₀cycloalkyl, P(O)(Me)₂, or C(O)-morpholinyl.

Embodiment 5. The compound or salt of any one of embodiments 1 to 4, wherein X¹ is CR¹, X² is CR³, and X³ is CR⁴.

Embodiment 6. The compound or salt of any one of embodiments 1 to 4, wherein one of X¹, X², and X³ is N.

Embodiment 7. The compound or salt of embodiment 6, wherein X¹ is N.

Embodiment 8. The compound or salt of embodiment 6, wherein X³ is N.

Embodiment 9. The compound or salt of embodiment 1, having the structure of Formula (Ia):

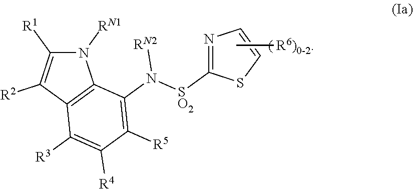

Embodiment 10. The compound or salt of any one of embodiments 1 to 9, wherein R^{N1} is H.

Embodiment 11. The compound or salt of any one of embodiments 1 to 10, wherein R^{N2} is H.

Embodiment 12. The compound or salt of any one of embodiments 1 to 6 and 8 to 11, wherein R¹ is H or C₁₋₆alkyl, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl.

Embodiment 13. The compound or salt of embodiment 12, wherein R¹ is H.

Embodiment 14. The compound or salt of any one of embodiments 1 to 13, wherein R² is H, C₁₋₆alkyl, halo, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl.

Embodiment 15. The compound or salt of embodiment 14, wherein R² is H, halo, or CN.

Embodiment 16. The compound or salt of embodiment 15, wherein R² is Cl.

Embodiment 17. The compound or salt of embodiment 16, wherein R² is CN.

Embodiment 18. The compound or salt of any one of embodiments 1 to 17, wherein R³ is H, C₁₋₆alkyl, or halo, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl.

Embodiment 19. The compound or salt of embodiment 18, wherein R³ is H.

Embodiment 20. The compound or salt of embodiment 18, wherein R³ is C₁₋₆alkyl, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl.

Embodiment 21. The compound or salt of embodiment 20, wherein R³ is methyl.

Embodiment 22. The compound or salt of embodiment 18, wherein R³ is halo.

Embodiment 23. The compound or salt of embodiment 22, wherein R³ is fluoro.

Embodiment 24. The compound or salt of any one of embodiments 1 to 7 and 9 to 23, wherein R⁴ is H.

Embodiment 25. The compound or salt of any one of embodiments 1 to 24, wherein R⁵ is H.

Embodiment 26. The compound or salt of embodiment 1, wherein X¹ is CH, R² is H or CN, X² is CH or CMe, X³ is CH, R⁵ is H, and Het is thiazolyl, imidazolyl, isooxazolyl, 1,2,4-triazolyl, or oxazolyl, and Het is unsubstituted, or substituted with 1 or 2 R⁶.

Embodiment 27. The compound or salt of embodiment 26, wherein Het is substituted with 1 R⁶.

Embodiment 28. The compound or salt of embodiment 26, wherein Het is substituted with 2 R⁶.

Embodiment 29. The compound or salt of any one of embodiments 1 to 28, wherein each R⁶ is independently halo, CN, C₁₋₆alkyl, C₁₋₆haloalkyl, C₁₋₆alkoxy, COOH, C(O)NR^{N}R^{N}, C₁₋₆alkylene-C(O)OR^{N}, P(O)(R^{N})(R^{N}), C(O)-5- or 6-membered heterocycloalkyl comprising 1, 2, or 3 ring heteroatoms selected from 0, S, and N, C₃₋₅ cycloalkyl, 5- or 6-membered heterocycloalkyl comprising 1, 2, or 3 ring heteroatoms selected from O, S, and N, C₃₋₁₀aryl, or 5- or 6-membered heteroaryl comprising 1, 2, or 3 ring heteroatoms selected from O, S, and N, wherein the C₃₋₅ cycloalkyl, 5- or 6-membered heterocycloalkyl, C₃₋₁₀aryl, or 5- or 6-membered heteroaryl can optionally be substituted with 1, 2, or 3 R⁷ and each C₁₋₆alkyl or C₁₋₆alkylene can be optionally substituted with 1 or 2 substituents independently selected from C₁₋₆alkoxy, OH, CN, CO₂H, NR^{N}R^{N}, and CO₂C₁₋₆alkyl.

Embodiment 30. The compound or salt of embodiment 29, wherein each R⁶ is independently halo, CN, C₁₋₆alkyl, C₁₋₆haloalkyl, C₁₋₆alkoxy, C₁₋₆alkylene-C(O)O—C₁₋₆alkyl, 5- or 6-membered heterocycloalkyl comprising 1, 2, or 3 ring heteroatoms selected from O, S, and N, C₃₋₁₀aryl, or 5- or 6-membered heteroaryl comprising 1, 2, or 3 ring heteroatoms selected from O, S, and N, wherein the 5- or 6-membered heterocycloalkyl, C₃₋₁₀aryl, or 5- or 6-membered heteroaryl can optionally be substituted with 1, 2, or 3 R⁷ and each C₁₋₆alkyl or C₁₋₆alkylene can be optionally substituted with 1 or 2 substituents independently selected from C₁₋₆alkoxy, OH, CN, CO₂H, NR^{N}R^{N}, and CO₂C₁₋₆alkyl.

Embodiment 31. The compound or salt of embodiment 30, wherein each R⁶ is independently halo, C₁₋₆alkyl, or 5- or 6-membered heteroaryl comprising 1, 2, or 3 ring heteroatoms selected from O, S, and N, wherein the 5- or 6-membered heteroaryl can optionally be substituted with 1, 2, or 3 R⁷ and each C₁₋₆alkyl can be optionally substituted with 1 or 2 substituents independently selected from C₁₋₆alkoxy, OH, CN, CO₂H, NR^{N}R^{N}, and CO₂C₁₋₆alkyl.

Embodiment 32. The compound or salt of embodiment 30, wherein at least one R⁶ is halo.

Embodiment 33. The compound or salt of embodiment 32, wherein at least one R⁶ is chloro or bromo.

Embodiment 34. The compound or salt of embodiment 30, wherein at least one R⁶ is C₁₋₆alkyl, and each C₁₋₆alkyl can be optionally substituted with 1 or 2 substituents independently selected from C₁₋₆alkoxy, OH, CN, CO₂H, NR^{N}R^{N}, and CO₂C₁₋₆alkyl.

Embodiment 35. The compound or salt of embodiment 34, wherein at least one R⁶ is methyl, ethyl, or isopropyl.

Embodiment 36. The compound or salt of embodiment 30, wherein each R⁶ is independently 5- or 6-membered heteroaryl comprising 1, 2, or 3 ring heteroatoms selected from O, S, and N, wherein the 5- or 6-membered heteroaryl can optionally be substituted with 1, 2, or 3 R⁷.

Embodiment 37. The compound or salt of embodiment 36, wherein at least one R⁶ is pyrazolyl, pyridinyl, pyridazinyl, or pyrimidinyl, each of which can optionally be substituted with 1, 2, or 3 R⁷.

Embodiment 38. The compound or salt of any one of embodiments 1 to 37, wherein each R⁷ is independently halo, C₁₋₆alkyl, or C₁₋₆haloalkyl.

Embodiment 39. The compound or salt of embodiment 38, wherein at least one R⁷ is C₁₋₆alkyl.

Embodiment 40. The compound or salt of embodiment 39, wherein at least one R⁷ is methyl.

Embodiment 41. A compound listed in Table A, or a pharmaceutically acceptable salt thereof.

Embodiment 42. A pharmaceutical composition comprising the compound or salt of any one of embodiments 1 to 41 and a pharmaceutically acceptable excipient.

Embodiment 43. A method of modulating an RBM39 protein, comprising contacting the RBM39 protein with the compound or salt of any one of embodiments 1 to 41 or the pharmaceutical composition of embodiment 42.

Embodiment 44. The method of embodiment 43, wherein modulating an RBM39 protein comprises degrading the RBM39 protein.

Embodiment 45. The method of embodiment 43 or 44, wherein the contacting of the compound or salt comprises administering to a subject.

Embodiment 46. The method of embodiment 45, wherein the subject is human.

Embodiment 47. A method of treating a disease associated with aberrant RBM39 activity in a subject, comprising administering to the subject a therapeutically effective amount of the compound or salt of any one of embodiments 1 to 41 or the pharmaceutical composition of embodiment 42.

Embodiment 48. The method of embodiment 47, wherein the disease is cancer.

Embodiment 49. The method of embodiment 48, wherein the cancer is renal cell carcinoma.

### EXAMPLES

### Example 1

General Method A

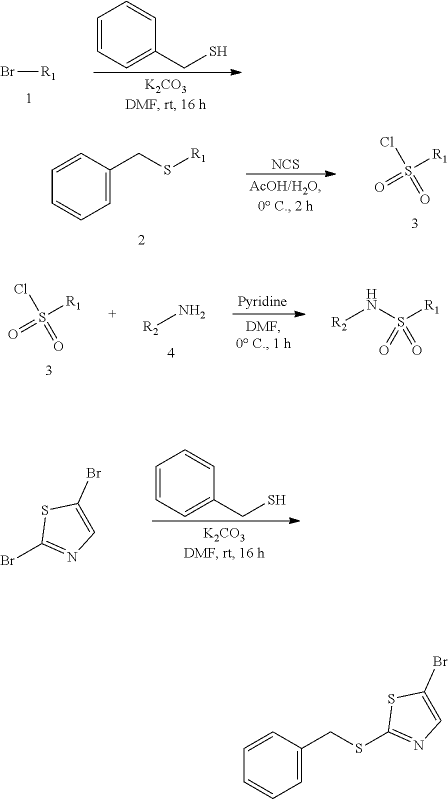

Synthesis of 2-(benzylthio)-5-bromothiazole. To a stirred suspension of phenylmethanethiol (0.62 g, 5.03 mmol and K₂CO₃ (1.39 g, 10.07 mmol)) in DMF (10 mL) was added compound 2-bromo-5-chlorothiazole (1.0 g, 5.03 mmol) at RT and stirred for 16 h. The progress of the reaction was monitored by TLC and LCMS. (R f values of SM and product were respectively 0.3 and 0.5; TLC system: 20% EtOAc and Pet ether). After completion of the reaction, RM was quenched by adding ice cold water (10 mL), extracted with ethyl acetate (30 mL). The organic layer was washed with brine (3×30 mL), dried over Na₂SO₄ and concentrated under reduced pressure to afford crude compound. The crude compound was purified by flash column chromatography (using 100-200 silica and eluted with 15% EtOAc in Pet ether) to afford the product.

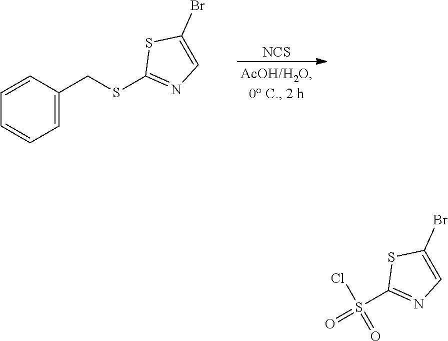 [SMILES: Brc1cnc(SCc2ccccc2)s1 ; "NCS" ; O=S(=O)(Cl)c1ncc(Br)s1]

Synthesis of 5-bromothiazole-2-sulfonyl chloride. To a stirred solution of 2-(benzylthio)-5-chlorothiazole (0.5 g, 2.06 mmol) in acetic acid:water (3:1) (10 mL) was added N-chlorosuccinimide (0.55 g, 4.13 mmol) at 0° C. and allowed to stir at RT for 2 h. After completion of the 2 h, reaction mass was concentrated under reduced pressure. The residue was dissolved in ethyl acetate (20 mL), washed with saturated aq. NaHCO₃ (2×20 mL), brine (15 mL), dried over Na₂SO₄ and concentrated under reduced pressure to give the title compound which was used in next step without any further purification.

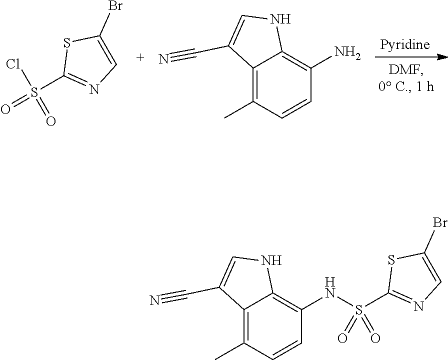 [SMILES: O=S(=O)(Cl)c1ncc(Br)s1 ; Cc1ccc(N)c2[nH]cc(C#N)c12 ; Cc1ccc(NS(=O)(=O)c2ncc(Br)s2)c2[nH]cc(C#N)c12]

Synthesis of 5-bromo-N-(3-cyano-4-methyl-1H-indol-7-yl)-1,3-thiazole-2-sulfonamide. To a stirred solution of 7-amino-4-methyl-1H-indole-3-carbonitrile (1 eq) and pyridine (5 eq) in DMF (7.0 mL) at 0° C., crude 5-chlorothiazole-2-sulfonyl chloride was added allowed to stir at RT for 1 h. The progress of the reaction was monitored by TLC and LCMS. After completion of the reaction, the mixture was quenched with water (15 mL) and extracted using ethyl acetate (2×30 mL). Combined organic layer was washed with brine (20 mL), dried over sodium sulfate, and concentrated under reduced pressure to afford crude compound. Crude compound was purified by prep HPLC/achiral SFC method to afford the corresponding final compound.

LCMS: 369.16 [M+H]+ ion present. ¹H-NMR (400 MHz, DMSO-d6): δ 12.11 (s, 1H), 11.00 (s, 1H), 8.21-8.16 (m, 3H), 7.68-7.59 (m, 2H), 6.75 (s, 2H), 2.56 (s, 3H)

### Example 2

### General Method B

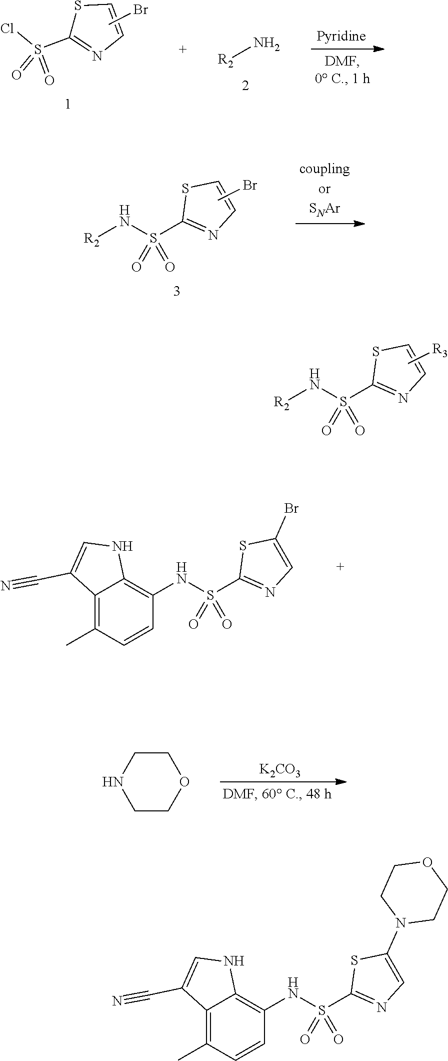

Synthesis of Preparation of N-(3-cyano-4-methyl-1H-indol-7-yl)-5-(morpholin-4-yl)-1,3-thiazole-2-sulfonamide. To a solution of 5-bromo-N-(3-cyano-4-methyl-1H-indol-7-yl)-1,3-thiazole-2-sulfonamide (300 mg, 0.755 mmol, 1.00 equiv) in DMF (6 mL) was added morpholine (98.7 mg, 1.13 mmol, 1.50 equiv) and K₂CO₃ (209 mg, 1.51 mmol, 2.00 equiv) and the resulting mixture was stirred at 60° C. under nitrogen atmosphere for 48 h. The mixture was allowed to cool down to room temperature. The residue was purified by reversed-phase flash chromatography to afford the title compound.

LCMS (ES) m/z: [M+H]⁺: 404, ¹H NMR (300 MHz, DMSO-d₆, ppm) δ 11.99 (s, 1H), 10.35 (bs, 1H), 8.18 (s, 1H), 7.27 (s, 1H), 6.85 (d, J=7.8 Hz, 1H), 6.75 (d, J=7.7 Hz, 1H), 3.70 (t, J=4.9 Hz, 4H), 3.16 (t, J=4.9 Hz, 4H), 2.59 (s, 3H).

General Method C

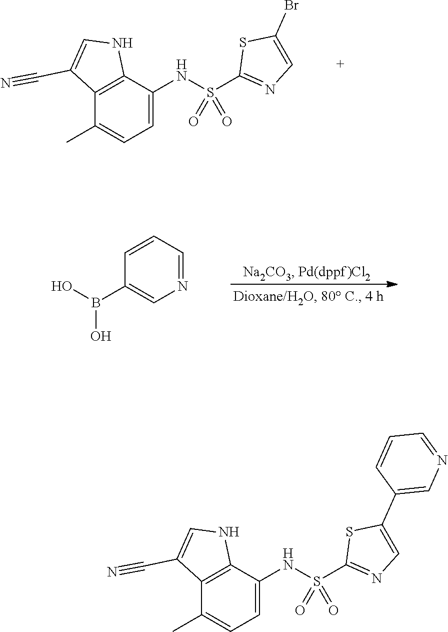 [SMILES: Cc1ccc(NS(=O)(=O)c2ncc(Br)s2)c2[nH]cc(C#N)c12 ; OB(O)c1cccnc1 ; "Na2CO3, Pd(dppf)Cl2" ; Cc1ccc(NS(=O)(=O)c2ncc(-c3cccnc3)s2)c2[nH]cc(C#N)c12]

Synthesis of N-(3-cyano-4-methyl-1H-indol-7-yl)-5-(pyridin-3-yl)-1,3-thiazole-2-sulfonamide. To a solution of 5-bromo-N-(3-cyano-4-methyl-1H-indol-7-yl)-1,3-thiazole-2-sulfonamide (150 mg, 0.378 mmol, 1 eq.) and pyridin-3-ylboronic acid (185 mg, 1.51 mmol, 4 eq.) in dioxane (2.4 mL) and H₂O (0.6 mL) were added Na₂CO₃ (200 mg, 1.89 mmol, 5 eq.) and Pd(dppf)Cl₂ (27.6 mg, 0.0380 mmol, 0.1 eq.). After stirring for 4 h at 80° C. under a nitrogen atmosphere, the resulting mixture was concentrated under reduced pressure. The residue was purified by reversed-phase flash chromatography to afford the title compound.

LCMS (ES) m/z: [M+H]⁺: 396.0, 1H NMR (300 MHz, DMSO-d6, ppm) δ 12.13 (s, 1H), 10.86 (s, 1H), 9.03-8.93 (m, 1H), 8.69-8.60 (m, 2H), 8.26-8.14 (m, 2H), 7.51 (dd, J=8.2, 4.8 Hz, 1H), 6.85 (d, J=7.9 Hz, 1H), 6.75 (d, J=7.8 Hz, 1H), 2.60 (s, 3H).

General Method D

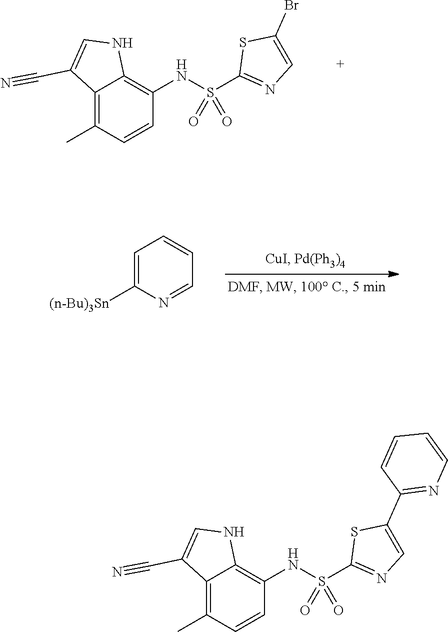

Synthesis of N-(3-cyano-4-methyl-1H-indol-7-yl)-5-(pyridin-2-yl)-1,3-thiazole-2-sulfonamide. To a solution of 5-bromo-N-(3-cyano-4-methyl-1H-indol-7-yl)thiazole-2-sulfonamide and 4-(tributylstannyl)pyridine (741 mg, 2.01 mmol, 4 equiv) in DMF (5 mL) was added CuI (28.8 mg, 0.151 mmol, 0.3 equiv) and Pd(PPh 3) 4 (116 mg, 0.101 mmol, 0.2 equiv) at room temperature under nitrogen atmosphere. The resulting mixture was stirred for 5 min at 100° C. in the Microwave irradiation. The mixture was allowed to cool down to room temperature. The residue was purified by reversed-phase flash chromatography to afford the title compound.

LCMS (ES) m/z): [M+H]⁺: 396.0, ¹H NMR (300 MHz, DMSO-d₆, ppm) δ 12.13 (d, J=3.2 Hz, 1H), 10.82 (s, 1H), 8.77 (s, 1H), 8.59 (dt, J=4.8, 1.5 Hz, 1H), 8.23 (d, J=3.0 Hz, 1H), 8.17-8.07 (m, 1H), 7.96 (td, J=7.8, 1.7 Hz, 1H), 7.43 (ddd, J=7.5, 4.9, 1.1 Hz, 1H), 6.85 (d, J=7.8 Hz, 1H), 6.74 (d, J=7.7 Hz, 1H), 2.59 (s, 3H).

General Procedure E

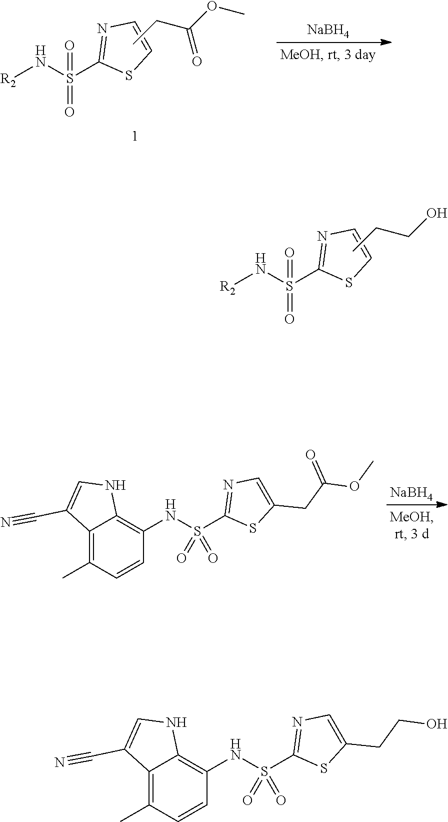

Synthesis of N-(3-cyano-4-methyl-1H-indol-7-yl)-5-(2-hydroxyethyhthiazole-2-sulfonamide. To a solution of methyl 2-(2-(N-(3-cyano-4-methyl-1H-indol-7-yl)sulfamoyl)thiazol-5-yl)acetate (0.38 mmol, 1.00 equiv) in MeOH (3 mL) was added NaBH 4 (11.5 mmol, 30 equiv) at 0° C. and the resulting mixture was stirred for additional 3 days at room temperature. The residue was purified by prep HPLC/achiral SFC method to afford the corresponding final compound.

LCMS: (LCMS (ES) m/z): [M+1]⁺: 363, ¹H NMR (300 MHz, DMSO-d₆, ppm) δ 12.05 (d, J=3.2 Hz, 1H), 10.63 (s, 1H), 8.20 (d, J=3.0 Hz, 1H), 7.85 (s, 1H), 6.84 (d, J=7.8 Hz, 1H), 6.72 (d, J=7.7 Hz, 1H), 5.01 (t, J=5.0 Hz, 1H), 3.59 (q, J=5.6 Hz, 2H), 3.00 (t, J=6.0 Hz, 2H), 2.60 (s, 3H).

General Procedure F

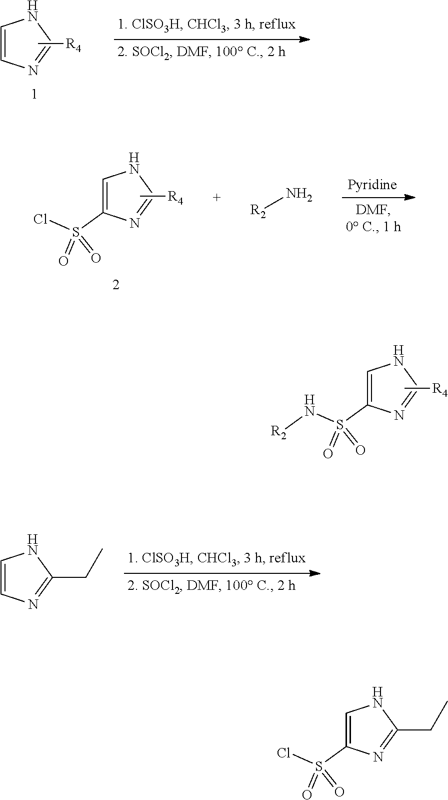

Synthesis of 2-ethyl-1H-imidazole-4-sulfonyl chloride. To a solution of 2-ethyl-1H-imidazole (20 g, 208 mmol, 1 equiv) in CHCl₃ (1600 mL) was added and chlorosulfonic acid (80 mL) and the resulting mixture was stirred overnight at reflux under nitrogen atmosphere. To the above mixture was added SOCl₂ (600 mL) and the resulting mixture was stirred for additional 2 h at 100° C. and then was concentrated under reduced pressure. The above mixture was poured into ice water. The resulting mixture was extracted with DCM (4×150 mL), dried over anhydrous Na₂SO₄. After filtration, the filtrate was concentrated under reduced pressure to afford the title compound which was used in next step without any further purification.

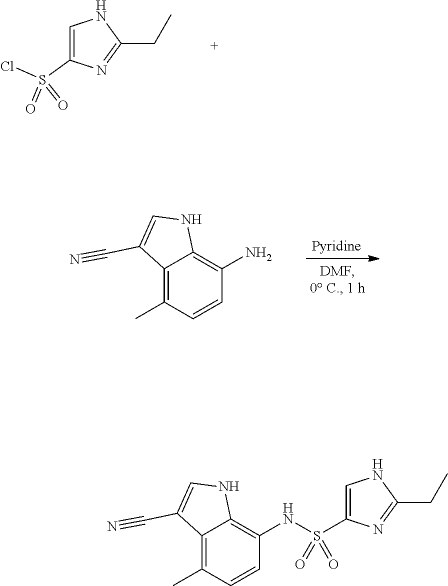 [SMILES: CCc1nc(S(=O)(=O)Cl)c[nH]1 ; Cc1ccc(N)c2[nH]cc(C#N)c12 ; CCc1nc(S(=O)(=O)Nc2ccc(C)c3c(C#N)c[nH]c23)c[nH]1]

Synthesis of N-(3-cyano-4-methyl-1H-indol-7-yl)-2-ethyl-1H-imidazole-4-sulfonamide. To a solution of 7-amino-4-methyl-1H-indole-3-carbonitrile (40.8 mmol, 1 equiv) and pyridine (122 mmol, 3 equiv) in DCM (100 mL) was added 2-ethyl-1H-imidazole-4-sulfonyl chloride (49.0 mmol, 1.2 equiv) in DCM (20 mL) dropwise at 0° C. The resulting mixture was stirred for 1 h at room temperature under nitrogen atmosphere. The resulting mixture was concentrated under reduced pressure. The residue was purified by reverse-phase chromatography to afford the title compound.

LCMS (LCMS (ES) m/z): [M+1]⁺: 330, ¹H NMR (400 MHz, DMSO-d₆, ppm) δ 12.41 (s, 1H), 12.16-12.11 (m, 1H), 9.77 (s, 1H), 8.20 (d, J=3.0 Hz, 1H), 7.55 (d, J=1.7 Hz, 1H), 6.81 (s, 2H), 2.65 (q, J=7.6 Hz, 2H), 2.57 (s, 3H), 1.20 (t, J=7.6 Hz, 3H).

General Procedure G

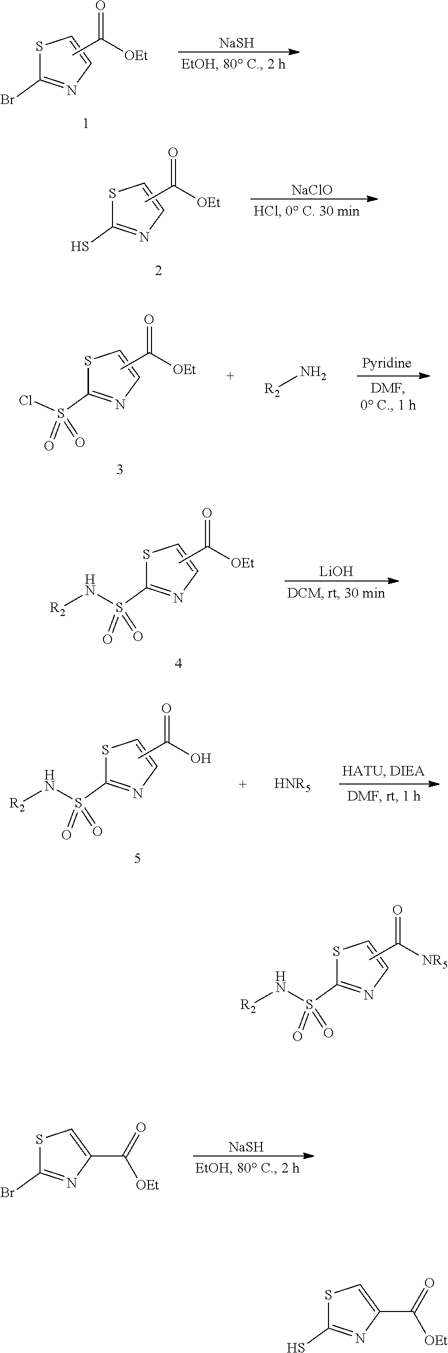

Synthesis of ethyl 2-mercaptothiazole-4-carboxylate. To a stirred solution of ethyl 2-bromo-1,3-thiazole-4-carboxylate (2.5 g, 10.590 mmol, 1 equiv) in EtOH (50.00 mL) was added the mixture of NaSH (2.97 g, 53.0 mmol, 5 equiv) in EtOH (25 mL) dropwise at 80° C. The resulting mixture was stirred for 2 h at 80° C. The resulting mixture was concentrated under reduced pressure. The residue was dissolved in water (50 mL) and was acidified to pH 3 with HCl (aq.). The precipitated solids were collected by filtration and washed with water (3×5 mL) to afford the title compound which was used in next step without any further purification.

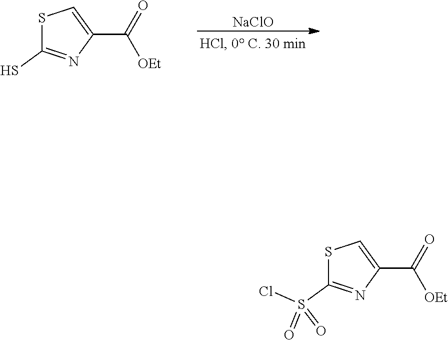 [SMILES: CCOC(=O)c1csc(S)n1 ; "NaClO" ; CCOC(=O)c1csc(S(=O)(=O)Cl)n1]

Synthesis of ethyl 2-(chlorosulfonyhthiazole-4-carboxylate. To a solution of ethyl 2-sulfanyl-1,3-thiazole-4-carboxylate (1.40 g, 7.40 mmol, 1 equiv) in conc.HCl (14 mL) was added NaClO (14.3 mL, 192 mmol, 26 equiv, 10% w.t in water) dropwise at 0° C. The resulting mixture was stirred for 30 min at 0° C. and 15 min at room temperature. The precipitated solids were collected by filtration and washed with water (3×5 mL) to afford the title compound which was used in next step without any further purification.

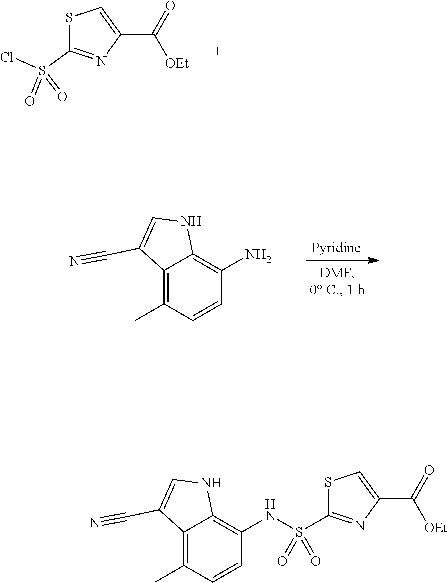 [SMILES: CCOC(=O)c1csc(S(=O)(=O)Cl)n1 ; Cc1ccc(N)c2[nH]cc(C#N)c12 ; CCOC(=O)c1csc(S(=O)(=O)Nc2ccc(C)c3c(C#N)c[nH]c23)n1]

Synthesis of ethyl 2-(N-(3-cyano-4-methyl-1H-indol-7-yl)sulfamoyl)thiazole-4-carboxylate. To a solution of 7-amino-4-methyl-1H-indole-3-carbonitrile (0.83 g, 4.85 mmol, 1 equiv) and pyridine (1.15 g, 14.5 mmol, 3 equiv) in DCM (25 mL) was treated with ethyl 2-(chlorosulfonyl)-1,3-thiazole-4-carboxylate (1.24 g, 4.85 mmol, 1 equiv) (146-2) in DCM (10 mL) at 0° C. The resulting mixture was stirred for 2 h at room temperature. The residue was concentrated under vacuum, purified by silica gel chromatography to afford the title compound.

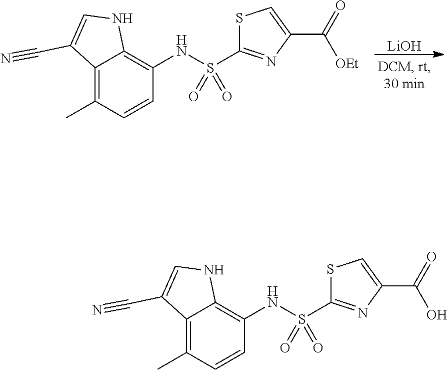

Synthesis of 2-(N-(3-cyano-4-methyl-1H-indol-7-yl)sulfamoyl)thiazole-4-carboxylic acid. To a solution of ethyl 2-[(3-cyano-4-methyl-1H-indol-7-yl)sulfamoyl]-1,3-thiazole-4-carboxylate (200 mg, 0.51 mmol, 1 equiv) in THF (2 mL) was added 1 M LiOH in water (2 mL, 2.00 mmol, 3.90 equiv) dropwise at room temperature and the resulting mixture was stirred for 30 min at room temperature. The resulting mixture was washed with DCM (1×10 mL) and the aqueous layer was acidified to pH 3 with 1 M HCl. The precipitated solids were collected by filtration and washed with water (3×3 mL). The material was purified on reverse-phase chromatography to afford the title compound.

LCMS: (LCMS (ES) m/z): [M+1]⁺: 363, ¹H NMR (300 MHz, DMSO-d₆, ppm) 13.56 (s, 1H), 12.18 (d, J=3.1 Hz, 1H), 10.91 (s, 1H), 8.71 (s, 1H), 8.22 (d, J=3.1 Hz, 1H), 6.85 (dd, J=7.7, 1.0 Hz, 1H), 6.64 (d, J=7.7 Hz, 1H), 2.60 (s, 3H).

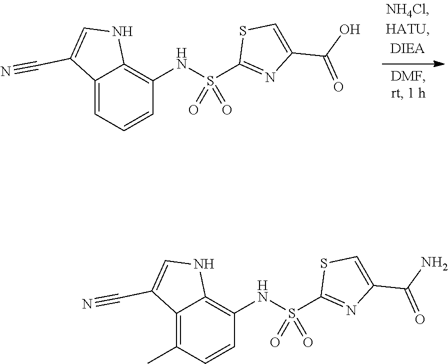 [SMILES: N#Cc1c[nH]c2c(NS(=O)(=O)c3nc(C(=O)O)cs3)cccc12 ; Cc1ccc(NS(=O)(=O)c2nc(C(N)=O)cs2)c2[nH]cc(C#N)c12]

Synthesis of 2-(N-(3-cyano-4-methyl-1H-indol-7-yl)sulfamoyl)thiazole-4-carboxamide. To a solution of 2-[(3-cyano-4-methyl-1H-indol-7-yl) sulfamoyl]-1,3-thiazole-4-carboxylic acid (0.41 mmol, 1 equiv) in DMF was added DIEA (2.07 mmol, 5.00 equiv), ammonium chloride (5 equiv) and HATU (0.62 mmol, 1.5 equiv) and the resulting mixture was stirred for additional 1 h at room temperature. Crude material was purified by reversed-phase chromatography to afford the title compound.

LCMS (LCMS (ES) m/z): [M+1]⁺: 362, ¹H NMR (400 MHz, DMSO-d₆, ppm) δ 12.12 (d, J=3.0 Hz, 1H), 10.86 (s, 1H), 8.55 (s, 1H), 8.21 (d, J=3.2 Hz, 1H), 7.81 (s, 1H), 7.71 (s, 1H), 6.87 (d, J=7.8 Hz, 1H), 6.75 (d, J=7.7 Hz, 1H), 2.61 (s, 3H).

General Procedure H

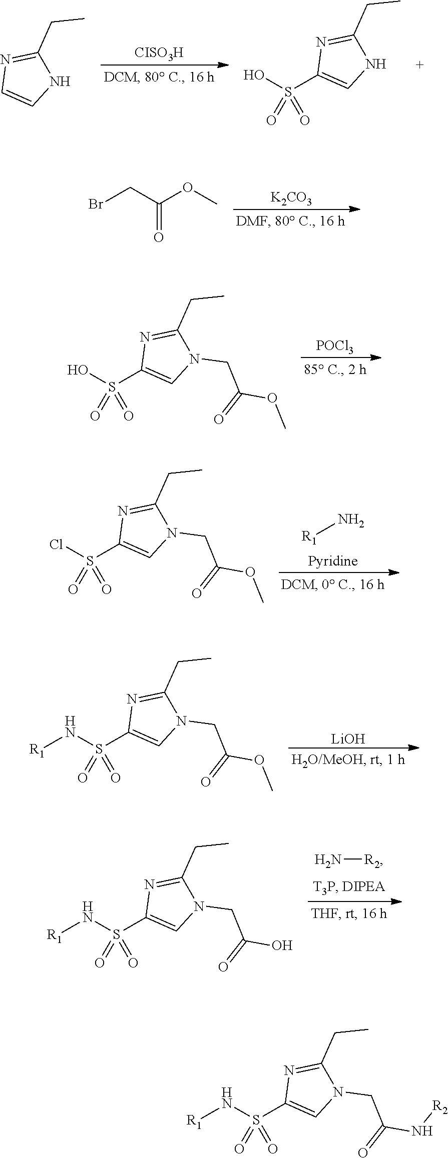

Preparation of 2-ethyl-1H-imidazole-4-sulfonic acid. To a solution of 2-ethyl-1H-imidazole (2 g, 20.8 mmol, 1 eq) in CHCl₃ (16.0 mL) was added HSO₃Cl (8 mL) and the resulting mixture was stirred overnight at 80° C. under nitrogen atmosphere. The resulting mixture was concentrated under vacuum and was diluted with Et₂O (200 mL) and EtOH (20 mL) at room temperature and the resulting mixture was stirred for 0.5 h at room temperature. The resulting mixture was filtered, the filter cake was washed with Et₂O, and was dried under vacuum to give the crude product which was used without further purification.

Preparation of 2-ethyl-1-(2-methoxy-2-oxoethyl)imidazole-4-sulfonic acid. To a solution of 2-ethyl-1H-imidazole-4-sulfonic acid (3.30 g, 18.7 mmol, 1 eq) in DMF (66.0 mL) was added methyl 2-bromoacetate (3.44 g, 22.4 mmol, 1.2 eq) and K₂CO₃ (7.77 g, 56.1 mmol, 3 eq) and the resulting mixture was stirred overnight at 80° C. under nitrogen atmosphere. The reaction mixture diluted with MeOH, filtered, acidified with HCl in MeOH(4M, 40 mL), concentrated under vacuum to give the crude product which was used without further purification.

Preparation of 2-[4-(chlorosulfonyl)-2-ethylimidazol-1-yl]acetate. A solution of 2-ethyl-1-(2-methoxy-2-oxoethyl)imidazole-4-sulfonic acid (6.30 g, 25.3 mmol, 1 eq) in POCl₃ (126 mL) was stirred at 85° C. for 2 h. The mixture was concentrated under reduced pressure, diluted with water, and extracted with DCM. The combined organic layers were washed with brine, dried over anhydrous Na₂SO₄, and concentrated under reduced pressure to give the crude product which was used without further purification. LCMS (ES, m/z): [M+1]+: 267

Preparation of methyl 2-{4-[(3-cyano-4-methyl-1H-indol-7-yl)sulfamoyl]-2-ethylimidazol-1-yl}acetate. To a solution of 7-amino-4-methyl-1H-indole-3-carbonitrile (0.45 g, 2.62 mmol, 1 eq) and pyridine (0.62 g, 7.88 mmol, 3 eq) in DCM (14 mL) was added methyl 2-[4-(chlorosulfonyl)-2-ethylimidazol-1-yl]acetate (0.70 g, 2.62 mmol, 1 eq) in DCM (4 mL) dropwise at 0° C. and the resulting mixture was stirred overnight at room temperature. The reaction mixture was concentrated under vacuum. The crude residue was purified by silica gel column chromatography to give the product. LCMS (ES, m/z): [M+1]+: 402

Preparation of {4-[(3-cyano-4-methyl-1H-indol-7-yl)sulfamoyl]-2-ethylimidazol-1-yl}acetic acid. To a solution of methyl 2-{4-[(3-cyano-4-methyl-1H-indol-7-yl)sulfamoyl]-2-ethylimidazol-1-yl}acetate (0.50 g, 1.24 mmol, 1 eq) in MeOH (10 mL) and H₂O (10 mL) was added LiOH (0.03 g, 1.24 mmol, 1 eq) and the resulting mixture was stirred for 1 h at room temperature. The resulting mixture was concentrated under vacuum and the crude residue was purified by reversed-phase flash chromatography to give the product. LCMS (ES, m/z): [M+1]+: 388, ¹H NMR (300 MHz, DMSO-d₆, ppm) δ 13.32 (s, 1H), 12.15 (s, 1H), 9.85 (s, 1H), 8.20 (d, J=3.1 Hz, 1H), 7.63 (s, 1H), 6.81 (s, 2H), 4.83 (s, 2H), 2.59 (d, J=7.4 Hz, 5H), 1.21 (t, J=7.5 Hz, 3H).

Preparation of 2-(4-(N-(3-cyano-4-methyl-1H-indol-7-yl)sulfamoyl)-2-ethyl-1H-imidazol-1-yl)-N-cyclopropylacetamide. To a solution of {4-[(3-cyano-4-methyl-1H-indol-7-yl)sulfamoyl]-2-ethylimidazol-1-yl}acetic acid (150 mg, 0.38 mmol, 1 eq) and aminocyclopropane (111 mg, 1.93 mmol, 5 eq) in THF (3 mL) was added T₃P (185 mg, 0.58 mmol, 1.50 eq) and DIPEA (251 mg, 1.93 mmol, 5 eq) and the resulting mixture was stirred overnight at room temperature. The residue was purified by reversed-phase flash chromatography to give the product. LCMS (ES, m/z): [M+1]+: 426, ¹H NMR (300 MHz, DMSO-d₆, ppm) δ 12.18 (s, 1H), 9.85 (s, 1H), 8.34 (d, J=4.1 Hz, 1H), 8.20 (d, J=2.8 Hz, 1H), 7.61 (s, 1H), 6.90-6.77 (m, 2H), 4.57 (s, 2H), 2.69-2.51 (m, 6H), 1.20 (t, J=7.5 Hz, 3H), 0.76-0.58 (m, 2H), 0.47-0.35 (m, 2H).

General Procedure I

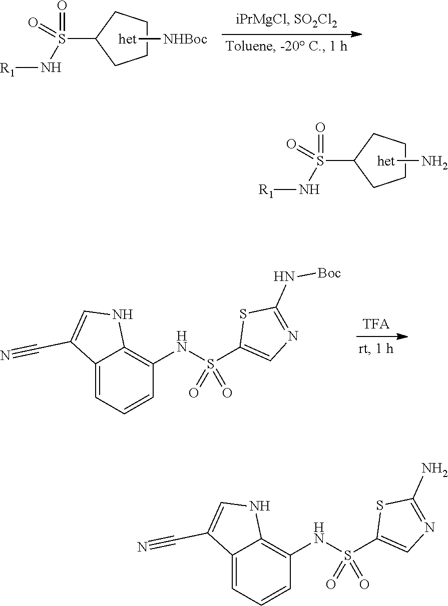

Preparation of 7-(2-amino-1,3-thiazol-5-ylsulfonylamino)-3-indolecarbonitrile. A solution of tert-butyl N-{5-[(3-cyano-1H-indol-7-yl)sulfamoyl]-1,3-thiazol-2-yl}carbamate (200 mg, 0.48 mmol, 1 eq) in TFA (4 mL) was stirred at room temperature for 1 h. The resulting mixture was concentrated under reduced pressure. The crude residue was purified by reversed-phase flash chromatography to afford the title compound. LCMS (ES) m/z: [M+1]+: 320, ¹H NMR (400 MHz, DMSO-d₆, ppm) δ 11.87 (d, J=3.1 Hz, 1H), 10.01 (s, 1H), 8.22 (d, J=3.1 Hz, 1H), 7.85 (s, 2H), 7.47 (d, J=7.9 Hz, 1H), 7.30 (s, 1H), 7.18 (t, J=7.8 Hz, 1H), 7.02 (d, J=7.7 Hz, 1H).

General Procedure N

### Preparation of Methyl 2-((5-(chlorosulfonyl)thiazol-2-yl)oxy)acetate

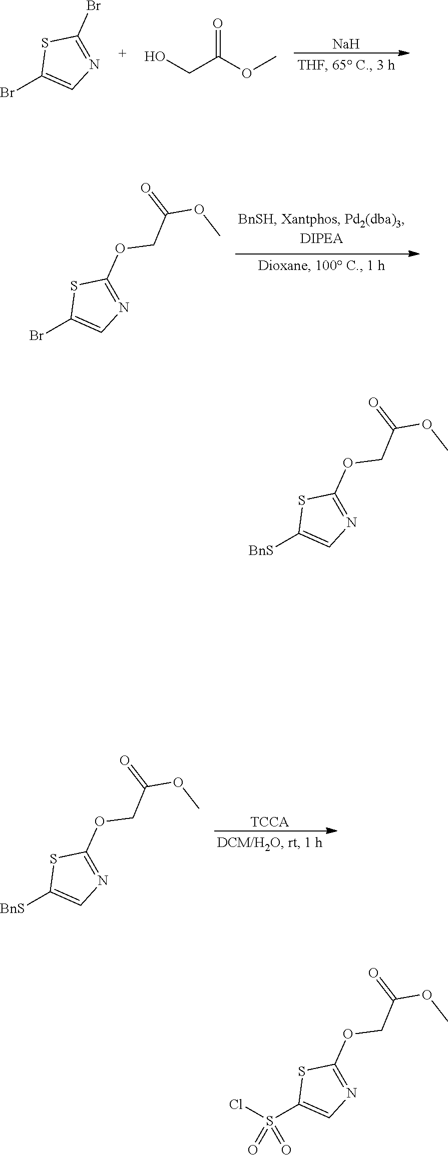

Preparation of methyl 2-[(5-bromo-1,3-thiazol-2-yl)oxy]acetate. To a solution of methyl 2-hydroxyacetate (1.11 g, 12.3 mmol, 1.5 eq) in THF (40 mL) was added NaH (0.41 g, 17.2 mmol, 2.1 eq) and 2,5-dibromo-1,3-thiazole (2 g, 8.23 mmol, 1 eq) at 0° C. and the resulting mixture was stirred for 3 h at 65° C. The reaction was quenched with water and extracted with EtOAc. The combined organic layers were washed with brine, dried over Na₂SO₄, and concentrated under reduced pressure. The crude residue was purified by reversed-phase flash chromatography to give the product. LCMS (ES, m/z): [M+1]⁺: 252

Preparation of methyl 2-{[5-(benzylsulfanyl)-1,3-thiazol-2-yl]oxy}acetate. To a solution of methyl 2-[(5-bromo-1,3-thiazol-2-yl)oxy]acetate (400 mg, 1.58 mmol, 1.0 eq) in dioxane (8 mL) was added XantPhos (91.8 mg, 0.159 mmol, 0.1 eq), Pd₂(dba)₃ (72.6 mg, 0.079 mmol, 0.05 eq), benzyl mercaptan (236 mg, 1.90 mmol, 1.2 eq) and DIPEA (615 mg, 4.76 mmol, 3 eq) and the resulting mixture was stirred for 1 h at 100° C. under nitrogen atmosphere. The reaction was quenched with water and extracted with EtOAc. The combined organic layers were washed with brine, dried over Na₂SO₄, and concentrated under reduced pressure. The crude residue was purified by reversed-phase flash chromatography to give the product. LCMS (ES, m/z): [M+1]⁺: 296

Preparation of methyl 2-((5-(chlorosulfonyl)thiazol-2-yl)oxy)acetate. To a solution of methyl 2-{[5-(benzylsulfanyl)-1,3-thiazol-2-yl]oxy}acetate (420 mg, 1.42 mmol, 1.0 eq) in DCM (3 mL) and H₂O (7 mL) was added 1,3,5-trichloro-1,3,5-triazinane-2,4,6-trione (363 mg, 1.56 mmol, 1.1 eq) at 0° C. and the resulting mixture was stirred for 1 h at room temperature. The reaction was quenched with water and extracted with DCM. The combined organic layers were washed with brine, dried over Na₂SO₄, and concentrated under reduced pressure to give the product which was used without further purification. LCMS (ES, m/z): [M+1]⁺: 272

General Procedure J

### Preparation of Methyl 2-((5-(N-(3-cyano-1H-indol-7-yl)sulfamoyl)thiazol-2-yl)oxy)acetate & N-(3-cyano-1H-indol-7-yl)-2-(2-hydroxy-2-methylpropoxy)thiazole-5-sulfonamide

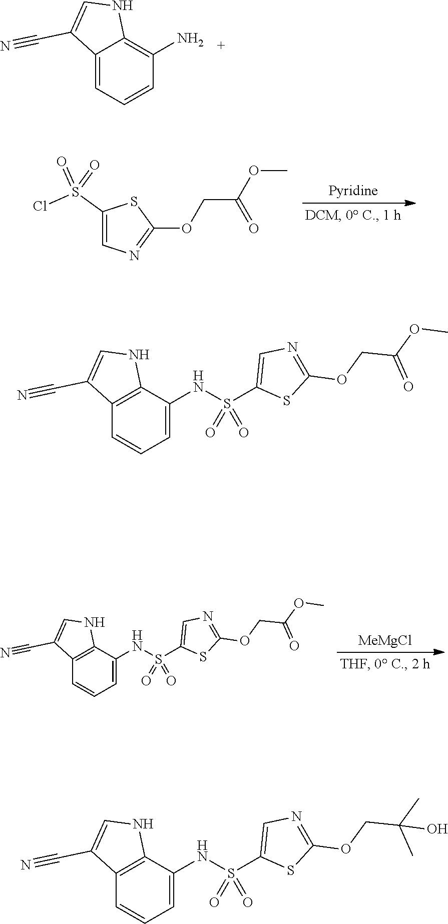

Preparation of methyl 2-((5-(N-(3-cyano-1H-indol-7-yl)sulfamoyl)thiazol-2-yl)oxy)acetate Methyl 2-((5-(chlorosulfonyl)thiazol-2-yl)oxy)acetate and 7-amino-1H-indole-3-carbonitrile (150 mg, 0.954 mmol, 1 eq) were reacted in the presence of pyridine in a dichloromethane solution at 0° C. for 1 h to form the sulfonamide intermediate. The intermediate was purified by reversed-phase flash chromatography with the following conditions: column, C18 silica gel; mobile phase, MeCN in Water (0.1% FA), 10% to 50% gradient in 10 min; detector, UV 254 nm. The resulting mixture was concentrated under reduced pressure to afford the title compound. LCMS (ES, m/z): [M+1]⁺: 393, ¹H NMR (400 MHz, DMSO-d₆, ppm) δ 12 (s, 1H), 10.41 (s, 1H), 8.22 (d, J=3.0 Hz, 1H), 7.60-7.48 (m, 2H), 7.17 (t, J=7.8 Hz, 1H), 6.94 (d, J=7.6 Hz, 1H), 5.12 (s, 2H), 3.69 (s, 3H).

Preparation of N-(3-cyano-1H-indol-7-yl)-2-(2-hydroxy-2-methylpropoxy)thiazole-5-sulfonamide The sulfonamide intermediate from above was reacted with MeMgCl in THE at 0° C. for 2 h to provide the desired compound. The compound was purified by reversed-phase flash chromatography with the following conditions: column, C18 silica gel; mobile phase, MeCN in Water (0.1% FA), 10% to 50% gradient in 10 min; detector, UV 254 nm. The resulting mixture was concentrated under reduced pressure to afford the title compound.

LCMS (ES, m/z): [M+1]⁺: 391, ¹H NMR (400 MHz, DMSO-d₆, ppm) δ 11.99 (d, J=3.1 Hz, 1H), 10.38 (s, 1H), 8.22 (d, J=3.1 Hz, 1H), 7.60-7.48 (m, 2H), 7.18 (t, J=7.8 Hz, 1H), 6.96 (dd, J=7.7, 1.0 Hz, 1H), 4.81 (s, 1H), 4.18 (s, 2H), 1.14 (s, 6H).

### Preparation of N-(3-cyano-1H-indol-7-yl)-3-(2-hydroxy-2-methylpropyl)-2-oxo-2,3-dihydrothiazole-5-sulfonamide

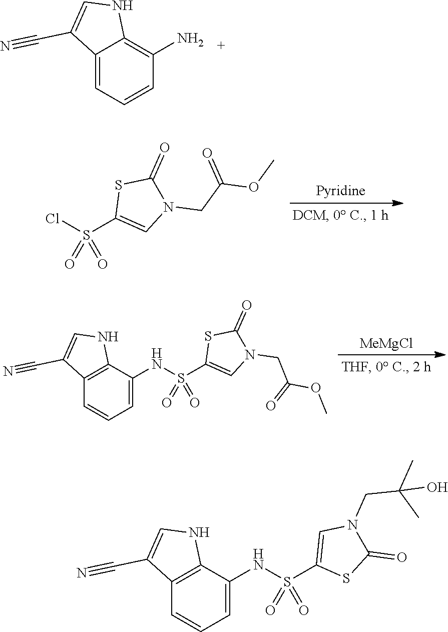

Methyl 2-(5-(chlorosulfonyl)-2-oxothiazol-3(2H)-yl)acetate and 7-amino-1H-indole-3-carbonitrile (100 mg, 0.636 mmol, 1 eq) were reacted in a fashion similar to that described above. The intermediate was purified by reversed-phase flash chromatography with the following conditions: column, C18 silica gel; mobile phase, MeCN in Water (0.1% FA), 10% to 50% gradient in 10 min; detector, UV 254 nm. The resulting mixture was concentrated under reduced pressure to afford the desired compound. LCMS (ES, m/z): [M+1]⁺: 391, ¹H NMR (400 MHz, DMSO-d₆, ppm) δ 11.99 (s, 1H), 10.32 (s, 1H), 8.22 (d, J=2.8 Hz, 1H), 7.56-7.44 (m, 2H), 7.18 (t, J=7.8 Hz, 1H), 7.03 (d, J=7.5 Hz, 1H), 4.67 (s, 1H), 3.57 (s, 2H), 0.96 (s, 6H).

Preparation of Intermediate Sulfonyl Chlorides

Preparation of 5-(2-hydroxy-2-methylpropyl)-1,3-thiazole-2-sulfonyl chloride

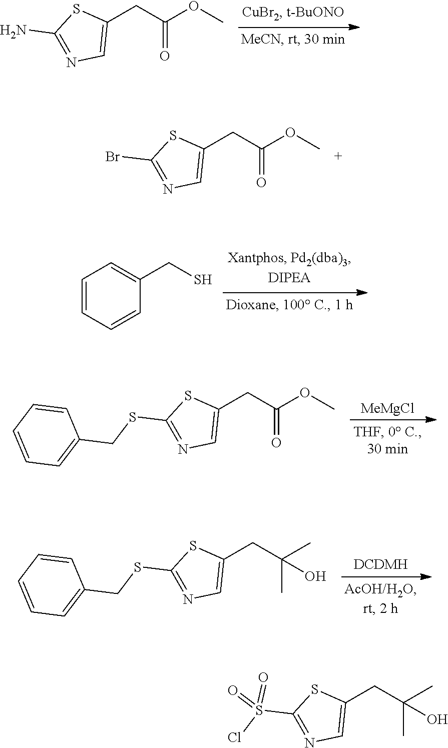

Preparation of methyl 2-(2-bromo-1,3-thiazol-5-yl) acetate. To a solution of methyl 2-(2-amino-1,3-thiazol-5-yl) acetate (700 mg, 4.06 mmol, 1 eq) and CuBr₂ (998 mg, 4.47 mmol, 1.1 eq) in MeCN (35 mL) was added t-BuONO (628 mg, 6.09 mmol, 1.5 eq) dropwise at −10° C. and the resulting mixture was stirred for 30 min at room temperature. The resulting mixture was extracted with DCM. The combined organic layers were dried over anhydrous Na₂SO₄ and concentrated under reduced pressure. The crude residue was purified by reversed-phase flash chromatography to give the product. LCMS: (ES, m/z): [M+1]+: 236

Preparation of methyl 2-[2-(benzylsulfanyl)-1,3-thiazol-5-yl]acetate. To a solution of methyl 2-(2-bromo-1,3-thiazol-5-yl)acetate (356 mg, 1.50 mmol, 1 eq) and benzyl mercaptan (224 mg, 1.81 mmol, 1.2 eq) in dioxane (7 mL) was added DIPEA (584 mg, 4.52 mmol, 3 eq), XantPhos (87.3 mg, 0.15 mmol, 0.1 eq), and Pd₂(dba)₃ (69.0 mg, 0.07 mmol, 0.05 eq) and the resulting mixture was stirred for 1 h at 100° C. under nitrogen atmosphere. The reaction was quenched with water and was extracted with DCM. The combined organic layers washed with brine, dried over anhydrous Na₂SO₄, and concentrated under reduced pressure. The crude residue was purified by reversed-phase flash chromatography to give the product. LCMS: (ES, m/z): [M+1]⁺: 280

Preparation of 1-[2-(benzylsulfanyl)-1,3-thiazol-5-yl]-2-methylpropan-2-ol. To a solution of methyl 2-[2-(benzylsulfanyl)-1,3-thiazol-5-yl]acetate (400 mg, 1.43 mmol, 1 eq) in THF (8 mL) was added CH₃MgCl (1.5 mL, 4.29 mmol, 3.0 eq) dropwise at 0° C. and the resulting mixture was stirred for 30 min at 0° C. under nitrogen atmosphere. The reaction was quenched with sat. NH₄Cl and was extracted with DCM. The combined organic layers washed with brine, dried over anhydrous Na₂SO₄, and concentrated under reduced pressure. The crude residue was purified by reversed-phase flash chromatography to give the product. LCMS: (ES, m/z): [M+1]⁺: 280

Preparation of 5-(2-hydroxy-2-methylpropyl)-1,3-thiazole-2-sulfonyl chloride. To a solution of 1-[2-(benzylsulfanyl)-1,3-thiazol-5-yl]-2-methylpropan-2-ol (202 mg, 0.72 mmol, 1 eq) in AcOH (2 mL) and H₂O (2 mL) was added 1,3-dichloro-5,5-dimethylimidazolidine-2,4-dione (285 mg, 1.44 mmol, 2 eq) and the resulting mixture was stirred for 2 h at 40° C. under nitrogen atmosphere. The reaction was quenched with water at room temperature and extracted with DCM. The combined organic layers were washed with brine, dried over anhydrous Na₂SO₄, and concentrated under reduced pressure to give the crude product which was used without further purification.

### Preparation of oxazole-5-sulfonyl chloride

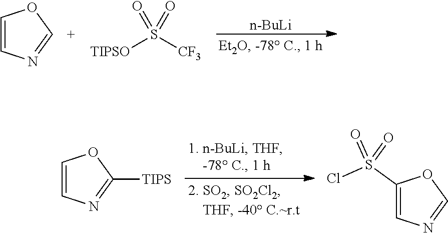

Preparation of 2-(triisopropylsilyl)oxazole. To a solution of oxazole (1.0 g, 14.4 mmol, 1.0 eq) in Et₂O (20 mL) was added n-BuLi (12.5 mL, 132 mmol, 1.1 eq, 2.5M) dropwise at −78° C. and the resulting mixture was stirred for 1 h at −78° C. under nitrogen atmosphere. To the mixture was added triisopropylsilyl trifluoromethanesulfonate (4.44 g, 14.4 mmol, 1.0 eq) at −78° C. and the resulting mixture was stirred for 1 h at room temperature. The reaction was quenched with water and was extracted with EtOAc. The combined organic layers were washed with brine, dried over anhydrous Na₂SO₄, and concentrated under reduced pressure to give the crude product which was used without further purification. LCMS: (ES, m/z): [M+1]⁺: 226

Preparation of oxazole-5-sulfonyl chloride. To a solution of 2-(triisopropylsilyl)oxazole (2.0 g, 8.87 mmol, 1.0 eq) in THE (40 mL) was added n-BuLi (4.2 mL, 9.76 mmol, 1.1 eq, 2.5M) dropwise at −78° C. and the resulting mixture was stirred for 1 h at −78° C. under nitrogen atmosphere. To the mixture was added SO₂ and SO₂Cl₂ (2.39 g, 17.7 mmol, 2.0 eq) dropwise over 3 min at −40° C. and the resulting mixture was stirred for 2 h at room temperature. The reaction was quenched with water and was extracted with DCM. The combined organic layers were washed with brine, dried over anhydrous Na₂SO₄, and concentrated under reduced pressure to give the crude product which was used without further purification. LCMS: (ES, m/z): [M+1]⁺: 168

### Preparation of 2-fluorothiazole-5-sulfonyl Chloride

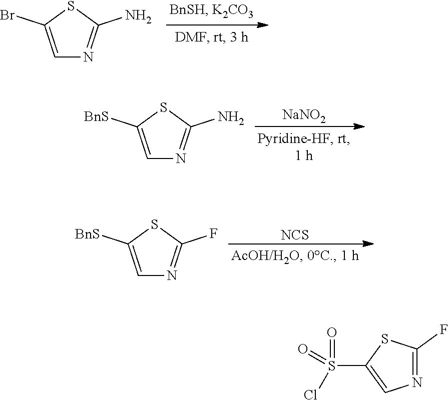

Preparation of 5-(benzylsulfanyl)-1,3-thiazol-2-amine. To a solution of 5-bromo-1,3-thiazol-2-amine (30.0 g, 167 mmol, 1.0 eq) and benzyl mercaptan (25.0 g, 201 mmol, 1.2 eq) in DMF (300 mL) was added K₂CO₃ (34.7 g, 251 mmol, 1.5 eq) and the resulting mixture was stirred for 3 h at room temperature. The resulting mixture was quenched with water and extracted with EtOAc. The combined organic layers were washed with brine, dried over anhydrous Na₂SO₄, and concentrated under reduced pressure. The residue was purified by silica gel column chromatography to give the product. LCMS (ES, m/z): [M+1]⁺: 223.

Preparation of 5-(benzylsulfanyl)-2-fluoro-1,3-thiazole. A solution of 5-(benzylsulfanyl)-1,3-thiazol-2-amine (10.0 g, 45.0 mmol, 1.0 eq) in pyridine hydrofluoride (100 mL) was stirred for 1 h at 0° C. under a nitrogen atmosphere. To the mixture was added NaNO₂ (3.7 g, 54.0 mmol, 1.2 eq) and the resulting mixture was stirred for 1 h at room temperature. The resulting mixture was quenched with water and extracted with EtOAc. The combined organic layers were washed with brine, dried over anhydrous Na₂SO₄, and concentrated under reduced pressure. The crude product was purified by reverse phase flash chromatography to give the product. LCMS (ES, m/z): [M+1]⁺: 226.

Preparation of 2-fluorothiazole-5-sulfonyl chloride. To a solution of 5-(benzylsulfanyl)-2-fluoro-1,3-thiazole (900 mg, 4.0 mmol, 1.0 eq) in AcOH (9 mL) and H₂O (4.5 mL) was added NCS (1600 mg, 12.0 mmol, 3.0 eq) and the resulting mixture was stirred for 1 h at room temperature under a nitrogen atmosphere. The resulting mixture was extracted with DCM. The combined organic layers were washed with brine, dried over anhydrous Na₂SO₄, and concentrated under reduced pressure to give the crude product which was used without further purification. LCMS (ES, m/z): [M+1]⁺: 202.

### Preparation of 2-substituted 1,3-oxazole-4-sulfonyl Chlorides

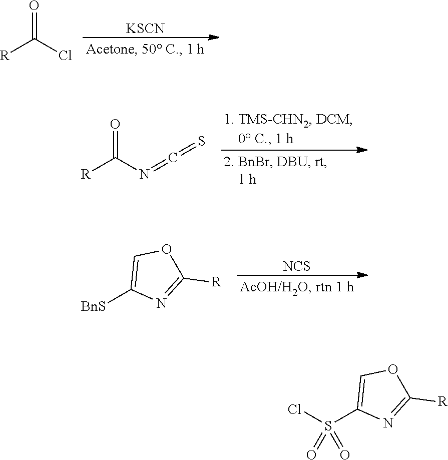

Preparation of propanecarbonyl isothiocyanate. To a solution of propanoyl chloride (6.0 g, 64.9 mmol, 1.0 eq) in acetone (60 mL) was added potassium thiocyanate (6.3 g, 64.8 mmol, 1.0 eq) and the resulting mixture was stirred for 1 h at 50° C. under a nitrogen atmosphere. The resulting mixture was concentrated under reduced pressure to give the crude product which was without further purification. LCMS (ES, m/z): [M+1]⁺: 116.

Preparation of 4-(benzylsulfanyl)-2-ethyl-1,3-oxazole. To a solution of propanecarbonyl isothiocyanate (313-1) (7.3 g, 63.4 mmol, 1.0 eq) in DCM (73 mL) was added TMS-CHN₂ (7.2 g, 63.4 mmol, 1.0 eq) and the resulting mixture was stirred for 1 h at 0° C. under a nitrogen atmosphere. To the mixture was added DBU (19.3 g, 126 mmol, 2.0 eq) and BnBr (10.8 g, 63.4 mmol, 1.0 eq) and the resulting mixture was stirred for 1 h at room temperature. The reaction mixture was quenched with water and was extracted with DCM. The combined organic layers were washed with brine, dried over anhydrous Na₂SO₄, and was concentrated under reduced pressure. The crude product was purified by reverse phase flash chromatography to give the product. LCMS (ES, m/z): [M+1]⁺: 220.

Preparation of 2-ethyl-1,3-oxazole-4-sulfonyl chloride. To a solution of 4-(benzylsulfanyl)-2-ethyl-1,3-oxazole (1.3 g, 5.9 mmol, 1.0 eq) in AcOH (13 mL) and H₂O (6.5 mL) was added NCS (2.8 g, 20.7 mmol, 3.5 eq) and the resulting mixture was stirred for 1 h at room temperature under a nitrogen atmosphere. The resulting mixture was extracted with DCM. The combined organic layers were washed with brine, dried over anhydrous Na₂SO₄, and concentrated under reduced pressure to give the crude product which was used without further purification. LCMS (ES, m/z): [M+1]⁺: 196.

### Preparation of 5-methylisoxazole-3-sulfonyl Chloride

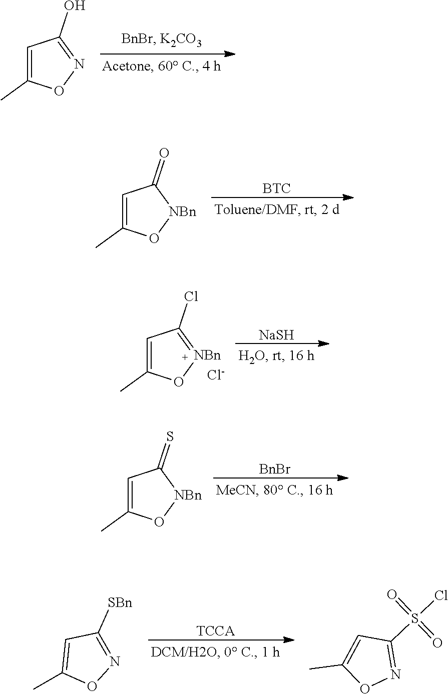

Preparation of 2-benzyl-5-methyl-1,2-oxazol-3-one. To a solution of 3-hydroxy-5-methylisoxazole (10.0 g, 101 mmol, 1 eq) in acetone (200 mL) was added benzyl bromide (19.0 g, 111 mmol, 1.1 eq) and K₂CO₃ (20.9 g, 151 mmol, 1.5 eq) and the resulting mixture was stirred for 4 h at 60° C. under nitrogen atmosphere. The reaction was quenched with water and was extracted with EtOAc. The combined organic layers were washed with brine, dried over anhydrous Na₂SO₄, and concentrated under reduced pressure to give the crude product which was used without further purification. LCMS (ES, m/z): [M−Cl]+: 190

Preparation of 2-benzyl-3-chloro-5-methyl-1,2-oxazol-2-ium chloride. To a solution of 2-benzyl-1,2-oxazol-3-one (7.20 g, 41.1 mmol, 1 eq) in toluene (216 mL) and triphosgene (12.2 g, 41.1 mmol, 1 eq) was added DMF (1.20 g, 10% BTC w.t) and the resulting mixture was stirred for 2 days at room temperature under nitrogen atmosphere. The precipitated solids were collected by filtration and washed with toluene. The filter cake was dried under vacuum to give the crude product which was used without further purification. LCMS (ES, m/z): [M+1]⁺: 208

Preparation of 2-benzyl-5-methyl-1,2-oxazole-3-thione. To a solution of benzyl-3-chloro-5-methyl-1,2-oxazol-2-ium (6.80 g, 32.6 mmol, 1 eq) in water was added NaSH (3.65 g, 65.2 mmol, 2.0 eq) in H₂O (10 mL) dropwise at 0° C. and the resulting mixture was stirred overnight at room temperature. The reaction mixture was extracted with DCM. The combined organic layers were washed with brine, dried over anhydrous Na₂SO₄, concentrated under reduced pressure to give the crude product which was used without further purification. LCMS (ES, m/z): [M+1]⁺: 206

Preparation of 3-(benzylsulfanyl)-5-methyl-1,2-oxazole. To a solution of 2-benzyl-5-methyl-1,2-oxazole-3-thione (4.40 g, 21.4 mmol, 1 eq) in MeCN (110 mL) was added benzyl bromide (3.67 g, 21.4 mmol, 1 eq) and the resulting mixture was stirred overnight at 80° C. under nitrogen atmosphere. The reaction mixture was concentrated under vacuum. The crude residue was purified by silica gel column chromatography, to give the product. LCMS (ES, m/z): [M+1]⁺: 206

Preparation of 5-methylisoxazole-3-sulfonyl chloride. To a solution of 3-(benzylsulfanyl)-5-methyl-1,2-oxazole (400 mg, 1.94 mmol, 1 eq) in DCM (2 mL) and H₂O (6 mL) was added trichloroisocyanuric acid (679 mg, 2.92 mmol, 1.5 eq) and the resulting mixture was stirred for 1 h at 0° C. under nitrogen atmosphere. The reaction was quenched with water and extracted with DCM. The combined organic layers were washed with brine, dried over anhydrous Na₂SO₄, and concentrated under reduced pressure to give the crude product which was used without further purification. LCMS (ES, m/z): [M+aniline-Cl+1]⁺: 239

### Preparation of 1,3,4-oxadiazole-2-sulfonyl Chloride

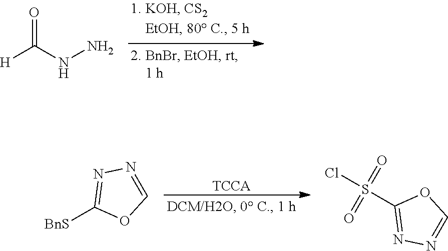

Preparation of 2-(benzylthio)-1,3,4-oxadiazole. To a solution of N-formylhydrazine (5 g, 83.2 mmol, 1 eq) and KOH (5.14 g, 91.5 mmol, 1.1 eq) in EtOH (100 mL) was added CS₂ (6.97 g, 91.5 mmol, 1.1 eq) dropwise at 0° C. and the resulting mixture was stirred for 5 h at 80° C. under nitrogen atmosphere. To the mixture was added benzyl bromide (4.98 g, 29.1 mmol, 0.35 eq) and the resulting mixture was stirred for 1 h at room temperature. The reaction mixture was concentrated under reduced pressure. The residue was purified by reversed-phase flash chromatography to give the product. LCMS (ES, m/z): [M+1]⁺: 193.

Preparation of 1,3,4-oxadiazole-2-sulfonyl chloride. To a solution of 2-(benzylsulfanyl)-1,3,4-oxadiazole (500 mg, 2.60 mmol, 1 eq) in H₂O (15 mL) and DCM (5 mL) was added trichloro-1,3,5-triazinane-2,4,6-trione (906 mg, 3.90 mmol, 1.5 eq) at 0° C. and the resulting mixture was stirred for 1 h at room temperature. The reaction mixture was diluted with water and extracted with DCM. The combined organic layers were washed with brine, dried over anhydrous Na₂SO₄, and concentrated under reduced pressure to give the crude product which was used without further purification.

### Preparation of isoxazole-4-sulfonyl Chloride

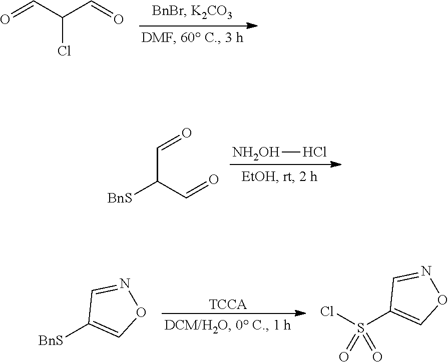

Preparation of 2-(benzylsulfanyl)propanedial. To a solution of 2-chloropropanedial (8 g, 75.1 mmol, 1 eq) in DMF (160 mL) was added benzyl mercaptan (11.2 g, 90.1 mmol, 1.2 eq) and K₂CO₃ (31.1 g, 225 mmol, 3 eq) and the resulting mixture was stirred for 3 h at 60° C. under nitrogen atmosphere. The reaction mixture was quenched with water and extracted with DCM. The combined organic layers were washed with brine, dried over anhydrous Na₂SO₄, and concentrated under reduced pressure to give the crude product which was used without further purification. LCMS (ES, m/z): [M+1]⁺: 195

Preparation of 4-(benzylsulfanyl)-1,2-oxazole. To a solution of 2-(benzylsulfanyl)propanedial (2.50 g, 12.9 mmol, 1 eq) in EtOH (50 mL) was added NH₂OH·HCl (0.89 g, 12.9 mmol, 1 eq) and the resulting mixture was stirred at 80° C. for 2 h under nitrogen atmosphere. The reaction mixture was quenched with water and was extracted with DCM. The reaction mixture was quenched with water and extracted with DCM. The combined organic layers were washed with brine, dried over anhydrous Na₂SO₄, and concentrated under reduced pressure. The crude residue was purified by reversed-phase flash chromatography to give the product. LCMS (ES, m/z): [M+1]⁺: 192

Preparation of 1,2-oxazole-4-sulfonyl chloride. To a solution of 4-(benzylsulfanyl)-1,2-oxazole (1.50 g, 7.84 mmol, 1 eq) in DCM (30 mL) and H₂O (90 mL) was added trichloroisocyanuric acid (2.73 g, 11.8 mmol, 1.5 eq) and the resulting mixture was stirred for 1 h at room temperature under nitrogen atmosphere. The reaction mixture was quenched with water and extracted with DCM. The combined organic layers were washed with brine, dried over anhydrous Na₂SO₄, and concentrated under reduced pressure to give the crude product which was used without further purification. LCMS (ES, m/z): [M+aniline-Cl+1]⁺: 225

### Preparation of 1,2,5-oxadiazole-3-sulfonyl Chloride

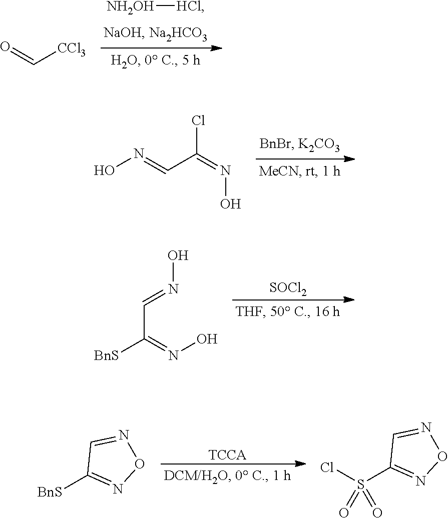

Preparation of (1E,2E)-N-hydroxy-2-(hydroxyimino)acetimidoyl chloride. To a solution of NH₂OH·HCl (28.3 g, 407 mmol, 3 eq) in H₂O (300 mL) was added NaHCO₃ (17.1 g, 203 mmol, 1.5 eq) in H₂O (300 mL) and 2,2,2-trichloroacetaldehyde (20.0 g, 135 mmol, 1 eq) and the resulting mixture was stirred for 4 h at 0° C. To the mixture was added NaOH (21.7 g, 542 mmol, 4 eq) in H₂O (200 mL) and the resulting mixture was stirred for 5 h at 0° C. The reaction mixture quenched with H₂SO₄ and extracted with EtOAc. The combined organic layers were washed with brine, dried over anhydrous Na₂SO₄, and concentrated under reduced pressure to give the crude product which was used without further purification. LCMS (ES, m/z): [M+1]+: 123

Preparation of (E)-N-[(2E)-2-(benzylsulfanyl)-2-(hydroxyimino)ethylidene]hydroxylamine. To a solution of (1E,2E)-N-hydroxy-2-(hydroxyimino)ethanecarbonimidoyl chloride (3 g, 24.4 mmol, 1 eq) in MeCN (60 mL) was added benzyl mercaptan (3.7 g, 29.3 mmol, 1.2 eq) and K₂CO₃ (10.1 g, 73.4 mmol, 3 eq) and the resulting mixture was stirred for 1 h at room temperature under nitrogen atmosphere. The crude residue was purified by reversed-phase flash chromatography to give the product. LCMS (ES, m/z): [M+1]+: 211

Preparation of 3-(benzylsulfanyl)-1,2,5-oxadiazole. To a solution of (E)-N-[(2E)-2-(benzylsulfanyl)-2-(hydroxyimino)ethylidene]hydroxylamine (2 g, 9.51 mmol, 1 eq) in THE (20 mL) was added SOCl₂ (11.3 g, 95.1 mmol, 10 eq) and the resulting mixture was stirred at 50° C. overnight. The reaction was quenched with sat. NaHCO₃ and extracted with DCM. The combined organic layers were washed with brine, dried over anhydrous Na₂SO₄, and concentrated under reduced pressure to give the crude product which was used without further purification. LCMS (ES, m/z): [M+1]+: 193

Preparation of 1,2,5-oxadiazole-3-sulfonyl chloride. To a solution of 3-(benzylsulfanyl)-1,2,5-oxadiazole (1.20 g, 6.24 mmol, 1 eq) in DCM (7 mL) and H₂O (22 mL) was added trichloroisocyanuric acid (1.45 g, 6.24 mmol, 1 eq) and the resulting mixture was stirred at 40° C. for 1 h. The reaction mixture was extracted with DCM. The combined organic layers were washed with brine, dried over anhydrous Na₂SO₄, and concentrated under reduced pressure to give the crude product which was used without further purification. LCMS (ES, m/z): [M+1]+: 169

### Preparation of 3-cyclopropylisothiazole-5-sulfonyl Chloride

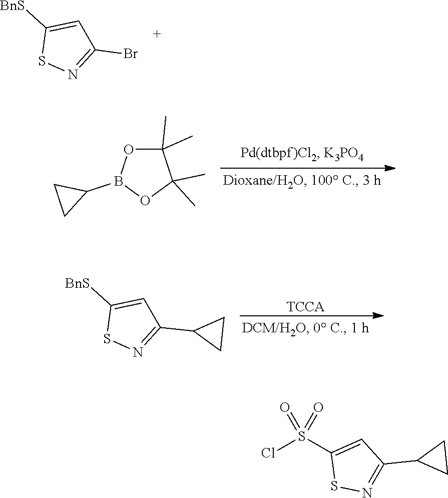

Preparation of 5-(benzylsulfanyl)-3-cyclopropyl-1,2-thiazole. To a solution of 5-(benzylsulfanyl)-3-bromo-1,2-thiazole (400 mg, 1.39 mmol, 1 eq) and 2-cyclopropyl-4,4,5,5-tetramethyl-1,3,2-dioxaborolane (704 mg, 4.19 mmol, 3 eq) in dioxane (3 mL) and H₂O (1 mL) was added K₃PO₄ (889 mg, 4.19 mmol, 3 eq) and Pd(dtbpf)Cl₂ (273 mg, 0.419 mmol, 0.3 eq) and the resulting mixture was stirred at 100° C. for 3 h. The reaction mixture was filtered and concentrated under reduced pressure. The crude residue was purified by reversed-phase flash chromatography to give the product. LCMS (ES, m/z): [M+1]⁺: 248

Preparation of 3-cyclopropyl-1,2-thiazole-5-sulfonyl chloride. To a solution of 5-(benzylsulfanyl)-3-cyclopropyl-1,2-thiazole (120 mg, 0.485 mmol, 1 eq) in DCM (1.8 mL) and H₂O (0.6 mL) was added 1,3,5-trichloro-1,3,5-triazinane-2,4,6-trione (169 mg, 0.728 mmol, 1.5 eq) at 0° C. and the resulting mixture was stirred for 1 h room temperature. The reaction was quenched with water and extracted with DCM. The combined organic layers were washed with brine, dried over anhydrous Na₂SO₄, and concentrated under reduced pressure to give the crude product which was used without further purification. LCMS (ES, m/z): [M+1]⁺: 224

### Preparation of 3-(difluoromethyl)isothiazole-5-sulfonyl Chloride

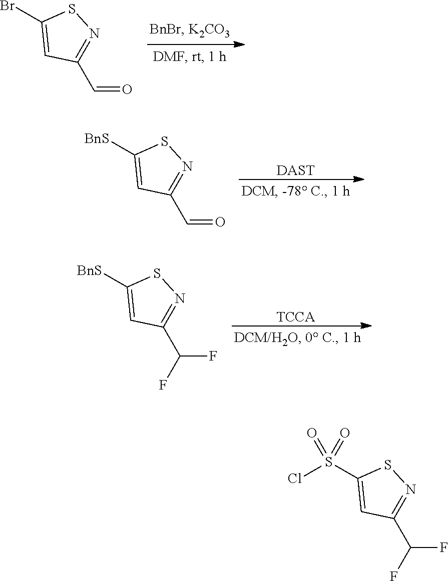

Preparation of 5-(benzylsulfanyl)-1,2-thiazole-3-carbaldehyde. To a solution of 5-bromo-1,2-thiazole-3-carbaldehyde (500 mg, 2.60 mmol, 1 eq) in DMF (10 mL) was added benzyl mercaptan (388 mg, 3.12 mmol, 1.2 eq), K₂CO₃ (1.07 g, 7.80 mmol, 3 eq) and the resulting mixture was stirred for 1 h at room temperature. The reaction mixture was quenched with water and extracted with EtOAc. The combined organic layers were washed with brine, dried over anhydrous Na₂SO₄, and concentrated under reduced pressure. The crude residue was purified by reversed-phase flash chromatography to give the product. LCMS (ES, m/z): [M+1]⁺: 236

Preparation of 5-(benzylsulfanyl)-3-(difluoromethyl)-1,2-thiazole. To a solution of 5-(benzylsulfanyl)-1,2-thiazole-3-carbaldehyde (300 mg, 1.27 mmol, 1 eq) in DCM (6 mL) was added DAST (226.0 mg, 1.40 mmol, 1.1 eq) was added dropwise at −78° C. and the resulting mixture was stirred for 1 h at room temperature. The reaction mixture was concentrated under reduced pressure. The crude residue was purified by reversed-phase flash chromatography to give the product. LCMS (ES, m/z): [M+1]⁺: 258

Preparation of 3-(difluoromethyl)isothiazole-5-sulfonyl chloride. To a solution of 5-(benzylsulfanyl)-3-(difluoromethyl)-1,2-thiazole (240 mg, 0.933 mmol, 1 eq) in DCM (1.5 mL) and H₂O (4.5 mL) was added 1,3,5-trichloro-1,3,5-triazinane-2,4,6-trione (238 mg, 1.02 mmol, 1.1 eq) at 0° C. and the resulting mixture was stirred for 1 h at room temperature. The reaction was quenched with water and extracted with DCM. The combined organic layers were washed with brine, dried over anhydrous Na₂SO₄, and concentrated under reduced pressure to give the crude product which was used without further purification. LCMS (ES, m/z): [M+1]⁺: 234

### Preparation of 5-sulfamoylthiophene-2-sulfonyl Chloride

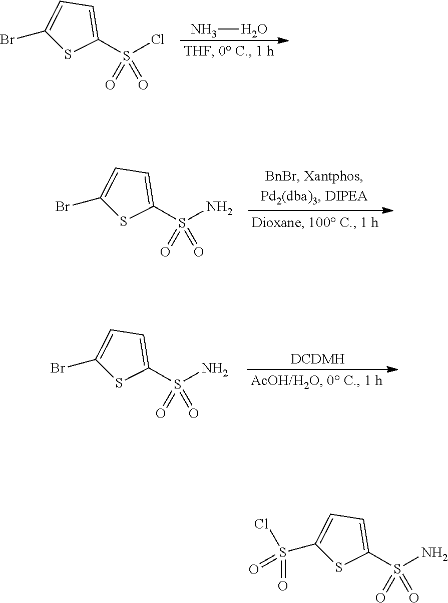

Preparation of 5-bromothiophene-2-sulfonamide. To a solution of 5-bromothiophene-2-sulfonyl chloride (500 mg, 1.91 mmol, 1 eq) in THF (10 mL) was added NH₃·H₂O (100 mg, 2.86 mmol, 1.5 eq) dropwise at 0° C. and the resulting mixture was stirred for 1 h at room temperature under nitrogen atmosphere. The reaction mixture was concentrated under reduced pressure to give the crude product which was used without further purification. LCMS (ES, m/z): [M+1]+: 242.

Preparation of 5-(benzylsulfanyl)thiophene-2-sulfonamide. To a solution of 5-bromothiophene-2-sulfonamide (460 mg, 1.90 mmol, 1 eq) in dioxane (10 mL) was added DIPEA (736 mg, 5.70 mmol, 3 eq), benzyl mercaptan (283 mg, 2.28 mmol, 1.2 eq), XantPhos (109 mg, 0.19 mmol, 0.1 eq), and Pd₂(dba)₃ (86.9 mg, 0.09 mmol, 0.05 eq) and the resulting mixture was stirred for 1 h at 100° C. under nitrogen atmosphere. The crude residue was purified by reversed-phase flash chromatography to give the product. LCMS (ES, m/z): [M+1]+: 286.

Preparation of 5-sulfamoylthiophene-2-sulfonyl chloride. To a solution of 5-(benzylsulfanyl)thiophene-2-sulfonamide (320 mg, 1.12 mmol, 1 eq) in AcOH (4 mL) and water (2 mL) was added 1,3-dichloro-5,5-dimethylimidazolidine-2,4-dione (331 mg, 1.68 mmol, 1.5 eq) 0° C. and the resulting mixture was stirred for 1 h at room temperature under nitrogen atmosphere. The reaction mixture was extracted with DCM. The combined organic layers were washed with brine, dried over anhydrous Na₂SO₄, and concentrated under reduced pressure to give the crude product which was used without further purification. LCMS (ES, m/z): [M+1]+: 262.

### Preparation of 5-sulfamoylthiophene-3-sulfonyl Chloride

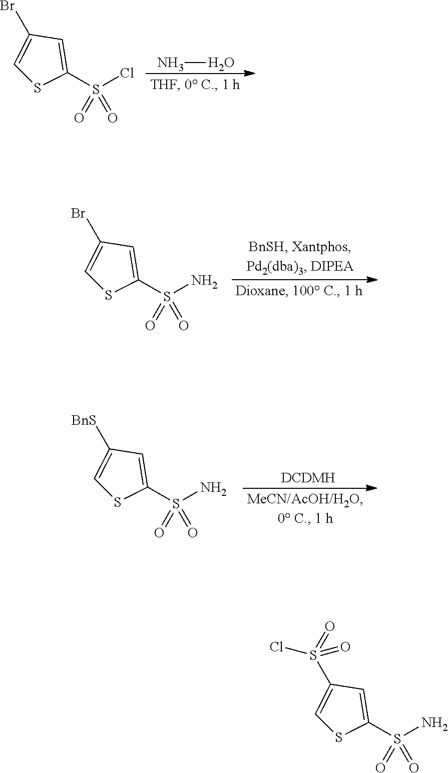

Preparation of 4-bromothiophene-2-sulfonamide. To a solution of 4-bromothiophene-2-sulfonyl chloride (800 mg, 3.06 mmol, 1 eq) in THE (16 mL) was added NH₃·H₂O (78.1 mg, 4.59 mmol, 1.5 eq) at 0° C. and the resulting mixture was stirred for 1 h at room temperature. The resulting mixture was concentrated under reduced pressure to give the crude product which was used without further purification. LCMS (ES, m/z): [M+1]⁺: 242

Preparation of 4-(benzylsulfanyl)thiophene-2-sulfonamide. To a solution of 4-bromothiophene-2-sulfonamide (796 mg, 3.28 mmol, 1 eq) in dioxane (15 mL) was added benzyl mercaptan (490 mg, 3.94 mmol, 1.2 eq), Pd₂(dba)₃ (150 mg, 0.16 mmol, 0.05 eq), XantPhos (190 mg, 0.33 mmol, 0.1 eq), and DIPEA (1.27 g, 9.86 mmol, 3 eq) and the resulting mixture was stirred for 1 h at 100° C. The crude residue was purified by reversed-phase flash chromatography to give the product. LCMS (ES, m/z): [M+1]⁺: 286

Preparation of 5-sulfamoylthiophene-3-sulfonyl chloride. To a solution of 4-(benzylsulfanyl)thiophene-2-sulfonamide (835 mg, 2.92 mmol, 1 eq) in H₂O (0.4 mL), AcOH (0.5 mL), and MeCN (12.5 mL) was added DCDMH (864 mg, 4.38 mmol, 1.2 eq) at 0° C. and the resulting mixture was stirred for 1 h at room temperature. The reaction mixture quenched with water and extracted with DCM. The combined organic layers were washed with brine, dried over anhydrous Na₂SO₄, and concentrated under reduced pressure to give the crude product which was used without further purification. LCMS (ES, m/z): [M+1]⁺: 262

### Preparation of 2-sulfamoylthiazole-5-sulfonyl Chloride

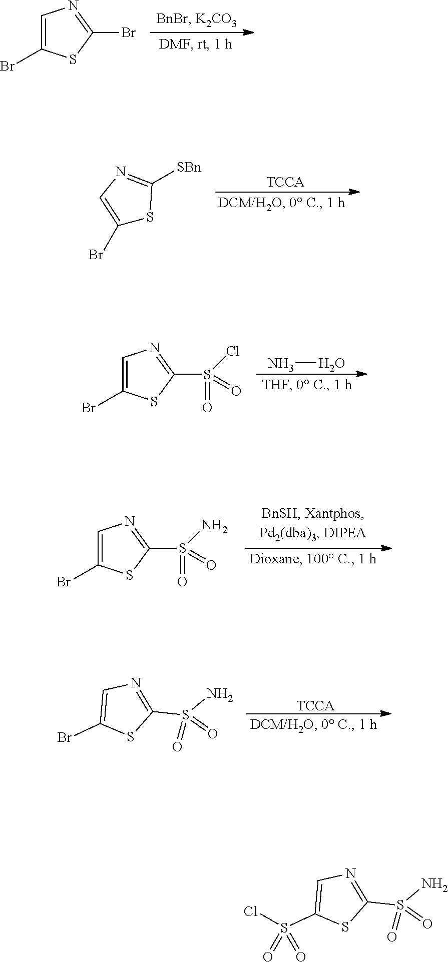

Preparation of 2-(benzylsulfanyl)-5-bromo-1,3-thiazole. To a solution of 2,5-dibromo-1,3-thiazole (4.1 g, 16.8 mmol, 1 eq) and K₂CO₃ (4.67 g, 33.7 mmol, 2 eq) in DMF (82 mL) was added benzyl mercaptan (2.52 g, 20.2 mmol, 1.2 eq) and the resulting mixture was stirred for 1 h at room temperature under nitrogen atmosphere. The reaction mixture was diluted with and extracted with EtOAc. The combined organic layers were washed with brine, dried over anhydrous Na₂SO₄, and concentrated under reduced pressure. The crude residue was purified by silica gel column chromatography to give the product. LCMS (ES, m/z): [M+1]+:286

Preparation of 5-bromo-1,3-thiazole-2-sulfonyl chloride. To a solution of 2-(benzylsulfanyl)-5-bromo-1,3-thiazole (2.5 g, 8.73 mmol, 1 eq) in DCM (12.5 mL) and H₂O (37.5 mL) was added trichloroisocyanuric acid (3.04 g, 13.1 mmol, 1.5 eq) at 0° C. and the resulting mixture was stirred for 1 h at room temperature under nitrogen atmosphere. The reaction mixture was filtered and extracted with DCM. The combined organic layers were washed with brine, dried over anhydrous Na₂SO₄, and concentrated under reduced pressure to give the crude product which was used without further purification. LCMS (ES, m/z): [M+1]+:262

Preparation of 5-bromo-1,3-thiazole-2-sulfonamide. To a solution of 5-bromo-1,3-thiazole-2-sulfonyl chloride (1.71 g, 6.53 mmol, 1 eq) in THE (30 mL) was added NH₃·H₂O (0.92 g, 26.1 mmol, 4 eq) and the resulting mixture was stirred for 1 h at room temperature under nitrogen atmosphere. The reaction mixture was diluted with water and extracted with EtOAc. The combined organic layers were washed with brine, dried over anhydrous Na₂SO₄, and concentrated under reduced pressure to give the crude product which was used without further purification. LCMS (ES, m/z): [M+1]+:243

Preparation of 5-(benzylsulfanyl)-1,3-thiazole-2-sulfonamide. To a solution of 5-bromo-1,3-thiazole-2-sulfonamide (0.8 g, 3.29 mmol, 1 eq) in dioxane (16 mL) was added benzyl mercaptan (0.49 g, 3.94 mmol, 1.2 eq), DIPEA (1.28 g, 9.87 mmol, 3 eq), Xantphos (0.19 g, 0.329 mmol, 0.1 eq), and Pd₂(dba)₃ (0.15 g, 0.165 mmol, 0.05 eq) and the resulting mixture was stirred for 1 h at 100° C. under nitrogen atmosphere. The crude residue was purified by reversed-phase flash chromatography to give the product. LCMS (ES, m/z): [M+1]+:287

Preparation of 2-sulfamoyl-1,3-thiazole-5-sulfonyl chloride. To a solution of 5-(benzylsulfanyl)-1,3-thiazole-2-sulfonamide (740 mg, 2.58 mmol, 1 eq) in DCM (12 mL) and H₂O (38 mL) was added trichloroisocyanuric acid (898 mg, 3.87 mmol, 1.5 eq) at 0° C. and the resulting mixture was stirred for 1 h at room temperature under nitrogen atmosphere. The reaction mixture was extracted with DCM. The combined organic layers were washed with brine, dried over anhydrous Na₂SO₄, and concentrated under reduced pressure to give the crude product which was used without further purification. LCMS (ES, m/z): [M+1]+:263

### Preparation of 4-sulfamoylthiophene-2-sulfonyl Chloride

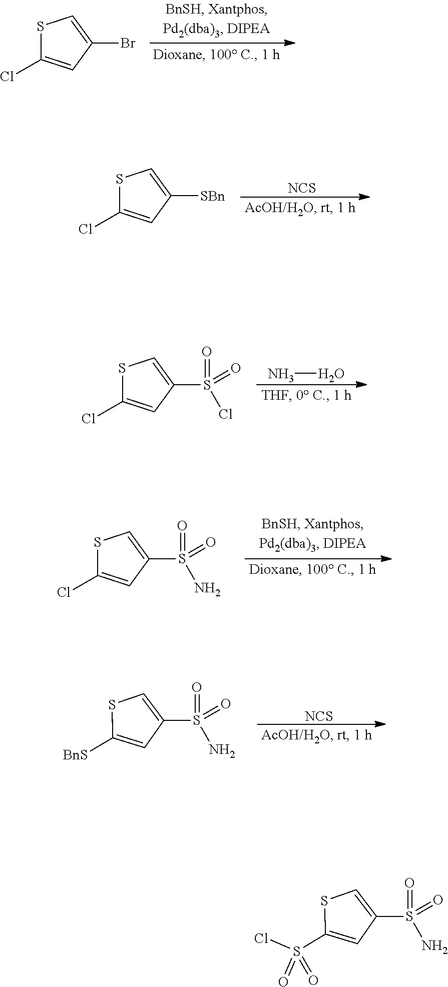

Preparation of 4-(benzylsulfanyl)-2-chlorothiophene. To a solution of 4-bromo-2-chlorothiophene (200 mg, 1.03 mmol, 1 eq) in dioxane (4 mL) was added benzyl mercaptan (151 mg, 1.21 mmol, 1.2 eq), DIPEA (392 mg, 3.04 mmol, 3 eq), XantPhos (58.6 mg, 0.101 mmol, 0.1 eq), and Pd₂(dba)₃ (46.4 mg, 0.051 mmol, 0.05 eq) and the resulting mixture was stirred for 1 h at 100° C. under nitrogen atmosphere. The reaction mixture was diluted with water and extracted with DCM. The combined organic layers were washed with brine, dried over anhydrous Na₂SO₄, and concentrated under reduced pressure. The residue was purified by reversed-phase flash chromatography to give the product. LCMS (ES, m/z): [M+1]⁺: 241

Preparation of 5-chlorothiophene-3-sulfonyl chloride. To a solution of 4-(benzylsulfanyl)-2-chlorothiophene (180 mg, 0.748 mmol, 1 eq) in AcOH (3 mL) and H₂O (1 mL) was added NCS (300 mg, 2.24 mmol, 3 eq) at 0° C. and the resulting mixture was stirred for 1 h at room temperature. The reaction mixture was diluted with water and extracted with DCM. The combined organic layers were washed with brine, dried over anhydrous Na₂SO₄, and concentrated under reduced pressure to give the crude product which was used without further purification. LCMS (ES, m/z): [M+1]⁺: 217

Preparation of 5-chlorothiophene-3-sulfonamide. To a solution of 5-chlorothiophene-3-sulfonyl chloride in THE (10 mL) was added NH₃·H₂O (10 mL) at 0° C. and the resulting mixture was stirred for 1 h at room temperature. The reaction mixture was concentrated under reduced pressure. The crude residue was purified by reversed-phase flash chromatography to give the product. LCMS (ES, m/z): [M+1]⁺: 198

Preparation of 5-(benzylsulfanyl) thiophene-3-sulfonamide. To a solution of 5-chlorothiophene-3-sulfonamide (100 mg, 0.50 mmol, 1 eq) in dioxane (2 mL) was added benzyl mercaptan (75.4 mg, 0.607 mmol, 1.2 eq), DIPEA (196 mg, 1.52 mmol, 3 eq), XantPhos (29.3 mg, 0.051 mmol, 0.1 eq), and Pd₂(dba)₃ (23.2 mg, 0.025 mmol, 0.05 eq) and the resulting mixture was stirred overnight at 100° C. under nitrogen atmosphere. The reaction mixture was diluted with water and extracted with DCM. The combined organic layers were washed with brine, dried over anhydrous Na₂SO₄, and concentrated under reduced pressure. The residue was purified by reversed-phase flash chromatography to give the product. LCMS (ES, m/z): [M+1]⁺: 286

Preparation of sulfamoylthiophene-2-sulfonyl chloride. To a solution of 5-(benzylsulfanyl) thiophene-3-sulfonamide (55 mg, 0.19 mmol, 1 eq) in AcOH (1 mL) and H₂O (0.1 mL) was added NCS (77.2 mg, 0.58 mmol, 3 eq) at 0° C. and the resulting mixture was stirred for 1 h at room temperature. The reaction mixture was diluted with water and extracted with DCM. The combined organic layers were washed with brine, dried over anhydrous Na₂SO₄, and concentrated under reduced pressure to give the crude product which was used without further purification. LCMS (ES, m/z): [M+1]⁺: 262

### Preparation of 2-(2-hydroxy-2-methylpropyl)thiazole-5-sulfonyl Chloride

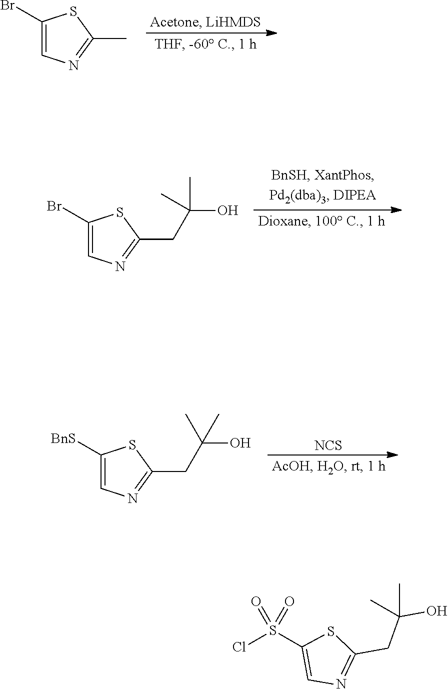

Preparation of 1-(5-bromo-1,3-thiazol-2-yl)-2-methylpropan-2-ol. To a solution of 5-bromo-2-methyl-1,3-thiazole (10.0 g, 56.2 mmol, 1 eq) in THE (100 mL) was added LiHMDS (73.0 mL, 73.0 mmol, 1.3 eq) and the resulting mixture was stirred for 30 min at −60° C. under nitrogen atmosphere. To the mixture was added acetone (3.91 g, 67.4 mmol, 1.2 eq) dropwise at −60° C. and the resulting mixture was stirred for 1 h at 0° C. under nitrogen atmosphere. The reaction was quenched with water and extracted with EtOAc. The combined organic layers were washed with brine, dried over anhydrous Na₂SO₄, and concentrated under reduced pressure. The residue was purified by reversed-phase flash chromatography to give the product. LCMS (ES) m/z: [M+1]⁺: 236

Preparation of 1-[5-(benzylsulfanyl)-1,3-thiazol-2-yl]-2-methylpropan-2-ol. To a solution of 1-(5-bromo-1,3-thiazol-2-yl)-2-methylpropan-2-ol (120 mg, 0.51 mmol, 1 eq) in dioxane (3 mL) was added benzyl mercaptan (75.7 mg, 0.61 mmol, 1.2 eq), DIPEA (197 mg, 1.52 mmol, 3.00 eq), XantPhos (29.4 mg, 0.05 mmol, 0.10 eq), and Pd₂(dba)₃ (23.3 mg, 0.03 mmol, 0.05 and the resulting mixture was stirred for 1 h at 100° C. under nitrogen atmosphere. The reaction mixture was concentrated under reduced pressure. The crude residue was purified by reversed-phase flash chromatography to give the product. LCMS (ES) m/z: [M+1]⁺: 280

Preparation of 2-(2-hydroxy-2-methylpropyl)-1,3-thiazole-5-sulfonyl chloride. To a solution of 1-[5-(benzylsulfanyl)-1,3-thiazol-2-yl]-2-methylpropan-2-ol (114 mg, 0.41 mmol, 1 eq) in AcOH (1 mL) and H₂O (1 mL) was added NCS (196.2 mg, 1.23 mmol, 3.0 eq) and the resulting mixture was stirred for 1 h at room temperature under nitrogen atmosphere. The reaction mixture was quenched with water and extracted with DCM. The reaction was quenched with water and extracted with EtOAc. The combined organic layers were washed with brine, dried over anhydrous Na₂SO₄, and concentrated under reduced pressure to give the crude product which was used without further purification. LCMS: [M+aniline-Cl+1]⁺: 313

### Preparation of tert-butyl (5-(chlorosulfonyl)thiazol-2-yl)carbamate

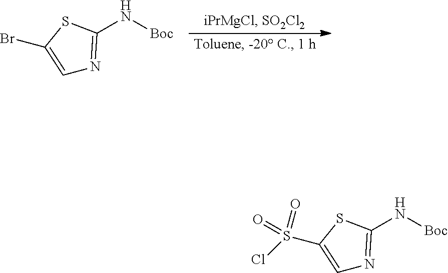 [SMILES: CC(C)(C)OC(=O)Nc1ncc(Br)s1 ; CC(C)(C)OC(=O)Nc1ncc(S(=O)(=O)Cl)s1]

Preparation of tert-butyl (5-(chlorosulfonyl)thiazol-2-yl)carbamate. To a solution of tert-butyl N-(5-bromo-1,3-thiazol-2-yl)carbamate (1 g, 3.58 mmol, 1 eq) in toluene (20 mL) was added iPrMgCl·LiCl (9 mL, 6.44 mmol, 1.8 eq) at −20° C. and the resulting mixture was stirred at room temperature for 1 h. To the mixture was added SO₂Cl₂ (1.21 g, 8.96 mmol, 2.5 eq) and the resulting mixture was stirred at room temperature for 1 h. The reaction was quenched with water and extracted with EtOAc. The combined organic layers were washed with brine, dried over anhydrous Na₂SO₄, and concentrated under reduced pressure to give the crude product which was used without further purification. LCMS (ES, m/z): [M+1]⁺: 299

### Preparation of tert-butyl (2-(chlorosulfonyl)thiazol-4-yl)carbamate

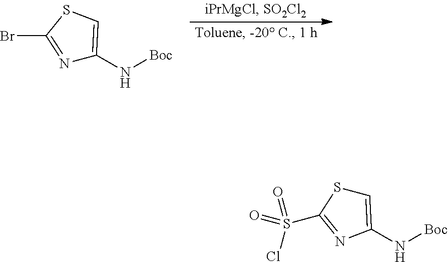 [SMILES: CC(C)(C)OC(=O)Nc1csc(Br)n1 ; CC(C)(C)OC(=O)Nc1csc(S(=O)(=O)Cl)n1]

Preparation of tert-butyl (2-(chlorosulfonyl)thiazol-4-yl)carbamate. To a solution of tert-butyl N-(2-bromo-1,3-thiazol-4-yl)carbamate (600 mg, 2.14 mmol, 1 eq) in toluene (12 mL) was added iPrMgCl·LiCl (2.98 mL, 3.86 mmol, 1.8 eq, 1.3M in THF) was added dropwise at −20° C. and the resulting mixture was stirred for 1 h at room temperature under nitrogen atmosphere. To the mixture was added SO₂Cl₂ (725 mg, 5.37 mmol, 2.5 eq) and the resulting mixture was stirred for 30 min at room temperature under nitrogen atmosphere. The reaction mixture was quenched with sat. NH₄Cl and was extracted with DCM. The combined organic layers were washed with brine, dried over anhydrous Na₂SO₄, and concentrated under reduced pressure to give the crude product which was used without further purification. LCMS (ES, m/z): [M+1]⁺: 299

### Preparation of 2-((dimethylamino)methyl)thiazole-5-sulfonyl Chloride

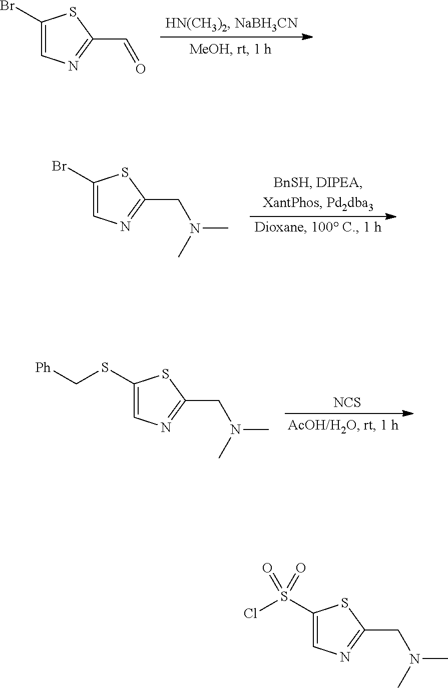

Preparation of 1-(5-bromothiazol-2-yl)-N,N-dimethylmethanamine. To a solution of 5-bromo-1,3-thiazole-2-carbaldehyde (400 mg, 2.0 mmol, 1.0 eq) in MeOH (8 mL) was added dimethylamine (2 M in MeOH) (375 mg) and AcOH (0.5 mL) and the resulting mixture was stirred for 30 min at room temperature under nitrogen atmosphere. To the mixture was added NaBH₃CN (392 mg, 6.24 mmol, 3 eq) and the resulting mixture was stirred for 1 h at room temperature. The reaction mixture was brought to pH 9 with saturated NaHCO₃ and extracted with DCM. The combined organic layers were washed with brine, dried over anhydrous Na₂SO₄, and concentrated under reduced pressure to give the product which was used without further purification. LCMS: (ES) m/z: [M+1]⁺: 221

Preparation of 1-(5-(benzylthio)thiazol-2-yl)-N,N-dimethylmethanamine. To a solution of 1-(5-bromothiazol-2-yl)-N,N-dimethylmethanamine (300 mg, 1.35 mmol, 1.0 eq) in dioxane (6 mL) was added DIPEA (526 mg, 4.07 mmol, 3 eq), benzyl mercaptan (202 mg, 1.63 mmol, 1.2 eq), XantPhos (78.5 mg, 0.136 mmol, 0.10 eq) and Pd₂(dba)₃ (62.1 mg, 0.06 mmol, 0.05 eq) and the resulting mixture was stirred for 1 h at 100° C. under nitrogen atmosphere. The reaction mixture was concentrated under vacuum. The crude residue was purified by reversed-phase flash chromatography to give the product. LCMS: (ES) m/z: [M+1]⁺: 265

Preparation of 2-((dimethylamino)methyl)thiazole-5-sulfonyl chloride. To a solution of 1-(5-(benzylthio)thiazol-2-yl)-N,N-dimethylmethanamine (200 mg, 0.75 mmol, 1.0 eq) in AcOH (4 mL) and H₂O (0.4 mL) was added NCS (303 mg, 2.26 mmol, 3 eq) at 0° C. and the resulting mixture was stirred for 1 h at room temperature. The reaction mixture was quenched with water and was extracted with DCM. The combined organic layers were washed with brine, dried over anhydrous Na₂SO, and concentrated under reduced pressure to give the product which was used without further purification.

### Preparation of 2-(1,1-difluoroethyl)thiazole-5-sulfonyl chloride

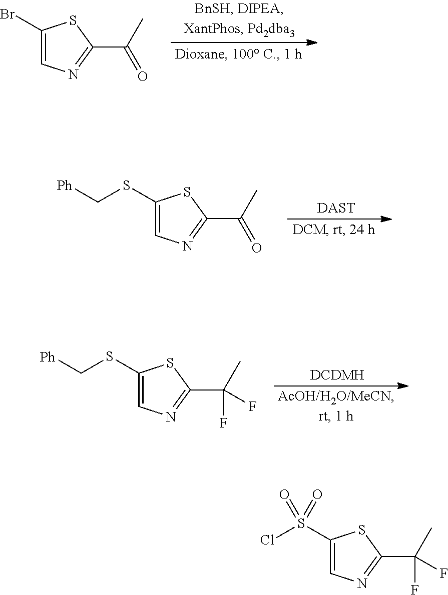

Preparation of 1-(5-(benzylthio)thiazol-2-yl)ethan-1-one. To a solution of 1-(5-bromo-1,3-thiazol-2-yl)ethanone (500 mg, 2.42 mmol, 1.0 eq) in dioxane (10 mL) was added DIPEA (940 mg, 7.27 mmol, 3 eq), benzyl mercaptan (331 mg, 2.66 mmol, 1.1.0 eq), XantPhos (140 mg, 0.24 mmol, 0.10 eq), and Pd₂(dba)₃ (111 mg, 0.12 mmol, 0.05 eq) and the resulting mixture was stirred for 1 h at 100° C. under nitrogen atmosphere. The reaction mixture was concentrated under reduced pressure. The crude residue was purified by reversed-phase flash chromatography to give the product. LCMS: (ES) m/z: [M+1]⁺: 249

Preparation of 5-(benzylthio)-2-(1,1-difluoroethyl)thiazole. To a solution of 1-(5-(benzylthio)thiazol-2-yl)ethan-1-one (475 mg, 1.90 mmol, 1.0 eq) in DCM (3 mL) was added DAST (2.4 mL) and the resulting mixture was stirred for 24 h at room temperature under nitrogen atmosphere. The reaction was quenched with water extracted with DCM. The combined organic layers were washed with brine, dried over anhydrous Na₂SO₄, and concentrated under reduced pressure to give the product which was used without further purification. LCMS: (ES) m/z: [M+1]⁺: 272

Preparation of 2-(1,1-difluoroethyl)thiazole-5-sulfonyl chloride. To a solution of 5-(benzylthio)-2-(1,1-difluoroethyl)thiazole (435 mg, 1.60 mmol, 1.0 eq) in MeCN (4.3 mL), AcOH (0.3 mL), H₂O (0.2 mL) was added 1,3-dichloro-5,5-dimethylimidazolidine-2,4-dione (473 mg, 2.40 mmol, 1.5 eq) and the resulting mixture was stirred for 1 h at room temperature under nitrogen atmosphere. The reaction mixture was diluted with water and extracted with DCM. The combined organic layers were washed with brine, dried over anhydrous Na₂SO₄, and concentrated under reduced pressure to give the product which was used without further purification. LCMS: (ES) m/z: [M+1]⁺: 247

### Preparation of 2-(3-hydroxyoxetan-3-yl)thiazole-5-sulfonyl chloride and 5-(3-hydroxyoxetan-3-yl)thiazole-2-sulfonyl chloride

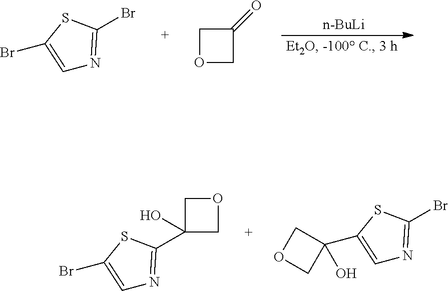

Preparation of 3-(5-bromo-1,3-thiazol-2-yl)oxetan-3-ol and 3-(2-bromothiazol-5-yl)oxetan-3-ol. To a solution of 2,5-dibromo-1,3-thiazole (3 g, 12.3 mmol, 1.0 eq) in Et₂O (150 mL) was added n-BuLi (7.4 mL, 18.4 mmol, 1.5 eq, 2.5 M in hexane) at −100° C. and the resulting mixture was stirred at −100° C. for 1 h. To the solution was added oxetan-3-one (1.36 g, 18.4 mmol, 1.5 eq) in Et₂O (10 mL) and the resulting mixture was stirred at −100° C. for 3 h. The reaction mixture was quenched with MeOH and was concentrated under reduced pressure. The crude residue was purified by reversed-phase flash chromatography to give the products. LCMS: (ES) m/z: [M+1]⁺: 236

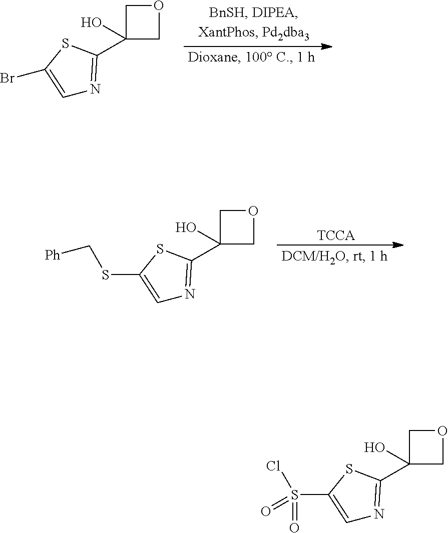

Preparation of 3-(5-(benzylthio)thiazol-2-yl)oxetan-3-ol. To a solution of 3-(5-bromo-1,3-thiazol-2-yl)oxetan-3-ol (750 mg, 3.17 mmol, 1.0 eq) in dioxane (15 mL) was added DIPEA (1.23 g, 9.5 mmol, 3 eq), XantPhos (183 mg, 0.31 mmol, 0.10 eq), Pd₂(dba)₃ (145 mg, 0.15 mmol, 0.05 eq) and benzyl mercaptan (473 mg, 3.81 mmol, 1.2 eq) and the resulting mixture was stirred for 1 h at 100° C. under nitrogen atmosphere. The reaction mixture was quenched with water and extracted with EtOAc. The crude residue was purified by reversed-phase flash chromatography to give the product. LCMS: (ES) m/z: [M+1]⁺: 280

Preparation of 2-(3-hydroxyoxetan-3-yl)-1,3-thiazole-5-sulfonyl chloride. To a solution of 3-(5-(benzylthio)thiazol-2-yl)oxetan-3-ol (800 mg, 2.86 mmol, 1.0 eq) in DCM (5 mL) and H₂O (15 mL) was added trichloroisocyanuric acid (732 mg, 3.15 mmol, 1.1.0 eq) at 0° C. and the resulting mixture was stirred for 1 h at room temperature. The reaction mixture was quenched with water and extracted with DCM. The combined organic layers were washed with brine, dried over anhydrous Na₂SO₄, and concentrated under reduced pressure to give the product which was used without further purification.

### Preparation of 5-(3-hydroxyoxetan-3-yl)thiazole-2-sulfonyl chloride

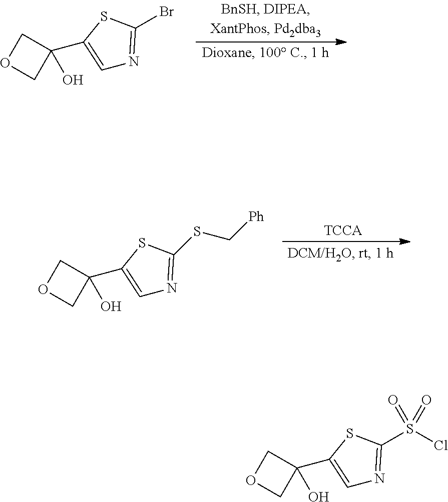

Preparation of 3-(2-(benzylthio)thiazol-5-yl)oxetan-3-ol. To a solution of 3-(2-bromo-1,3-thiazol-5-yl)oxetan-3-ol (1 g, 4.23 mmol, 1.0 eq) in dioxane (20 mL) was added XantPhos (0.25 g, 0.42 mmol, 0.10 eq), Pd₂(dba)₃ (0.19 g, 0.21 mmol, 0.05 eq), DIPEA (1.64 g, 12.7 mmol, 3 eq) and benzyl mercaptan (0.63 g, 5.08 mmol, 1.2 eq) and the resulting mixture was stirred for 1 h at 100° C. under nitrogen atmosphere. The reaction mixture was quenched with water and extracted with EtOAc. The combined organic layers were washed with brine, dried over anhydrous Na₂SO₄, and concentrated under reduced pressure. The crude residue was purified by reversed-phase flash chromatography to give the product. LCMS: (ES) m/z: [M+1]⁺: 280

Preparation of 5-(3-hydroxyoxetan-3-yl)-1,3-thiazole-2-sulfonyl chloride. To a solution of 3-[2-(benzylsulfanyl)-1,3-thiazol-5-yl]oxetan-3-ol (1.05 g, 3.75 mmol, 1.0 eq) in DCM (6.3 mL) and H₂O (18.9 mL) was added trichloroisocyanuric acid (0.96 g, 4.13 mmol, 1.1.0 eq) at 0° C. and the resulting mixture was stirred for 1 h at room temperature. The reaction mixture was quenched with water and extracted with DCM. The combined organic layers were washed with brine, dried over anhydrous Na₂SO₄, and concentrated under reduced pressure to give the product which was used without further purification.

Preparation of Intermediate Indoles and Indazoles

### Preparation of 7-amino-4-methyl-1H-indole-3-carbonitrile

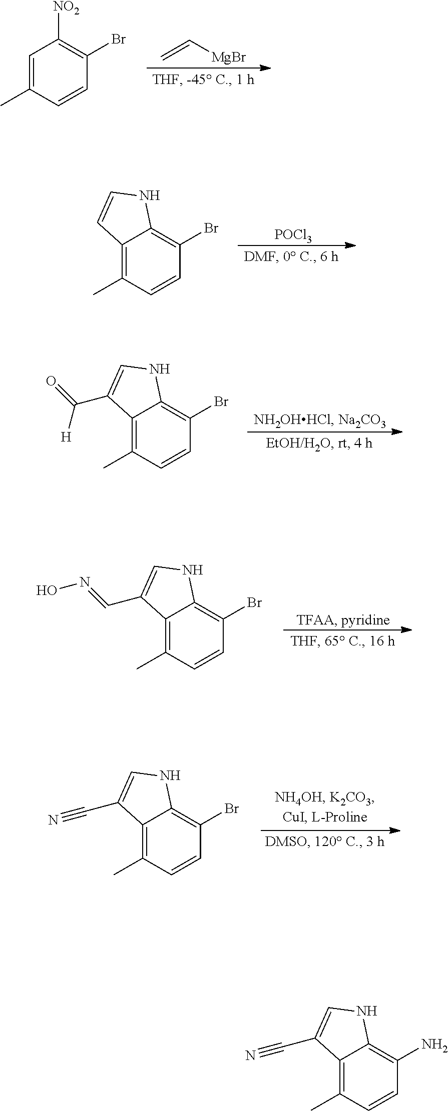

Preparation of 7-bromo-4-methyl-1H-indole. To a solution of 1-bromo-4-methyl-2-nitrobenzene (100 g, 462 mmol, 1 eq) in THF (2 L) was added vinylmagnesium bromide (1.39 L, 1.39 mol, 1 M, 3 eq) at −45° C. and the resulting mixture was stirred at −45° C. for 1 h. The reaction mixture was quenched with sat. NH4Cl and extracted with EtOAc. The reaction mixture was diluted with water and extracted with DCM. The combined organic layers were washed with brine, dried over anhydrous Na₂SO₄, and concentrated under reduced pressure. The crude residue was purified by silica gel column chromatography to give the product.

Preparation of 7-bromo-4-methyl-1H-indole-3-carbaldehyde. To a solution of POCl₃ (26.7 mL, 286 mmol, 1.1 eq) in DMF (250 mL) was added 7-bromo-4-methyl-1H-indole (50 g, 238 mmol, 1 eq) in DMF (250 mL) at 0° C. and the resulting mixture was allowed stirred at room temperature for 6 h. The reaction mixture was quenched with water and the precipitated solids were collected by filtration and washed with water to give the crude product which was used without further purification. LCMS (ES, m/z): 239.9 [M+1].

Preparation of (E)-7-bromo-4-methyl-1H-indole-3-carbaldehyde oxime. To a solution of 7-bromo-4-methyl-1H-indole-3-carbaldehyde (52 g, 218 mmol, 1 eq) in EtOH (1 L) was added Na₂CO₃ (46.3 g, 437 mmol, 2 eq) in H₂O (130 mL) and NH₂OH·HCl (30.3 g, 437 mmol, 2 eq) and the resulting mixture was stirred at room temperature for 4 h. The reaction mixture was quenched with water and the precipitated solids were collected by filtration, washed with water, and dried under reduced pressure to give the crude product which was used without further purification. LCMS (ES, m/z): 255.0 [M+2H]⁺.

Preparation of 7-amino-1H-indole-3-carbonitrile. To a solution of (E)-7-bromo-4-methyl-1H-indole-3-carbaldehyde oxime (51 g, 201 mmol, 1 eq) in THE (50 mL) was added pyridine (33 mL, 403 mmol, 2 eq) and TFAA (143 mL, 403 mmol, 2 eq) at 0° C. and the resulting mixture was stirred at 65° C. for 16 h. The reaction mixture was concentrated, diluted with water, and extracted with EtOAc. The combined organic layers were washed with brine, dried over anhydrous Na₂SO₄, and concentrated under reduced pressure to give the crude product which was used without further purification. LCMS (ES, m/z): 232.9 [M−H]⁻

Preparation of 7-amino-4-methyl-1H-indole-3-carbonitrile. To a solution of 7-bromo-4-methyl-1H-indole-3-carbonitrile (25 g, 106 mmol, 1 eq) in DMSO (125 mL) was added K₂CO₃ (36.7 g, 266 mmol, 2.5 eq), CuI (4.05 g, 22 mmol, 0.2 eq), L-proline (2.45 g, 22 mmol, 0.2 eq), and aqueous NH₄OH (500 mL) and the resulting mixture was stirred at 120° C. for 3 h. The reaction mixture was filtered and extracted with EtOAc. The combined organic layers were washed with brine, dried over anhydrous Na₂SO₄, and concentrated under reduced pressure. The crude residue was purified by silica gel column chromatography to give the product. LCMS (ES, m/z): 169.9 [M−H]⁺

### Preparation of 7-amino-1H-indole-3-carbonitrile

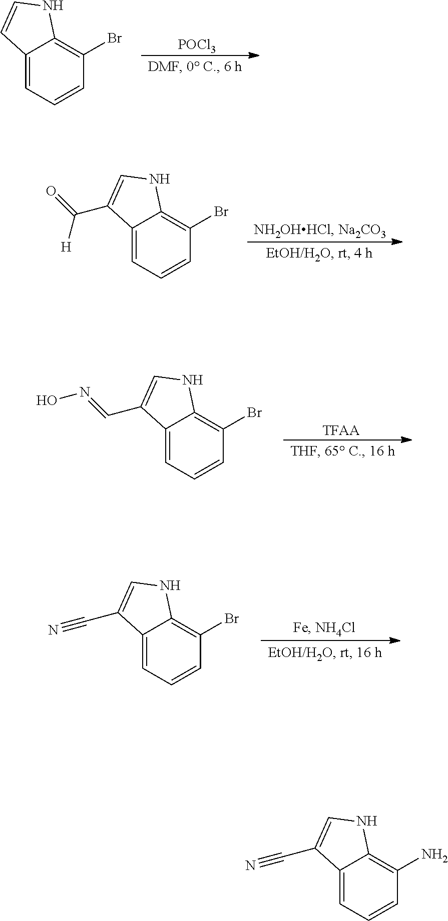

Preparation of 7-nitro-1H-indole-3-carbaldehyde. To a solution of POCl₃ (34.6 mL, 320 mmol, 1.1 eq) in DMF (50 mL) was added 7-nitro-1H-indole (50 g, 308 mmol, 1 eq) in DMF (500 mL) at 0° C. and the resulting mixture was allowed stirred at room temperature for 5 h. The reaction mixture was quenched with water and the precipitated solids were collected by filtration, washed with water, and dried under reduced pressure to give the crude product which was used without further purification. LCMS (ES, m/z): 190.98 [M+1]⁺.

Preparation of (E)-7-nitro-1H-indole-3-carbaldehyde oxime. To a solution of 7-nitro-1H-indole-3-carbaldehyde (60 g, 315 mmol, 1 eq) in EtOH (1 L) was added Na₂CO₃ (100 g, 945 mmol, 3 eq) in H₂O (240 mL), NH₂OH·HCl (54.7 g, 687 mmol, 2.5 eq) and the resulting mixture was stirred at room temperature for 4 h The reaction mixture was quenched with water and the precipitated solids were collected by filtration, washed with water, and dried under reduced pressure to give the crude product which was used without further purification. LCMS (ES, m/z): 205.98 [M+1]⁺.

Preparation of 7-nitro-1H-indole-3-carbonitrile. To a solution of (E)-7-nitro-1H-indole-3-carbaldehyde oxime (60 g, 292 mmol, 1 eq) in THF (1.2 L) was added pyridine (70 mL, 877 mmol, 3 eq) and TFAA (143 mL, 1024 mmol, 3.5 eq) and the resulting mixture was stirred at 65° C. for 16 h. The reaction mixture was concentrated, diluted with water, and extracted with EtOAc. The combined organic layers were washed with brine, dried over anhydrous Na₂SO₄, and concentrated under reduced pressure to give the crude product which was used without further purification. LCMS (ES, m/z): 185.97 [M−H]⁻.

Preparation of 7-amino-1H-indole-3-carbonitrile. To a solution of 7-nitro-1H-indole-3-carbonitrile (50 g, 267 mmol, 1 eq) in EtOH (500 mL) and H₂O (500 mL) was added iron powder (37.24 g, 667 mmol, 3 eq) and NH₄Cl (71.4 g, 1336 mmol, 5 eq) at 0° C. and the resulting mixture was stirred at room temperature for 16 h. The reaction mixture was filtered and extracted with DCM. The combined organic layers were washed with brine, dried over anhydrous Na₂SO₄, and concentrated under reduced pressure. The crude residue was purified by silica gel column chromatography to give the product. LCMS (ES, m/z): 157.98 [M+1]⁺.

### Preparation of 7-amino-4-(trifluoromethyl)-1H-indole-3-carbonitrile

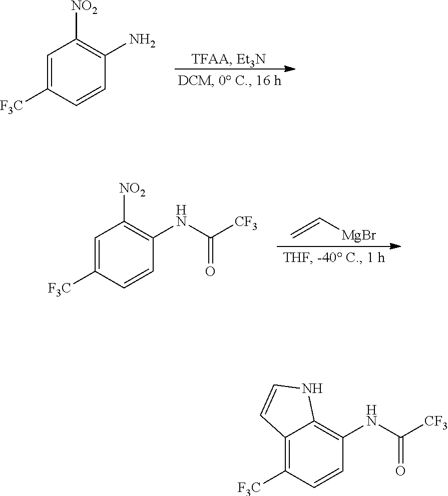

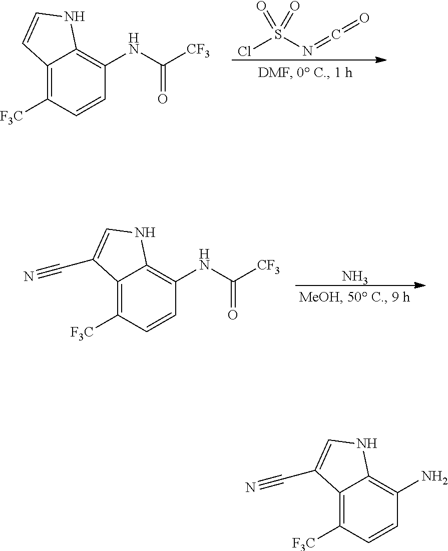

Preparation of 2,2,2-trifluoro-N-[2-nitro-4-(trifluoromethyl)phenyl]acetamide. To a solution of 2-nitro-4-(trifluoromethyl)aniline (13.5 g, 65.5 mmol, 1 eq) (SM1), and Et3N (19.9 g, 196 mmol, 3 eq) in DCM (270 mL) was added TFAA (27.5 g, 131 mmol, 2 eq) dropwise at 0° C. under nitrogen atmosphere and the resulting mixture was stirred at room temperature overnight. The mixture was diluted with diluted with water and extracted with DCM. The combined organic layers were washed with brine, dried over Na₂SO₄, and concentrated under reduced pressure to afford crude product which was used without further purification.

Preparation of 2,2,2-trifluoro-N-[4-(trifluoromethyl)-1H-indol-7-yl]acetamide. To a solution of 2,2,2-trifluoro-N-[2-nitro-4-(trifluoromethyl)phenyl]acetamide (22.7 g, 75.13 mmol, 1 eq) in THF (450 mL) was added vinylmagnesium bromide (451 mL, 451 mmol, 6 eq, 1M in THF) dropwise at −40° C. under nitrogen atmosphere and the resulting mixture was stirred at −40° C. for 1 h. The reaction was quenched with sat. NH₄Cl and was extracted with EtOAc. The combined organic layers were washed with brine, dried over Na₂SO₄, and concentrated under reduced pressure. The residue was purified by silica gel column chromatography to give the product. LCMS (ES, m/z): [M+1]⁺: 297

Preparation of N-[3-cyano-4-(trifluoromethyl)-1H-indol-7-yl]-2,2,2-trifluoroacetamide. To a solution of 2,2,2-trifluoro-N-[4-(trifluoromethyl)-1H-indol-7-yl]acetamide (3.70 g, 12.5 mmol, 1 eq) in DMF (75 mL) was added chlorosulfonyl isocyanate (5.30 g, 37.5 mmol, 3 eq) dropwise at 0° C. under nitrogen atmosphere and the resulting mixture was stirred at 0° C. for 1 h. The reaction was quenched with water and the precipitated solids were collected by filtration and washed with water to give the crude product which was used without further purification. LCMS (ES, m/z): [M+1]⁺: 322

Preparation of 7-amino-4-(trifluoromethyl)-1H-indole-3-carbonitrile. To a solution of N-[3-cyano-4-(trifluoromethyl)-1H-indol-7-yl]-2,2,2-trifluoroacetamide (4.30 g, 13.4 mmol, 1 eq) in MeOH (50 mL) was added NH₃(g) in MeOH (50.0 mL, 3M) and the resulting mixture was stirred at 50° C. for 9 h. The resulting mixture was concentrated under vacuum. The residue was purified by silica gel column chromatography to give the product. LCMS (ES, m/z): [M+1]⁺: 226

### Preparation of 3-(trifluoromethyl)-1H-indol-7-amine analogues

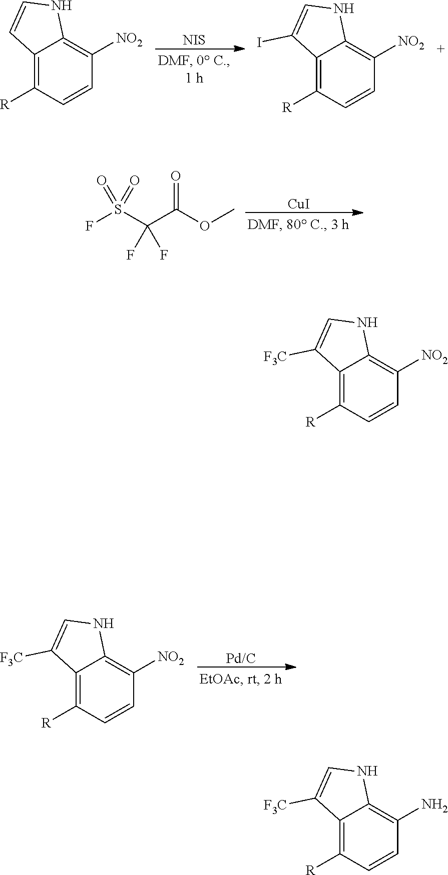

Preparation of 3-iodo-4-methyl-7-nitro-1H-indole. To a solution of 4-methyl-7-nitro-1H-indole (650 mg, 3.70 mmol, 1 eq) in DMF (10 mL) was added NIS (875 mg, 3.88 mmol, 1.05 eq) at 0° C. and the resulting mixture was stirred at room temperature for 1 h. The resulting mixture was diluted with water and the precipitated solids were collected by filtration and washed with water and dried under vacuum to give the product which was used without further purification.

Preparation of 4-methyl-7-nitro-3-(trifluoromethyl)-1H-indole. To a solution of 3-iodo-4-methyl-7-nitro-1H-indole (940 mg, 3.12 mmol, 1 eq) and methyl 2,2-difluoro-2-sulfoacetate (3 g, 15.6 mmol, 5 eq) in DMF (20 mL) was added CuI (118 mg, 0.62 mmol, 0.2 eq) and the resulting mixture was stirred at 80° C. for 3 h under nitrogen atmosphere. The mixture was allowed to cool to room temperature and the precipitated solids were collected by filtration, washed with water, and were dried under vacuum to give the product.

Preparation of 4-methyl-3-(trifluoromethyl)-1H-indol-7-amine. To a solution of 4-methyl-7-nitro-3-(trifluoromethyl)-1H-indole (476 mg, 1.95 mmol, 1 eq) in EtOAc (50 mL) was added Pd/C (10% w.t) and the resulting mixture was stirred for 2 hours at room temperature under hydrogen atmosphere. The precipitated solids were collected by filtration, washed with water, and were dried under vacuum to give the product.

### Preparation of 3-chloro-1H-indol-7-amine Analogues

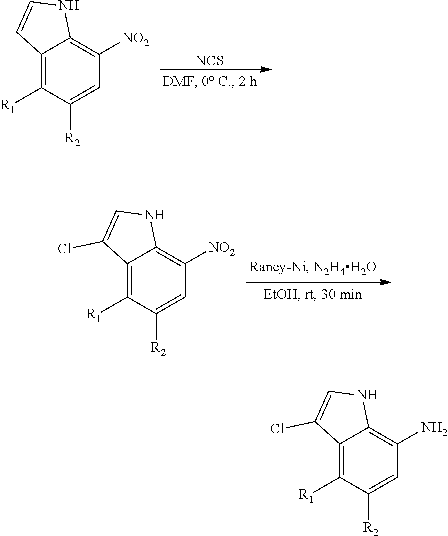

Preparation of 3-chloro-4-fluoro-7-nitro-1H-indole. To a solution of 4-fluoro-7-nitro-1H-indole (1 g, 5.55 mmol, 1 eq) in DMF (20 mL) was added NCS (815 mg, 6.10 mmol, 1.1 eq) in DMF (1 mL) dropwise at 0° C. and the resulting mixture was stirred at room temperature for 2 h. The reaction was quenched with water and the precipitated solids were collected by filtration, washed with water, and were dried under vacuum to give the product which was used without further purification.

Preparation of 3-chloro-4-fluoro-1H-indol-7-amine. To a solution of 3-chloro-4-fluoro-7-nitro-1H-indole (1.1 g, 5.12 mmol, 1 eq) in EtOH (55 mL) was added and Raney-Ni (440 mg, 5.12 mmol, 1 eq) and hydrazine hydrate (330 mg, 10.2 mmol, 2 eq) at room temperature and the resulting mixture was stirred at room temperature for 30 min. the precipitated solids were collected by filtration, washed with EtOH, and were dried under vacuum to give the product which was used without further purification.

### Preparation of 7-amino-1H-indole-3-carbonitrile analogues

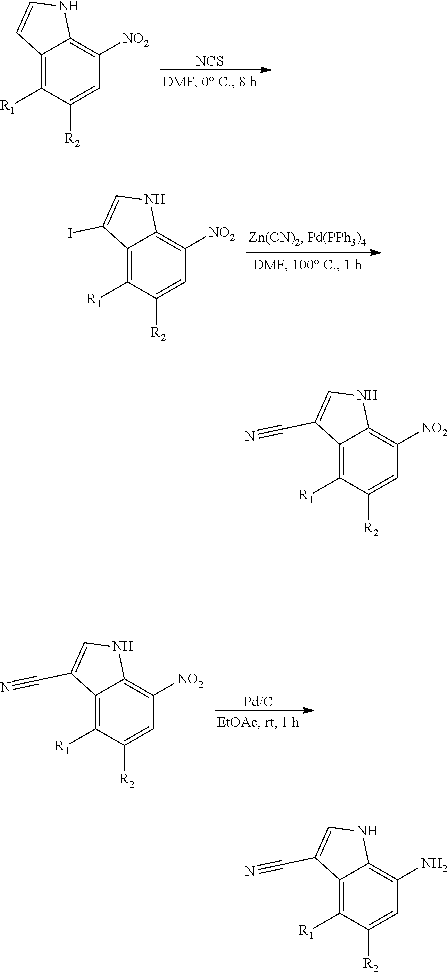

Preparation of 4-fluoro-3-iodo-7-nitro-1H-indole. To a solution of 4-fluoro-7-nitro-1H-indole (1 g, 5.55 mmol, 1 eq) in DMF (20 mL) was added and NIS (1.4 g, 6.10 mmol, 1.1 eq) and the resulting mixture was stirred at room temperature for 8 h. The reaction was quenched with water and the precipitated solids were collected by filtration, washed with water, and were dried under vacuum to give the product which was used without further purification.

Preparation of 4-fluoro-7-nitro-1H-indole-3-carbonitrile. To a solution of 4-fluoro-3-iodo-7-nitro-1H-indole (1.5 g, 4.90 mmol, 1 eq) in DMF (20 mL) was added Zn(CN)₂ (345 mg, 2.94 mmol, 0.6 eq) and Pd(PPh₃)₄ (566 mg, 0.49 mmol, 0.1 eq) and the resulting mixture was stirred at 100° C. for 1 h under nitrogen atmosphere. The reaction was quenched with sat. NH₄Cl and was extracted with DCM. The combined organic layers were washed with brine, dried over Na₂SO₄, and concentrated under reduced pressure. The residue was purified by silica gel column chromatography to give the product.

Preparation of 7-amino-4-fluoro-1H-indole-3-carbonitrile. To a solution of 4-fluoro-7-nitro-1H-indole-3-carbonitrile (308 mg, 1.5 mmol, 1 eq) and Pd/C (10% w.t) in EtOAc (10 mL V) was stirred at room for 1 h temperature under hydrogen atmosphere. The resulting mixture was filtered and concentrated under reduced pressure to give the product which was used without further purification.

### Preparation of 7-amino-1H-indazole-3-carbonitrile Analogues

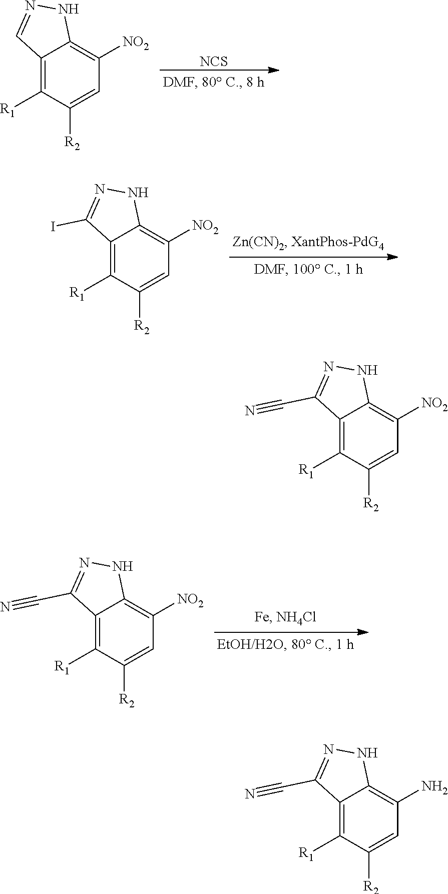

Preparation of 3-iodo-7-nitro-1H-indazole. To a solution of 7-nitroindazole (4 g, 24 mmol, 1 eq) in DMF (80 mL) was added and NIS (6.6 g, 29 mmol, 1.2 eq) and the resulting mixture was stirred at 80° C. for 1 h under nitrogen atmosphere. The reaction was quenched with water and the precipitated solids were collected by filtration, washed with water, and were dried under vacuum to give the product which was used without further purification. LCMS (ES, m/z): [M+1]⁺: 290

Preparation of 7-nitro-1H-indazole-3-carbonitrile. To a solution of 3-iodo-7-nitro-1H-indazole (4.2 g, 14.5 mmol, 1 eq) in DMF (80 mL) was added Zn(CN)₂ (1.02 g, 8.71 mmol, 0.6 eq) and XantPhos-Pd-G4 (1.29 g, 1.45 mmol, 0.1 eq) and the resulting mixture was stirred at 100° C. for 1 h under nitrogen atmosphere. The reaction was quenched with water and the precipitated solids were collected by filtration, washed with water, and were dried under vacuum to give the product which was used without further purification. LCMS (ES, m/z): [M+1]⁺:189

Preparation of 7-amino-1H-indazole-3-carbonitrile. To a solution of 7-nitro-1H-indazole-3-carbonitrile (2.8 g, 14.8 mmol, 1 eq) in EtOH (70 mL) and H₂O (14 mL) was added NH₄Cl (7.96 g, 148 mmol, 10 eq) and Fe (8.31 g, 148 mmol, 10 eq) and the resulting mixture was stirred at 80° C. for 1 h under nitrogen atmosphere. The resulting mixture was filtered, and the solids were washed with EtOH and concentrated under reduced pressure. The crude residue was dissolved in water and extracted with EtOAc. The combined organic layers were washed with brine, dried over anhydrous Na₂SO₄, and concentrated under reduced pressure to give the product. LCMS (ES, m/z): [M+1]⁺:159

### Preparation of 3-chloro-1H-indazol-7-amine

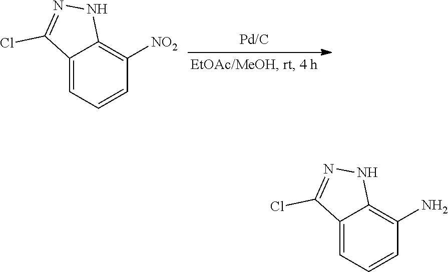 [SMILES: O=[N+]([O-])c1cccc2c(Cl)n[nH]c12 ; Nc1cccc2c(Cl)n[nH]c12]

Preparation of 3-chloro-1H-indazol-7-amine. To a solution of 3-chloro-7-nitro-1H-indazole (1 g, 5.06 mmol, 1 eq) in MeOH (10 mL) and EtOAc (10 mL) was added Pd/C (0.20 g, 20% w.t.) at room temperature under hydrogen atmosphere and the resulting mixture was stirred at room temperature for 4 h. The resulting mixture was filtered and concentrated under reduced pressure. The crude residue was purified by reverse flash chromatography to give the product. LCMS: (ES, m/z): [M+1]⁺: 168

### Preparation of 7-amino-4-ethyl-1H-indole-3-carbonitrile

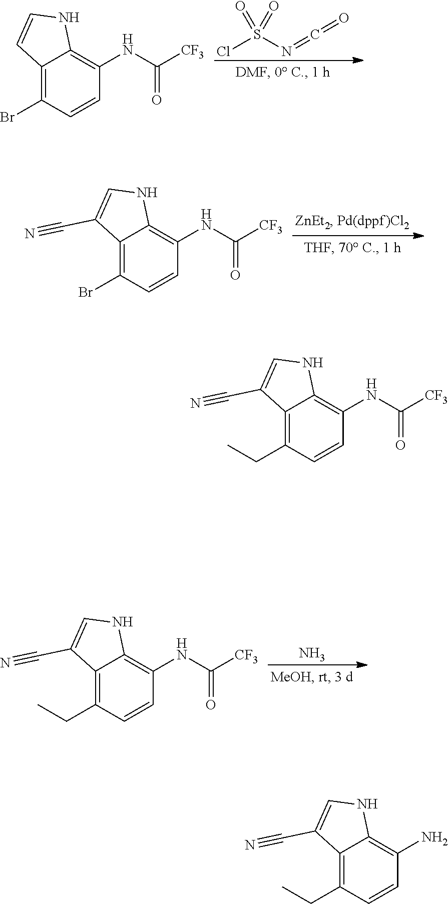

Preparation of N-(4-bromo-3-cyano-1H-indol-7-yl)-2,2,2-trifluoroacetamide. To a solution of N-(4-bromo-1H-indol-7-yl)-2,2,2-trifluoroacetamide (2.50 g, 8.14 mmol, 1) in DMF (50 mL) was added chlorosulfonyl isocyanate (3.50 g, 24.4 mmol, 3 eq) dropwise at 0° C. and the resulting mixture was stirred at 0° C. for 1 h. The resulting mixture was extracted with EtOAc. The combined organic layers were washed with brine, dried over anhydrous Na₂SO₄, and concentrated under reduced pressure. The residue was purified by reversed-phase flash chromatography to give the product. LCMS (ES, m/z): [M+1]⁺: 332

Preparation of N-(3-cyano-4-ethyl-1H-indol-7-yl)-2,2,2-trifluoroacetamide. To a solution of N-(4-bromo-3-cyano-1H-indol-7-yl)-2,2,2-trifluoroacetamide (1 g, 3 mmol, 1 eq) in THE (20 mL) was added diethylzinc (1.12 g, 9 mmol, 3 eq) and Pd(dppf)Cl₂ (0.11 g, 0.15 mmol, 0.05 eq) and the resulting mixture was stirred at 70° C. for 1 h under nitrogen atmosphere. The reaction mixture was concentrated under reduced pressure and the crude was purified by reversed-phase flash chromatography to give the product. LCMS (ES, m/z): [M+1]⁺: 282

Preparation of 7-amino-4-ethyl-1H-indole-3-carbonitrile. To a solution of N-(3-cyano-4-ethyl-1H-indol-7-yl)-2,2,2-trifluoroacetamide (750 mg, 2.7 mmol, 1 eq) in MeOH (5 mL) was added NH₃ (g) in MeOH (7.5 mL) at room temperature and the resulting mixture was stirred for 3 days at room temperature. The resulting mixture was concentrated under vacuum to give the crude product which was used without further purification. LCMS (ES, m/z): [M+1]⁺: 186

### Preparation of 7-amino-4-methoxy-1H-indole-3-carbonitrile

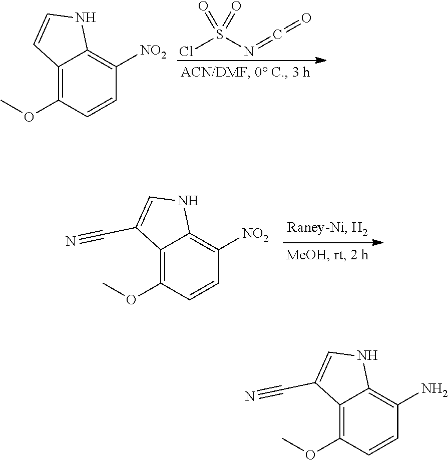

Preparation of 4-methoxy-7-nitro-1H-indole-3-carbonitrile. To a solution of 4-methoxy-7-nitro-1H-indole (500 mg, 2.6 mmol, 1 eq) in ACN (10 mL) and DMF (5 mL) was added chlorosulfonyl isocyanate (1.10 g, 7.81 mmol, 3 eq) dropwise portions at 0° C. and the resulting mixture was stirred at 0° C. for 3 h under nitrogen atmosphere. The reaction was quenched with water and the precipitated solids were collected by filtration and washed with water to give the crude product which was used without further purification. LCMS (ES, m/z): [M+1]⁺: 218.

Preparation of 7-amino-4-methoxy-1H-indole-3-carbonitrile. To a solution of 4-methoxy-7-nitro-1H-indole-3-carbonitrile (450 mg, 2.1 mmol, 1 eq) in MeOH (100 mL) was added Raney-Ni (500 mg, 100% w.t) and the resulting mixture was stirred for 2 h at room temperature under hydrogen atmosphere. The residue was concentrated under reduced pressure to give the product which was used without further purification. LCMS (ES, m/z): [M+1]⁺: 188

### Preparation of 7-amino-4-methoxy-1H-indole-3-carbonitrile

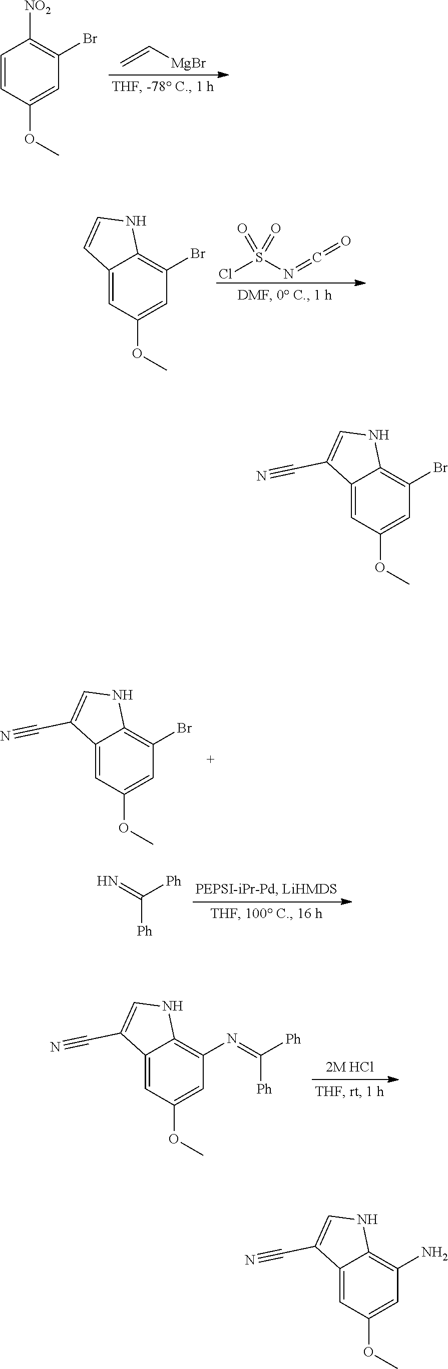

Preparation of 7-bromo-5-methoxy-1H-indole. To a solution of 2-bromo-4-methoxy-1-nitrobenzene (10 g, 43 mmol, 1 eq) in THF (200 mL) was added vinylmagnesium bromide (57.3 mL, 172 mmol, 4 eq, 3M in THF) dropwise at −78° C. under a nitrogen atmosphere and the resulting mixture was stirred at −78° C. for 1 h. The reaction mixture was quenched with water and was extracted with EtOAc. The combined organic layers were washed with brine, dried over anhydrous Na₂SO₄, and concentrated under reduced pressure. The crude residue was purified by silica gel column chromatography to give the product. LCMS: (ES, m/z): [M+1]⁺: 226

Preparation of 7-bromo-5-methoxy-1H-indole-3-carbonitrile. To a solution of 7-bromo-5-methoxy-1H-indole (5 g, 22.1 mmol, 1 eq) in DMF (100 mL) was added chlorosulfonyl isocyanate (6.26 g, 44.2 mmol, 2 eq) dropwise at 0° C. under a nitrogen atmosphere and the resulting mixture was stirred at 0° C. for 1 h. The reaction mixture was quenched with water and was extracted with EtOAc. The combined organic layers were washed with brine, dried over anhydrous Na₂SO₄, and concentrated under reduced pressure. The crude residue was purified by reverse flash chromatography to give the product. LCMS: (ES, m/z): [M+1]⁺: 251

Preparation of 7-((diphenylmethylene)amino)-5-methoxy-1H-indole-3-carbonitrile. To a solution of 7-bromo-5-methoxy-1H-indole-3-carbonitrile (2.6 g, 10.3 mmol, 1 eq) in THF (50 mL) was added diphenylmethanimine (3.75 g, 20.7 mmol, 2 eq), PEPPSI-iPr-Pd (1.01 g, 1.03 mmol, 0.1 eq) and LiHMDS (31 mL, 31.1 mmol, 3 eq, 1 M in THF) under a nitrogen atmosphere and the resulting mixture was stirred at 100° C. overnight. The reaction mixture was quenched with sat. NH₄Cl and was extracted with EtOAc. The combined organic layers were washed with brine, dried over anhydrous Na₂SO₄, and concentrated under reduced pressure to give the crude product which was used without further purification. LCMS: (ES, m/z): [M+1]⁺: 352

Preparation of 7-amino-5-methoxy-1H-indole-3-carbonitrile. To a solution of 7-((diphenylmethylene)amino)-5-methoxy-1H-indole-3-carbonitrile (3.5 g, 9.96 mmol, 1 eq) in THF (35 mL) was added HCl (35 mL, 70 mmol, 7 eq, 2M in THF) dropwise at room temperature under a nitrogen atmosphere and the resulting mixture was stirred at room temperature for 1 h. The resulting mixture was concentrated under reduced pressure and the residue was purified by reverse flash chromatography to give the product. LCMS: (ES, m/z): [M+1]⁺: 188

### Preparation of 7-amino-5-methyl-1H-indole-3-carbonitrile

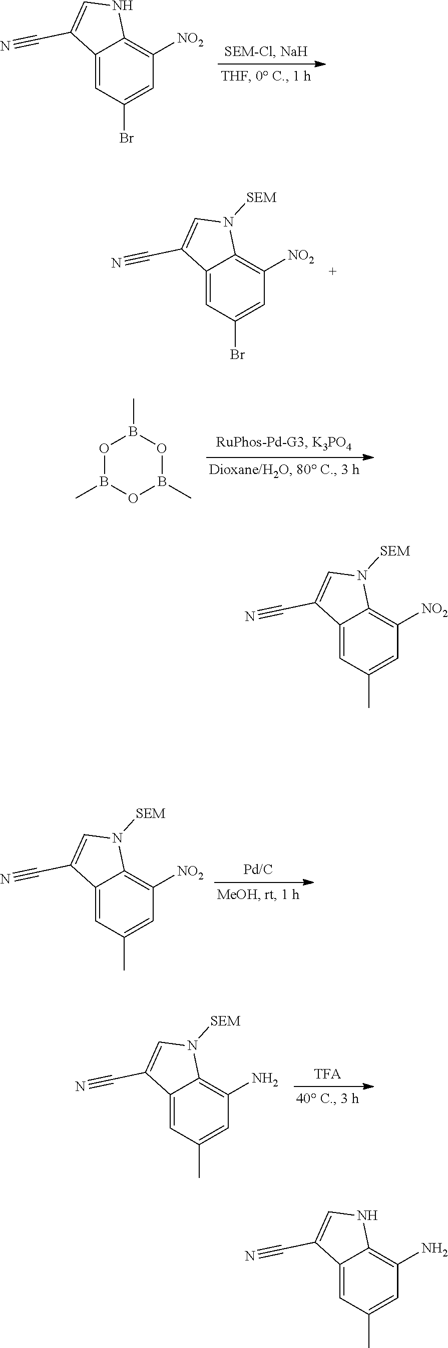

Preparation of 5-bromo-7-nitro-1-((2-(trimethylsilyl)ethoxy)methyl)-1H-indole-3-carbonitrile. To a solution of 5-bromo-7-nitro-1H-indole-3-carbonitrile (4.5 g, 16.9 mmol, 1 eq) in THE (90 mL) was added NaH (0.49 g, 20.3 mmol, 1.2 eq) and SEM-Cl (3.67 g, 21.9 mmol, 1.3 eq) dropwise at 0° C. under nitrogen atmosphere and the resulting mixture was stirred at room temperature under nitrogen atmosphere for 1 h. The reaction mixture was extracted with EtOAc. The combined organic layers were washed with brine, dried over anhydrous Na₂SO₄, and concentrated under reduced pressure to give the crude product which was used without further purification. LCMS (ES, m/z): [M+1]⁺: 396.

Preparation of 5-methyl-7-nitro-1-((2-(trimethylsilyl)ethoxy)methyl)indole-3-carbonitrile. To a solution of 5-bromo-7-nitro-1-((2-(trimethylsilyl)ethoxy)methyl)-1H-indole-3-carbonitrile (6.2 g, 15.6 mmol, 1 eq) in dioxane (100 mL) and H₂O (25 mL) was added K₃PO₄ (9.96 g, 46.9 mmol, 3 eq), RuPhos-Pd-G3 (650 mg, 0.8 mmol, 0.05 eq), and trimethyl-1,3,5,2,4,6-trioxatriborinane (5.89 g, 47 mmol, 3 eq) and the resulting mixture was stirred at 80° C. for 3 h under nitrogen atmosphere. The reaction mixture was extracted with EtOAc. The combined organic layers were washed with brine, dried over anhydrous Na₂SO₄, and concentrated under reduced pressure. The crude residue was purified by silica gel column chromatography to give the product. LCMS (ES, m/z): [M+1]⁺: 322.

Preparation of 7-amino-5-methyl-1-((2-(trimethylsilyl)ethoxy)methyl)indole-3-carbonitrile. To a solution of 5-methyl-7-nitro-1-((2-(trimethylsilyl)ethoxy)methyl)indole-3-carbonitrile (3.9 g, 11.7 mmol, 1 eq) in MeOH (160 mL) was added 10% Pd/C (0.63 g, 50% w.t) at room temperature and the resulting mixture was stirred for 1 h at room temperature under hydrogen atmosphere. The reaction mixture was filtered and concentrated under reduced pressure to give the crude product which was used without further purification. LCMS (ES, m/z): [M+1]⁺: 302.

Preparation of 7-amino-5-methyl-1H-indole-3-carbonitrile. A solution of 7-amino-5-methyl-1-((2-(trimethylsilyl)ethoxy)methyl)indole-3-carbonitrile (2.5 g, 8.29 mmol, 1 eq) in TFA (50 mL) was stirred at 40° C. for 3 h. The reaction mixture was concentrated under reduced pressure. The crude residue was mixture was brought to pH 8 with saturated NaHCO₃ and the precipitated solids were collected by filtration and washed with water to give the crude product which was used without further purification. LCMS (ES, m/z): [M+1]⁺: 172.

### Preparation of 3,4-dichloro-1H-indol-7-amine

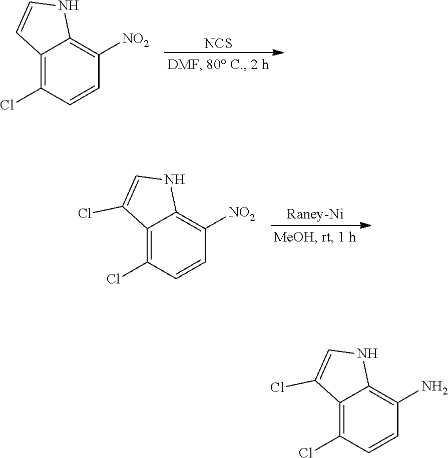 [SMILES: O=[N+]([O-])c1ccc(Cl)c2cc[nH]c12 ; "NCS" ; O=[N+]([O-])c1ccc(Cl)c2c(Cl)c[nH]c12 ; "Raney-Ni" ; Nc1ccc(Cl)c2c(Cl)c[nH]c12]

Preparation of 3,4-dichloro-7-nitro-1H-indole. To a solution of 4-chloro-7-nitro-1H-indole (2 g, 10.1 mmol, 1 eq) in DMF (40 mL) was added NCS (1.49 g, 11.1 mmol, 1.1 eq) at 0° C. and the resulting mixture was stirred for 1 h at 80° C. The reaction was quenched with water. The precipitated solids were collected by filtration and washed with water to give the crude product which was used without further purification. LCMS (ES, m/z): [M+1]⁺: 231

Preparation of 3,4-dichloro-1H-indol-7-amine. To a solution of 3,4-dichloro-7-nitro-1H-indole (2.05 g, 8.87 mmol, 1 eq) in MeOH (50 mL) was added Raney-Ni (20% w.t) and the resulting mixture was stirred for 1 h at room temperature under hydrogen atmosphere. The resulting mixture was filtered and concentrated under reduced pressure. The crude residue was purified by reversed-phase flash chromatography to give the product. LCMS (ES, m/z): [M+1]⁺: 201

### Preparation of 7-amino-3-chloro-1H-indole-4-carbonitrile

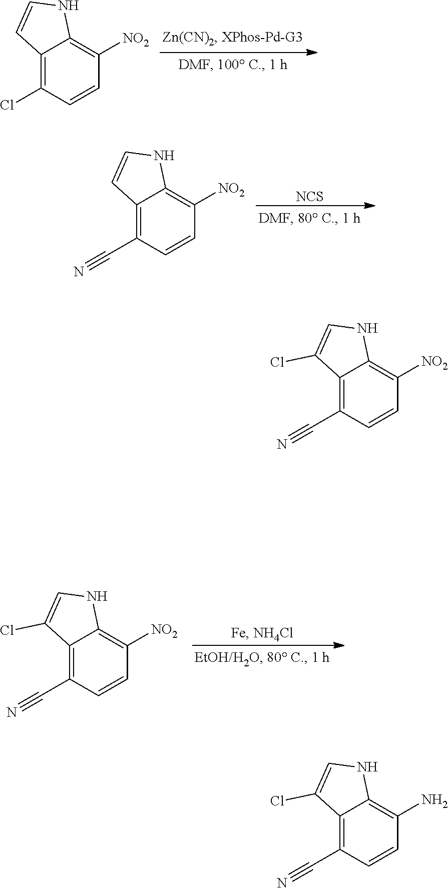

Preparation of 7-nitro-1H-indole-4-carbonitrile. To a solution of 4-chloro-7-nitro-1H-indole (8.1 g, 41.2 mmol, 1 eq) in DMF (160 mL) was added Zn(CN)₂ (5.81 g, 49.4 mmol, 1.2 eq), and XPhos-Pd-G3 (3.49 g, 4.12 mmol, 0.1 eq) and the resulting mixture was stirred for 1 h at 100° C. under nitrogen atmosphere. The reaction mixture was quenched with water and extracted with EtOAc. The combined organic layers were washed with brine, dried over anhydrous Na₂SO₄, and concentrated under reduced pressure to give the crude product which was used without further purification. LCMS: (ES, m/z): [M−1]⁻: 186

Preparation of 3-chloro-7-nitro-1H-indole-4-carbonitrile. To a solution of 7-nitro-1H-indole-4-carbonitrile (1.02 g, 5.45 mmol, 1 eq) in DMF (10 mL) was added NCS (0.80 g, 6 mmol, 1.1 eq) and the resulting mixture and was stirred for 1 h at 80° C. under nitrogen atmosphere. The reaction mixture was quenched with water and extracted with EtOAc. The combined organic layers were washed with brine, dried over anhydrous Na₂SO₄, and was concentrated under reduced pressure to give the crude product which was used without further purification. LCMS: (ES, m/z): [M−1]⁻: 220

Preparation of -amino-3-chloro-1H-indole-4-carbonitrile. To a solution of 3-chloro-7-nitro-1H-indole-4-carbonitrile (1.2 g, 5.4 mmol, 1 eq) in EtOH (18 mL) and H₂O (6 mL) was added NH₄Cl (2.90 g, 54.2 mmol, 10 eq), Fe (3.02 g, 54.2 mmol, 10 eq) at room temperature and the resulting mixture was stirred for 1 h at 80° C. under nitrogen atmosphere. The reaction mixture was concentrated under reduced pressure, diluted with water, and extracted with EtOAc. The combined organic layers were washed with brine, dried over anhydrous Na₂SO₄, and was concentrated under reduced pressure to give the crude product which was used without further purification. LCMS: (ES, m/z): [M+1]⁺:192

### Preparation of 7-amino-3-methyl-1H-indole-4-carbonitrile

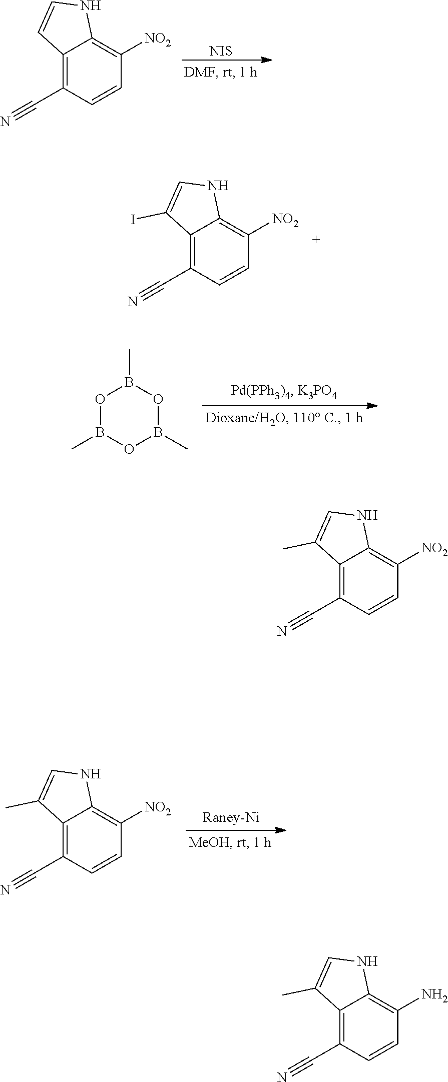

Preparation of 3-iodo-7-nitro-1H-indole-4-carbonitrile. To a solution of 7-nitro-1H-indole-4-carbonitrile (1 g, 5.3 mmol, 1 eq) in DMF (25 mL) was added NIS (1.32 g, 5.88 mmol, 1.1 eq) in portions at 0° C. and the resulting mixture was stirred for 1 h at room temperature. The reaction mixture was diluted with water. The precipitated solids were collected by filtration, washed with water, and dried under reduced pressure to give the crude product which was used without further purification. LCMS: (ES, m/z): [M−1]−: 312

Preparation of 3-methyl-7-nitro-1H-indole-4-carbonitrile. To a solution of 3-iodo-7-nitro-1H-indole-4-carbonitrile (1.53 g, 4.89 mmol, 1 eq) in dioxane (24 mL) and H₂O (6 mL) was added K₃PO₄ (3.1 g, 15 mmol, 3 eq), Pd(PPh₃)₄(0.56 g, 0.48 mmol, 0.1 eq), and trimethyl-1,3,5,2,4,6-trioxatriborinane (6.14 g, 48.8 mmol, 10 eq) at room temperature and the resulting mixture was stirred at 110° C. for 1 h. The reaction mixture was diluted with water. The precipitated solids were collected by filtration, washed with water, and dried under reduced pressure to give the crude product which was used without further purification. LCMS: (ES, m/z): [M−1]⁻: 200

Preparation of 7-amino-3-methyl-1H-indole-4-carbonitrile. To a solution of 3-methyl-7-nitro-1H-indole-4-carbonitrile (1.20 g, 6 mmol, 1 eq) in MeOH (60 mL) was added Raney-Ni (0.48 g, 40% wt) and the mixture was stirred for 1 h at room temperature under hydrogen atmosphere. The reaction mixture was filtered and concentrated under reduced pressure. The crude residue was purified by reversed-phase flash chromatography to give the product. LCMS: (ES, m/z): [M+1]⁺: 172

### Preparation of 3-amino-4H-thieno[3,2-b]pyrrole-6-carbonitrile

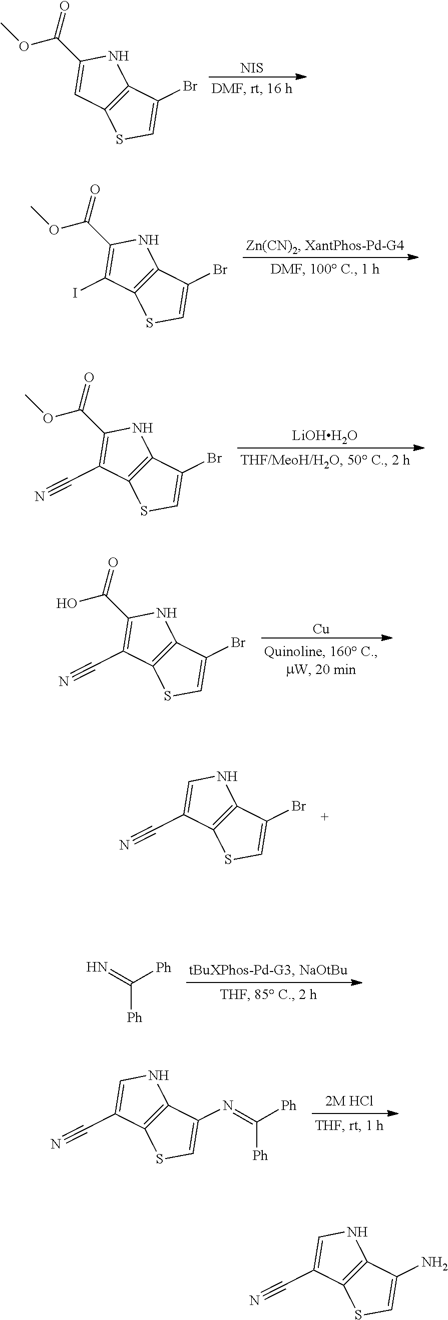

Preparation of methyl 3-bromo-6-iodo-4H-thieno[3,2-b]pyrrole-5-carboxylate. To a solution of methyl 3-bromo-4H-thieno[3,2-b]pyrrole-5-carboxylate (5 g, 19.2 mmol, 1.0 eq) in DMF (100 mL) was added NIS (4.76 g, 21.1 mmol, 1.1.0 eq) at 0° C. and the resulting mixture was stirred overnight at room temperature. The reaction mixture was quenched with water extracted with EtOAc. The combined organic layers were washed with brine, dried over anhydrous Na₂SO₄, and concentrated under reduced pressure to give the product which was used without further purification. LCMS: (ES) m/z: [M−1]⁻: 386

Preparation of methyl 3-bromo-6-cyano-4H-thieno[3,2-b]pyrrole-5-carboxylate. To a solution of methyl 3-bromo-6-iodo-4H-thieno[3,2-b]pyrrole-5-carboxylate (7 g, 18.1 mmol, 1.0 eq) in DMF (40 mL) was added, Zn(CN)₂ (1.28 g, 10.8 mmol, 0.6 eq), XantPhos-Pd-G4 (1.61 g, 1.81 mmol, 0.10 eq) and the resulting mixture was stirred for 1 h at 100° C. under nitrogen atmosphere. The reaction mixture was filtered and extracted with EtOAc. The combined organic layers were washed with, dried over anhydrous Na₂SO₄, and concentrated under reduced pressure. The crude residue was purified by reversed-phase flash chromatography to give the product. LCMS: (ES) m/z: [M−1]⁻: 285

Preparation of 3-bromo-6-cyano-4H-thieno[3,2-b]pyrrole-5-carboxylic acid. To a solution of methyl 3-bromo-6-cyano-4H-thieno[3,2-b]pyrrole-5-carboxylate (3.4 g, 11.9 mmol, 1.0 eq) in THF (28 mL), MeOH (14 mL) and H₂O (20 mL) was added LiOH—H₂O (8.01 g, 190 mmol, 16 eq) and the resulting mixture was stirred for 2 h at 50° C. The reaction mixture was cooled to room temperature, washed with EtOAc, and brought to pH 3 with 2M HCl (aq). The precipitated solids were collected by filtration and washed with water to give the product which was used without further purification. LCMS: (ES) m/z: [M+1]⁺: 271

Preparation of 3-bromo-4H-thieno[3,2-b]pyrrole-6-carbonitrile. To a solution of 3-bromo-6-cyano-4H-thieno[3,2-b]pyrrole-5-carboxylic acid (744 mg, 2.74 mmol, 1.0 eq) in quinoline (40 mL) was added Cu (34.9 mg, 0.549 mmol, 0.2 eq) and the resulting mixture was stirred at 160° C. for 20 min under microwave irradiation. The reaction mixture diluted with 2M HCl and extracted with EtOAc. The combined organic layers were washed with brine, dried over anhydrous Na₂SO₄, and concentrated under reduced pressure. The crude residue was purified by reversed-phase flash chromatography to give the product. LCMS: (ES) m/z: [M+1]⁺: 227

Preparation of 3-[(diphenylmethylidene)amino]-4H-thieno[3,2-b]pyrrole-6-carbonitrile. To a solution of 3-bromo-4H-thieno[3,2-b]pyrrole-6-carbonitrile (300 mg, 1.32 mmol, 1.0 eq) in THF (6 mL) was added t-BuXPhos-Pd-G3 (104 mg, 0.132 mmol, 0.10 eq), t-BuONa (380 mg, 3.96 mmol, 3 eq), and diphenylmethanimine (478 mg, 2.64 mmol, 2 eq) and the resulting mixture was stirred for 2 h at 85° C. under nitrogen atmosphere. The reaction mixture was quenched with water and extracted with EtOAc. The combined organic layers were washed with brine, dried over anhydrous Na₂SO₄, and concentrated under reduced pressure to give the product which was used without further purification. LCMS: (ES) m/z: [M+1]⁺: 328

Preparation of 3-amino-4H-thieno[3,2-b]pyrrole-6-carbonitrile. To a solution of 3-[(diphenylmethylidene)amino]-4H-thieno[3,2-b]pyrrole-6-carbonitrile (1.3 g, 3.97 mmol, 1.0 eq) in THF (13 mL) was added 2.0 M HCl (aq) (13 mL, 26 mmol, 6.5 eq) at 0° C. and the resulting mixture was stirred for 1 h at room temperature. The reaction mixture was concentrated under reduced pressure. The crude reside was purified by reversed-phase flash chromatography to give the product. LCMS: (ES) m/z: [M+1]⁺: 164.

### Preparation of 2-(7-amino-1H-indol-3-yl)acetonitrile

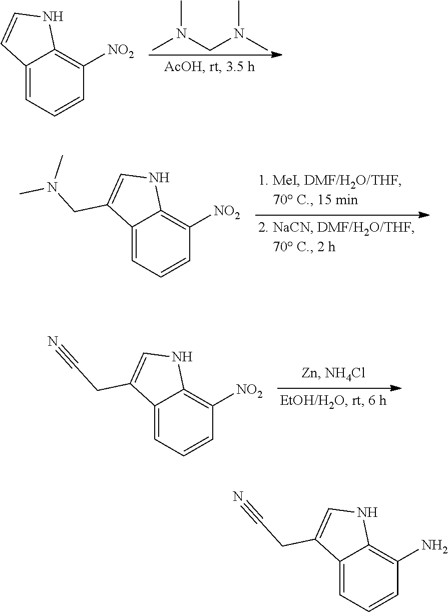

Preparation of N,N-dimethyl-1-(7-nitro-1H-indol-3-yl)methanamine. To a solution of [(dimethylamino)methyl]dimethylamine (1.39 g, 13.6 mmol, 1.1 eq) in AcOH (30 mL) was added 7-nitroindole (2.0 g, 12.3 mmol, 1.0 eq) in AcOH (30 mL) and the resulting mixture was stirred for 3.5 h at room temperature under nitrogen atmosphere. The reaction mixture was quenched with NaOH and extracted with DCM. The combined organic layers were washed with brine, dried over anhydrous Na₂SO₄, and concentrated under reduced pressure to give the product which was used without further purification. LCMS: (ES) m/z: [M−1]⁻: 220

Preparation of 2-(7-nitro-1H-indol-3-yl)acetonitrile. To a solution of N,N-dimethyl-1-(7-nitro-1H-indol-3-yl)methanamine (300 mg, 1.36 mmol, 1.0 eq) in DMF (0.3 mL), H₂O (0.3 mL), and THF (15 mL) was added and Mel (485 mg, 3.42 mmol, 2.5 eq) and the resulting mixture was stirred for 15 min at 70° C. under nitrogen atmosphere. To mixture was added NaCN (335 mg, 6.84 mmol, 5 eq) and the resulting mixture was stirred at 70° C. for 2 h. The reaction mixture filtered, washed with THF, and concentrated under reduced pressure to give the product which was used without further purification. LCMS: (ES) m/z: [M−1]⁻: 202

Preparation of 2-(7-amino-1H-indol-3-yl)acetonitrile. To a solution of 2-(7-nitro-1H-indol-3-yl)acetonitrile (250 mg, 1.24 mmol, 1.0 eq) in EtOH (7.5 mL) and H₂O (2.5 mL) was added NH₄Cl (664 mg, 12.4 mmol, 10 eq) and Zn (812 mg, 12.4 mmol, 10 eq) and the resulting mixture was stirred for 6 h at room temperature under nitrogen atmosphere. The reaction mixture was filtered, washed with EtOH, and concentrated under reduced pressure. The residue was dissolved in water and extracted with DCM. The combined organic layers were washed with brine, dried over anhydrous Na₂SO₄, and concentrated under reduced pressure to give the product which was used without further purification. LCMS: (ES) m/z: [M−1]⁻: 172

### Example 3: Preparation of Specific Final Compounds

### Preparation of N-(3-cyano-4-methyl-1H-indol-7-yl)-5-(dimethylphosphoryl)thiazole-2-sulfonamide (Compound 111)

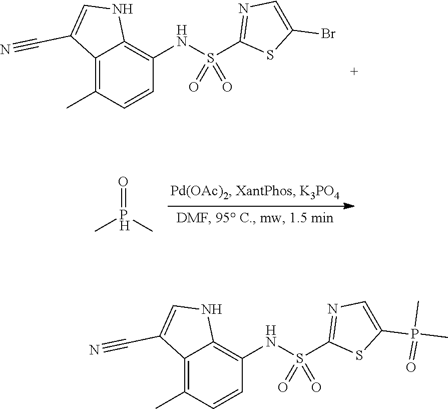

Preparation of N-(3-cyano-4-methyl-1H-indol-7-yl)-5-(dimethylphosphoryl)thiazole-2-sulfonamide. To a solution of 5-bromo-N-(3-cyano-4-methyl-1H-indol-7-yl)-1,3-thiazole-2-sulfonamide (200 mg, 0.50 mmol, 1 eq) and (methylphosphonoyl)methane (197 mg, 2.51 mmol, 5 eq) in DMF (10 mL) was added Pd(OAc)₂ (11.3 mg, 0.05 mmol, 0.10 eq), XantPhos (29.0 mg, 0.050 mmol, 0.1 eq), and K₃PO₄ (160.29 mg, 0.754 mmol, 1.5 eq) and the resulting mixture was stirred at 95° C. for 1.5 min microwave irradiation. The residue was purified by reversed-phase flash chromatography to give the product. LCMS (ES, m/z): [M+1]+: 395, ¹H NMR (300 MHz, DMSO-d₆, ppm) δ 12.13 (d, J=3.1 Hz, 1H), 10.94 (s, 1H), 8.44 (d, J=3.3 Hz, 1H), 8.22 (d, J=3.1 Hz, 1H), 6.86 (d, J=7.8 Hz, 1H), 6.72 (d, J=7.7 Hz, 1H), 2.61 (s, 3H), 1.83 (d, J=14.1 Hz, 6H).

### Preparation of N-(3-cyano-1H-indol-7-yl)-2-(2-hydroxy-2-methylpropoxy)-1-methyl-1H-imidazole-5-sulfonamide (Compound 394)

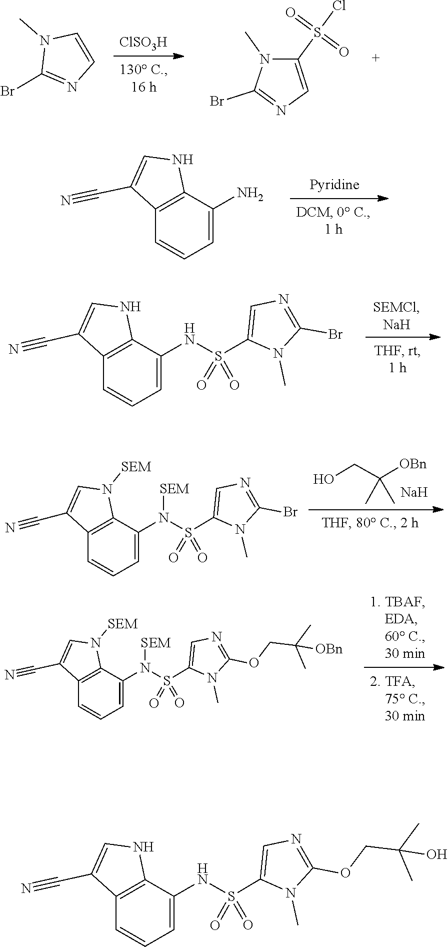

Preparation of 2-bromo-1-methylimidazole-4-sulfonyl chloride. A solution of 2-bromo-1-methylimidazole (2 g, 12.4 mmol, 1 eq) in HSO₃Cl (16.0 mL) was stirred overnight at 130° C. under nitrogen atmosphere. The reaction mixture was diluted with water and extracted with DCM. The combined organic layers were washed with brine, dried over anhydrous Na₂SO₄, and concentrated under reduced pressure. The residue was purified by reversed-phase flash chromatography to give the product. LCMS (ES, m/z): [M+1]⁺: 259, ¹H NMR (300 MHz, DMSO-d6, ppm) δ 7.76 (s, 1H), 3.69 (s, 3H).

Preparation of 2-bromo-N-(3-cyano-1H-indol-7-yl)-1-methylimidazole-4-sulfonamide. To a stirred mixture of 7-amino-1H-indole-3-carbonitrile (350 mg, 2.22 mmol, 1 eq) in THF (10 mL) was added 2-bromo-1-methylimidazole-4-sulfonyl chloride (636 mg, 2.45 mmol, 1.10 eq) and pyridine (528 mg, 6.70 mmol, 3 eq) and the resulting mixture was stirred for 1 h at room temperature under nitrogen atmosphere. The reaction mixture was diluted with water and extracted with DCM. The combined organic layers were washed with brine, dried over anhydrous Na₂SO₄, and concentrated under reduced pressure to give the crude product which was used without further purification. LCMS [M+1]⁺: 380

Preparation of 2-bromo-N-(3-cyano-1-((2-(trimethylsilyl)ethoxy)methyl)-1H-indol-7-yl)-1-methyl-N-((2-(trimethylsilyl)ethoxy)methyl)-1H-imidazole-5-sulfonamide. To a stirred mixture of 2-bromo-N-(3-cyano-1H-indol-7-yl)-1-methylimidazole-4-sulfonamide (1 g, 2.63 mmol, 1 eq) in THE (20.0 mL) was added NaH (158 mg, 6.58 mmol, 2.50 eq) at 0° C. under nitrogen atmosphere and the resulting mixture was stirred for 30 min at room temperature under nitrogen atmosphere. To the mixture was added SEM-Cl (965 mg, 5.79 mmol, 2.20 eq) and the resulting mixture was stirred for 1 h at room temperature under nitrogen atmosphere. The reaction mixture was diluted with water and extracted with EtOAc. The combined organic layers were washed with brine, dried over anhydrous Na₂SO₄, and concentrated under reduced pressure to give the crude product which was used without further purification. LCMS (ES, m/z): [M+1]+: N/A

Preparation of 2-(2-(benzyloxy)-2-methylpropoxy)-N-(3-cyano-1-((2-(trimethylsilyl)ethoxy)methyl)-1H-indol-7-yl)-1-methyl-N-((2-(trimethylsilyl)ethoxy)methyl)-1H-imidazole-5-sulfonamide. To a solution of 2-(benzyloxy)-2-methylpropan-1-ol (281 mg, 1.56 mmol, 2 eq) in THE (10.0 mL) was added NaH (37.5 mg, 1.56 mmol, 2 eq) at 0° C. and the resulting mixture was stirred for 10 min at 80° C. under nitrogen atmosphere. To the mixture was added 2-bromo-N-(3-cyano-1-((2-(trimethylsilyl)ethoxy)methyl)-1H-indol-7-yl)-1-methyl-N-((2-(trimethylsilyl)ethoxy)methyl)-1H-imidazole-5-sulfonamide (500 mg, 0.780 mmol, 1 eq) in THE (1 mL) and the resulting mixture was stirred for 2 h at 80° C. under nitrogen atmosphere. The reaction mixture was diluted with water and extracted with EtOAc. The combined organic layers were washed with brine, dried over anhydrous Na₂SO₄, and concentrated under reduced pressure to give the crude product which was used without further purification. LCMS [M+1]⁺: 740

Preparation of N-(3-cyano-1H-indol-7-yl)-2-(2-hydroxy-2-methylpropoxy)-1-methyl-1H-imidazole-5-sulfonamide. To a solution of 2-(2-(benzyloxy)-2-methylpropoxy)-N-(3-cyano-1-((2-(trimethylsilyl)ethoxy)methyl)-1H-indol-7-yl)-1-methyl-N-((2-(trimethylsilyl)ethoxy)methyl)-1H-imidazole-5-sulfonamide (200 mg, 0.33 mmol, 1 eq) in TBAF (2 mL, 1M in THF) was added 1,2-ethylenediamine (29.6 mg, 0.49 mmol, 1.50 eq) and the resulting mixture was stirred for 30 min at 65° C. under nitrogen atmosphere. The reaction mixture was diluted with water and extracted with EtOAc. The combined organic layers were washed with brine, dried over anhydrous Na₂SO₄, and concentrated under reduced pressure. To the crude residue was added TFA (6 mL) and the resulting mixture was stirred for 0.5 h at 75° C. under nitrogen atmosphere. The reaction mixture was concentrated under vacuum. The crude residue was purified by reversed-phase flash chromatography to give the title compound. LCMS (ES, m/z): [M+1]⁺: 390, ¹H NMR (400 MHz, DMSO-d₆, ppm) δ 11.92 (s, 1H), 10.21 (s, 1H), 8.14 (s, 1H), 7.32 (s, 1H), 7.16-7 (m, 2H), 6.91 (dd, J=7.7, 1.0 Hz, 1H), 4.71 (s, 1H), 4.05 (s, 2H), 3.44 (s, 3H), 1.14 (s, 6H).

### Preparation of Methyl 2-(5-(chlorosulfonyl)-2-oxothiazol-3(2H)-yl)acetate

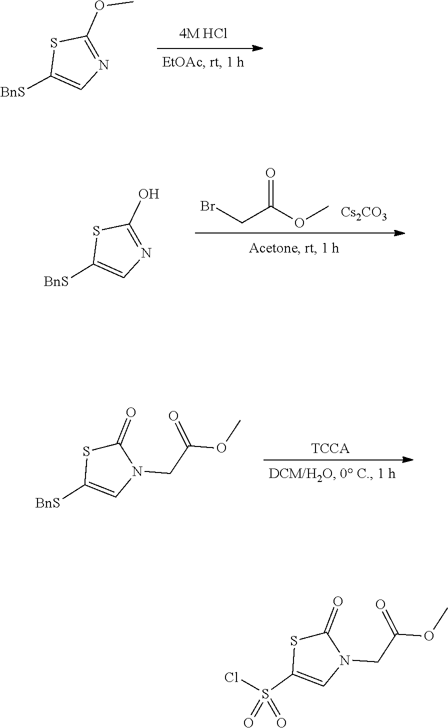

Preparation of 5-(benzylsulfanyl)-1,3-thiazol-2-ol. To a solution of 5-(benzylsulfanyl)-2-methoxy-1,3-thiazole (1 g, 4.21 mmol, 1 eq) in EtOAc (10 mL) was added 4M HCl (10 mL, 40 mmol, 10 eq) and the resulting mixture was stirred for 1 h at room temperature. The resulting mixture was concentrated under reduced pressure to give the crude product which was used without further purification. LCMS (ES, m/z): [M+1]⁺: 224

Preparation of methyl 2-[5-(benzylsulfanyl)-2-oxo-1,3-thiazol-3-yl]acetate. To a solution of 5-(benzylsulfanyl)-1,3-thiazol-2-ol (860 mg, 3.85 mmol, 1 eq) in acetone (20 mL) was added methyl 2-bromoacetate (706 mg, 4.62 mmol, 1.2 eq) and Cs₂CO₃ (1.88 g, 5.77 mmol, 1.5 eq) and the resulting mixture was stirred for 1 h at room temperature. The reaction mixture was diluted with water and extracted with EtOAc. The combined organic layers were washed with brine, dried over anhydrous Na₂SO₄, and concentrated under reduced pressure to give the crude product which was used without further purification. LCMS (ES, m/z): [M+1]⁺: 296

Preparation of methyl 2-[5-(chlorosulfonyl)-2-oxo-1,3-thiazol-3-yl]acetate. To a solution of methyl 2-[5-(benzylsulfanyl)-2-oxo-1,3-thiazol-3-yl]acetate (900 mg, 3.04 mmol, 1 eq) in DCM (5 mL) and H₂O (16 mL) was added 1,3,5-trichloro-1,3,5-triazinane-2,4,6-trione (778 mg, 3.35 mmol, 1.5 eq) at 0° C. and the resulting mixture was stirred for 1 h at room temperature. The reaction mixture was diluted with water and extracted with DCM. The combined organic layers were washed with brine, dried over anhydrous Na₂SO₄, and concentrated under reduced pressure to give the crude product which was used without further purification. LCMS (ES, m/z): [M+1]⁺: 272

### Preparation of N-(3-cyano-1H-indol-7-yl)thiazole-5-sulfonamide (Compound 50)

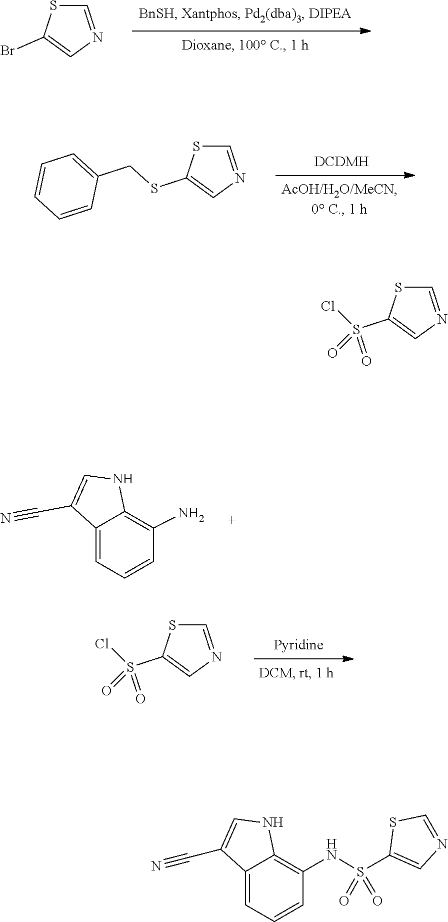 [SMILES: Brc1cncs1 ; c1ccc(CSc2cncs2)cc1 ; O=S(=O)(Cl)c1cncs1 ; N#Cc1c[nH]c2c(N)cccc12 ; O=S(=O)(Cl)c1cncs1 ; N#Cc1c[nH]c2c(NS(=O)(=O)c3cncs3)cccc12]

Preparation of 5-(benzylsulfanyl)-1,3-thiazole. To a solution of 5-bromo-1,3-thiazole (30 g, 184 mmol, 1 eq) in dioxane (600 mL) was added benzyl mercaptan (27 g, 219 mmol, 1.2 eq), DIPEA (71 g, 552 mmol, 3 eq), XantPhos (10.6 g, 18.3 mmol, 0.10 eq), and Pd₂(dba)₃ (8.37 g, 9.15 mmol, 0.05 eq) and the resulting mixture was stirred for 1 h at 100° C. under nitrogen atmosphere. The reaction was quenched with water and extracted with EtOAc. The combined organic layers were with brine, dried over anhydrous Na₂SO₄, and concentrated under reduced pressure. The crude residue was purified by silica gel column chromatography to give the product (38 g, 92%). LCMS (ES, m/z): [M+1]⁺: 208

Preparation of thiazole-5-sulfonyl chloride. To a solution of 5-(benzylsulfanyl)-1,3-thiazole (30 g, 145 mmol, 1 eq) in MeCN (300 mL), AcOH (17 mL) and H₂O (13 mL) was added 1,3-dichloro-5,5-dimethylimidazolidine-2,4-dione (43 g, 217 mmol, 1.50 eq) at 0° C. and the resulting mixture was stirred for 1 h at room temperature. The reaction mixture quenched water extracted with DCM. The combined organic layers were washed with brine, dried over anhydrous Na₂SO₄, and concentrated under reduced pressure to give the crude product which was used without further purification. LCMS ES, m/z: [M+Aniline+1]⁺: 240

Preparation of N-(3-cyano-1H-indol-7-yl)-1,3-thiazole-5-sulfonamide. To a solution of 7-amino-1H-indole-3-carbonitrile (15 g, 95.4 mmol, 1 eq) in DCM (300 mL) was added pyridine (23 g, 286 mmol, 3 eq) and 1,3-thiazole-5-sulfonyl chloride (21 g, 115 mmol, 1.2 eq) in DCM (150 mL) at 0° C. and the resulting mixture was stirred for 1 h at room temperature under nitrogen atmosphere. The reaction was quenched with water and extracted with DCM. The combined organic layers were washed with brine, dried over anhydrous Na₂SO₄, and concentrated under reduced pressure. The crude residue was purified by reversed-phase flash chromatography to give the title compound (10 g, 34%). LCMS ES, m/z: [M+1]⁺: 305, ¹H NMR—PH-ROP-2021-03-259-0: (300 MHz, DMSO-d₆, ppm) δ 12.01 (s, 1H), 10.49 (s, 1H), 9.34 (d, J=0.9 Hz, 1H), 8.27-8.18 (m, 2H), 7.53 (d, J=7.9 Hz, 1H), 7.16 (t, J=7.8 Hz, 1H), 6.86 (dd, J=7.6, 1.0 Hz, 1H). ¹³C NMR—PH-ROP-2021-03-259-0: (75 MHz, DMSO-d₆, ppm) δ 161.28, 147.82, 136.72, 135.54, 131.25, 129.04, 122.52, 122.33, 119.33, 117.69, 116.35, 85.42

### Preparation of N-(3-cyano-1H-indol-7-yl)-2-oxo-2,3-dihydrothiazole-5-sulfonamide (Compound 353)

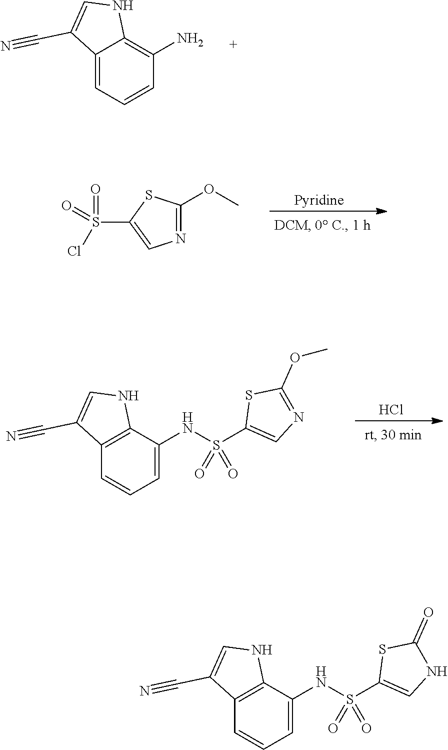 [SMILES: N#Cc1c[nH]c2c(N)cccc12 ; COc1ncc(S(=O)(=O)Cl)s1 ; "Pyridine" ; COc1ncc(S(=O)(=O)Nc2cccc3c(C#N)c[nH]c23)s1 ; Cl ; N#Cc1c[nH]c2c(NS(=O)(=O)c3c[nH]c(=O)s3)cccc12]

Preparation of N-(3-cyano-1H-indol-7-yl)-2-methoxythiazole-5-sulfonamide. To a solution of 7-amino-1H-indole-3-carbonitrile (200 mg, 1.27 mmol, 1 eq) and Pyridine (301 mg, 3.81 mmol, 3 eq) in DCM (4 mL) was added 2-methoxy-1,3-thiazole-5-sulfonyl chloride (326 mg, 1.52 mmol, 1.2 eq) at 0° C. and the resulting mixture was stirred for 1 h at room temperature. The reaction mixture was diluted with water and extracted with EtOAc. The combined organic layers were washed with bring, dried with Na₂SO₄, and concentrated under reduced pressure. The crude residue was purified by reversed-phase flash to give the product. LCMS: (ES, m/z): [M+1]⁺: 335, ¹H NMR (300 MHz, DMSO-d₆, ppm) δ 11.98 (s, 1H), 10.36 (s, 1H), 8.22 (d, J=3.1 Hz, 1H), 7.61-7.47 (m, 2H), 7.18 (t, J=7.8 Hz, 1H), 6.97 (dd, J=7.6, 1.1 Hz, 1H), 4.06 (s, 3H).

Preparation of N-(3-cyano-1H-indol-7-yl)-2-oxo-2,3-dihydrothiazole-5-sulfonamide. A solution of N-(3-cyano-1H-indol-7-yl)-2-methoxy-1,3-thiazole-5-sulfonamide (150 mg, 0.449 mmol, 1 eq) in conc. HCl (3 mL) was stirred for 30 min at room temperature. The reaction mixture was neutralized to pH 7 with saturated Na₂CO₃ and extracted with EtOAc (2×5 mL). The combined organic layers were washed with bring, dried with Na₂SO₄, and concentrated under reduced pressure. The crude residue was purified by reversed-phase flash to give the product. LCMS (ES, m/z): [M+1]⁺: 319, ¹H NMR (400 MHz, DMSO-d₆, ppm) δ 11.84-12.33 (s, 2H), 10.30 (s, 1H), 8.22 (dd, J=3.1, 1.7 Hz, 1H), 7.56-7.45 (m, 2H), 7.20 (td, J=7.8, 1.7 Hz, 1H), 7.04 (dt, J=7.6, 1.3 Hz, 1H).

### Preparation of N-(3-cyano-1H-indol-7-yl)-1-(2-hydroxy-2-methylpropyl)-2-oxo-2,3-dihydro-1H-imidazole-4-sulfonamide (Compound 408)

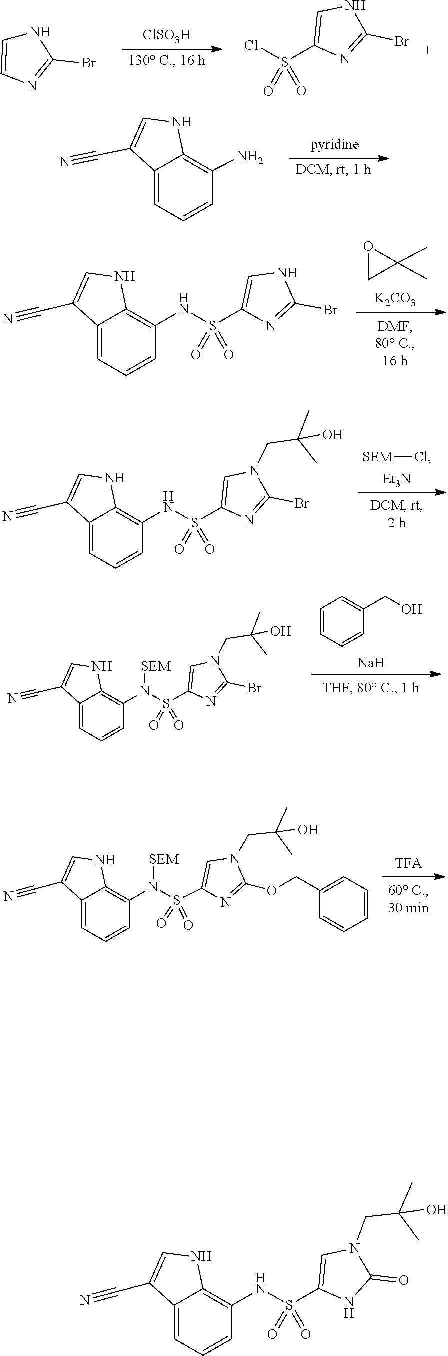

Preparation of 2-bromo-1H-imidazole-4-sulfonyl chloride. A solution of 2-bromo-1H-imidazole (8 g, 54.4 mmol, 1.0 eq) in HSO₃Cl (80 mL) was stirred at 130° C. for 16 h. The reaction mixture was quenched with water extracted with EtOAc. The combined organic layers were washed with brine, dried over anhydrous Na₂SO₄, and concentrated under reduced pressure to give the product which was used without further purification. LCMS: (ES) m/z: [M+1]⁺: 245

Preparation of 2-bromo-N-(3-cyano-1H-indol-7-yl)-1H-imidazole-4-sulfonamide. To a solution of 7-amino-1H-indole-3-carbonitrile (1 g, 6.36 mmol, 1.0 eq) and pyridine (1.51 g, 19.0 mmol, 3 eq) in DCM (10 mL) was added 2-bromo-1H-imidazole-4-sulfonyl chloride (2.34 g, 9.54 mmol, 1.5 eq) in DCM (10 mL) and the resulting mixture was stirred for 1 h at room temperature under nitrogen atmosphere. The reaction mixture was concentrated under reduced pressure. The crude residue was purified by reversed-phase flash chromatography to give the product. LCMS: (ES) m/z: [M+1]⁺: 366

Preparation of 2-bromo-N-(3-cyano-1H-indol-7-yl)-1-(2-hydroxy-2-methylpropyl) imidazole-4-sulfonamide. To a solution of 2-bromo-N-(3-cyano-1H-indol-7-yl)-1H-imidazole-4-sulfonamide (1 g, 2.73 mol, 1.0 eq) in DMF (20 mL) was added K₂CO₃ (1.13 g, 8.19 mmol, 3 eq) and 2,2-dimethyloxirane (0.39 g, 5.46 mmol, 2 eq) and the resulting mixture was stirred overnight at 80° C. under nitrogen atmosphere. The reaction mixture was diluted with water and extracted with DCM. The combined organic layers were washed with brine, dried over anhydrous Na₂SO₄, and concentrated under reduced pressure. The crude residue was purified by reversed-phase flash chromatography to give the product.

LCMS: (ES) m/z: [M+1]⁺: 438, HNMR: (300 MHz, DMSO-d₆, ppm) δ 11.93 (s, 1H), 9.64 (s, 1H), 8.20 (s, 1H), 7.72 (s, 1H), 7.41 (dd, J=8.0, 1.0 Hz, 1H), 7.08 (t, J=7.8 Hz, 1H), 6.91 (dd, J=7.7, 1.0 Hz, 1H), 4.83 (s, 1H), 3.85 (s, 2H), 0.99 (s, 6H).

Preparation of 2-bromo-N-(3-cyano-1H-indol-7-yl)-1-(2-hydroxy-2-methylpropyl)-N-{[2-(trimethylsilyl)ethoxy]methyl}imidazole-4-sulfonamide. To a solution of 2-bromo-N-(3-cyano-1H-indol-7-yl)-1-(2-hydroxy-2-methylpropyl)imidazole-4-sulfonamide (460 mg, 1.05 mol, 1.0 eq) in DCM (20 mL) was added Et₃N (318 mg, 3.15 mmol, 3 eq) and SEM-Cl (524 mg, 3.15 mmol, 3 eq) and the resulting mixture was stirred for 2 h at room temperature under nitrogen atmosphere. The reaction mixture was concentrated under reduced pressure and extracted with EtOAc. The combined organic layers were washed with brine, dried over anhydrous Na₂SO₄, and concentrated under reduced pressure to give the product which was used without further purification. LCMS: (ES) m/z: [M+1]⁺: 568

Preparation of 2-(benzyloxy)-N-(3-cyano-1H-indol-7-yl)-1-(2-hydroxy-2-methylpropyl)-N-{[2-(trimethylsilyl)ethoxy]methyl}-2,3-dihydroimidazole-4-sulfonamide. To a solution of benzyl alcohol (608 mg, 5.63 mmol, 5 eq) in THE (10 mL) was added NaH (135 mg, 5.63 mmol, 5 eq) and 2-bromo-N-(3-cyano-1H-indol-7-yl)-1-(2-hydroxy-2-methylpropyl)-N-{[2-(trimethylsilyl)ethoxy]methyl}imidazole-4-sulfonamide (640 mg, 1.12 mmol, 1.0 eq) in THE (10 mL) at 0° C. and the resulting mixture was stirred for 1 h at room temperature under nitrogen atmosphere. The reaction mixture was quenched with water and extracted with EtOAc. The combined organic layers were washed with brine, dried over anhydrous Na₂SO₄, and concentrated under reduced pressure. The crude residue was purified by reversed-phase flash chromatography to give the product. LCMS: (ES) m/z: [M+1]⁺: 598

Preparation of N-(3-cyano-1H-indol-7-yl)-1-(2-hydroxy-2-methylpropyl)-2-oxo-3H-imidazole-4-sulfonamide. A solution of 2-(benzyloxy)-N-(3-cyano-1H-indol-7-yl)-1-(2-hydroxy-2-methylpropyl)-N-{[2-(trimethylsilyl)ethoxy]methyl}-2,3-dihydroimidazole-4-sulfonamide (130 mg, 0.217 mmol, 1.0 eq) in TFA (4 mL) was stirred for 30 min at 60° C. under nitrogen atmosphere. The reaction mixture was concentrated under vacuum. The crude residue was purified by reversed-phase flash chromatography to give the product. LCMS: (ES) m/z: [M+1]⁺: 376, ¹H NMR (400 MHz, DMSO-d₆, ppm) δ 11.97-11.92 (m, 1H), 11.01 (s, 1H), 9.89 (s, 1H), 8.24 (s, 1H), 7.47 (d, J=8.0 Hz, 1H), 7.15 (t, J=7.8 Hz, 1H), 7.03 (s, 1H), 6.98 (d, J=7.6 Hz, 1H), 4.64 (s, 1H), 3.39 (s, 2H), 0.96 (s, 6H).

### Preparation of N-(3-cyano-4-methyl-1H-indol-7-yl)-2-ethyl-1H-imidazole-4-sulfonamide (Compound 8)

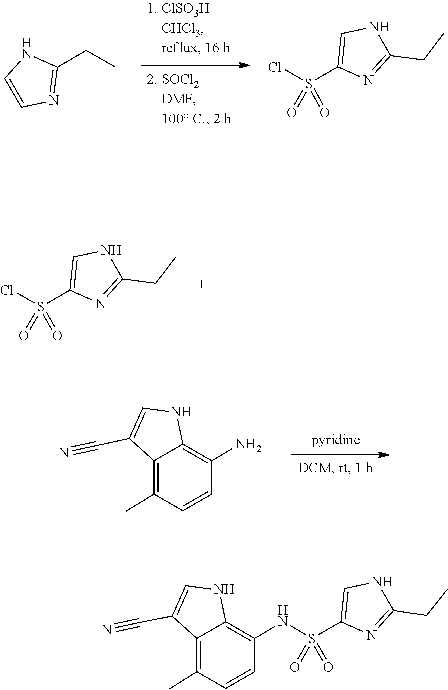

Preparation of 2-ethyl-1H-imidazole-4-sulfonyl chloride. To a solution of 2-ethyl-1H-imidazole (20 g, 208 mmol, 1.0 eq) in CHCl₃ (1.6 L) was added chlorosulfonic acid (80 mL) and the resulting mixture was stirred overnight at reflux under nitrogen atmosphere. To the mixture was added SOCl₂ (600 mL), DMF (3 drops, catalyst) and the resulting mixture was stirred for additional 2 h at 100° C. The reaction mixture was concentrated under reduced pressure, diluted with water, and extracted with DCM. The combined organic layers were washed with brine, dried over anhydrous Na₂SO₄, and concentrated under reduced pressure to give the product which was used without further purification. LCMS: (ES, m/z): [M+1]⁺: 195

Preparation of N-(3-cyano-4-methyl-1H-indol-7-yl)-2-ethyl-1H-imidazole-4-sulfonamide. To a solution of 7-amino-4-methyl-1H-indole-3-carbonitrile (7 g, 40.9 mmol, 1 eq) and pyridine (7.32 g, 122 mmol, 3 eq) in DCM (100 mL) was added 2-ethyl-1H-imidazole-4-sulfonyl chloride (9.56 g, 49.0 mmol, 1.2 eq) in DCM (40 mL) and the resulting mixture was stirred for 1 h at room temperature under nitrogen atmosphere. The reaction mixture was concentrated under reduced pressure. The crude residue was purified by reversed-phase flash chromatography to give the product. LCMS: (ES, m/z): [M+1]⁺: 330, ¹H NMR: (400 MHz, DMSO-d₆, ppm) δ 12.41 (s, 1H), 12.16-12.11 (m, 1H), 9.77 (s, 1H), 8.20 (d, J=3.0 Hz, 1H), 7.55 (d, J=1.7 Hz, 1H), 6.81 (s, 2H), 2.65 (q, J=7.6 Hz, 2H), 2.57 (s, 3H), 1.20 (t, J=7.6 Hz, 3H).

### Preparation of N-(3-cyano-4-methyl-1H-indol-7-yl)-4-(trifluoromethyl)-1,3-thiazole-2-sulfonamide (Compound 12)

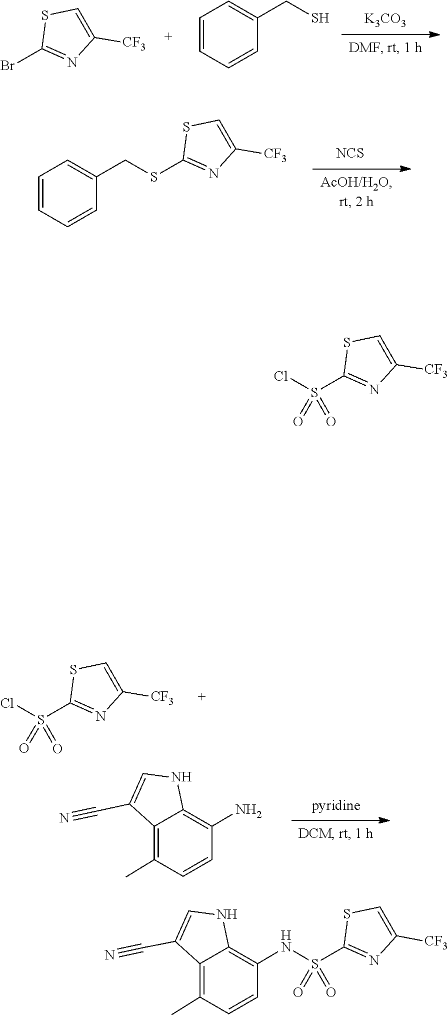

Preparation of 2-(benzylsulfanyl)-4-(trifluoromethyl)-1,3-thiazole. To a solution of 2-bromo-4-(trifluoromethyl)-1,3-thiazole (15.0 g, 64.6 mmol, 1.0 eq) in DMF (300 mL) was added benzyl mercaptan (9.64 g, 77.5 mmol, 1.2 eq) and K₂CO₃ (13.4 g, 96.9 mmol, 1.5 eq) and the resulting mixture was stirred for additional 1 h at room temperature. The reaction mixture was quenched with water and extracted with DCM. The combined organic layers were washed with brine, dried over anhydrous Na₂SO₄, and concentrated under reduced pressure. The residue was purified by silica gel column chromatography to give the product. LCMS (ES, m/z): [M+1]⁺: 276

Preparation of 4-(trifluoromethyl)-1,3-thiazole-2-sulfonyl chloride. To a solution of 2-(benzylsulfanyl)-4-(trifluoromethyl)-1,3-thiazole (13.0 g, 47.2 mmol, 1.00 eq) in AcOH (260 mL) and H₂O (26 mL) was added NCS (25.2 g, 188 mmol, 4.0 eq) in portions and the resulting mixture was stirred for additional 2 h at room temperature. The reaction mixture was quenched with water and extracted with DCM. The combined organic layers were washed with brine, dried over anhydrous Na₂SO₄, and concentrated under reduced pressure to give the product (12 g, 87% yield) which was used without further purification. LCMS (ES, m/z): [M+1]⁺: 252

Preparation of N-(3-cyano-4-methyl-1H-indol-7-yl)-4-(trifluoromethyl)-1,3-thiazole-2-sulfonamide. To a solution of 7-amino-4-methyl-1H-indole-3-carbonitrile (7 g, 40.9 mmol, 1.0 eq) in DCM (140 mL) was added pyridine (16.2 g, 204 mmol, 5.0 eq) and 4-(trifluoromethyl)-1,3-thiazole-2-sulfonyl chloride (11.3 g, 44.9 mmol, 1.1 eq) in DCM (5 mL) dropwise at 0° C. and the resulting mixture was stirred for additional 1 h at room temperature. The reaction mixture was extracted with DCM. The combined organic layers were washed with brine, dried over anhydrous Na₂SO₄, concentrated under reduced pressure. The residue was purified by reversed-phase flash chromatography to give the product. LCMS (ES, m/z): [M+1]⁺: 387, ¹H NMR (300 MHz, DMSO-d6, ppm) δ 12.23 (d, J=3.1 Hz, 1H), 11.02 (s, 1H), 8.85 (s, 1H), 8.25 (d, J=3.1 Hz, 1H), 6.86 (d, J=7.7 Hz, 1H), 6.60 (d, J=7.6 Hz, 1H), 2.61 (s, 3H).

### Preparation of N-(3-chloro-4-fluoro-1H-indol-7-yl)-4-(trifluoromethyl)-1,3-thiazole-2-sulfonamide (Compound 159)

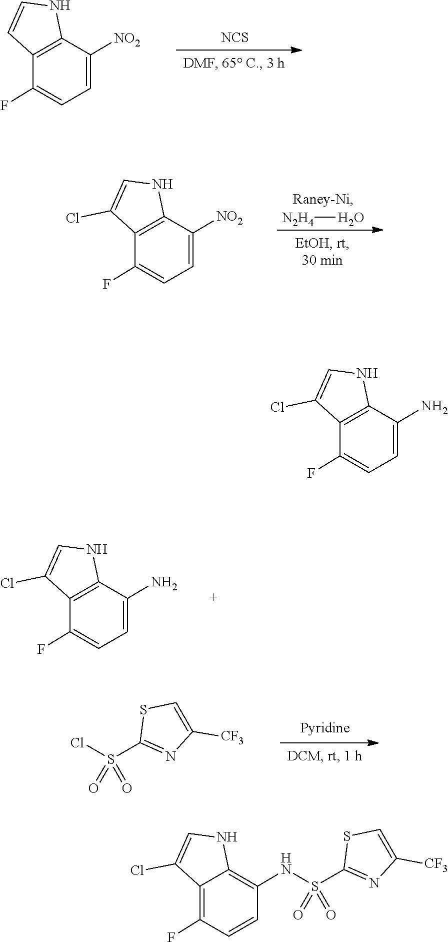

Preparation of 3-chloro-4-fluoro-7-nitro-1H-indole. To A solution of 4-fluoro-7-nitro-1H-indole (1 g, 5.55 mmol, 1 eq) in DMF (20 mL) was added NCS (815 mg, 6.10 mmol, 1.1 eq) and the resulting mixture was stirred for 3 h at 65° C. under nitrogen atmosphere. The crude residue was purified by trituration with water. The precipitated solids were collected by filtration, washed with water, and dried under reduced pressure to give the product which was used without further purification. LCMS (ES, m/z): [M+1]⁺: 214.

Preparation of 3-chloro-4-fluoro-1H-indol-7-amine. To a solution of 3-chloro-4-fluoro-7-nitro-1H-indole (1.1 g, 5.12 mmol, 1 eq) in EtOH (55 mL) was added Raney-Ni (1.1 g, 100% w.t) and hydrazine hydrate (0.40 g, 10.2 mmol, 2.0 eq, 80% solution) and the resulting mixture was stirred for 30 min at room temperature. The reaction mixture was filtered, the filter cake was washed with EtOH, and the filtrate was concentrated under reduced pressure. The crude residue was purified by reversed-phase flash chromatography to give the product. LCMS (ES, m/z): [M+1]⁺: 185.

Preparation of N-(3-chloro-4-fluoro-1H-indol-7-yl)-4-(trifluoromethyl)-1,3-thiazole-2-sulfonamide. To a solution of 3-chloro-4-fluoro-1H-indol-7-amine (150 mg, 0.81 mmol, 1.0 eq) in DCM (3 mL) was added pyridine (146 mg, 2.43 mmol, 3.0 eq) and 4-(trifluoromethyl)-1,3-thiazole-2-sulfonyl chloride (245 mg, 0.97 mmol, 1.2 eq) in DCM (0.5 mL) dropwise at 0° C. and the resulting mixture was stirred for 1 h at room temperature under nitrogen atmosphere. The reaction mixture was concentrated under reduced pressure. The residue was purified by reversed-phase flash chromatography to give the product. LCMS (ES, m/z): [M+1]⁺: 400, ¹H NMR (300 MHz, DMSO-d₆, ppm) δ 11.69 (s, 1H), 10.96 (s, 1H), 8.86 (s, 1H), 7.54 (d, J=2.7 Hz, 1H), 6.76 (dd, J=10.8, 8.3 Hz, 1H), 6.59 (dd, J=8.4, 4.3 Hz, 1H).

### Preparation of N-(3-cyano-4-ethyl-1H-indol-7-yl)-1,3-thiazole-2-sulfonamide (Compound 216)

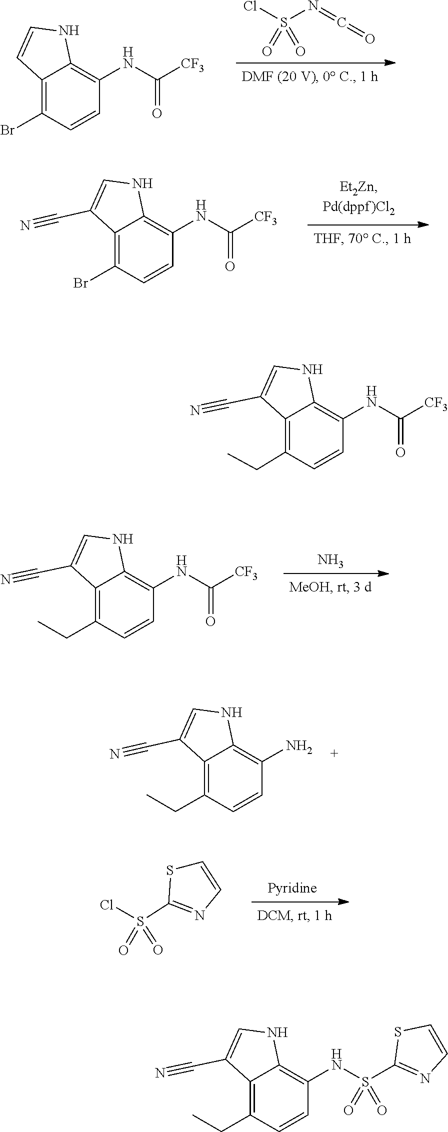

Preparation of N-(4-bromo-3-cyano-1H-indol-7-yl)-2,2,2-trifluoroacetamide. To a solution of N-(4-bromo-1H-indol-7-yl)-2,2,2-trifluoroacetamide (2.50 g, 8.14 mmol, 1.0 eq) in DMF (50 mL) was added chlorosulfonyl isocyanate (3.50 g, 24.4 mmol, 3.00 eq) dropwise at 0° C. and the resulting mixture was stirred at 0° C. for 1 h. The reaction mixture was diluted with water and extracted with EtOAc. The combined organic layers were washed with brine, dried over anhydrous Na₂SO₄, and concentrated under reduced pressure. The residue was purified by reversed-phase flash chromatography to give the product. LCMS (ES, m/z): [M+1]⁺: 332

Preparation of N-(3-cyano-4-ethyl-1H-indol-7-yl)-2,2,2-trifluoroacetamide. To a solution of N-(4-bromo-3-cyano-1H-indol-7-yl)-2,2,2-trifluoroacetamide (1.00 g, 3.01 mmol, 1.0 eq) in THE (20 mL) was added diethylzinc (1.12 g, 9.03 mmol, 3.0 eq) and Pd(dppf)Cl₂ (0.11 g, 0.15 mmol, 0.05 eq) and the resulting mixture was stirred for 1 h at 70° C. under nitrogen atmosphere. The reaction mixture was concentrated under reduced pressure. The crude residue was purified by reversed-phase flash chromatography to give the product. LCMS (ES, m/z): [M+1]⁺: 282

Preparation of 7-amino-4-ethyl-1H-indole-3-carbonitrile. To a solution of N-(3-cyano-4-ethyl-1H-indol-7-yl)-2,2,2-trifluoroacetamide (750 mg, 2.66 mmol, 1.0 eq) and NH₃ (g) in MeOH (8 mL) and the resulting mixture was stirred for 3 days at room temperature. The resulting mixture was concentrated under vacuum to give the product which was used without further purification. LCMS (ES, m/z): [M+1]⁺: 186

Preparation of N-(3-cyano-4-ethyl-1H-indol-7-yl)-1,3-thiazole-2-sulfonamide. To a solution of 7-amino-4-ethyl-1H-indole-3-carbonitrile (200 mg, 1.08 mmol, 1.0 eq) in DCM (2 mL) was added pyridine (257 mg, 3.24 mmol, 3.0 eq) and 1,3-thiazole-2-sulfonyl chloride (238 mg, 1.29 mmol, 1.2 eq) in DCM (2 mL) dropwise at 0° C. and the resulting mixture was stirred for additional 1 h at room temperature. The reaction mixture was concentrated under reduced pressure. The crude residue was purified by reversed-phase flash chromatography to give the product. LCMS (ES, m/z): [M+1]⁺: 333, ¹H NMR (300 MHz, DMSO-d₆, ppm) δ 12.13 (d, J=3.2 Hz, 1H), 10.74 (s, 1H), 8.23 (d, J=3.1 Hz, 1H), 8.13 (s, 2H), 6.87 (d, J=7.8 Hz, 1H), 6.69 (d, J=7.8 Hz, 1H), 2.98 (q, J=7.5 Hz, 2H), 1.24 (t, J=7.5 Hz, 3H).

### Preparation of N-(3-cyano-1H-indazol-7-yl)-1,3-thiazole-5-sulfonamide (Compound 244)

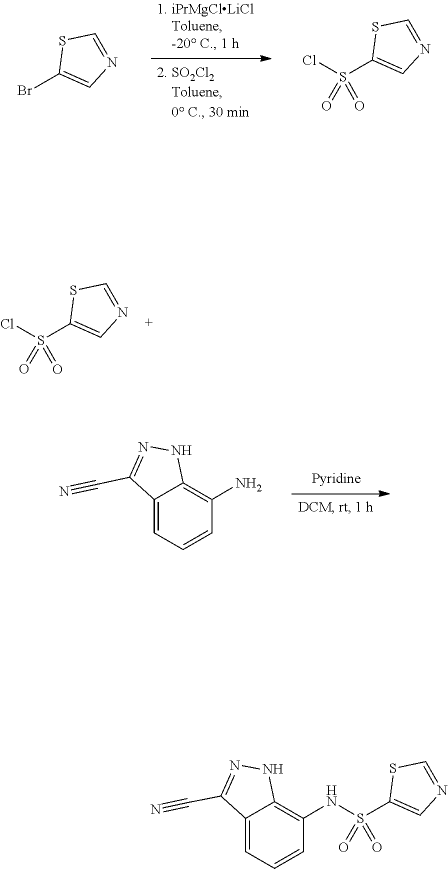 [SMILES: Brc1cncs1 ; O=S(=O)(Cl)c1cncs1 ; O=S(=O)(Cl)c1cncs1 ; N#Cc1n[nH]c2c(N)cccc12 ; N#Cc1n[nH]c2c(NS(=O)(=O)c3cncs3)cccc12]

Preparation of 1,3-thiazole-5-sulfonyl chloride. To a solution of 5-bromo-1,3-thiazole (1.5 g, 9.14 mmol, 1.0 eq) in toluene (15 mL) was added iPrMgCl-LiCl (9.1 mL, 11.9 mmol, 1.3 eq, 1.3M in THF) dropwise at −20° C. and the resulting mixture was stirred for 1 h at room temperature under nitrogen atmosphere. To the reaction mixture was added SO₂Cl₂ (1.11 mL, 13.7 mmol, 1.5 eq) in toluene (15 mL) dropwise at 0° C. and the resulting mixture was stirred at room temperature for 30 min. The reaction was quenched with water and extracted with EtOAc. The combined organic layers were washed with brine, dried over anhydrous Na₂SO₄, and concentrated under reduced pressure to give the product which was used without further purification. LCMS (ES, m/z): [M+1]⁺: 184.

Preparation of N-(3-cyano-1H-indazol-7-yl)-1,3-thiazole-5-sulfonamide. To a solution of 7-amino-1H-indazole-3-carbonitrile (150 mg, 0.94 mmol, 1.0 eq) in DCM (3 mL) was added pyridine. (225 mg, 2.84 mmol, 3.0 eq) and 1,3-thiazole-5-sulfonyl chloride (348 mg, 1.89 mmol, 2.0 eq) dropwise at 0° C. and the resulting mixture was stirred for 1 h at room temperature under nitrogen atmosphere. The reaction mixture was concentrated under reduced pressure. The crude residue was purified by reversed-phase flash chromatography to give the product. LCMS (ES, m/z): [M+1]⁺: 306, ¹H NMR—PH-ROP-2021-03-340-0: (400 MHz, DMSO-d₆, ppm) δ 14.38 (s, 1H), 10.76 (s, 1H), 9.38 (s, 1H), 8.23 (s, 1H), 7.79 (d, J=8.2 Hz, 1H), 7.30 (t, J=7.8 Hz, 1H), 6.99 (d, J=7.4 Hz, 1H).

### Preparation of N-(3-cyano-1H-indol-7-yl)-3-methyl-1,2-thiazole-5-sulfonamide (Compound 256)

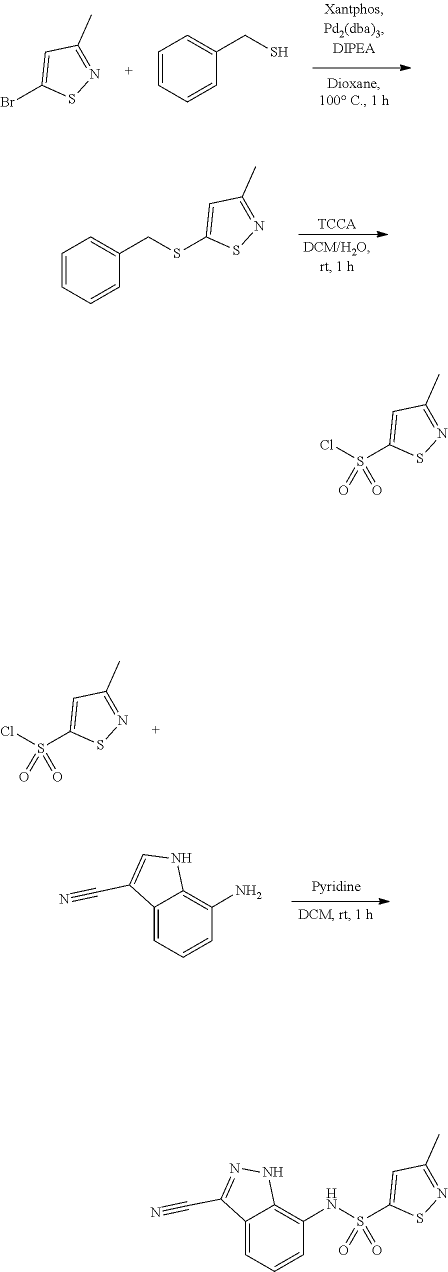

Preparation of 5-(benzylsulfanyl)-3-methyl-1,2-thiazole. To a solution of 5-bromo-3-methyl-1,2-thiazole (15 g, 84.2 mmol, 1.0 eq) in dioxane (300 mL) was added benzyl mercaptan (12.5 g, 101 mmol, 1.2 eq), DIPEA (32.5 g, 252 mmol, 3.0 eq), XantPhos (4.87 g, 8.42 mmol, 0.1 eq) and Pd₂(dba)₃ (3.86 g, 4.21 mmol, 0.05 eq) and the resulting mixture was stirred for 1 h at 100° C. under nitrogen atmosphere. The reaction mixture was quenched with water and extracted with EtOAc. The combined organic layers were washed with brine, dried over anhydrous Na₂SO₄, and concentrated under reduced pressure. The residue was purified by silica gel column chromatography to give the product. LCMS: (ES, m/z): [M+1]⁺: 222

Preparation of 3-methyl-1,2-thiazole-5-sulfonyl chloride. To a solution of 5-(benzylsulfanyl)-3-methyl-1,2-thiazole (18 g, 85.8 mmol, 1.0 eq) in DCM (108 mL) and H₂O (324 mL) was added trichloroisocyanuric acid (29.9 g, 129 mmol, 1.5 eq) at 0° C. and the resulting mixture was stirred for 1 h at room temperature. The reaction mixture was filtered, the filter cake was washed with DCM, and the filtrate was extracted with DCM. The combined organic layers were washed with brine, dried over anhydrous Na₂SO₄, and concentrated under reduced pressure to give the product which was used without further purification.

Preparation of Preparation of N-(3-cyano-1H-indol-7-yl)-3-methyl-1,2-thiazole-5-sulfonamide. To a solution of 7-amino-1H-indole-3-carbonitrile (9.0 g, 57.2 mmol, 1.0 eq) in DCM (180 mL) was added pyridine (13.6 g, 172 mmol, 3.0 eq) and 3-methyl-1,2-thiazole-5-sulfonyl chloride (12.4 g, 63.0 mmol, 1.1 eq) at 0° C. and the resulting mixture was stirred for 1 h at room temperature. The reaction mixture was quenched with water and extracted with DCM. The combined organic layers were washed with brine, dried over anhydrous Na₂SO₄, and concentrated under reduced pressure. The crude residue was purified by reversed-phase flash chromatography to give the product. LCMS: (ES, m/z): [M+1]⁺: 319, ¹H NMR (300 MHz, DMSO-d₆, ppm) δ 12.07 (s, 1H), 10.70 (s, 1H), 8.24 (d, J=3.1 Hz, 1H), 7.60-7.51 (m, 2H), 7.17 (t, J=7.8 Hz, 1H), 6.88 (dd, J=7.7, 1.0 Hz, 1H), 2.44 (s, 3H).

### Preparation of N-(3-cyano-1H-indol-7-yl)-2-cyclopropyl-1,3-oxazole-5-sulfonamide (Compound 107)

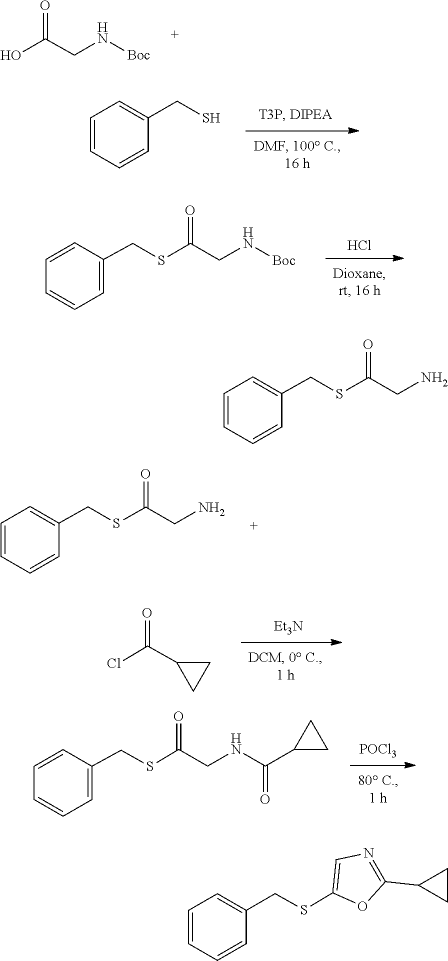

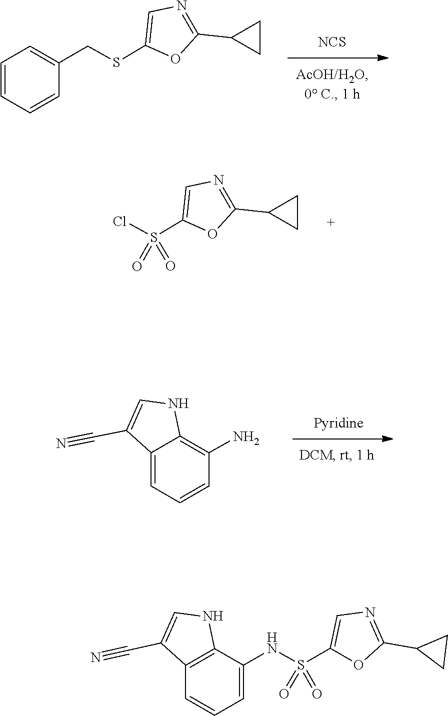 [SMILES: c1ccc(CSc2cnc(C3CC3)o2)cc1 ; "NCS" ; O=S(=O)(Cl)c1cnc(C2CC2)o1 ; N#Cc1c[nH]c2c(N)cccc12 ; "Pyridine" ; N#Cc1c[nH]c2c(NS(=O)(=O)c3cnc(C4CC4)o3)cccc12]

Preparation of tert-butyl N-[2-(benzylsulfanyl)-2-oxoethyl]carbamate. To a solution of (tert-butoxycarbonyl)glycine (60 g, 0.34 mol, 1.0 eq) in DMF (1.2 L) was added DIPEA (133 g, 1.03 mol, 3.0 eq), benzyl mercaptan (51.0 g, 0.41 mol, 1.2 eq) and T3P (327 g, 0.51 mol, 1.5 eq, 50% EtOAc solution) and the resulting mixture was stirred overnight at 100° C. under nitrogen atmosphere. The reaction mixture was diluted with water and extracted with EtOAc. The combined organic layers were washed with brine, dried over anhydrous Na₂SO₄, concentrated under reduced pressure to give the product which was used without further purification. LCMS (ES, m/z): [M+1]⁺: 282

Preparation of S-benzyl 2-aminoethanethioate. A solution of tert-butyl N-[2-(benzylsulfanyl)-2-oxoethyl]carbamate (200 g, 0.71 mol, 1.0 eq) in HCl/dioxane (4 L, 4 M, 4.0 5.7 eq) was stirred overnight at room temperature. The reaction mixture was concentrated under reduced pressure. The crude residue was purified by reversed-phase flash chromatography to give the product. LCMS (ES, m/z): [M+1]⁺: 182

Preparation of N-[2-(benzylsulfanyl)-2-oxoethyl]cyclopropanecarboxamide. To a solution of S-benzyl 2-aminoethanethioate (98 g, 0.34 mol, 1.0 eq) in DCM (1 L) was added Et₃N (104 g, 1.03 mol, 3.0 eq) and cyclopropanecarbonyl chloride (42.7 g, 0.41 mol, 1.2 eq) at 0° C. and the resulting mixture was stirred for 1 h at room temperature. The resulting mixture was concentrated under reduced pressure. The crude residue was purified by reversed-phase flash chromatography to give the product. LCMS (ES, m/z): [M+1]⁺: 250

Perparation of 5-(benzylsulfanyl)-2-cyclopropyl-1,3-oxazole. A solution of N-[2-(benzylsulfanyl)-2-oxoethyl]cyclopropanecarboxamide (22.7 g, 0.091 mol, 1.0 eq) and POCl₃ (230 mL) was stirred for 2 h at 80° C. under nitrogen atmosphere. The reaction mixture was concentrated under reduced pressure, diluted with water, and extracted with DCM. The combined organic layers were washed with brine, dried over anhydrous Na₂SO₄, and concentrated under reduced pressure to give the product which was used without further purification. LCMS (ES, m/z): [M+1]⁺: 232

Preparation of 2-cyclopropyl-1,3-oxazole-5-sulfonyl chloride. To a solution of 5-(benzylsulfanyl)-2-cyclopropyl-1,3-oxazole (20 g, 86 mmol, 1.0 eq) (450-2) in AcOH (265 mL) and H₂O (135 mL) was added NCS (40.4 g, 303 mol, 3.5 eq) at 0° C. and the resulting mixture was stirred for 1 h at room temperature. The reaction mixture was diluted with water and extracted with DCM. The combined organic layers were washed with brine, dried over anhydrous Na₂SO₄, and concentrated under reduced pressure to give the product which was used without further purification. LCMS (ES, m/z): [M+1]⁺: 207 Preparation of N-(3-cyano-1H-indol-7-yl)-2-cyclopropyl-1,3-oxazole-5-sulfonamide. To a solution of 7-amino-1H-indole-3-carbonitrile (8.0 g, 51 mmol, 1.0 eq) in DCM (120 mL) was added Pyridine (120 mL) and 2-cyclopropyl-1,3-oxazole-5-sulfonyl chloride (12.7 g, 611 mmol, 1.2 eq) in DCM (20 mL) at 0° C. and the resulting mixture was stirred for 1 h at room temperature. The reaction mixture was concentrated under reduced pressure. The crude residue was purified by reversed-phase flash chromatography to give the product. LCMS (ES, m/z): [M+1]⁺: 329, ¹H NMR (400 MHz, DMSO-d₆, ppm) δ 12.09 (d, J=3.2 Hz, 1H), 10.63 (s, 1H), 8.25 (d, J=3.1 Hz, 1H), 7.60-7.51 (m, 2H), 7.18 (t, J=7.8 Hz, 1H), 6.85 (dd, J=7.6, 0.9 Hz, 1H), 2.19 (tt, J=8.4, 4.8 Hz, 1H), 1.13 (dt, J=8.4, 3.5 Hz, 2H), 0.98-0.89 (m, 2H).

### Preparation of N-(3-cyano-1H-indol-7-yl)-4-(hydroxymethyl) thiophene-2-sulfonamide (Compound 306)

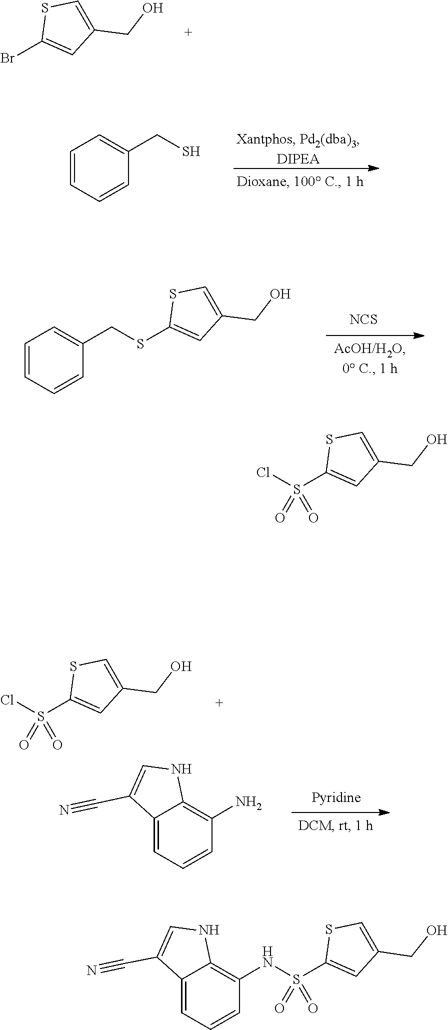

Preparation of [5-(benzylsulfanyl) thiophen-3-yl]methanol. To a solution of (5-bromothiophen-3-yl) methanol (600 mg, 3.1 mmol, 1.0 eq) in dioxane (12 mL) was added XantPhos (179 mg, 0.311 mmol, 0.1 eq), dioxane (12 mL), DIPEA (1.20 g, 9.32 mmol, 3 eq), benzyl mercaptan (462 mg, 3.73 mol, 1.2 eq) and Pd2(dba)₃ (142 mg, 0.155 mmol, 0.05 eq) and the resulting mixture was stirred for 1 h at 100° C. under nitrogen atmosphere. The crude residue was purified by reversed-phase flash chromatography to give the product. LCMS: (ES, m/z): [M+1]⁺: 237

Preparation of 4-(hydroxymethyl) thiophene-2-sulfonyl chloride. To a solution of [5-(benzylsulfanyl) thiophen-3-yl]methanol (500 mg, 2.1 mmol, 1.0 eq) in AcOH (10 mL) and H₂O (1 mL) was added NCS (847 mg, 6.34 mmol, 3.0 eq) at 0° C. and the resulting mixture was stirred for 1 h at room temperature. The reaction mixture was quenched with water and extracted with. The combined organic layers were washed with brine, dried over anhydrous Na₂SO₄, and concentrated under reduced pressure to give the product which was used without further purification. LCMS: (ES, m/z): [M+1]⁺: 213

Preparation of N-(3-cyano-1H-indol-7-yl)-4-(hydroxymethyl) thiophene-2-sulfonamide. To a solution of 7-amino-1H-indole-3-carbonitrile (150 mg, 0.95 mmol, 1.0 eq) in DCM (4 mL) was added pyridine (226 mg, 2.86 mmol, 3.0 eq) and 4-(hydroxymethyl) thiophene-2-sulfonyl chloride (243 mg, 1.14 mmol, 1.2 eq) at 0° C. and the resulting mixture was stirred for 1 h at room temperature. The reaction mixture was concentrated under reduced pressure. The crude residue was purified by reverse-phase flash chromatography to give the product. LCMS (ES, m/z): [M−1]⁻: 332, ¹H NMR (400 MHz, DMSO-d₆, ppm) δ 11.97-11.90 (m, 1H), 10.26 (s, 1H), 8.22 (d, J=2.7 Hz, 1H), 7.64 (d, J=1.6 Hz, 1H), 7.52-7.41 (m, 2H), 7.14 (t, J=7.8 Hz, 1H), 6.90 (dd, J=7.7, 0.9 Hz, 1H), 5.27 (t, J=5.8 Hz, 1H), 4.39 (d, J=5.6 Hz, 2H).

### Preparation of N-(3-cyano-4-fluoro-1H-indol-7-yl)-3-methyl-1,2-oxazole-5-sulfonamide (Compound 354)

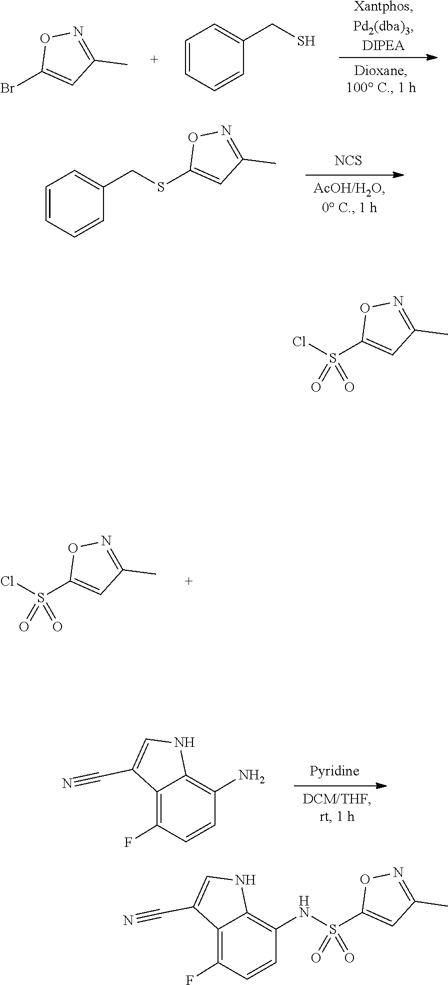

Preparation of 5-(benzylthio)-3-methylisoxazole. To a solution of 5-bromo-3-methylisoxazole (200 mg, 1.23 mmol, 1.0 eq) in dioxane (4 mL) was added benzyl mercaptan (184 mg, 1.48 mmol, 1.2 eq), Pd2(dba)₃ (114 mg, 0.12 mmol, 0.1 eq), XantPhos (142 mg, 0.24 mmol, 0.2 eq) and DIPEA (319 mg, 2.40 mmol, 2.0 eq) and the resulting mixture was stirred for 1 h at 80° C. under nitrogen atmosphere. The reaction was quenched with water and extracted with EtOAc. The combined organic layers were washed with brine, dried over anhydrous Na2SO4, and concentrated under reduced pressure. The crude residue was purified by reverse-phase flash chromatography to give the product. LCMS: (ES, m/z): [M+1]⁺: 192

Preparation of 3-methylisoxazole-5-sulfonyl chloride. To a solution of 5-(benzylthio)-3-methylisoxazole (110 mg, 0.53 mmol, 1.0 eq) in MeCN (1.1 mL), AcOH (0.11 mL), and H₂O (0.11 mL), was added NCS (85.8 mg, 0.64 mmol, 1.2 eq) at 0° C. and the resulting mixture was stirred for 1 h at room temperature under a nitrogen atmosphere. The reaction was quenched with water and extracted with EtOAc. The combined organic layers were washed with brine, dried over anhydrous Na₂SO₄, and concentrated under reduced pressure to give the product which was used without further purification. LCMS: (ES, m/z): [M+1]⁺: 182

Preparation of N-(3-cyano-4-fluoro-1H-indol-7-yl)-3-methyl-1,2-oxazole-5-sulfonamide. To a solution of 3-methyl-1,2-oxazole-5-sulfonyl chloride (124 mg, 0.68 mmol, 2.0 eq) in THE (1.2 mL) was added 7-amino-4-fluoro-1H-indole-3-carbonitrile (60 mg, 0.343 mmol, 1.0 eq) and pyridine (81.3 mg, 1.03 mmol, 3.0 eq) at 0° C. and the resulting mixture was stirred for 1 h at room temperature. The reaction mixture was concentrated under reduced pressure. The crude residue was purified by preparative reverse-phase chromatography to give the product. LCMS (ES, m/z): [M+1]⁺: 321, ¹H NMR (300 MHz, DMSO-d₆, ppm) δ 12.56 (s, 1H), 11.00 (s, 1H), 8.33 (d, J=2.9 Hz, 1H), 6.97 (d, J=9.1 Hz, 2H), 6.75 (dd, J=8.5, 4.3 Hz, 1H), 2.29 (s, 3H). ¹⁹F NMR (300 MHz, DMSO-d₆, ppm) δ −124.8.

### Preparation of N-(3-cyano-4-methyl-1H-indazol-7-yl)-1,3-thiazole-5-sulfonamide (Compound 342)

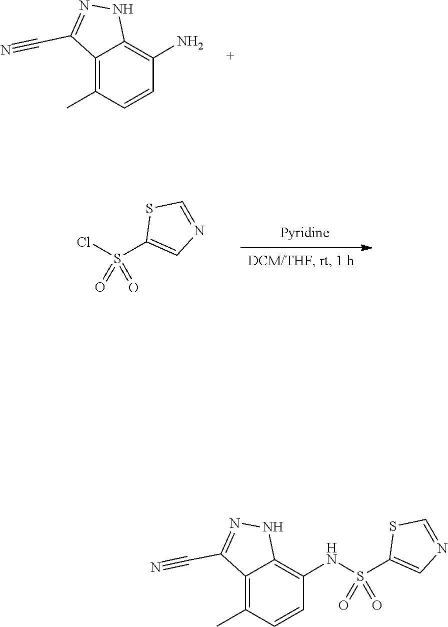 [SMILES: Cc1ccc(N)c2[nH]nc(C#N)c12 ; O=S(=O)(Cl)c1cncs1 ; Cc1ccc(NS(=O)(=O)c2cncs2)c2[nH]nc(C#N)c12]

To a solution of 7-amino-4-methyl-1H-indazole-3-carbonitrile (300 mg, 1.74 mmol, 1.0 eq) in THE (3 mL) and DCM (3 mL) was added pyridine (689 mg, 8.71 mmol, 5.0 eq) and 1,3-thiazole-5-sulfonyl chloride (383 mg, 2.09 mmol, 1.2 eq) at 0° C. and the resulting mixture was stirred for 1 h at room temperature under nitrogen atmosphere. The reaction mixture was concentrated under reduced pressure. The residue was purified by preparative reverse-phase chromatography to give the product. LCMS (ES, m/z): [M+1]⁺: 320, ¹H NMR (400 MHz, DMSO-d₆, ppm) δ 14.36 (s, 1H), 10.60 (s, 1H), 9.38 (d, J=0.9 Hz, 1H), 8.20 (d, J=0.9 Hz, 1H), 7.02 (dd, J=7.5, 1.1 Hz, 1H), 6.81 (d, J=7.5 Hz, 1H), 2.68-2.64 (m, 3H).

TABLE B
Synthetic procedures for compounds disclosed herein

| | General | |
|---|---|---|
| Compound | Procedure | Analytical Report |
| 1 | A | LCMS: m/z [M + H]+ 395.2. 1H-NMR (400 MHZ, DMSO-d6): δ 12.12 (s, 1H), |
| | | 10.80 (s, 1H), 8.45 (s, 1H), 8.21 (d, J = 2.8 Hz, 1H), 7.92 (d, J = 1.6 Hz, 1H), |
| | | 7.90 (s, 1H), 7.50-7.41 (m, 3H), 6.85 (d, J = 8.0 Hz, 1H), 6.75 (d, J = 8.0 Hz, |
| | | 1H), 2.59 (s, 3H) |
| 2 | A | LCMS: m/z [M + H]+ 319.1. 1HNMR-400 MHZ, DMSO-d6: δ 12.05 (s, 1H), 10.70 |
| | | (s, 1H), 8.19 (d, J = 2.8 Hz, 1H), 8.09 (s, 2H), 6.81 (d, J = 8.0 Hz, 1H), 6.65 (d, |
| | | J = 8.0 Hz, 1H), 2.58 (s, 3H). |
| 3 | A | LCMS: m/z [M − H]− 351.2. 1H-NMR (400 MHZ, DMSO-d6): δ 11.85 (s, 1H), 7.89 |
| | | (s, 1H), 7.82 (s, 1H), 6.88 (d, J = 7.6 Hz, 2H), 6.64 (d, J = 7.2 Hz, 1H), 2.51 (s, 3H) |
| 4 | A | LCMS: m/z [M + H]+ 369.2. 1H-NMR (400 MHz, DMSO-d6): δ 12.11 (s, 1H), |
| | | 11.00 (s, 1H), 8.21-8.16 (m, 3H), 7.68-7.59 (m, 2H), 6.75 (s, 2H), 2.56 (s, 3H) |
| 5 | A | LCMS: m/z [M − H]− 395.2. 1H-NMR (400 MHZ, DMSO-d6): δ 12.00 (s, 1H), |
| | | 10.88 (s, 1H), 8.06 (s, 2H), 6.77 (s, 2H), 2.55 (s, 3H) |
| 6 | A | LCMS: m/z [M − H]− 342.2. 1H-NMR (400 MHZ, DMSO-d6): δ 12.13 (s, 1H), |
| | | 11.02 (s, 1H), 9.07 (s, 1H), 8.16 (s, 1H), 6.83 (d, J = 6.8 Hz, 1H), 6.68 (d, J = 6.4 Hz, 1H), |
| | | 2.59 (s, 3H) |
| 7 | A | LCMS (ES) m/z: 353.10 [M + H]+, 1H-NMR (DMSO-d6, 400 MHZ, ppm) δ 12.08 |
| | | (s, 1H), 10.50 (s, 1H), 8.21 (d, 1H, J = 3.2 Hz), 7.97 (s, 1H), 6.90 (d, 1H, J = 8.0 Hz), |
| | | 6.73 (d, 1H, J = 8.0 Hz), 2.62 (s, 3H). |
| 8 | F | LCMS (ES) m/z: [M + 1]+: 330 |
| | | 1H NMR (400 MHZ, DMSO-d6, ppm) δ 12.41 (s, 1H), 12.16-12.11 (m, 1H), |
| | | 9.77 (s, 1H), 8.20 (d, J = 3.0 Hz, 1H), 7.55 (d, J = 1.7 Hz, 1H), 6.81 (s, 2H), 2.65 |
| | | (q, J = 7.6 Hz, 2H), 2.57 (s, 3H), 1.20 (t, J = 7.6 Hz, 3H). |
| 9 | A | LCMS (ES) m/z: 333.05 [M + H]+, 1H-NMR (DMSO-d6, 400 MHZ, ppm) δ 12.04 |
| | | (s, 1H), 10.64 (s, 1H), 8.19 (d, 1H, J = 4 Hz), 7.8 (s, 1H), 6.82 (d, 1H, J = 8 Hz), |
| | | 6.69 (s, 1H, J = 8 Hz), 2.59 (s, 2H), 2.49 (bs, 3H). |
| 10 | A | LCMS (ES) m/z: [M + H]+: 358 |
| | | 1H NMR: (300 MHZ, DMSO-d6, ppm) δ 12.20 (s, 1H), 11.00 (s, 1H), 8.24 (d, |
| | | J = 3.1 Hz, 1H), 6.88 (d, J = 7.8 Hz, 1H), 6.69 (d, J = 7.7 Hz, 1H), 2.68 (s, 3H), |
| | | 2.61 (s, 3H). |
| 11 | A | LC-MS (ES) m/z: 411.00 [M + H]+, 1H-NMR (DMSO-d6, 400 MHZ, ppm)-δ 12.14 |
| | | (s, 1H), 10.83 (s, 1H), 8.21 (d, 1H, J = 2.8 Hz), 6.86 (d, 1H, J = 8.0 Hz), 6.65 |
| | | (d, 1H, J = 7.6 Hz), 2.60 (s, 3H,), 2.42 (s, 3H) |
| 12 | A | LCMS (ES) m/z: [M + H]+: 387 |
| | | 1H NMR: (400 MHZ, DMSO-d6, ppm) δ 12.17 (s, 1H), 11.01 (s, 1H), 8.79 (s, 1H), |
| | | 8.18 (d, J = 3.1 Hz, 1H), 6.82 (d, J = 7.7 Hz, 1H), 6.65 (d, J = 7.7 Hz, 1H), 2.59 (s, 3H). |
| 13 | A | LC-MS (ES) m/z: 397 [M + H]+, 1H-NMR (DMSO-d6, 400 MHz, ppm) δ 12.15 (s, |
| | | 1H), 10.87 (s, 1 H), 8.22 (s, 2H), 6.87 (d, 1H, J = 7.6 Hz), 6.62 (d, 1H, J = 8.0 Hz), |
| | | 2.6 (s, 3H). |
| 14 | C | LCMS (ES) m/z: 413.15 [M + H]+, 1H-NMR (DMSO-d6, 400 MHZ, ppm) δ 12.05 |
| | | (s 1H), 10.69 (s 1H), 8.20 (d, 1H, J = 2.8 Hz), 8.08 (s, 1H), 7.75 (s, 1H), 6.85 (d, |
| | | 1H, J = 7.6 Hz), 6.77 (s, 1H, J = 8 Hz), 3.91 (s, 3H), 2.59 (s, 3H), 2.53 (s, 3H). |
| 15 | C | LCMS (ES) m/z: 410.15 [M + H]+, 1H-NMR (DMSO-d6, 400 MHZ, ppm) δ 12.05 |
| | | (s, 1H), 10.81 (s, 1H), 8.69 (d, 2H, J = 6 Hz), 8.09 (bs, 1H), 7.70-7.55 (m, 2H), |
| | | 6.81 (bs, 1H), 2.67 (s, 3H), 2.57 (s, 3H). |
| 16 | C | LCMS (ES) m/z: 410.20 [M + H]+, 1H-NMR (DMSO-d6, 400 MHZ, ppm) δ 12.08 |
| | | (s, 1H), 10.75 (s, 1H), 8.83 (s, 1H), 8.64-8.63 (m, 1H), 8.20 (d, 1H, J = 4 Hz), |
| | | 8.01 (d, 2H, J = 8 Hz), 7.55 (m, 1H), 6.87 (d, 1H, J = 8 Hz), 6.79 (d, 1H, J = 8 Hz), |
| | | 2.62 (s, 3H), 2.61 (s, 3H). |
| 17 | C | LCMS (ES) m/z: 399.10 [M + H]+, 1H-NMR (DMSO-d6, 400 MHZ, ppm) δ 13.17 |
| | | (s 1H), 12.05 (s 1H), 10.68 (s 1H), 8.20 (d, 1H, J = 3.22 Hz), 8.09 (bs, 1H), 7.83 |
| | | (bs, 1H), 6.85 (d, 1H, J = 8 Hz), 6.87 (d, 1H, J = 7.6 Hz), 2.59 (s, 3H), 2.54 (s, 3H). |
| 18 | C | LCMS (ES) m/z: 399.05 [M + H]+, 1H-NMR (DMSO-d6, 400 MHZ, ppm) δ 12.1 |
| | | (s, 1H), 10.74 (s, 1H), 8.20 (s, 1H), 8.11 (s, 1H), 7.99 (s, 1H), 7.84 (s, 1H), 6.83 (d, |
| | | 1H, J = 7.6 Hz), 6.72 (d, 1H, J = 7.6 Hz), 3.89 (s, 3H), 2.58 (s, 3H). |
| 19 | A | LC-MS (ES) m/z: 333.1 [M + H]+, 1H-NMR (DMSO-d6, 400 MHZ, ppm)-δ 12.07 |
| | | (s, 1H), 10.64 (s, 1H), 8.20 (d, 1H, J = 3.2 Hz), 7.66 (s, 1H), 6.83 (d, 1H, J = 8 Hz), |
| | | 6.65 (d, 1H, J = 8 Hz), 2.58 (s, 3H), 2.44 (s, 3H). |
| 20 | C | LCMS (ES) m/z: 395.90 [M + H]+, 1H-NMR (DMSO-d6, 400 MHZ, ppm) δ 12.10 |
| | | (s, 1H), 10.84 (s, 1 H), 9.13 (s, 1H), 8.62-8.59 (m, 2H), 8.2 (d, 1H, J = 8.0 Hz), |
| | | 8.17 (s, 1H), 7.54-7.51 (m, 1H), 6.83 (d, 1H, J = 7.6 Hz), 6.76 (d, 1H, J = 8 Hz), |
| | | 2.58 (s, 3H). |
| 21 | B | LCMS (ES) m/z: 411.10 [M + H]+, 1H-NMR (DMSO-d6, 400 MHZ, ppm) δ 12.11 |
| | | (s, 1H), 10.84 (s, 1H), 9.48 (s, 1H), 9.36 (d, 1H, J = 5.2 Hz), 8.21 (s, 1H), 7.92-7.90 |
| | | (m, 1H), 6.87 (d, 1H, J = 8Hz), 6.81 (d, 1H, J = 8.0 Hz), 2.76 (s, 3H), 2.61 (s, 3H). |
| 22 | B | LCMS (ES) m/z: 385.10 [M + H]+, 1H-NMR (DMSO-d6, 400 MHZ, ppm)-δ 13.1 |
| | | (s, 1H), 12.16 (s, 1H), 10.8 (s, 1H), 8.21-8.19 (m, 2H), 7.85 (bs, 1H), 6.84 (d, 1H, |
| | | J = 8 Hz), 6.73 (d, 1H, J = 8 Hz), 6.65 (s, 1H), 2.58 (s, 3H). |
| 23 | B | LCMS (ES) m/z: [M + H]+: 404 |
| | | 1H NMR: (300 MHZ, DMSO-d6, ppm) δ 11.99 (s, 1H) , 10.35 (bs, 1H), 8.18 (s, 1H), |
| | | 7.27 (s, 1H), 6.85 (d, J = 7.8 Hz, 1H), 6.75 (d, J = 7.7 Hz, 1H), 3.70 (t, J = 4.9 Hz, 4H), |
| | | 3.16 (t, J = 4.9 Hz, 4H), 2.59 (s, 3H). |
| 24 | A | LCMS (ES) m/z: 385.15 [M − H]−, 1H-NMR (DMSO-d6, 400 MHZ, ppm) δ 12.13 |
| | | (s, 1H), 10.72 (s, 1H), 8.40 (s, 1H), 8.21 (d, 1H, J = 3.2 Hz), 6.9 (d, 1H, J = 7.6 Hz), |
| | | 6.70 (s, 1H, J = 7.6 Hz). |
| 25 | A | LC-MS (ES) m/z: 384.0 [M − H]−, 1H-NMR (DMSO-d6, 400 MHZ, ppm)-δ 12.2 |
| | | (s, 1H), 11.1 (s, 1H), 8.78 (s, 1H), 8.22 (d, 1H, J = 3.2 Hz), 6.87 (d, 1H, J = 7.6 Hz), |
| | | 6.70 (d, 1H, J = 7.6 Hz,), 2.61 (s, 3H). |
| 26 | C | LCMS (ES) m/z: 385.10 [M + H]+, 1H-NMR (DMSO-d6, 400 MHZ, ppm) δ 13.10 |
| | | (s, 1H), 12.1 (s, 1 H), 10.75 (s, 1H), 8.18 (d, 2H, J = 4.8 Hz), 8.01 (s, 1H), 7.90 |
| | | (s, 1H), 6.83 (d, 1H, J = 7.6 Hz), 6.73 (d, 1H, J = 7.6 Hz), 2.58 (s, 3H). |
| 27 | A | LCMS (ES) m/z: 303.05 [M − H]−, 1H-NMR (DMSO-d6, 400 MHZ, ppm) δ 12.07 |
| | | (s, 1H), 10.86 (s, 1H), 8.23 (d, 1H, J = 2.8 Hz), 8.03 (m, 2H), 7.49 (d, 1H, J = 8 Hz), |
| | | 7.08 (t, 1H, J = 7.6 Hz), 6.83 (d, 1H, J = 7.6 Hz). |
| 28 | B | LCMS (ES) m/z: 399.15 [M + H]+, 1H-NMR (DMSO-d6, 400 MHZ, ppm) δ 13.15 |
| | | (s, 1H), 12.03 (s, 1 H), 10.67 (s, 1H), 8.18 (s, 1H), 8.06 (bs, 1H), 7.83 (bs, 1H), |
| | | 6.86 (d, 1H, J = 7.6 Hz), 6.79 (d, 1H, J = 7.6 Hz), 2.59 (s, 3H), 2.53 (s, 3H). |
| 29 | B | LCMS (ES) m/z: 411.0 [M + H]+, 1H-NMR (DMSO-d6, 400 MHZ, ppm) δ 12.07 |
| | | (s, 1H), 10.79 (s, 1 H), 9.25 (s, 1H), 9.04 (s, 1H), 8.19 (d, 1H, J = 4.0 Hz), 6.87 |
| | | (d, 1H, J = 8.0 Hz), 6.81 (d, 1H, J = 8.0 Hz), 3.31 (s, 1H), 2.65 (s, 3H), 2.60 (s, 3H). |
| 30 | B | LCMS (ES) m/z: 397.15 [M + H]+, 1H-NMR (DMSO-d6, 400 MHZ, ppm) δ 12.1 |
| | | (s, 1H), 10.87 (s, 1H), 9.27 (s, 2H), 9.22 (s, 1H), 8.70 (s, 1H), 8.16 (s, 1H), 6.84-6.78 |
| | | (m, 2H,), 2.58 (s, 3H). |
| 31 | E | LCMS (ES) m/z: [M + H]+: 391 |
| | | 1H NMR: (300 MHZ, DMSO-d6, ppm): δ 12.11 (s, 1H), 10.72 (s, 1H), 8.21 |
| | | (d, J = 3.1 Hz, 1H), 7.88 (s, 1H), 6.81 (d, J = 7.8 Hz, 1H), 6.65 (d, J = 7.7 Hz, 1H), |
| | | 3.99 (s, 2H), 3.68 (s, 3H), 2.60 (s, 3H). |
| 32 | C | LCMS (ES) m/z: [M + H]+: 396 |
| | | 1H NMR: (300 MHZ, DMSO-d6, ppm) δ 12.13 (s, 1H), 10.86 (s, 1H), 9.03-8.93 |
| | | (m, 1H), 8.69-8.60 (m, 2H), 8.26-8.14 (m, 2H), 7.51 (dd, J = 8.2, 4.8 Hz, 1H), |
| | | 6.85 (d, J = 7.9 Hz, 1H), 6.75 (d, J = 7.8 Hz, 1H), 2.60 (s, 3H). |
| 33 | B | LC-MS (ES) m/z: 371.10 [M + H]+, 1H-NMR (DMSO-d6, 400 MHZ, ppm)-δ 13.09 |
| | | (s, 1H), 12.07 (s, 1H), 10.89 (s, 1H), 8.23 (s, 1H,), 8.11 (bs, 1H,), 8.03 (s, 1H), |
| | | 7.93 (bs, 1H), 7.48 (d, 1H, J = 6.8 Hz), 7.19-7.08 (m, 2H,). 6.93 (d, 1H, J = 7.6 Hz). |
| 34 | A | LCMS (ES) m/z: 370.95 [M − H]−, 1H-NMR (DMSO-d6, 400 MHZ, ppm) δ 12.12 |
| | | (s, 1H), 10.87 (s, 1H), 8.42 (s, 1H), 8.23 (d, 1H, J = 2.8 Hz), 7.56 (d, 1H, J = 8 Hz), |
| | | 7.16 (t, 1H, J =7.6 Hz), 6.88 (d, 1H, J = 7.2 Hz). |
| 35 | E | LCMS (ES) m/z: [M + H]+: 391 |
| | | 1H NMR: (300 MHz, DMSO-d6, ppm) δ 12.09 (s, 1H), 10.72 (s, 1H), 8.22 |
| | | (d, J = 3.2 Hz, 1H), 7.93 (s, 1H), 6.84 (d, J = 7.8 Hz, 1H), 6.70 (d, J = 6.9 Hz, 1H), |
| | | 4.15 (s, 2H), 3.70-3.63 (m, 3H), 2.60 (s, 3H). |
| 36 | C | LCMS (ES) m/z: [M + H]+: 396 |
| | | 1H NMR: (300 MHZ, DMSO-d6, ppm) δ 12.17 (d, J = 3.1 Hz, 1H), 10.92 (s, 1H), |
| | | 8.78 (s, 1H), 8.71-8.63 (m, 2H), 8.23 (d, J = 3.1 Hz, 1H), 7.81-7.73 (m, 2H), |
| | | 6.85 (d, J = 7.7 Hz, 1H), 6.72 (d, J = 7.7 Hz, 1H), 2.60 (s, 3H). |
| 37 | D | LCMS (ES) m/z: [M + H]+: 396 |
| | | 1H NMR: (300 MHZ, DMSO-d6, ppm) δ 12.13 (d, J = 3.2 Hz, 1H), 10.82 (s, 1H), |
| | | 8.77 (s, 1H), 8.59 (dt, J = 4.8, 1.5 Hz, 1H), 8.23 (d, J = 3.0 Hz, 1H), 8.17-8.07 |
| | | (m, 1H), 7.96 (td, J = 7.8, 1.7 Hz, 1H), 7.43 (ddd, J = 7.5, 4.9, 1.1 Hz, 1H), 6.85 |
| | | (d, J = 7.8 Hz, 1H), 6.74 (d, J = 7.7 Hz, 1H), 2.59 (s, 3H). |
| 38 | F | LCMS (ES) m/z: [M + H]+: 316 |
| | | 1H NMR (300 MHZ, DMSO-d6, ppm) δ 12.45 (s, 1H), 12.14 (d, J = 2.9 Hz, 1H), |
| | | 9.96 (s, 1H), 8.23 (d, J = 3.0 Hz, 1H), 7.62 (d, J = 2.1 Hz, 1H), 7.38 (d, |
| | | J = 7.8 Hz, 1H), 7.10 (t, J = 7.8 Hz, 1H), 7.01 (d, J = 7.6 Hz, 1H), 2.64 (q, |
| | | J = 7.6 Hz, 2H), 1.19 (t, J = 7.6 Hz, 3H). |
| 39 | A | LCMS (ES) m/z: [M + H]+: 344.20, 1H-NMR (DMSO-d6, 400 MHZ, ppm) δ 12.38 |
| | | (s, 1H), 12.15 (s, 1H), 9.74 (s, 1H), 8.18 (d, 1H, J = 3.2 Hz), 7.52 (d, 1H, J = 2.4 Hz), |
| | | 6.81 (s, 2H), 3.00-2.93 (m, 1H), 2.57 (s, 3H), 1.22 (d, 6H, J = 6.8 Hz). |
| 40 | A | LCMS (ES) m/z: [M + H]+: 396.95, 1HNMR (DMSO-d6, 400 MHZ, ppm)-δ 12.12 |
| | | (s, 1H), 10.87 (s, 1H), 8.23 (s, 1H), 8.20 (d, 1H, J = 3.2 Hz), 6.87 (d, 1H, J = 8 Hz), |
| | | 6.68 (d, 1H, J = 7.6 Hz,), 2.60 (s, 3H). |
| 41 | E | LCMS (ES) m/z: [M + H]+: 363 |
| | | 1H NMR (300 MHZ, DMSO-d6, ppm) δ 12.05 (s, 1H), 10.65 (s, 1H), 8.20 (d, J = |
| | | 2.9 Hz, 1H), 7.70 (s, 1H), 6.83 (d, J = 7.8 Hz, 1H), 6.67 (d, J = 7.7 Hz, 1H), 4.76 |
| | | (t, J = 5.3 Hz, 1H), 3.70 (q, J = 6.3 Hz, 2H), 2.92 (t, J = 6.6 Hz, 2H), 2.60 (s, 3H). |
| 42 | A | LCMS (ES) m/z: [M + H]+: |
| | | 319.05, 1H-NMR (DMSO-d6, 400 MHZ, ppm) δ 11.98 (s, 1H), |
| | | 10.31 (s, 1H), 9.33 (s, 1H), 8.17 (d, 1H, J = 3.2 Hz), 8.16 (s, 1H,), |
| | | 6.87 (d, 1H, J = 7.6 Hz), 6.67 (d, 1H, J = 7.6 Hz), 2.61 (s, 3H). |
| 43 | A | LCMS (ES) m/z: [M − H]−: 329.05, 1H-NMR (DMSO-d6, 400 MHZ, ppm)-δ 12.01 |
| | | (s, 1H), 10.05 (s, 1H), 8.21 (d, 1H, J = 3.2 Hz), 6.89 (d, 1H, J = 8 Hz), 6.62 (d, 1H, |
| | | J = 7.6 Hz), 2.62 (s, 3H), 2.20 (s, 3H), 2.03 (s, 3H). |
| 44 | E | LCMS (ES) m/z: [M + 1]+: 363 |
| | | 1H NMR (300 MHz, DMSO-d6, ppm) δ 12.05 (d, J = 3.2 Hz, 1H), 10.63 (s, 1H), |
| | | 8.20 (d, J = 3.0 Hz, 1H), 7.85 (s, 1H), 6.84 (d, J = 7.8 Hz, 1H), 6.72 (d, J = 7.7 Hz, 1H), |
| | | 5.01 (t, J = 5.0 Hz, 1H), 3.59 (q, J = 5.6 Hz, 2H), 3.00 (t, J = 6.0 Hz, 2H), 2.60 (s, 3H). |
| 45 | C | LCMS (ES) m/z: [M + 1]+: 397 |
| | | 1H NMR (300 MHZ, DMSO-d6, ppm) δ 12.12 (s, 1H), 10.92 (s, 1H), 9.20 (s, 1H), |
| | | 8.95 (d, J = 6.1 Hz, 2H), 8.18 (s, 2H), 6.84-6.72 (m, 2H), 2.58 (s, 3H). |
| 46 | B | LCMS (ES) m/z: [M + H]+: 397.10, 1H-NMR (DMSO-d6, 400 MHZ, ppm) δ 12.10 |
| | | (s, 1H), 10.91 (s, 1H), 9.73 (s, 1H), 9.36 (d, 1H, J = 4.0 Hz), 8.93 (s, 1H), 8.18 (s, 1H), |
| | | 8.08-8.06 (m, 1H), 6.84 (d, 1H, J = 8.0 Hz), 6.77 (d, 1H, J = 8.0 Hz), 2.59 (s, 3H). |
| 47 | A | LCMS (ES) m/z: [M − H]−: 384.95, 1H-NMR (DMSO-d6, 400 MHZ, ppm)-δ 12.0 |
| | | (s, 1H), 10.5 (s, 1H), 8.60 (s, 1H), 8.20 (d, 1H, J = 3.2 Hz), 6.81 (d, 1H, J = 7.6 Hz), |
| | | 6.54 (d, 1H, J = 8.0 Hz), 2.58 (s, 3H). |
| 48 | A | LCMS (ES) m/z: [M + H]+: 333.1, 1H-NMR (DMSO-d6, 400 MHZ, ppm)-δ 11.97 |
| | | (s, 1H), 10.3 (s, 1H), 8.18 (d, 1H, J = 3.2 Hz), 7.89 (s, 1H), 6.87 (d, 1H, J = 3.2 Hz), |
| | | 6.71 (d, 1H, J = 7.6 Hz), 2.67 (s, 3H), 2.60 (s, 3H). |
| 49 | A | LCMS (ES) m/z: [M + H]+: 373.10, 1H-NMR (DMSO-d6, 400 MHZ, ppm)-δ 12.06 |
| | | (s, 1H), 10.65 (s, 1H), 8.65 (s, 1H), 8.23 (d, 1H, J = 3.2Hz), 7.49 (d, 1H, J = 7.6 Hz), |
| | | 7.09 (t, 1H, J = 8 Hz), 6.75 (d, 1H, J = 7.2 Hz). |
| 50 | A | LCMS (ES) m/z: [M + H]+: 305.05, 1H-NMR (DMSO-d6, 400 MHZ, ppm) δ 11.98 |
| | | (s, 1H), 10.47 (s, 1H), 9.33 (s, 1H), 8.22-8.19 (m, 2H), 7.52 (d, 1H, J = 7.6 Hz), |
| | | 7.15 (t, 1H, J = 8.0 Hz), 6.85 (d, 1H, J = 8.0 Hz). |
| 51 | A | LCMS (ES) m/z: [M + H]+: 319.05, 1H-NMR (DMSO-d6, 400 MHZ, ppm) δ 11.96 |
| | | (s, 1H), 10.43 (s, 1H), 8.21 (d, 1H, J = 4.0 Hz), 7.94 (s, 1H), 7.50 (d, 1H, J = 8.0 Hz), |
| | | 7.16 (t, 1H, J = 8 Hz), 6.90 (s, 1H, J = 8.0 Hz), 2.67 (s, 3H). |
| 52 | C | LCMS (ES) m/z: [M + H]+: 385.15, 1H-NMR (DMSO-d6, 400 MHZ, ppm)-δ 13.26 |
| | | (s, 1H), 12.07 (s, 1H), 10.69 (s, 1H), 8.29 (bs, 1H), 8.21-8.19 (m, 2H), 7.90 (bs, 1H), |
| | | 6.84 (d, 1H, J = 7.6 Hz), 6.72 (d, 1H, J = 7.6 Hz), 2.58 (s, 3H). |
| 53 | C | LCMS (ES) m/z: [M + H]+: 399.10, 1H-NMR (DMSO-d6, 400 MHZ, ppm)-δ 12.04 |
| | | (s, 1H), 10.84 (s, 1H), 8.23 (d, 1H, J = 2.8 Hz), 8.07 (s, 1H,), 7.73 (s, 1H,), 7.49 |
| | | (d, 1H, J = 7.6 Hz), 7.14 (t, 1H, J = 7.6 Hz), 6.97 (d, 1H, J = 7.6 Hz), 3.90 (s, 3H,). |
| | | 2.53 (s, 3H). |
| 54 | A | LCMS (ES) m/z: [M + H]+: 317.15, 1HNMR (DMSO-d6, 400 MHZ, ppm)-δ 12.01 |
| | | (s, 1H), 10.07 (s, 1H), 8.68 (s, 1H), 8.20 (d, 1H, J = 3.2 Hz), 6.90 (d, 1H, J = 8 Hz), |
| | | 6.70 (d, 1H, J = 8 Hz), 2.62 (s, 3H), 2.24 (s, 3H). |
| 55 | A | LCMS (ES) m/z: [M + H]+: 397.0, 1H-NMR (DMSO-d6, 400 MHZ, ppm)-δ 12.14 |
| | | (s, 1H), 11.98 (s, 1H), 8.25 (d, 1H, J = 3.2 Hz), 7.53 (d, 1H, J = 7.6 Hz), 7.14 |
| | | (t, 1H, J = 7.6 Hz), 6.84 (d, 1H, J = 7.2 Hz), 2.42 (s, 3H). |
| 56 | A | LCMS (ES) m/z: [M + H]+: 317.10, 1H-NMR (DMSO-d6, 400 MHZ, ppm) δ 12.04 |
| | | (s, 1H), 10.22 (s, 1H), 8.23 (d, 1H, J = 4.0 Hz), 7.55 (d, 1H, J = 8.0 Hz), 7.16 |
| | | (t, 1H, J = 8.0 Hz), 6.77 (d, 1H, J = 7.2 Hz), 2.21 (s, 3H,), 2.03 (s, 3H). |
| 57 | G | LCMS (ES) m/z: [M + 1]+: 363 |
| | | 1H NMR (300 MHZ, DMSO-d6, ppm) δ 13.56 (s, 1H), 12.18 (d, J = 3.1 Hz, 1H), |
| | | 10.91 (s, 1H), 8.71 (s, 1H), 8.22 (d, J = 3.1 Hz, 1H), 6.85 (dd, J = 7.7, 1.0 Hz, 1H), |
| | | 6.64 (d, J = 7.7 Hz, 1H), 2.60 (s, 3H). |
| 58 | G | LCMS (ES) m/z: [M + 1]+: 376 |
| | | 1H NMR (300 MHZ, DMSO-d6, ppm) δ 12.13 (s, 1H), 10.84 (s, 1H), 8.53 (s, 1H), |
| | | 8.33 (d, J = 5.0 Hz, 1H), 8.22 (d, J = 3.1 Hz, 1H), 6.92-6.83 (m, 1H), 6.72 |
| | | (d, J = 7.7 Hz, 1H), 2.81 (d, J = 4.7 Hz, 3H), 2.61 (s, 3H). |
| 59 | G | LCMS (ES) m/z: [M + 1]+: 390 |
| | | 1H NMR (400 MHZ, DMSO-d6, ppm) δ 12.11 (d, J = 3.2 Hz, 1H), 10.81 (s, 1H), |
| | | 8.39 (s, 1H), 8.21 (d, J = 3.1 Hz, 1H), 6.85 (dd, J = 7.7, 1.0 Hz, 1H), 6.69 |
| | | (d, J = 7.7 Hz, 1H), 2.99 (s, 3H), 2.92 (s, 3H), 2.60 (s, 3H). |
| 60 | G | LCMS (ES) m/z: [M + 1]+: 416 |
| | | 1H NMR (300 MHZ, DMSO-d6, ppm) δ 12.03 (d, J = 2.9 Hz, 1H), 10.76 (s, 1H), |
| | | 8.50 (s, 1H), 8.21 (d, J = 3.1 Hz, 1H), 6.91-6.83 (m, 1H), 6.76 (d, J = 7.7 Hz, 1H), |
| | | 3.53-3.41 (m, 2H), 3.33-3.24 (m, 2H), 2.61 (s, 3H), 1.87-1.71 (m, 4H). |
| 61 | G | LCMS (ES) m/z: [M + 1]+: 432 |
| | | 1H NMR (300 MHz, DMSO-d6, ppm) δ 12.08 (s, 1H), 10.81 (s, 1H), 8.46 (s, 1H), |
| | | 8.23 (d, J = 3.1 Hz, 1H), 6.87 (d, J = 7.7 Hz, 1H), 6.71 (d, J = 7.7 Hz, 1H), 3.62 |
| | | (s, 4H), 2.61 (s, 3H). |
| 62 | A | LCMS (ES) m/z: [M − H]−: 356.0, 1H-NMR (DMSO-d6, 400 MHZ, ppm)-δ 12.18 |
| | | (s, 1H), 11.08 (s, 1H), 8.22 (d, 1H, J = 3.2 Hz), 6.86 (d, 1H, J = 7.6 Hz), 6.67 (d, 1H, |
| | | J = 7.6 Hz), 2.61 (s, 6H). |
| 63 | A | LCMS (ES) m/z: [M − H]−: 316.95, 1H-NMR (DMSO-d6, 400 MHZ, ppm)-δ 11.95 |
| | | (s, 1H), 10.23 (s, 1H), 9.26 (d, 1H, J = 4 Hz), 8.31 (d, 1H, J = 4 Hz), 8.17 (s, 1H), |
| | | 6.78 (d, 1H, J = 7.6 Hz), 6.62 (d, 1H, J = 7.6 Hz), 2.58 (s, 3H). |
| 64 | A | LCMS (ES) m/z: [M − H]−: 302.95, 1H-NMR (DMSO-d6, 400 MHZ, ppm)-δ 11.94 |
| | | (s, 1H), 10.39 (s, 1H), 9.26 (d, 1H, J = 2.0 Hz), 8.37 (d, 1H, J = 2.0 Hz), 8.21 |
| | | (d, 1H, J = 3.2 Hz), 7.42 (d, 1H, J = 7.6 Hz,), 7.06 (t 1H, J = 8 Hz), |
| | | 6.83 (d, 1H, J = 7.2 Hz). |
| 65 | A | LCMS (ES) m/z: [M − H]−: 380.85, 1H-NMR (DMSO-d6, 400 MHZ, ppm)-δ 12.17 |
| | | (s, 1H), 11.03 (s, 1H), 8.26 (d, 1H, J = 3.2, Hz,), 8.23 (s, 1H), 7.53 (d, 1H, J = 8 Hz), |
| | | 7.14 (t, 1H, J = 8.0 Hz,), 6.79 (d, 1H, J = 7.2 Hz,). |
| 66 | A | LCMS (ES) m/z: [M + H]+: 333.05, 1H-NMR (DMSO-d6, 400 MHZ, ppm) δ 10.78 |
| | | (s, 1H), 8.24 (s, 1H), 8.18 (d, 1H, J = 3.2 Hz), 8.17 (d, 1H J = 2.8 Hz), 6.83 (d, 1H, |
| | | J = 7.6 Hz), 6.47 (d, 1H, J = 7.6 Hz), 4.06 (s, 3H), 2.60 (s, 3H). |
| 67 | C | LCMS (ES) m/z: [M + 1]+: 397 |
| | | 1H NMR (300 MHz, DMSO-d6, ppm) δ 12.15 (d, J = 3.2 Hz, 1H), 10.93 (s, 1H), |
| | | 9.23 (s, 4H), 8.70 (s, 1H), 8.23 (d, J = 3.1 Hz, 1H), 6.90-6.81 (m, 1H), 6.76 |
| | | (d, J = 7.7 Hz, 1H), 2.60 (s, 3H). |
| 68 | C | LCMS (ES) m/z: [M + 1]+: 397 |
| | | 1H NMR (300 MHZ, DMSO-d6, ppm) δ 12.17 (s, 1H), 10.98 (s, 1H), 9.70 (s, 1H), |
| | | 9.32 (d, J = 5.5 Hz, 1H), 8.91 (s, 1H), 8.23 (d, J = 3.1 Hz, 1H), 8.05 (dd, J = 5.6, |
| | | 2.5 Hz, 1H), 6.85 (d, J = 7.7 Hz, 1H), 6.74 (d, J = 7.8 Hz, 1H), 2.60 (s, 3H), 2.08 (s, OH). |
| 69 | C | LCMS (ES) m/z: [M + 1]+: 385 |
| | | 1H NMR (300 MHZ, DMSO-d6, ppm) δ 13.24 (s, 1H), 12.11 (d, J = 3.1 Hz, 1H), |
| | | 10.75 (s, 1H), 8.39 (s, 1H), 8.21 (d, J = 3.0 Hz, 1H), 7.93-7.85 (m, 1H), 6.84 |
| | | (dd, J = 5.2, 3.2 Hz, 2H), 6.72 (d, J = 7.7 Hz, 1H), 2.59 (s, 3H). |
| 70 | C | LCMS (ES) m/z: [M + 1]+: 385 |
| | | 1H NMR (400 MHZ, DMSO-d6, ppm) δ 13.28 (s, 1H), 12.11 (d, J = 3.1 Hz, 1H), |
| | | 10.72 (s, 1H), 8.32 (s, 1H), 8.26-8.19 (m, 2H), 7.96 (s, 1H), 6.84 (dd, J = 7.7, |
| | | 1.0 Hz, 1H), 6.71 (d, J = 7.7 Hz, 1H), 2.59 (s, 3H). |
| 71 | C | LCMS (ES) m/z: [M + 1]+: 386 |
| | | 1H NMR (300 MHz, DMSO-d6, ppm) δ 12.11 (s, 1H), 10.82 (s, 1H), 8.76 (d, |
| | | J = 1.0 Hz, 1H), 8.59 (d, J = 1.0 Hz, 1H), 8.39 (s, 1H), 8.20 (d, J = 3.0 Hz, 1H), |
| | | 6.84 (d, J = 7.7 Hz, 1H), 6.72 (d, J = 7.7 Hz, 1H), 2.59 (s, 3H). |
| 72 | G | LCMS (ES) m/z: [M + 1]+: 362 |
| | | 1H NMR (400 MHZ, DMSO-d6, ppm) δ 12.12 (d, J = 3.0 Hz, 1H), 10.86 (s, 1H), |
| | | 8.55 (s, 1H), 8.21 (d, J = 3.2 Hz, 1H), 7.81 (s, 1H), 7.71 (s, 1H), 6.87 (d, J = 7.8 Hz, |
| | | 1H), 6.75 (d, J = 7.7 Hz, 1H), 2.61 (s, 3H). |
| 73 | G | LCMS (ES) m/z: [M + 1]+: 418 |
| | | 1H NMR (300 MHz, DMSO-d6, ppm) δ 12.10 (s, 1H), 10.79 (s, 1H), 8.39 (s, 1H), |
| | | 8.22 (d, J = 3.1 Hz, 1H), 6.84 (d, J = 7.8 Hz, 1H), 6.67 (d, J = 7.7 Hz, 1H), 3.41 |
| | | (d, J = 7.2 Hz, 2H), 3.23 (q, J = 6.9 Hz, 2H), 2.60 (s, 3H), 1.13 (t, J = 7.1 Hz, 3H), |
| | | 0.93 (t, J = 7.0 Hz, 3H). |
| 74 | G | LCMS (ES) m/z: [M + 1]+: 406 |
| | | 1H NMR-PH-ROP-03-151-0 (300 MHz, DMSO-d6, ppm) δ 12.15 (d, J = 3.3 Hz, |
| | | 1H), 10.87 (s, 1H), 8.55 (s, 1H), 8.25-8.13 (m, 2H), 6.87 (d, J = 7.7 Hz, 1H), |
| | | 6.71 (d, J = 7.7 Hz, 1H), 4.86 (s, 1H), 3.55 (d, J = 7.6 Hz, 2H), 3.37 (d, J = 5.9 Hz, |
| | | 2H), 2.61 (s, 3H). |
| 75 | G | LCMS (ES) m/z: [M + 1]+: 420 |
| | | 1H NMR (300 MHZ, DMSO-d6, ppm) δ 12.06 (s, 1H), 10.86 (s, 1H), 8.49 (s, 1H), |
| | | 8.22-8.10 (m, 2H), 6.87-6.73 (m, 2H), 3.46 (d, J = 2.9 Hz, 4H), 3.29 (s, 3H), |
| | | 2.58 (s, 3H). |
| 76 | G | LCMS (ES) m/z: [M + 1]+: 432 |
| | | 1H NMR (300 MHZ, DMSO-d6, ppm) δ 12.08 (dd, J = 18.2, 3.2 Hz, 1H), 10.79 |
| | | (s, 1H), 8.51 (d, J = 3.3 Hz, 1H), 8.21 (d, J = 3.1 Hz, 1H), 6.87 (d, J = 7.8 Hz, 1H), |
| | | 6.73 (dd, J = 14.5, 7.7 Hz, 1H), 5.01 (d, J = 3.7 Hz, 1H), 4.28 (s, 1H), 3.63-3.37 |
| | | (m, 4H), 2.61 (s, 3H), 1.94-1.72 (m, 2H). |
| 77 | G | LCMS (ES) m/z: [M + 1]+: 430 |
| | | 1H NMR (300 MHZ, DMSO-d6, ppm) δ 12.10 (d, J = 3.3 Hz, 1H), 10.82 (s, 1H), |
| | | 8.36 (s, 1H), 8.22 (d, J = 3.2 Hz, 1H), 6.85 (dd, J = 7.7, 1.0 Hz, 1H), 6.71 (dd, |
| | | J = 7.7, 1.4 Hz, 1H), 3.57 (t, J = 5.4 Hz, 2H), 3.31-3.19 (m, 2H), 2.60 (s, 3H), |
| | | 1.69-1.46 (m, 4H), 1.37 (s, 2H). |
| 78 | G | LCMS (ES) m/z: [M + 1]+: 431 |
| | | 1H NMR (400 MHZ, DMSO-d6, ppm) δ 11.84 (s, 1H), 8.54 (s, 2H), 8.21 (s, 1H), |
| | | 7.85 (s, 1H), 6.99 (d, J = 7.7 Hz, 1H), 6.62 (d, J = 7.8 Hz, 1H), 3.76 (d, J = 16.7 Hz, |
| | | 4H), 3.02 (d, J = 74.1 Hz, 4H), 2.52 (d, J = 1.8 Hz, 3H). |
| 79 | G | LCMS (ES) m/z: [M + 1]+: 445 |
| | | 1H NMR (300 MHz, DMSO-d6, ppm) δ 12.07 (d, J = 3.1 Hz, 1H), 8.45 (s, 1H), |
| | | 8.18 (d, J = 2.8 Hz, 1H), 6.84 (d, J = 7.7 Hz, 1H), 6.75 (d, J = 7.7 Hz, 1H), 2.95 |
| | | (s, 2H), 2.78 (s, 2H), 2.61 (s, 6H). |
| 80 | G | LCMS (ES) m/z: [M + 1]+: 363 |
| | | 1H NMR(400 MHZ, DMSO-d6, ppm) δ 12.16 (d, J = 3.2 Hz, 1H), 10.98 (s, 1H), |
| | | 8.58 (s, 1H), 8.23 (d, J = 3.1 Hz, 1H), 6.86 (dd, J = 7.7, 1.0 Hz, 1H), 6.68 (d, |
| | | J = 7.7 Hz, 1H), 2.61 (s, 3H). |
| 81 | A | LCMS (ES) m/z: [M + 1]+: 343.10, 1H-NMR (DMSO-d6, 400 MHZ, ppm) δ 12.18 |
| | | (s, 1H), 10.84 (s, 1H), 8.22 (d, 1H, J = 3.2 Hz), 6.89 (d, 1H, J = 8.4 Hz), 6.86 (s, 1H), |
| | | 6.63 (d, 1H, J = 7.6 Hz), 2.61 (s, 3H), 2.07-2.02 (m, 1H), 1.05-1.00 (m, 2H), |
| | | 0.81-0.78 (m, 2H). |
| 82 | A | LCMS (ES) m/z: [M + 1]+: 347.10, 1H-NMR (DMSO-d6, 400 MHZ, ppm) δ 11.90 |
| | | (s, 1H), 10.07 (s, 1H), 8.19 (d, 1H, J = 4.0 Hz), 6.81 (d, 1H, J = 7.6 Hz), 6.66 (d, 1H, |
| | | J = 7.6 Hz), 2.59 (s, 3H), 2.58 (s, 3H), 2.25 (s, 3H). |
| 83 | A | LCMS (ES) m/z: [M − H]−: 375.00, 1H-NMR (DMSO-d6, 400 MHZ, ppm) δ 12.21 |
| | | (s, 1H), 11.13 (s, 1H), 8.23 (d, 1H, J = 4.0 Hz), 6.90 (d, 1H, J = 8.0 Hz), 6.66 (d, |
| | | 1H, J = 8.0 Hz), 2.62 (s, 3H), 1.97-1.90 (m, 1H,), 1.12-1.05 (m, 2H), 0.97-0.94 |
| | | (m, 2H). |
| 84 | A | LCMS (ES) m/z: [M + 1]+: 349.10, 1H-NMR (DMSO-d6, 400 MHZ, ppm)-δ 11.97 |
| | | (s, 1H), 10.19 (s, 1H), 8.19 (d, 1H, J = 3.6 Hz), 7.52 (s, 1H), 6.89 (d, 1H, J = 8 Hz), |
| | | 6.78 (d, 1H, J = 8 Hz), 4.06 (s, 3H), 2.61 (s, 3H). |
| 85 | A | LCMS (ES) m/z: [M + H]+: 347.10, 1H-NMR (DMSO-d6, 400 MHZ, ppm) δ 11.95 |
| | | (s, 1H), 10.26 (s, 1H), 8.18 (d, 1H, J = 3.2 Hz), 7.93 (s, 1H), 6.87 (d, 1H, J = 8 Hz), |
| | | 6.72 (d, 1H, J = 7.6 Hz), 3.01 (q, 2H, J = 7.6 Hz), 2.61 (s, 3H), 1.25 (t, 3H, J = 8 Hz). |
| 86 | A | LCMS (ES) m/z: [M − H]−: 315.00, 1H-NMR (DMSO-d6, 400 MHZ, ppm) δ 12.03 |
| | | (s, 1H), 10.19 (s, 1H), 9.36 (s, 1H), 8.20 (d, 1H, J = 2.8 Hz), 6.88 (d, 1H, J = 7.6 Hz), |
| | | 6.67 (d, 1H, J = 7.6 Hz), 2.61 (s, 3H), 2.15 (s, 3H). |
| 87 | A | LCMS (ES) m/z: [M + H]+: 329.20, 1H-NMR (DMSO-d6, 400 MHZ, ppm) δ 12.18 |
| | | (s, 1H), 10.99 (s, 1H), 8.25 (d, 1H, J = 3.2 Hz), 7.56 (d, 1H, J = 8 Hz), 7.17 (t, 1H, |
| | | J = 7.6Hz), 6.89 (s, 1H), 6.80 (d, 1H, J = 7.6 Hz), 2.07-2.02 (m, 1H), 1.05-1.00 |
| | | (m, 2H), 0.81-0.76 (m, 2H). |
| 88 | A | LCMS (ES) m/z: [M + H]+: 333.10, 1H-NMR (DMSO-d6, 400 MHZ, ppm) δ 11.91 |
| | | (s, 1H), 10.24 (s, 1H), 8.22 (d, 1H, J = 2.8 Hz), 7.44 (d, 1H, J = 8.0 Hz), 7.10 |
| | | (t, 1H, J = 7.6 Hz), 6.84 (d, 1H, J = 6.8 Hz), 2.59 (s, 3H), 2.28 (s, 3H). |
| 89 | A | LCMS (ES) m/z: [M − H]−: 360.90, 1H-NMR (DMSO-d6, 400 MHZ, ppm) δ 12.21 |
| | | (s, 1H), 11.27 (s, 1H), 8.26 (d, 1H, J = 4.0 Hz), 7.58 (d, 1H, J = 8.0 Hz), 7.17 |
| | | (t, 1H, J = 8.0 Hz), 6.83 (d, 1H, J = 8.0 Hz), 1.96-1.89 (m, 1H,), 1.11-1.07 (m, 2H,), |
| | | 0.97-0.95 (m, 2H). |
| 90 | A | LCMS (ES) m/z: [M + H]+: 335.00, 1H-NMR (DMSO-d6, 400 MHZ, ppm) δ 11.96 |
| | | (s, 1H), δ 10.35 (s, 1H), 8.21 (d, 1H, J = 2.8 Hz), 7.58 (s, 1H), 7.51 (d, 1H, J = 8 Hz), |
| | | 7.17 (t, 1H, J = 8 Hz), 6.96 (d, 1H, J = 7.6 Hz), 4.05 (s, 3H). |
| 91 | A | LCMS (ES) m/z: [M + H]+: 333.05, 1H-NMR (DMSO-d6, 400 MHz, ppm) δ 11.94 |
| | | (s, 1H), 10.43 (s, 1H), 8.21 (d, 1H, J = 3.2 Hz), 7.97 (s, 1H), 7.51 (d, 1H, J = 7.6 |
| | | Hz), 7.15 (t, 1H, J = 8 Hz), 6.90 (d, 1H, J = 7.6 Hz), 2.99 (q, 2H, J = 7.6 Hz), 1.24 |
| | | (t, 3H, J = 7.6 Hz). |
| 92 | A | LCMS (ES) m/z: [M − H]−: 301.00, 1H-NMR (DMSO-d6, 400 MHZ, ppm) δ 12.0 |
| | | (s, 1H), 10.35 (s, 1H), 9.40 (s, 1H), 8.21 (d, 1H, J = 2.4 Hz), 7.51 (d, 1H, J = 8.0 Hz), |
| | | 7.15 (t, 1H, J = 8.0 Hz), 6.86 (d, 1H, J = 7.6 Hz), 2.13 (s, 3H). |
| 93 | A | LCMS (ES) m/z: 333.10 [M + H]+, 1H NMR (DMSO-d6, 400 MHZ, ppm) δ 11.90 |
| | | (s, 1H), 10.14 (s, 1H), 8.97 (s, 1H), 8.17 (d, 1H, J =2.8 Hz), 6.80 (d, 1H, J = 7.6 Hz), |
| | | 6.64 (d, 1H, J = 8 Hz), 2.57 (s, 3H), 2.35 (s, 3H). |
| 94 | A | LCMS (ES) m/z: 357.05 [M − H]−, 1H NMR (DMSO-d6, 400 MHZ, ppm) δ 12.18 |
| | | (s, 1H), 10.81 (s, 1H), 8.22 (d, 1H, J = 3.2 Hz), 7.15 (s, 1H), 6.88 (d, 1H, J = 7.6 Hz), |
| | | 6.60 (d, 1H, J = 7.6 Hz), 2.61 (s, 3H), 1.24 (s, 9H). |
| 95 | A | LC-MS (ES) m/z: 366.95 [M − H]−, 1H NMR (DMSO-d6, 400 MHZ, ppm)-δ 12.02 |
| | | (bs, 1H), 10.41 (s, 1H), 8.48 (s, 1H), 8.18 (d, 1H, J = 2.8 Hz), 7.42 (t, 1H, J = 54 Hz), |
| | | 6.79 (d, 1H, J = 8.0 Hz), 6.56 (d, 1H, J = 7.6 Hz), 2.58 (s, 3H). |
| 96 | A | LCMS (ES) m/z: 359.15 [M + H]+, |
| | | 1H NMR (DMSO-d6, 400 MHZ, ppm)-δ 14.35 (s, 1H), 11.95 (s, 1H), 10.31 |
| | | (s, 1H), 8.20 (d, 1H, J = 3.2 Hz), 6.83 (s, 2H), 2.58 (s, 3H), 1.31 (s, 9H). |
| 97 | A | LCMS (ES) m/z: 343.10 [M − H]−, 1H NMR (DMSO-d6, 400 MHZ, ppm) δ 12.06 |
| | | (s, 1H), 10.45 (s, 1H), 8.21 (d, 1H, J = 3.2 Hz), 7.51 (s, 1H), 6.88 (d, 1H, J = 8.4 Hz), |
| | | 6.65 (d, 1H, J = 7.6 Hz), 2.61 (s, 3H), 2.22-2.15 (m, 1H), 1.14 (m, 2H), 0.96-0.95 |
| | | (m, 2H). |
| 98 | A | LCMS (ES) m/z: 314.95 [M − H]−, 1H NMR (DMSO-d6, 400 MHZ, ppm) δ 12.07 |
| | | (s, 1H), 10.51 (s, 1H), 8.20 (d, 1H, J = 3.2 Hz), 7.53 (s, 1H), 6.87 (d, 1H, J = 7.6 Hz), |
| | | 6.69 (d, 1H, J = 7.6 Hz), 2.61 (s, 3H), 2.51 (s, 3H). |
| 99 | A | LCMS (ES) m/z: 413.05 [M + H]+, 1H NMR (DMSO-d6, 400 MHZ, ppm) δ 12.05 |
| | | (s, 1H), 10.44 (s, 1H), 8.20 (d, 1H, J = 3.2 Hz), 6.90 (d, 1H, J = 7.6 Hz), 6.67 (d, 1H |
| | | J = 8.0 Hz), 2.63 (s, 3H) 2.19 (s, 3H). |
| 100 | A | LCMS (ES) m/z: 359.15 [M + H]+, 1H NMR (DMSO-d6, 400 MHZ, ppm) δ 12.07 |
| | | (s, 1H), 10.48 (s, 1H), 8.20 (d, 1H, J = 3.2 Hz), 7.55 (s, 1H), 6.88 (d, 1H, J = 8 Hz), |
| | | 6.64 (d, 1H, J = 7.6 Hz), 2.60 (s, 3H), 1.29 (s, 9H). |
| 101 | A | LCMS (ES) m/z: 319.00 [M + H]+, 1H NMR (DMSO-d6, 400 MHZ, ppm) δ 12.03 |
| | | (s, 1H), 10.79 (s, 1H), 8.22 (d, 1H, J = 3.2 Hz), 7.80 (bs, 1H,), 7.49 (d, 1H, J = 8 Hz), |
| | | 7.11 (t, 1H, J = 8 Hz), 6.89 (d, 1H, J = 7.2 Hz), 2.48 (s, 3H). |
| 102 | A | LC-MS(ES) m/z: 279.00 [M − H]−, |
| | | 1H NMR (DMSO-d6, 400 MHZ, ppm)-δ 12.86 (s, 1H), 10.83 (bs, 1H), 8.03 |
| | | (s, 3H), 7.50 (bs, 1H), 6.95 (m, 2H). |
| 103 | A | LCMS (ES) m/z: 306.15 [M + H]+, 1H NMR (DMSO-d6, 400 MHZ, ppm) δ 11.91 |
| | | (s, 1H), 10.31 (s, 1H), 8.96 (s, 1H), 8.20 (d, 1H, J = 2.8 Hz), 7.43 (d, 1H, J = 8 Hz), |
| | | 7.08 (t, 1H, J = 8 Hz), 6.82 (d, 1H, J = 7.6 Hz), 2.38 (s, 3H). |
| 104 | A | LCMS (ES) m/z: 343.00 [M − H]−, 1H NMR (DMSO-d6, 400 MHZ, ppm) δ 12.18 |
| | | (s, 1H), 10.97 (s, 1H), 8.25 (d, 1H, J = 2.8 Hz), 7.55 (d, 1H, J = 8.0 Hz), 7.17-7.13 |
| | | (m, 2H), 6.74 (d, 1H, J = 7.6 Hz), 1.13 (s, 9H). |
| 105 | A | LC-MS(ES) m/z: 355.00 [M + H]+, 1H NMR (DMSO-d6, 400 MHZ, ppm)-δ 12.01 |
| | | (s, 1H), 10.57 (s, 1H), 8.54 (s, 1H), 8.21 (d, 1H, J = 2.8 Hz), 7.54-7.27 (m, 2H), |
| | | 7.07 (t, 1H, J = 8.0 Hz), 6.77 (d, 1H, J = 7.6 Hz). |
| 106 | A | LCMS (ES) m/z: 345.20 [M + H]+, |
| | | 1H NMR (DMSO-d6, 400 MHZ, ppm)-δ 14.38 (s, 1H), 11.96 (s, 1H), 10.48 |
| | | (s, 1H), 8.22 (d, 1H, J = 3.2 Hz), 7.45 (d, 1H, J = 8.0 Hz), 7.11 (t, 1H, J = 8.0 Hz,), |
| | | 7.00 (d, 1H, J = 7.6 Hz,), 1.30 (s, 9H). |
| 107 | A | LCMS (ES) m/z: 329.10 [M − H]−, 1H NMR (DMSO-d6, 400 MHZ, ppm) δ 12.06 |
| | | (s, 1H), 10.61 (s, 1H), 8.24 (d, 1H, J = 3.2 Hz), 7.58 (s, 1H), 7.55 (d, 1H, J = 8.4 Hz), |
| | | 7.16 (t, 1H, J = 7.6 Hz), 6.84 (d, 1H, J = 6.8 Hz), 2.21-2.14 (m, 1H), 1.19-1.09 (m, 2H), |
| | | 0.94-0.90 (m, 2H). |
| 108 | A | LCMS (ES) m/z: 301.00 [M − H]−, 1H NMR (DMSO-d6, 400 MHZ, ppm) δ 12.07 |
| | | (s, 1H), 10.67 (s, 1H), 8.23, (d, 1H, J = 3.2 Hz),7.58 (s, 1H), 7.52 (d, 1H, J = 8 Hz), |
| | | 7.15 (t, 1H, J = 7.6 Hz), 6.87 (d, 1H), 2.50 (s, 3H). |
| 109 | A | LCMS (ES) m/z: 397.00 [M + H]+, 1HNMR (DMSO-d6, 400 MHZ, ppm) δ 12.06 |
| | | (s, 1H), 10.59 (s, 1H), 8.23 (d, 1H, J = 3.2 Hz), 8.01 (s, 1H), 7.57 (d, 1H, J = 7.6 Hz), |
| | | 7.17 (t, 1H, J = 7.6 Hz), 6.83 (d 1H J = 7.6 Hz), 2.16 (s, 3H). |
| 110 | A | LCMS (ES) m/z: 345.15 [M + H]+, 1H NMR (DMSO-d6, 400 MHZ, ppm) δ 12.06 |
| | | (s, 1H), 10.83 (s, 1H), 8.23 (d, 1H, J = 3.2 Hz), 7.59 (s, 1H,), 7.53 (d, 1H, J = 8 Hz), |
| | | 7.15 (t, 1H, J = 8 Hz), 6.83 (d, 1H, J = 7.2 Hz), 1.26 (s, 9H). |
| 111 | I | LCMS (ES) m/z: [M + 1]+: 395 |
| | | 1H NMR (300 MHZ, DMSO-d6, ppm) δ 12.13 (d, J = 3.1 Hz, 1H), 10.94 (s, 1H), |
| | | 8.44 (d, J = 3.3 Hz, 1H), 8.22 (d, J = 3.1 Hz, 1H), 6.86 (d, J = 7.8 Hz, 1H), 6.72 |
| | | (d, J = 7.7 Hz, 1H), 2.61 (s, 3H), 1.83 (d, J = 14.1 Hz, 6H). |
| 112 | G | LCMS (ES) m/z: [M + 1]+: 432 |
| | | 1H NMR (300 MHZ, DMSO-d6, ppm) δ 12.13 (d, J = 3.0 Hz, 1H), 10.90 (s, 1H), |
| | | 8.35 (s, 1H), 8.22 (d, J = 3.1 Hz, 1H), 6.86 (d, J = 7.8 Hz, 1H), 6.72 (d, J = 7.7 Hz, |
| | | 1H), 3.61 (d, J = 13.1 Hz, 8H), 2.61 (s, 3H). |
| 113 | A | LCMS (ES) m/z: [M + 1]+: 332 |
| | | 1H NMR (300 MHZ, DMSO-d6, ppm) δ 11.56 (s, 1H), 10.67 (s, 1H), 8.12 (s, 2H), |
| | | 7.52 (d, J = 2.7 Hz, 1H), 6.74 (dd, J = 10.7, 8.4 Hz, 1H), 6.64 (dd, J = 8.4, 4.4 Hz, 1H). |
| 114 | A | LCMS (ES) m/z: [M + 1]+: 323 |
| | | 1H NMR (300 MHz, DMSO-d6, ppm) δ 12.49 (s, 1H), 10.85 (s, 1H), 8.31 (d, |
| | | J = 3.0 Hz, 1H), 8.14 (d, J = 0.8 Hz, 2H), 6.93 (dd, J = 10.2, 8.4 Hz, 1H), |
| | | 6.70 (dd, J = 8.4, 4.4 Hz, 1H). |
| 115 | A | LCMS (ES) m/z: [M + 1]+: 332 |
| | | 1H NMR (300 MHZ, DMSO-d6, ppm) δ 11.22 (s, 1H), 11.01 (s, 1H), 8.17-8.06 |
| | | (m, 2H), 7.59 (d, J = 2.8 Hz, 1H), 7.12-7.03 (m, 1H), 6.79 (dd, J = 10.7, 2.4 Hz, 1H). |
| 116 | A | LCMS (ES) m/z: 336.90 [M − H]−, 1H NMR (DMSO-d6, 400 MHZ, ppm) δ 12.11 |
| | | (s, 1H), 11.03 (s, 1H), 8.23 (d, 1H, J = 2.8 Hz), 7.53 (d, 1H, J = 8 Hz), 7.14 (t, 1H, |
| | | J = 8 Hz), 6.88 (d, 1H, J = 7.6 Hz), 1.23 (s, 1H). |
| 117 | A | LCMS (ES) m/z: 366.95 [M − H]−, 1HNMR (DMSO-d6, 400 MHZ, ppm) δ 12.08 |
| | | (s, 1H), 10.57 (s, 1H), 8.28 (s, 1H), 8.20 (d, 1H, J = 3.2 Hz), 7.38 (t, 1H, J = 53.6 Hz), |
| | | 6.89 (d, 1H, J = 8.0 Hz), 6.68 (d, 1H, J = 7.6 Hz), 2.62 (s, 3H). |
| 118 | A | LCMS (ES) m/z: 331.15 [M + H]+, 1H NMR (DMSO-d6, 400 MHZ, ppm) δ 12.06 |
| | | (s, 1H), 10.49 (s, 1H), 8.20 (d, 1H, J = 2.8 Hz), 7.55 (s, 1H), 6.87 (d, 1H, J = 8 Hz), |
| | | 6.68 (d, 1H, J = 7.6 Hz), 2.83 (q, 2H, J = 7.6 Hz), 2.61 (s, 3H) 1.21 (t, 3H, J = 8 Hz). |
| 119 | A | LCMS (ES) m/z: 329.00 [M − H]−, |
| | | 1H NMR (DMSO-d6, 400 MHZ, ppm) δ 12.03 (s, 1H), 10.20 (s, 1H), 9.36 (s, 1H), |
| | | 8.20 (d, 1H, J = 3.2 Hz), 6.88 (d, 1H, J = 7.6 Hz), 6.66 (d, 1H, J = 7.6 Hz), 2.63 |
| | | (q 2H, J = 7.6 Hz), 2.61 (s, 3H), 1.14 (t, 3H, J = 7.2 Hz). |
| 120 | A | LCMS (ES) m/z: 352.90 [M − H]−, 1H NMR (DMSO-d6, 400 MHZ, ppm) δ 12.09 |
| | | (s, 1H), 10.74 (s, 1H), 8.31 (s, 1H), 8.24 (d, 1H, J = 3.2 Hz), 7.55 (d, 1H, J = 8 Hz), |
| | | 7.38 (t, 1H, J = 54 Hz), 7.17 (t, 1H, J = 7.6 Hz), 6.85 (d, 1H, J = 7.6 Hz). |
| 121 | A | LCMS (ES) m/z: 413.65 [M + H]+, 1H NMR (DMSO-d6, 400 MHZ, ppm) δ 12.06 |
| | | (s, 1H), 10.69 (s, 1H), 8.22 (d, 1H, J = 3.2 Hz), 7.51 (s, 1H), 7.53 (d, 1H, J = 8 Hz), |
| | | 7.15 (t, 1H, J = 8 Hz), 6.86 (d, 1H, J = 7.6 Hz), 2.82 (q, 3H, J = 7.6 Hz), 1.19 |
| | | (t, 3H, J = 7.6 Hz). |
| 122 | A | LCMS (ES) m/z: 315.00 [M − H]−, |
| | | 1H NMR (DMSO-d6, 400 MHZ, ppm) δ 12.02 (s, 1H), 10.38 (s, 1H), 9.41 (s, 1H), |
| | | 8.22 (bs, 1H), 7.50 (d, 1H, J = 8 Hz), 7.15 (t, 1H, J = 7.6 Hz), 6.85 (d, 1H, J = 7.6 Hz), |
| | | 2.60 (q, 2H, J = 14.8 Hz), 1.11 (t, 3H, J = 7.6 Hz). |
| 123 | A | LCMS (ES) m/z: 316.95 [M − H]−, |
| | | 1H NMR (DMSO-d6, 400 MHZ, ppm) δ 12.90 (s, 1H), 12.44 (s, 1H), 9.92 (s, 1H), |
| | | 8.04 (s, 1H), 7.66 (s, 1H), 7.46 (d, 1H, J = 8 Hz), 7.09 (d, 1H, J = 7.2 Hz), |
| | | 6.96 (t, 1H, J = 7.6 Hz), 2.63 (q, 2H, J = 7.6Hz), 1.18 (t, 3H). |
| 124 | A | LCMS (ES) m/z: 349.05 [M + H]+, 1H NMR (DMSO-d6, 400 MHZ, ppm) δ 13.02 |
| | | (s, 1H), 11.10 (s, 1H), 8.82 (s, 1H), 8.09 (s, 1H), 7.64 (d, 1H, J = 5.6 Hz), 6.99 |
| | | (t, 1H, J = 7.6 Hz), 6.88 (d, 1H, J = 7.2 Hz). |
| 125 | A | LCMS (ES) m/z: 302.95 [M − H]−, 1H NMR (DMSO-d6, 400 MHZ, ppm) δ 13.27 |
| | | (s, 1H), 12.47 (s, 1H), 10.01 (s, 1H), 7.67 (s, 1H), 7.37 (d, 1H, J = 8 Hz), 7.14 (d, 1H, |
| | | J = 6.8 Hz), 7.08 (t, 1H, J = 8 Hz), 2.65 (q, 2H, J = 7.6 Hz), 1.19(t, 3H, J = 7.6 Hz). |
| 126 | G | LCMS (ES) m/z: [M + 1]+: 376 |
| | | 1H NMR (400 MHZ, DMSO-d6, ppm) δ 12.14 (d, J = 3.1 Hz, 1H), 10.88 (s, 1H), |
| | | 8.91 (t, J = 4.5 Hz, 1H), 8.50 (s, 1H), 8.22 (d, J = 3.2 Hz, 1H), 6.85 (dd, J = 7.7, |
| | | 1.1 Hz, 1H), 6.66 (d, J = 7.7 Hz, 1H), 2.78 (d, J = 4.6 Hz, 3H), 2.63-2.58 (m, 3H). |
| 127 | G | LCMS (ES) m/z: [M + 1]+: 390 |
| | | 1H NMR (300 MHZ, DMSO-d6, ppm) δ12.14 (d, J = 3.2 Hz, 1H), 10.89 (s, 1H), |
| | | 8.40 (s, 1H), 8.23 (d, J = 3.1 Hz, 1H), 6.86 (dd, J = 7.7, 1.0 Hz, 1H), 6.71 |
| | | (d, J = 7.7 Hz, 1H), 3.16 (s, 3H), 3.00 (s, 3H), 2.61 (s, 3H). |
| 128 | G | LCMS (ES) m/z: [M + 1]+: 418 |
| | | 1H NMR (300 MHZ, DMSO-d6, ppm) δ 12.13 (d, J = 3.1 Hz, 1H), 10.89 (s, 1H), |
| | | 8.31 (s, 1H), 8.23 (d, J = 3.1 Hz, 1H), 6.90-6.81 (m, 1H), 6.71 (d, J = 7.7 Hz, 1H), |
| | | 3.43 (t, J = 7.1 Hz, 4H), 2.61 (s, 3H), 1.13 (t, J = 7.0 Hz, 6H). |
| 129 | G | LCMS (ES) m/z: [M + 1]+: 406 |
| | | 1H NMR (400 MHZ, DMSO-d6, ppm) δ 12.14 (d, J = 3.2 Hz, 1H), 10.89 (s, 1H), |
| | | 8.97 (t, J = 5.6 Hz, 1H), 8.58 (s, 1H), 8.23 (d, J = 3.2 Hz, 1H), 6.89-6.83 (m, 1H), |
| | | 6.67 (d, J = 7.7 Hz, 1H), 4.80 (s, 1H), 3.51 (t, J = 5.9 Hz, 2H), 3.31 (d, J = 5.8 Hz, 2H), |
| | | 2.61 (s, 3H). |
| 130 | G | LCMS (ES) m/z: [M + 1]+: 416 |
| | | 1H NMR (300 MHz, DMSO-d6, ppm) δ 12.14 (s, 1H), 10.90 (s, 1H), 8.48 (s, 1H), |
| | | 8.23 (d, J = 3.1 Hz, 1H), 6.86 (d, J = 7.7 Hz, 1H), 6.70 (d, J = 7.7 Hz, 1H), 3.71 |
| | | (t, J = 6.6 Hz, 2H), 3.48 (t, J = 6.7 Hz, 2H), 2.61 (s, 3H), 1.89 (dp, J = 26.4, 6.8 Hz, 4H). |
| 131 | G | LCMS (ES) m/z: [M + 1]+: 445 |
| | | 1H NMR (300 MHz, DMSO-d6, ppm) δ 12.03 (d, J = 3.1 Hz, 1H), 8.23 (s, 1H), |
| | | 8.10 (d, J = 2.9 Hz, 1H), 6.86-6.73 (m, 2H), 3.70-3.60 (m, 4H), 2.66 (t, J = 5.1 Hz, |
| | | 4H), 2.57 (s, 3H), 2.41 (s, 3H). |
| 132 | A | LCMS (ES) m/z: [M + 1]+: 323 |
| | | 1H NMR (300 MHZ, DMSO-d6, ppm) δ 12.11 (s, 1H), 11.14 (s, 1H), 8.35-8.25 |
| | | (m, 1H), 8.14 (ddd, J = 12.1, 6.1, 3.2 Hz, 2H), 7.33 (d, J = 8.3 Hz, 1H), |
| | | 6.86-6.76 (m, 1H). |
| 133 | A | LCMS (ES) m/z: [M + 1]+: 314 |
| | | 1H NMR (300 MHz, DMSO-d6, ppm) δ 11.18 (s, 1H), 10.74 (s, 1H), 8.15-8.06 |
| | | (m, 2H), 7.51 (d, J = 2.7 Hz, 1H), 7.34 (d, J = 8.0 Hz, 1H), 6.99 (t, J = 7.8 Hz, 1H), |
| | | 6.82 (dd, J = 7.6, 1.0 Hz, 1H). |
| 134 | A | LCMS (ES) m/z: [M + 1]+: 373 |
| | | 1H NMR (300 MHz, DMSO-d6, ppm) δ 12.23 (s, 1H), 11.16 (s, 1H), 8.86 (d, |
| | | J = 0.9 Hz, 1H), 8.27 (d, J = 3.1 Hz, 1H), 7.56 (d, J = 8.0 Hz, 1H), 7.14 (t, |
| | | J = 7.8 Hz, 1H), 6.78 (dd, J = 7.7, 0.9 Hz, 1H). |
| 135 | A | LCMS (ES) m/z: 345.15 [M + H]+, 1H NMR (DMSO-d6, 400 MHZ, ppm) δ 12.19 |
| | | (s, 1H), 10.83 (s, 1H), 8.23 (d, 1H, J = 3.2 Hz), 7.08 (s, 1H), 6.88 (d, 1H J = 7.6 Hz), |
| | | 6.61 (d, 1H, J = 7.6 Hz), 3.05-2.99 (m, 1H,), 2.61 (s, 3H) 1.18 (6H, d, J = 6.8 Hz). |
| 136 | A | LCMS (ES) m/z: 336.90 [M − H]−, 1H NMR (DMSO-d6, 400 MHZ, ppm) δ 12.08 |
| | | (s, 1H), 10.65 (s, 1H), 8.23 (d, 1H, J = 2.8 Hz), 8.08 (s, 1H), 7.55 (d, 1H, J = 8.0 Hz), |
| | | 7.18 (t, 1H, J = 7.6 Hz), 6.90 (d, 1H, J = 8 Hz). |
| 137 | A | LCMS (ES) m/z: 315.05 [M + H]+, 1H NMR (DMSO-d6, 400 MHZ, ppm) δ 13.23 |
| | | (s, 1H), 10.93 (s, 1H), 8.14 (d,1H, J = 3.2 Hz), 8.11 (d, 1H, J = 3.2 Hz), 7.52, (d 1H, |
| | | J = 8 Hz), 7.11, (t 1H, J = 7.6 Hz), 7.00, (d 1H, J = 7.2 Hz). |
| 138 | A | LCMS (ES) m/z: 331.15 [M + H]+, 1H NMR (DMSO-d6, 400 MHZ, ppm) δ 12.19 |
| | | (s, 1H), 10.99 (s, 1H), 8.25 (d, 1H, J = 3.2 Hz), 7.56 (d, 1H, J = 8 Hz), 7.16 (t, 1H, |
| | | J = 8 Hz), 7.10 (s, 1H), 6.79 (d, 1H, J = 7.2 Hz), 2.98-3.05 (m, 1H), 1.18 (d, 6H, |
| | | J = 6.8 Hz). |
| 139 | H | LCMS (ES) m/z: [M + H]+: 427 |
| | | 1H NMR (300 MHZ, DMSO-d6, ppm) δ 12.18 (s, 1H), 9.85 (s, 1H), 8.34 (d, J = 4.1 Hz, |
| | | 1H), 8.20 (d, J = 2.8 Hz, 1H), 7.61 (s, 1H), 6.90-6.77 (m, 2H), 4.57 (s, 2H), 2.69-2.51 |
| | | (m, 6H), 1.20 (t, J = 7.5 Hz, 3H), 0.76-0.58 (m, 2H), 0.47-0.35 (m, 2H). |
| 140 | H | LCMS (ES) m/z: [M + H]+: 388 |
| | | 1H NMR (300 MHZ, DMSO-d6, ppm) δ 13.32 (s, 1H), 12.15 (s, 1H), 9.85 (s, 1H), |
| | | 8.20 (d, J = 3.1 Hz, 1H), 7.63 (s, 1H), 6.81 (s, 2H), 4.83 (s, 2H), 2.59 (d, J = 7.4 Hz, |
| | | 5H), 1.21 (t, J = 7.5 Hz, 3H). |
| 141 | G | LCMS (ES) m/z: [M + H]+: 362 |
| | | 1H NMR (300 MHZ, DMSO-d6, ppm) δ 12.14 (d, J = 3.2 Hz, 1H), 10.89 (s, 1H), |
| | | 8.54 (s, 1H), 8.38 (s, 1H), 8.23 (d, J = 3.1 Hz, 1H), 7.96 (s, 1H), 6.91-6.82 (m, 1H), |
| | | 6.68 (d, J = 7.7 Hz, 1H), 2.61 (s, 3H). |
| 142 | G | LCMS (ES) m/z: [M + 1]+: 420 |
| | | 1H NMR (400 MHZ, DMSO-d6, ppm) δ 12.14 (d, J = 3.2 Hz, 1H), 10.89 (s, 1H), |
| | | 9.05 (t, J = 5.2 Hz, 1H), 8.58 (s, 1H), 8.23 (d, J = 3.1 Hz, 1H), 6.86 (dd, J = 7.7, |
| | | 1.0 Hz, 1H), 6.67 (d, J = 7.6 Hz, 1H), 3.43 (dq, J = 9.8, 5.1 Hz, 4H), 3.40 (s, 3H), |
| | | 2.61 (s, 3H). |
| 143 | G | LCMS (ES) m/z: [M + 1]+: 432 |
| | | 1H NMR (300 MHZ, DMSO-d6, ppm) δ 12.13 (s, 1H), 10.90 (s, 1H), 8.48 (d, |
| | | J = 16.3 Hz, 1H), 8.21 (d, J = 3.0 Hz, 1H), 6.85 (d, J = 7.8 Hz, 1H), 6.71 (dd, J = 7.6, |
| | | 1.7 Hz, 1H), 5.09 (dd, J = 12.7, 3.4 Hz, 1H), 4.35 (d, J = 17.6 Hz, 1H), 3.90-3.42 |
| | | (m, 4H), 2.61 (s, 3H), 1.95 (ddd, J = 27.5, 18.1, 13.9 Hz, 2H). |
| 144 | G | LCMS (ES) m/z: [M + 1]+: 430 |
| | | 1H NMR (300 MHZ, DMSO-d6, ppm) δ 12.10 (s, 1H), 10.89 (s, 1H), 8.27 (s, 1H), |
| | | 8.20 (d, J = 3.1 Hz, 1H), 6.85 (d, J = 7.8 Hz, 1H), 6.73 (d, J = 7.7 Hz, 1H), 3.51 |
| | | (s, 4H), 2.60 (s, 3H), 1.62 (s, 2H), 1.58-1.50 (m, 4H). |
| 145 | G | LCMS (ES) m/z: [M + 1]+: 431 |
| | | 1H NMR (400 MHZ, CD3OD, ppm) δ 8.42 (s, 1H), 8.23 (s, 1H), 7.95 (s, 1H), |
| | | 6.84-6.80 (dd, J = 10.1, 8.5 Hz, 2H), 3.86-3.85 (t, J = 4.8 Hz, 4H), 3.33-3.32 |
| | | (t, J = 4.9 Hz, 4H), 2.67 (s, 3H). |
| 146 | F | LCMS (ES) m/z: [M + 1]+: 343 |
| | | 1H NMR (300 MHz, DMSO-d6, ppm) δ 12.42 (s, 1H), 11.60 (s, 1H), 9.71 (s, 1H), |
| | | 7.53 (dd, J = 9.3, 2.2 Hz, 2H), 6.77 (dd, J = 8.4, 4.6 Hz, 1H), 6.71 (dd, J = 10.6, |
| | | 8.4 Hz, 1H), 2.65 (q, J = 7.6 Hz, 2H), 1.21 (t, J = 7.6 Hz, 3H). |
| 147 | F | LCMS (ES) m/z: [M + 1]+: 334 |
| | | 1H NMR (300 MHZ, DMSO-d6, ppm) δ 12.52 (s, 1H), 12.45 (s, 1H), 9.87 (s, 1H), |
| | | 8.29 (d, J = 2.8 Hz, 1H), 7.54 (d, J = 1.8 Hz, 1H), 6.96-6.84 (m, 1H), 6.83 |
| | | (dd, J = 8.5, 4.5 Hz, 1H), 2.66 (q, J = 7.6 Hz, 2H), 1.21 (t, J = 7.6 Hz, 3H). |
| 148 | A | LCMS (ES) m/z: [M + 1]+: 391 |
| | | 1H NMR (300 MHz, DMSO-d6, ppm) δ 12.60 (s, 1H), 11.13 (s, 1H), 8.87 (d, |
| | | J = 1.0 Hz, 1H), 8.34 (d, J = 3.0 Hz, 1H), 6.96 (dd, J = 10.2, 8.4 Hz, 1H), 6.69 |
| | | (dd, J = 8.4, 4.3 Hz, 1H). |
| 149 | A | LCMS (ES) m/z: [M + 1]+: 400 |
| | | 1H NMR (300 MHZ, DMSO-d6, ppm) δ 11.40-11.33 (m, 2H), 8.86 (s, 1H), 7.62 |
| | | (d, J = 2.8 Hz, 1H), 7.17 (dd, J = 8.7, 2.4 Hz, 1H), 6.71 (dd, J = 10.4, 2.4 Hz, 1H). |
| 150 | A | LCMS (ES) m/z: [M + 1]+: 391 |
| | | 1H NMR (300 MHZ, DMSO-d6, ppm) δ 12.26 (s, 1H), 8.87 (s, 1H), 8.31 (d, |
| | | J = 3.2 Hz, 1H), 7.37 (d, J = 8.6 Hz, 1H), 6.76 (dd, J = 10.4, 2.3 Hz, 1H). |
| 151 | A | LCMS (ES) m/z: [M + 1]+: 382 |
| | | 1H NMR (300 MHz, DMSO-d6, ppm) δ 11.31 (d, J = 2.8 Hz, 1H), 11.01 (s, 1H), |
| | | 8.84 (d, J = 1.0 Hz, 1H), 7.53 (d, J = 2.7 Hz, 1H), 7.40 (d, J = 8.0 Hz, 1H), 7.01 |
| | | (t, J = 7.8 Hz, 1H), 6.74 (dd, J = 7.6, 1.0 Hz, 1H). |
| 152 | A | LCMS (ES) m/z: 333.10 [M + H]+, 1H NMR (DMSO-d6, 400 MHZ, ppm) δ 11.96 |
| | | (s, 1H), 10.17 (s, 1H), 8.18 (d, 1H, J = 3.2 Hz), 8.07 (s, 1H), 6.79 (d, 1H, J = 8 Hz), |
| | | 6.63 (d, 1H, J = 7.6 Hz), 2.71 (s, 3 H), 2.57 (s, 3H). |
| 153 | A | LCMS (ES) m/z: 319.05[M + H]+, 1H NMR (DMSO-d6, 400 MHZ, ppm) δ 11.96 |
| | | (s, 1H), 10.34 (s, 1H), 8.21 (d,1H, J = 2.8 Hz), 8.14 (s, 1H), 7.42 (d, 1H, J = 8 |
| | | Hz), 7.08 (t, 1H, J = 8 Hz), 6.84 (d, 1H, J = 7.6 Hz), 2.69 (s, 3H). |
| 154 | A | LCMS (ES) m/z: 380.90 [M − H]−, 1H NMR (DMSO-d6, 400 MHZ, ppm) δ 13.36 |
| | | (s, 1H), 11.23 (s, 1H), 8.85 (s, 1H), 7.57 (d, 1H, J = 8.4), 7.12 (t, 1H, J = 7.6 Hz), |
| | | 6.95 (d, 1H, J = 7.6 Hz). |
| 155 | C | LCMS (ES) m/z: [M + 1]+: 385 |
| | | 1H NMR (300 MHZ, DMSO-d6, ppm) δ 12.52 (s, 1H), 12.12-12.05 (m, 1H), |
| | | 10.67 (s, 1H), 8.22 (d, J = 6.2 Hz, 2H), 7.78 (s, 2H), 6.84 (d, J = 7.7 Hz, 1H), |
| | | 6.72 (d, J = 7.7 Hz, 1H), 2.59 (s, 3H). |
| 156 | F | LCMS (ES) m/z: [M + 1]+: 343 |
| | | 1H NMR (300 MHZ, DMSO-d6, ppm) δ 12.50 (s, 1H), 11.25 (s, 1H), 10.13 |
| | | (s, 1H), 7.77 (s, 1H), 7.59 (s, 1H), 6.93-6.87 (m, 2H), 2.61 (q, J = 7.6 Hz, 2H), |
| | | 1.16 (t, J = 7.6 Hz, 3H). |
| 157 | F | LCMS (ES) m/z: [M + 1]+: 334 |
| | | 1H NMR (300 MHZ, DMSO-d6, ppm) δ 12.53 (s, 1H), 12.11 (s, 1H), 10.25 |
| | | (s, 1H), 8.29 (d, J = 2.5 Hz, 1H), 7.78 (d, J = 1.7 Hz, 1H), 7.15 (dd, J = 8.7, 2.4 Hz, |
| | | 1H), 6.93 (dd, J = 11.2, 2.3 Hz, 1H), 2.62 (q, J = 7.6 Hz, 2H), 1.17 (t, J = 7.6 Hz, 3H). |
| 158 | F | LCMS (ES) m/z: [M + 1]+: 325 |
| | | 1H NMR (300 MHZ, DMSO-d6, ppm) δ 12.42 (s, 1H), 11.24 (s, 1H), 9.83 (s, 1H), |
| | | 7.62 (d, J = 2.2 Hz, 1H), 7.51 (d, J = 2.7 Hz, 1H), 7.21 (dd, J = 6.0, 3.0 Hz, 1H), |
| | | 6.98 (q, J = 3.8, 3.0 Hz, 2H), 2.63 (q, J = 7.6 Hz, 2H), 1.20 (q, J = 8.7, 7.6 Hz, 3H). |
| 159 | A | LCMS (ES) m/z: [M + 1]+: 400 |
| | | 1H NMR (300 MHZ, DMSO-d6, ppm) δ11.69 (s, 1H), 10.96 (s, 1H), 8.86 (s, 1H), |
| | | 7.54 (d, J = 2.7 Hz, 1H), 6.76 (dd, J = 10.8, 8.3 Hz, 1H), 6.59 (dd, J = 8.4, 4.3 Hz, 1H). |
| 160 | A | LCMS (ES) m/z: 333.10 [M + H]+, 1HNMR (DMSO-d6, 400 MHz, ppm) d 12.03 |
| | | (s, 1H), 10.74 (s, 1H), 8.23 (d, 1H, J = 2.8 Hz), 7.49 (d, 1H, J = 8 Hz), 7.12 (t, 1H, |
| | | J = 8 Hz), 6.88 (d, 1H, J =7.6 Hz), 2.37 (s, 3H), 2.32 (s, 3H). |
| 161 | A | LCMS (ES) m/z: 358.15 [M + H]+, 1H NMR (DMSO-d6, 400 MHZ, ppm) δ 11.90 |
| | | (s, 1H), 9.78 (s, 1H), 8.15 (d, 1H, J = 2.8 Hz), 7.80 (s, 1H), 7.64 (s, 1H), 6.74 (dd, |
| | | 2H, J = 8.0 Hz, 7.6 Hz), 3.74 (d, 2H, J = 6.8 Hz). 2.55 (s, 3H), 1.91-1.84 (m, 1H), |
| | | 0.68 (d, 6H, J = 6.8 Hz). |
| 162 | A | LCMS (ES) m/z: 344.15 [M + H]+, 1H NMR (DMSO-d6, 400 MHZ, ppm) δ 11.92 |
| | | (s, 1H), 9.79 (s, 1H), 8.16 (d, 1H, J = 3.2 Hz), 7.83 (s, 1H), 7.70 (s, 1H), 6.76 (q, 2H, |
| | | J = 8.0 Hz), 3.89 (t, 2H, J = 6.8 Hz). 2.55 (s, 3H), 1.67-1.58 (m, 2H), 0.67 (t, 3H, |
| | | J = 7.6 Hz). |
| 163 | A | LCMS (ES) m/z: [M + 1]+: 357 |
| | | 1H NMR (300 MHZ, DMSO-d6, ppm) δ 12.59 (s, 1H), 11.04 (s, 1H), 8.34 (d, |
| | | J = 3.0 Hz, 1H), 8.17 (s, 1H), 6.97 (t, J = 9.3 Hz, 1H), 6.70 (dd, J = 8.5, 4.3 Hz, 1H). |
| 164 | A | LCMS (ES) m/z: [M + 1]+: 362 |
| | | 1H NMR (300 MHZ, DMSO-d6, ppm) δ 11.79 (s, 1H), 10.69 (s, 1H), 8.12 (s, 2H), |
| | | 7.95-7.87 (m, 1H), 6.83 (d, J = 7.8 Hz, 1H), 6.67 (d, J = 7.7 Hz, 1H), 2.47 (s, 3H) |
| 165 | F | LCMS (ES) m/z: 317.10 [M + H]+, 1HNMR (DMSO-d6, 400 MHZ, ppm) δ 14.49 |
| | | (s, 1H), 12.51 (s, 1H), 10.18 (s, 1H), 7.66 (s, 1H), 7.61 (d, 1H, J = 8 Hz), 7.25 (t, |
| | | 1H, J = 7.6 Hz), 7.15 (d, 1H, J = 7.2 Hz), 2.65 (q, 2H, J = 7.6 Hz), 1.20 (t, 3H, |
| | | J = 7.6 Hz). |
| 166 | A | LCMS (ES) m/z: 316.2 [M + H]+, 1H NMR (DMSO-d6, 400 MHZ, ppm) δ 12.47 |
| | | (s, 1H), 11.93 (s, 1H), 9.70 (s, 1H), 8.16 (d, 1H, J = 2.8 Hz), 7.65 (s, 1H), 6.79 |
| | | (q, 2H, J = 8.0 Hz), 2.56 (s, 3H), 2.10 (s, 3H). |
| 167 | A | LCMS (ES) m/z: 330.2 [M + H]−, 1H NMR (DMSO-d6, 400 MHZ, ppm) δ 11.92 |
| | | (s, 1H), 9.82 (s, 1H), 8.14 (s, 1H), 7.84 (s, 1H), 7.74 (s, 1H), 6.74 (s, 2H), 3.96 |
| | | (q, 2H, J = 7.2 Hz), 2.55 (s, 3H), 1.27 (t, 3H, J = 7.2 Hz). |
| 168 | A | LCMS (ES) m/z: 344.3 [M + H]+, 1H NMR (DMSO-d6, 400 MHZ, ppm) δ 11.98 |
| | | (bs, 1H), 9.78 (bs, 1H), 8.13 (s, 1H), 7.89 (s, 1H), 7.76 (s, 1H), 6.76 (s, 2H) 4.42-4.37 |
| | | (m, 1H), 2.55 (s, 3H) 1.33 (d, 6H, J = 6.4 Hz). |
| 169 | A | LCMS (ES) m/z: 358.15 [M + H]+, 1H NMR (DMSO-d6, 400 MHZ, ppm) δ 12.11 |
| | | (s, 1H), 9.79 (s, 1H), 8.18 (s, 1H), 7.58 (s, 1H), 6.81 (q, 2H, J = 11.2 Hz), 3.56 |
| | | (s, 3H), 3.10-3.03 (m, 1H), 2.57 (s, 3H), 1.20 (d, 6H, J = 6.8 Hz). |
| 170 | A | LCMS (ES) m/z: 350.05 [M + H]+, 1H NMR (DMSO-d6, 400 MHZ, ppm) δ 11.90 |
| | | (s, 1H), 9.98 (s, 1H), 8.18 (d, 1H, J = 3.2 Hz), 7.84 (s, 1H), 6.83 (d, 1H, J = 8.0 Hz), |
| | | 6.77 (d, 1H, J = 7.6 Hz), 3.57 (s, 3H), 2.57 (s, 3H). |
| 171 | A | LCMS (ES) m/z: 330.10 [M + H]+, 1H NMR (DMSO-d6, 400 MHZ, ppm) δ 12.0 |
| | | (s, 1H), 9.78 (s, 1H), 8.19 (s, 1H), 7.63 (s, 1H), 6.85-6.80 (m, 2H), 3.52 (s, 3H), |
| | | 2.56 (s, 3H), 2.29 (s, 3H). |
| 172 | A | LCMS (ES) m/z: [M + H]+: 357 |
| | | 1H NMR (400 MHZ, DMSO-d6, ppm) δ 12.53 (s, 1H), 11.02 (s, 1H), 8.32 (d, |
| | | J = 3.1 Hz, 1H), 8.22 (s, 1H), 6.97 (dd, J = 10.2, 8.4 Hz, 1H), 6.78 (dd, J = 8.4, |
| | | 4.3 Hz, 1H). |
| 173 | A | LCMS (ES) m/z: 306.2 [M + H]+, 1H NMR (DMSO-d6, 400 MHZ, ppm) δ 14.42 |
| | | (s, 1H), 11.13 (s, 1H), 8.13 (d, 1H, J = 3.2 Hz), 8.12 (d, 1H, J = 2.8 Hz), 7.75 (d, 1H, |
| | | J = 8 Hz), 7.26 (t, 1H, J = 7.6 Hz), 7.01 (d, 1H, J = 7.6 Hz). |
| 174 | A | LCMS (ES) m/z: 314.3 [M − H]−, 1H NMR (DMSO-d6, 400 MHZ, ppm) δ 12.00 |
| | | (s, 1H), 10.15 (s, 1H), 8.20 (s, 1H), 7.87 (s, 1H), 7.29 (s, 1H), 6.83 (d, 1H, J = 8 Hz), |
| | | 6.58 (d, 1H, J = 8 Hz), 3.65 (s, 3H), 2.59 (s, 3H). |
| 175 | A | LCMS (ES) m/z: 330.15 [M − H]−, 1H NMR (DMSO-d6, 400 MHZ, ppm) δ 11.96 |
| | | (s, 1H), 10.20 (s, 1H), 8.20 (d, 1H, J = 2.8 Hz), 7.98 (s, 1H), 7.30 (s, 1H), 6.82 (d, |
| | | 1H, J = 7.6 Hz), 6.60 (d, 1H, J = 7.6 Hz), 4.12 (q, 2H, J = 14.4 Hz), 2.58 (s, 3H), |
| | | 1.31 (t, 3H, J = 7.2 Hz). |
| 176 | A | LCMS (ES) m/z: 344.3 [M − H]−, 1H NMR (DMSO-d6, 400 MHZ, ppm) δ 11.93 |
| | | (s, 1H), 10.19 (s, 1H), 8.20 (bs, 1H), 7.21 (s, 1H), 6.82 (d, 1H, J = 7.6 Hz), 6.66 |
| | | (d, 1H, J = 7.6 Hz),4.13 (q, 2H, J = 13.6 Hz), 2.58 (s, 3H), 2.34 (s, 3H), 1.22 (t, 3H, |
| | | J = 6.8 Hz). |
| 177 | A | LCMS (ES) m/z: 408.2 [M + H]+, 1H NMR (DMSO-d6, 400 MHZ, ppm) δ 11.92 |
| | | (s, 1H), 10.0 (s, 1H), 8.19 (s, 1H), 6.82 (q, 2H, J = 7.6 Hz), 3.47 (s, 3H), 2.56 (s, 3H), |
| | | 2.36 (3, 3H). |
| 178 | A | LCMS (ES) m/z: 350.2 [M + H]+, 1H NMR (DMSO-d6, 400 MHZ, ppm) δ 12.05 |
| | | (s, 1H), 10.33 (s, 1H), 8.21 (d, 1H, J = 3.2 Hz), 7.29 (s, 1H), 6.86 (d, 1H, J = 7.6 Hz), |
| | | 6.66 (d, 1H, J = 8 Hz), 3.65 (s, 3H), 2.60 (s, 3H). |
| 179 | A | CMS (ES) m/z: 316.2 [M + H]+, 1H NMR (DMSO-d6, 400 MHZ, ppm) δ 12.10 |
| | | (s, 1H), 10.47 (s, 1H), 8.21 (d, 1H, J = 2.8 Hz), 7.38 (s, 1H), 7.06 (s, 1H), 6.82 |
| | | (d, 1H, J = 7.6 Hz), 6.69 (d, 1H, J = 7.6 Hz), 3.61 (s, 3H), 2.59 (s, 3H). |
| 180 | A | LCMS (ES) m/z: 350.2 [M + H]+, 1H NMR (DMSO-d6, 400 MHZ, ppm) δ 11.88 |
| | | (s, 1H), 10.90 (s, 1H), 8.16 (s, 1H), 7.92 (s, 1H), 6.83-6.79(m, 2H), 3.56 (s, 3H), |
| | | 2.56 (s, 3H). |
| 181 | A | LCMS (ES) m/z: 347.1 [M − H]−, 1H NMR (DMSO-d6, 400 MHZ, ppm) δ 13.27 |
| | | (s, 1H), 10.76 (s, 1H), 8.04 (s, 1H), 7.57 (d, 1H, J = 8 Hz), 7.16 (t, 1H, J = 7.6 Hz), |
| | | 7.05 (d, 1H, J = 7.2 Hz). |
| 182 | A | LCMS (ES) m/z: 349.1 [M + H]+, 1H NMR (DMSO-d6, 400 MHZ, ppm) δ 13.33 |
| | | (s, 1H), 11.14 (s, 1H), 8.20 (s, 1H), 7.56 (d, 1H, J = 8 Hz), 7.14 (t, 1H, J = 8 Hz), |
| | | 7.04 (d, 1H, J = 7.2 Hz). |
| 183 | A | LCMS (ES) m/z: 339.95 [M + H]+, 1H NMR (DMSO-d6, 400 MHZ, ppm) δ 14.48 |
| | | (s, 1H), 11.33 (s, 1H), 8.20 (s, 1H), 7.77 (d, 1H, J = 8 Hz), 7.29 (t, 1H, J = 8 Hz), |
| | | 7.08 (d, 1H, J = 7.6 Hz). |
| 184 | A | LCMS (ES) m/z: [M + 1]+: 357 |
| | | 1H NMR (400 MHZ, DMSO-d6, ppm) δ 12.47 (s, 1H), 10.61 (s, 1H), 8.30 |
| | | (d, J = 2.2 Hz, 1H), 7.99 (s, 1H), 6.98 (dd, J = 10.2, 8.4 Hz, 1H), 6.81 (dd, |
| | | J = 8.4, 4.3 Hz, 1H). |
| 185 | A | LCMS (ES) m/z: [M + 1]+: 348 |
| | | 1H NMR (400 MHZ, DMSO-d6, ppm) δ 11.80-11.68 (m, 1H), 10.82 (s, 1H), |
| | | 8.20-8.10 (m, 2H), 7.94 (dd, J = 3.1, 1.5 Hz, 1H), 7.46 (d, J = 8.0 Hz, 1H), |
| | | 7.06 (t, J = 7.9 Hz, 1H), 6.86 (d, J = 7.6 Hz, 1H). |
| 186 | A | LCMS (ES) m/z: [M + 1]+: 348 |
| | | 1H NMR (400 MHZ, DMSO-d6, ppm) δ 11.68 (s, 1H), 10.44 (s, 1H), 9.34 (s, 1H), |
| | | 8.23 (s, 1H), 7.97-7.90 (m, 1H), 7.48 (d, J = 8.0 Hz, 1H), 7.11 (t, J = 7.8 Hz, 1H), |
| | | 6.88 (d, J = 7.6 Hz, 1H). |
| 187 | A | LCMS (ES) m/z: [M + 1]+: 332 |
| | | 1H NMR (300 MHZ, DMSO-d6, ppm) δ 11.47 (s, 1H), 10.27 (s, 1H), 9.34 (d, |
| | | J = 0.9 Hz, 1H), 8.19 (d, J = 1.0 Hz, 1H), 7.50 (d, J = 2.7 Hz, 1H), 6.78 (dd, |
| | | J = 10.8, 8.4 Hz, 1H), 6.66 (dd, J = 8.4, 4.2 Hz, 1H). |
| 188 | A | LCMS (ES) m/z: [M + 1]+: 346 |
| | | 1H NMR (300 MHz, DMSO-d6, ppm) δ 11.46 (s, 1H), 10.23 (s, 1H), 7.92 (s, 1H), |
| | | 7.51 (d, J = 2.7 Hz, 1H), 6.78 (dd, J = 10.7, 8.3 Hz, 1H), 6.70 (dd, J = 8.3, 4.4 Hz, |
| | | 1H), 2.69 (s, 3H), 2.59 (s, 1H). |
| 189 | A | LCMS (ES) m/z: 345.3 [M − H]−, 1HNMR (DMSO-d6, 400 MHZ, ppm) δ 11.78 |
| | | (s, 1H), 7.83 (s, 1H), 6.84 (d, 1H, J = 7.6 Hz), 6.57 (d, 1H, J = 7.6 Hz), 2.46 (s, 3H), |
| | | 2.26 (s, 3H), 2.18 (s, 3H). |
| 190 | A | LCMS (ES) m/z: 316.2 [M + H]+, 1H NMR (DMSO-d6, 400 MHZ, ppm) δ 11.93 |
| | | (s, 1H), 9.85 (s, 1H), 8.17 (d, 1H, J = 3.2 Hz), 7.78 (s, 1H), 7.70 (s, 1H), 6.81 |
| | | (br, 2H), 3.62 (s, 3H), 2.56 (s, 3H). |
| 191 | A | LCMS (ES) m/z: 344.3 [M + H]+, 1H NMR (DMSO-d6, 400 MHZ, ppm) δ12.04 |
| | | (s, 1H), 9.75 (s, 1H), 8.17 (d, 1H, J = 3.2 Hz), 7.65 (s, 1H), 6.80 (s, 2H), 3.88 |
| | | (q, 2H, J = 7.2 Hz), 2.56 (s, 3H), 2.32 (s, 3H), 1.21 (t, 3H, J = 7.2 Hz). |
| 192 | A | LCMS (ES) m/z: 330.2 [M + H]+, 1H NMR (DMSO-d6, 400 MHZ, ppm) δ 11.96 |
| | | (s, 1H), 10.11 (s, 1H), 8.19 (d, 1H, J = 2Hz), 7.17 (s, 1H), 6.82 (d, 1H, J = 8Hz), |
| | | 6.65 (d, 1H, J = 7.6 Hz), 3.60 (s, 3H), 2.50 (s, 3H), 2.31 (s, 3H). |
| 193 | A | LCMS (ES) m/z: 358.3 [M + H]+, 1H NMR (DMSO-d6, 400 MHZ, ppm) δ 12.13 |
| | | (s, 1H), 11.97 (s, 1H), 9.80 (s, 1H), 8.17 (d, 1H, J = 2.4 Hz), 7.59 (s, 1H), 6.85 |
| | | (d, 1H, J = 7.6 Hz), 6.77 (d, 1H, J = 8 Hz), 2.55 (s, 3H), 1.2 2(s, 9H). |
| 194 | A | LCMS (ES) m/z: 364.2 [M + H]+, 1HNMR (DMSO-d6, 400 MHZ, ppm) δ 11.92 |
| | | (s, 1H), 10.04 (s, 1H), 8.19 (d, 1H, J = 3.2 Hz), 6.85-6.81 (m, 2H), 3.45 (s, 3H), |
| | | 2.57 (s, 3H), 2.33 (s, 3H). |
| 195 | A | LCMS (ES) m/z: 384.3 [M + H]+, 1H NMR (DMSO-d6, 400 MHZ, ppm) δ 11.88 |
| | | (s, 1H), 10.14 (s, 1H), 8.20 (d, 1H, J = 3.2 Hz), 7.23 (s, 1H), 6.81 (d, 1H, J = 8 Hz), |
| | | 6.62 (d, 1H, J = 8 Hz), 3.98 (d, 1H, J = 6.8 Hz), 2.71-2.65 (m, 2H), 2.57 (s, 3H), |
| | | 1.21-1.16 (m, 4H), 0.47-0.42 (m, 2H), 0.39-0.36 (m, 2H). |
| 196 | A | LCMS (ES) m/z: 353.00 [M + H]+, 1H NMR (DMSO-d6, 400 MHZ, ppm) δ 7.79 |
| | | (s, 1H), 7.65 (s, 1H), 6.91 (d, 1H, J = 7.6 Hz), 6.57 (d, 1H, J = 8.0 Hz), 2.46 (s, 3H). |
| 197 | A | LCMS (ES) m/z: 337.2 [M − H]−, 1H NMR (DMSO-d6, 400 MHZ, ppm) δ 12.18 |
| | | (s, 1H), 11.05 (s, 1H), 8.25 (d, 1H, J = 2.8 Hz), 8.14 (s, 1H), 7.53 (d, 1H, J = 8.0 Hz), |
| | | 7.13 (t, 1H, J = 8.0 Hz), 6.8 (d, 1H, J = 7.2 Hz). |
| 198 | A | LC-MS (ES) m/z: [M − 1]−: 360 |
| | | 1H NMR (300 MHZ, DMSO-d6, ppm) δ 11.70 (s, 1H), 10.29 (s, 1H), 9.34 |
| | | (d, J = 0.9 Hz, 1H), 8.20 (d, J = 0.9 Hz, 1H), 7.89 (d, J = 3.1 Hz, 1H), 6.89 |
| | | (d, J = 7.8 Hz, 1H), 6.71 (d, J = 7.7 Hz, 1H), 2.49 (s, 3H). |
| 199 | A | LC-MS (ES) m/z: [M + 1]+: 323 |
| | | 1H NMR (300 MHz, DMSO-d6, ppm) δ 12.39 (s, 1H), 10.43 (s, 1H), 9.37 (d, |
| | | J = 0.9 Hz, 1H), 8.30 (d, J = 3.0 Hz, 1H), 8.18 (d, J = 0.9 Hz, 1H), 6.96 (dd, |
| | | J = 10.2, 8.4 Hz, 1H), 6.70 (dd, J = 8.4, 4.4 Hz, 1H). |
| 200 | A | LC-MS (ES) m/z: [M + 1]+: 328 |
| | | 1H NMR (300 MHZ, DMSO-d6, ppm) δ 11.07 (s, 1H), 10.32 (s, 1H), 7.96 (s, 1H), |
| | | 7.51 (d, J = 2.8 Hz, 1H), 7.35 (d, J = 7.9 Hz, 1H), 7.05 (t, J = 7.8 Hz, 1H), 6.91 |
| | | (dd, J = 7.6, 1.0 Hz, 1H), 2.67 (s, 3H). |
| 201 | A | LC-MS (ES) m/z: [M + 1]+: 337 |
| | | 1H NMR (300 MHZ, DMSO-d6, ppm) δ 12.38 (s, 1H), 10.39 (s, 1H), 8.30 (d, |
| | | J = 3.0 Hz, 1H), 7.91 (s, 1H), 6.97 (dd, J = 10.2, 8.4 Hz, 1H), 6.75 (dd, J = 8.4, |
| | | 4.3 Hz, 1H), 2.70 (s, 3H). |
| 202 | A | LC-MS (ES) m/z: [M + 1]+: 363 |
| | | 1H NMR (300 MHZ, DMSO-d6, ppm) δ 11.94 (s, 1H), 10.43 (s, 1H), 8.22 (d, |
| | | J = 3.0 Hz, 1H), 7.99 (s, 1H), 7.52 (d, J = 7.9 Hz, 1H), 7.17 (t, J = 7.8 Hz, 1H), |
| | | 6.92 (d, J = 7.6 Hz, 1H), 6.32 (s, 1H), 1.45 (s, 6H). |
| 203 | A | LC-MS (ES) m/z: [M + 1]+: 314 |
| | | 1H NMR (300 MHz, DMSO-d6, ppm) δ 11.09 (s, 1H), 10.35 (s, 1H), 9.32 (d, |
| | | J = 0.9 Hz, 1H), 8.21 (d, J = 0.9 Hz, 1H), 7.50 (d, J = 2.7 Hz, 1H), 7.36 (d, |
| | | J = 7.9 Hz, 1H), 7.04 (t, J = 7.8 Hz, 1H), 6.91-6.82 (m, 1H). |
| 204 | A | LC-MS (ES) m/z: [M + 1]+: 364 |
| | | 1H NMR (300 MHZ, DMSO-d6, ppm) δ 11.18 (s, 1H), 10.62 (s, 1H), 8.32 (d, |
| | | J = 1.5 Hz, 1H), 7.54-7.35 (m, 3H), 7.06 (t, J = 7.8 Hz, 1H), 6.90-6.84 (m, 1H). |
| 205 | B | LC-MS (ES) m/z: [M + 1]+: 390 |
| | | 1H NMR (300 MHZ, DMSO-d6, ppm) δ 11.89 (s, 1H), 10.20 (s, 1H), 8.21 (d, |
| | | J = 2.2 Hz, 1H), 7.53 (s, 1H), 7.45 (d, J = 8.0 Hz, 1H), 7.17 (t, J = 7.8 Hz, 1H), |
| | | 7.02 (dd, J = 7.7, 1.0 Hz, 1H), 3.67 (t, J = 4.9 Hz, 4H), 3.42 (t, J = 4.9 Hz, 4H). |
| 206 | C | LC-MS (ES) m/z: [M + 1]+: 372 |
| | | 1H NMR (300 MHZ, DMSO-d6, ppm) δ 12.08 (s, 1H), 10.60 (s, 1H), 8.83 (s, 1H), |
| | | 8.23 (d, J = 3.1 Hz, 1H), 8.16 (s, 1H), 7.54 (d, J = 7.9 Hz, 1H), 7.17 (t, |
| | | J = 7.8 Hz, 1H), 6.93 (d, J = 7.7 Hz, 1H). |
| 207 | A | LC-MS (ES) m/z: [M − 1]−: 379 |
| | | 1H NMR (300 MHz, DMSO-d6, ppm) δ 12.26 (s, 1H), 11.13 (s, 1H), 8.25 |
| | | (d, J =3.1 Hz, 1H), 7.50 (s, 1H), 6.97-6.88 (m, 1H), 6.68 (d, J = 7.7 Hz, 1H), |
| | | 2.63 (s, 3H). |
| 208 | A | LC-MS (ES) m/z: [M − 1]−: 371 |
| | | 1H NMR (300 MHz, DMSO-d6, ppm) δ 12.25 (s, 1H), 10.73 (s, 1H), 8.08 (d, |
| | | J = 1.6 Hz, 1H), 8.03 (s, 1H), 7.28 (t, J = 54.1 Hz, 1H), 6.85 (dd, J = 8.5, 4.3 Hz, |
| | | 1H), 6.75 (dd, J = 10.5, 8.5 Hz, 1H). |
| 209 | A | LC-MS (ES) m/z: [M + 1]+: 347 |
| | | 1H NMR (300 MHZ, DMSO-d6, ppm) δ 11.99-11.92 (m, 1H), 10.45 (s, 1H), |
| | | 8.22 (d, J = 3.1 Hz, 1H), 8.00 (s, 1H), 7.52 (d, J = 7.9 Hz, 1H), 7.17 (t, J = 7.8 |
| | | Hz, 1H), 6.91 (d, J = 7.6 Hz, 1H), 3.29 (q, J = 6.8 Hz, 1H), 1.28 (d, J = 6.9 Hz, 6H). |
| 210 | A | LC-MS (ES) m/z: [M + 1]+: 345 |
| | | 1H NMR (300 MHz, DMSO-d6, ppm) δ 12.00-11.93 (m, 1H), 10.42 (s, 1H), |
| | | 8.23 (d, J = 3.1 Hz, 1H), 7.89 (s, 1H), 7.52 (d, J = 8.0 Hz, 1H), 7.17 (t, |
| | | J = 7.8 Hz, 1H), 6.96-6.87 (m, 1H), 2.46 (dd, J = 8.3, 4.6 Hz, 1H), 1.17 |
| | | (dt, J = 8.0, 3.4 Hz, 2H), 1.13-0.96 (m, 2H). |
| 211 | A | LC-MS (ES) m/z: [M + 1]+: 333 |
| | | 1H NMR (300 MHZ, DMSO-d6, ppm) δ 11.99 (s, 1H), 10.37 (s, 1H), 8.24 (d, |
| | | J = 3.1 Hz, 1H), 7.53 (d, J = 7.9 Hz, 1H), 7.15 (t, J = 7.8 Hz, 1H), 6.81 (d, |
| | | J = 7.6 Hz, 1H), 2.59 (s, 3H), 2.15 (s, 3H). |
| 212 | A | LC-MS (ES) m/z: [M − 1]−: 380 |
| | | 1H NMR (300 MHZ, DMSO-d6, ppm) δ 11.56 (t, J = 2.4 Hz, 1H), 10.54 (s, 1H), |
| | | 8.30 (d, J = 1.4 Hz, 1H), 7.52 (d, J = 2.8 Hz, 1H), 7.39 (s, OH), 6.80 (dd, J = 10.7, |
| | | 8.4 Hz, 1H), 6.69 (dd, J = 8.4, 4.2 Hz, 1H). |
| 213 | A | LC-MS (ES) m/z: [M − 1]−: 304 |
| | | 1H NMR (300 MHZ, DMSO-d6, ppm) δ 12.20 (d, J = 3.0 Hz, 1H), 11.30 (s, 1H), |
| | | 9.86 (s, 1H), 8.27 (d, J = 3.1 Hz, 1H), 7.56 (d, J = 7.9 Hz, 1H), 7.15 (t, J = 7.8 Hz, |
| | | 1H), 6.88 (dd, J = 7.7, 0.9 Hz, 1H). |
| 214 | A | LC-MS (ES) m/z: [M − 1]−: 303 |
| | | 1H NMR (300 MHZ, DMSO-d6, ppm) δ 12.09 (s, 1H), 10.73 (s, 1H), 8.66 (d, |
| | | J = 1.8 Hz, 1H), 8.24 (d, J = 3.1 Hz, 1H), 7.72 (d, J = 1.8 Hz, 1H), 7.56 (d, |
| | | J = 8.0 Hz, 1H), 7.16 (t, J = 7.8 Hz, 1H), 6.84 (dd, J = 7.6, 1.0 Hz, 1H). |
| 215 | A | LC-MS (ES) m/z: [M − 1]−: 303 |
| | | 1H NMR (300 MHz, DMSO-d6, ppm) δ 12.02 (s, 1H), 10.28 (s, 1H), 9.44 (s, 1H), |
| | | 8.69 (s, 1H), 8.23 (d, J = 3.1 Hz, 1H), 7.51 (d, J = 8.0 Hz, 1H), 7.14 (t, J = 7.8 Hz, |
| | | 1H), 6.80 (dd, J = 7.7, 0.9 Hz, 1H). |
| 216 | A | LC-MS (ES) m/z: [M + 1]+: 333 |
| | | 1H NMR (300 MHZ, DMSO-d6, ppm) δ 12.13 (d, J = 3.2 Hz, 1H), 10.74 (s, 1H), |
| | | 8.23 (d, J = 3.1 Hz, 1H), 8.13 (s, 2H), 6.87 (d, J = 7.8 Hz, 1H), 6.69 (d, J = 7.8 Hz, |
| | | 1H), 2.98 (q, J = 7.5 Hz, 2H), 1.24 (t, J = 7.5 Hz, 3H). |
| 217 | A | LC-MS (ES) m/z: [M − 1]−: 331 |
| | | 1H NMR (300 MHZ, DMSO-d6, ppm) δ 12.03 (s, 1H), 10.34 (s, 1H), 9.35 (s, 1H), |
| | | 8.25-8.16 (m, 2H), 6.92 (d, J = 7.7 Hz, 1H), 6.71 (d, J = 7.7 Hz, 1H), 3.00 (q, |
| | | J = 7.5 Hz, 2H), 1.26 (t, J = 7.5 Hz, 3H). |
| 218 | B | LC-MS (ES) m/z: [M + 1]+: 408 |
| | | 1H NMR (300 MHZ, DMSO-d6, ppm) δ 12.29 (s, 1H), 10.11 (s, 1H), 8.29 (s, 1H), |
| | | 7.48 (s, 1H), 6.99 (dd, J = 10.1, 8.5 Hz, 1H), 6.86 (dd, J = 8.4, 4.3 Hz, 1H), 3.69 |
| | | (t, J = 4.8 Hz, 4H), 3.44 (t, J = 4.9 Hz, 4H). |
| 219 | A | LC-MS (ES) m/z: [M − 1]−: 335 |
| | | 1H NMR (300 MHz, DMSO-d6, ppm) δ 12.06-12.01 (m, 1H), 10.61 (s, 1H), |
| | | 8.20 (d, J = 2.8 Hz, 1H), 8.15 (d, J = 1.6 Hz, 1H), 7.48 (d, J = 7.9 Hz, 1H), 7.15 |
| | | (t, J = 7.8 Hz, 1H), 6.90 (dd, J = 7.7, 0.9 Hz, 1H), 5.79 (s, 1H), 5.67 (s, 1H). |
| 220 | A | LC-MS (ES) m/z: [M + 1]+: 335 |
| | | 1H NMR (300 MHZ, DMSO-d6, ppm) δ 11.99 (s, 1H), 10.38 (s, 1H), 8.22 (q, |
| | | J = 2.6 Hz, 1H), 7.61-7.55 (m, 1H), 7.54-7.47 (m, 1H), 7.18 (td, J = 7.8, 2.6 Hz, |
| | | 1H), 7.01-6.93 (m, 1H), 4.06 (d, J = 2.7 Hz, 3H). |
| 221 | A | LC-MS (ES) m/z: [M + 1]+: 349 |
| | | 1H NMR (300 MHZ, DMSO-d6, ppm) δ 11.99 (s, 1H), 10.21 (s, 1H), 8.19 (d, |
| | | J = 2.9 Hz, 1H), 7.53 (s, 1H), 6.90 (d, J = 7.8 Hz, 1H), 6.79 (d, J = 7.7 Hz, 1H), |
| | | 4.07 (s, 3H), 2.62 (s, 3H). |
| 222 | A | LC-MS (ES) m/z: [M + 1]+: 391 |
| | | 1H NMR (300 MHz, CD3OD, ppm) δ 7.95 (s, 1H), 7.51 (s, 1H), 6.80 (s, 2H), |
| | | 2.97 (s, 2H), 2.66 (s, 3H), 1.17 (s, 6H). |
| 223 | A | LC-MS (ES) m/z: [M + 1]+: 323 |
| | | 1H NMR (300 MHZ, DMSO-d6, ppm) δ 12.19-12.12 (m, 1H), 11.00 (s, 1H), |
| | | 8.26 (d, J = 3.1 Hz, 1H), 8.04 (d, J = 3.5 Hz, 1H), 7.55 (d, J = 7.9 Hz, 1H), 7.15 |
| | | (t, J = 7.8 Hz, 1H), 6.87 (d, J = 7.6 Hz, 1H). |
| 224 | A | LC-MS (ES) m/z: [M − 1]−: 335 |
| | | 1H NMR (300 MHZ, DMSO-d6, ppm) δ 12.15 (s, 1H), 10.84 (s, 1H), 8.23 (d, |
| | | J = 3.1 Hz, 1H), 8.03 (d, J = 3.5 Hz, 1H), 6.87 (d, J = 7.7 Hz, 1H), 6.69 (d, |
| | | J = 7.7 Hz, 1H), 2.61 (s, 3H). |
| 225 | A | LC-MS (ES) m/z: [M + 1]+: 345 |
| | | 1H NMR (300 MHZ, DMSO-d6, ppm) δ 11.92 (s, 1H), 10.29 (s, 1H), 8.22 (d, |
| | | J =1 .9 Hz, 1H), 8.05 (s, 1H), 7.43 (dd, J = 8.0, 1.0 Hz, 1H), 7.10 (t, J = 7.8 Hz, 1H), |
| | | 6.87 (dd, J = 7.7, 1.0 Hz, 1H), 2.49-2.40 (m, 1H), 1.15 (dt, J = 8.2, 3.4 Hz, 2H), |
| | | 0.97-0.89 (m, 2H). |
| 226 | A | LC-MS (ES) m/z: [M − 1]−: 315 |
| | | 1H NMR (300 MHZ, DMSO-d6, ppm) δ 8.17 (s, 1H), 7.87 (s, 1H), 6.99-6.80 |
| | | (m, 3H), 2.70 (q, J = 7.5 Hz, 2H), 1.18 (t, J = 7.5 Hz, 3H). |
| 227 | A | LC-MS (ES) m/z: [M-1] -: 327 |
| | | 1H NMR (300 MHz, DMSO-d6, ppm) δ 11.98 (s, 1H), 10.40 (s, 1H), 8.53 (s, 1H), |
| | | 8.23 (d, J = 3.0 Hz, 1H), 7.48 (d, J = 7.9 Hz, 1H), 7.14 (t, J = 7.8 Hz, 1H), 6.92 |
| | | (d, J = 7.6 Hz, 1H), 2.25-2.05 (m, 1H), 1.10 (dt, J = 8.1, 3.3 Hz, 2H), |
| | | 1.05-0.89 (m, 2H). |
| 228 | A | LC-MS (ES) m/z: [M − 1]−: 357 |
| | | 1H NMR (300 MHZ, DMSO-d6, ppm) δ 11.87 (s, 1H), 9.53 (s, 1H), 8.16 (s, 1H), |
| | | 7.96 (s, 1H), 6.80 (d, J = 8.4 Hz, 1H), 6.67 (d, J = 7.7 Hz, 1H), 2.64-2.54 (m, 3H), |
| | | 2.45 (dt, J = 8.2, 4.8 Hz, 1H), 1.26-1.07 (m, 2H), 1.06-0.87 (m, 2H). |
| 229 | A | LC-MS (ES) m/z: [M + 1]+: 331 |
| | | 1H NMR (300 MHZ, DMSO-d6, ppm) δ 11.98 (d, J = 2.9 Hz, 1H), 10.25 (s, 1H), |
| | | 8.55 (s, 1H), 8.20 (d, J = 3.1 Hz, 1H), 6.85 (dd, J = 7.7, 1.0 Hz, 1H), 6.75 (d, |
| | | J = 7.7 Hz, 1H), 2.83 (q, J = 7.5 Hz, 2H), 2.63-2.57 (m, 3H), 1.24 (t, J = 7.6 Hz, 3H). |
| 230 | A | LC-MS (ES) m/z: [M + 1]+: 343 |
| | | 1H NMR (300 MHZ, DMSO-d6, ppm) δ 11.99 (s, 1H), 10.23 (s, 1H), 8.47 (s, 1H), |
| | | 8.20 (d, J = 3.1 Hz, 1H), 6.87 (d, J = 7.8 Hz, 1H), 6.73 (d, J = 7.7 Hz, 1H), 2.60 |
| | | (s, 3H), 2.26-2.05 (m, 1H), 1.11 (dt, J = 8.0, 3.3 Hz, 2H), 1.01-0.90 (m, 2H). |
| 231 | A | LC-MS (ES) m/z: [M + 1]+: 391 |
| | | 1H NMR (300 MHZ, DMSO-d6, ppm) δ 12.06 (s, 1H), 10.57 (s, 1H), 8.20 (d, |
| | | J = 3.1 Hz, 1H), 7.81 (s, 1H), 6.81 (d, J = 7.7 Hz, 1H), 6.65 (d, J = 7.7 Hz, 1H), |
| | | 4.80 (s, 1H), 2.93 (s, 2H), 2.59 (s, 3H), 2.55 (s, 2H), 1.05 (s, 6H). |
| 232 | A | LC-MS (ES) m/z: [M + 1]+: 333 |
| | | 1H NMR (300 MHZ, DMSO-d6, ppm) δ 11.95 (s, 1H), 10.35 (s, 1H), 8.21 (s, 1H), |
| | | 8.16 (s, 1H), 7.41 (dd, J = 8.0, 1.0 Hz, 1H), 7.08 (t, J = 7.8 Hz, 1H), 6.88 (dd, |
| | | J = 7.7, 0.9 Hz, 1H), 3.02 (q, J = 7.5 Hz, 2H), 1.27 (t, J = 7.5 Hz, 3H). |
| 233 | A | LC-MS (ES) m/z: [M + 1]+: 347 |
| | | 1H NMR (300 MHZ, DMSO-d6, ppm) δ 11.95 (s, 1H), 10.17 (s, 1H), 8.17 (s, 1H), |
| | | 8.09 (s, 1H), 6.79 (dd, J = 7.7, 1.0 Hz, 1H), 6.67 (d, J = 7.7 Hz, 1H), 3.03 (q, |
| | | J = 7.5 Hz, 2H), 2.57 (d, J = 0.9 Hz, 3H), 1.29 (t, J = 7.5 Hz, 3H). |
| 234 | A | LC-MS (ES) m/z: [M − 1]−: 321 |
| | | 1H NMR (300 MHZ, DMSO-d6, ppm) δ 12.39 (s, 1H), 10.21 (s, 1H), 9.40 (s, 1H), |
| | | 8.67 (s, 1H), 8.29 (d, J = 2.9 Hz, 1H), 6.94 (dd, J = 10.2, 8.4 Hz, 1H), 6.64 (dd, |
| | | J = 8.4, 4.3 Hz, 1H). |
| 235 | A | LC-MS (ES) m/z: [M − 1]−: 330 |
| | | 1H NMR (300 MHZ, DMSO-d6, ppm) δ 11.47 (s, 1H), 10.05 (s, 1H), 9.40 (s, 1H), |
| | | 8.67 (s, 1H), 7.50 (d, J = 2.7 Hz, 1H), 6.76 (dd, J = 10.8, 8.3 Hz, 1H), 6.61 (dd, |
| | | J = 8.3, 4.3 Hz, 1H). |
| 236 | A | LC-MS (ES) m/z: [M + 1]+: 314 |
| | | 1H NMR (300 MHz, DMSO-d6, ppm) δ 11.31-10.89 (m, 1H), 10.15 (s, 1H), |
| | | 9.43 (s, 1H), 8.68 (s, 1H), 7.50 (d, J = 2.8 Hz, 1H), 7.39-7.30 (m, 1H), 7.02 |
| | | (t, J = 7.8 Hz, 1H), 6.82 (dd, J = 7.6, 1.0 Hz, 1H). |
| 237 | A | LC-MS (ES) m/z: [M-1] -[M − 1]−: 317 |
| | | 1H NMR (300 MHZ, DMSO-d6, ppm) δ 12.16-11.87 (m, 1H), 10.12 (s, 1H), |
| | | 9.40 (s, 1H), 8.67 (s, 1H), 8.20 (d, J = 3.1 Hz, 1H), 6.95-6.76 (m, 1H), 6.61 |
| | | (d, J = 7.7 Hz, 1H), 2.60 (s, 3H). |
| 238 | A | LC-MS (ES) m/z: [M + 1]+: 315 |
| | | 1H NMR (300 MHZ, DMSO-d6, ppm) δ 13.18 (s, 1H), 10.59 (s, 1H), 9.36 |
| | | (d, J = 0.9 Hz, 1H), 8.24 (d, J = 0.9 Hz, 1H), 7.55 (d, J = 8.1 Hz, 1H), 7.15 |
| | | (t, J = 7.8 Hz, 1H), 7.00 (dd, J = 7.5, 0.9 Hz, 1H). |
| 239 | A | LC-MS (ES) m/z: [M − 1]−: 321 |
| | | 1H NMR (300 MHZ, DMSO-d6, ppm) δ 12.17 (s, 1H), 11.34 (s, 1H), 8.31 |
| | | (t, J = 2.3 Hz, 2H), 7.61 (dd, J = 8.0, 1.0 Hz, 1H), 7.42 (d, J = 7.6 Hz, 1H), |
| | | 7.31 (t, J = 7.8 Hz, 1H). |
| 240 | A | LC-MS (ES) m/z: [M − 1]−: 301 |
| | | 1H NMR (300 MHZ, DMSO-d6, ppm) δ 11.99 (s, 1H), 10.43 (s, 1H), 8.60 (s, 1H), |
| | | 8.22 (d, J = 2.6 Hz, 1H), 7.46 (d, J = 7.9 Hz, 1H), 7.13 (t, J = 7.8 Hz, 1H), 6.95 |
| | | (dd, J = 7.7, 0.9 Hz, 1H), 2.47 (s, 3H). |
| 241 | A | LC-MS (ES) m/z: [M + 1]+: 317 |
| | | 1H NMR (300 MHZ, DMSO-d6, ppm) δ 11.99 (s, 1H), 10.25 (s, 1H), 8.54 (s, 1H), |
| | | 8.20 (d, J = 3.1 Hz, 1H), 6.85 (dd, J = 7.7, 1.0 Hz, 1H), 6.75 (d, J = 7.7 Hz, 1H), |
| | | 2.60 (s, 3H), 2.48 (s, 3H). |
| 242 | A | LC-MS (ES) m/z: [M + 1]+: 329 |
| | | 1H NMR (300 MHz, DMSO-d6, ppm) δ 13.15 (s, 1H), 10.55 (s, 1H), 7.99 (s, 1H), |
| | | 7.54 (d, J = 8.1 Hz, 1H), 7.15 (t, J = 7.8 Hz, 1H), 7.05 (dd, J = 7.4, 0.9 Hz, 1H), |
| | | 2.69 (s, 3H). |
| 243 | A | LC-MS (ES) m/z: [M + 1]+: 315 |
| | | 1H NMR (300 MHz, DMSO-d6, ppm) δ 13.19 (s, 1H), 10.39 (s, 1H), 9.49 (s, 1H), |
| | | 8.72 (s, 1H), 7.52 (d, J = 8.1 Hz, 1H), 7.12 (dd, J = 8.1, 7.4 Hz, 1H), 6.96 (dd, |
| | | J = 7.5, 0.9 Hz, 1H). |
| 244 | A | LC-MS (ES) m/z: [M + 1]+: 306 |
| | | 1H NMR (300 MHZ, DMSO-d6, ppm) δ 10.77 (s, 1H), 9.38 (s, 1H), 8.23 (s, 1H), |
| | | 7.79 (d, J = 8.2 Hz, 1H), 7.30 (t, J = 7.8 Hz, 1H), 6.99 (d, J = 7.4 Hz, 1H). |
| 245 | A | LC-MS (ES) m/z: [M + 1]+: 320 |
| | | 1H NMR (300 MHz, DMSO-d6, ppm) δ 14.32 (s, 1H), 10.70 (s, 1H), 7.96 (s, 1H), |
| | | 7.76 (d, J = 8.2 Hz, 1H), 7.30 (t, J = 7.8 Hz, 1H), 7.05 (d, J = 7.4 Hz, 1H), 2.69 |
| | | (s, 3H). |
| 246 | A | LC-MS (ES) m/z: [M + 1]+: 306 |
| | | 1H NMR (300 MHz, DMSO-d6, ppm) δ 14.38 (s, 1H), 10.55 (s, 1H), 9.48 (s, 1H), |
| | | 8.72 (s, 1H), 7.76 (d, J = 8.2 Hz, 1H), 7.27 (t, J = 7.8 Hz, 1H), 6.94 |
| | | (d, J = 7.4 Hz, 1H). |
| 247 | A | LC-MS (ES) m/z: [M + 1]+: 324 |
| | | 1H NMR (300 MHz, DMSO-d6, ppm) δ 14.81 (s, 1H), 10.71 (s, 1H), 9.40 (s, 1H), |
| | | 8.20 (s, 1H), 7.11 (dd, J = 9.8, 8.1 Hz, 1H), 6.86 (dd, J = 8.2, 4.1 Hz, 1H). |
| 248 | A | LC-MS (ES) m/z: [M + 1]+: 338 |
| | | 1H NMR (300 MHz, DMSO-d6, ppm) δ 14.78 (s, 1H), 10.66 (s, 1H), 7.92 (s, 1H), |
| | | 7.11 (dd, J = 9.9, 8.2 Hz, 1H), 6.91 (dd, J = 8.2, 4.1 Hz, 1H), 2.71 (s, 3H). |
| 249 | A | LC-MS (ES) m/z: [M − 1]−: 346 |
| | | 1H NMR (300 MHZ, DMSO-d6, ppm) δ 11.71 (d, J = 3.2 Hz, 1H), 10.24 (s, 1H), |
| | | 9.45 (s, 1H), 8.71 (s, 1H), 7.93 (dd, J = 3.1, 15Hz, 1H), 7.46 (d, J = 8.0 Hz, 1H), |
| | | 7.08 (t, J = 7.8 Hz, 1H), 6.81 (d, J = 7.6 Hz, 1H). |
| 250 | A | LC-MS (ES) m/z: [M − 1]−: 301 |
| | | 1H NMR (300 MHz, DMSO-d6, ppm) δ 12.14 (s, 1H), 10.66 (s, 1H), 8.72 (s, 1H), |
| | | 8.21 (d, J = 3.0 Hz, 1H), 7.72 (s, 1H), 6.86 (d, J = 7.7 Hz, 1H), 6.64 (d, J = 7.7 Hz, |
| | | 1H), 2.57-2.48 (m, 3H). |
| 251 | A | LC-MS (ES) m/z: [M − 1]−: 287 |
| | | 1H NMR (300 MHz, DMSO-d6, ppm) δ 12.17-12.12 (m, 1H), 10.80 (s, 1H), |
| | | 8.72 (d, J = 1.5 Hz, 1H), 8.25 (d, J = 3.1 Hz, 1H), 7.76 (d, J = 1.6 Hz, 1H), 7.53 |
| | | (d, J = 8.0 Hz, 1H), 7.15 (t, J = 7.8 Hz, 1H), 6.82 (dd, J = 7.6, 1.0 Hz, 1H). |
| 252 | A | LC-MS (ES) m/z: [M + 1]+: 337 |
| | | 1H NMR (300 MHz, DMSO-d6, ppm) δ 12.26-12.13 (m, 1H), 11.21 (s, 1H), |
| | | 8.29 (dd, J = 3.4, 2.3 Hz, 2H), 7.27 (d, J = 7.7 Hz, 1H), 7.04 (dd, J = 7.7, 1.0 Hz, |
| | | 1H), 2.68 (s, 3H). |
| 253 | A | LC-MS (ES) m/z: [M + 1]+: 356 |
| | | 1H NMR (300 MHZ, DMSO-d6, ppm) δ 12.03 (d, J = 3.2 Hz, 1H), 9.84 (s, 1H), |
| | | 8.20 (d, J = 3.1 Hz, 1H), 7.61 (s, 1H), 6.90 (d, J = 7.8 Hz, 1H), 6.83 (d, J = 8.3 |
| | | Hz, 1H), 3.92 (t, J = 5.5 Hz, 2H), 2.75 (t, J = 5.8 Hz, 2H), 2.58 (s, 3H), |
| | | 1.90-1.76 (m, 4H). |
| 254 | A | LC-MS (ES) m/z: [M − 1]−: 304 |
| | | 1H NMR (300 MHZ, DMSO-d6, ppm) δ 12.12 (s, 1H), 10.86 (s, 1H), 9.14 (s, 1H), |
| | | 8.25 (d, J = 3.1 Hz, 1H), 7.52 (d, J = 8.0 Hz, 1H), 7.12 (t, J = 7.8 Hz, 1H), 6.83 |
| | | (d, J = 7.6 Hz, 1H). |
| 255 | A | LC-MS (ES) m/z: [M − 1]−: 304 |
| | | 1H NMR (300 MHZ, DMSO-d6, ppm) δ 12.21-12.14 (m, 1H), 11.02 (s, 1H), |
| | | 9.25 (s, 1H), 8.26 (d, J = 3.2 Hz, 1H), 7.60 (d, J = 8.0 Hz, 1H), 7.18 (t, J = 7.8 Hz, |
| | | 1H), 6.81 (dd, J = 7.6, 1.0 Hz, 1H). |
| 256 | A | LC-MS (ES) m/z: [M + 1]+: 319 |
| | | 1H NMR (300 MHZ, DMSO-d6, ppm) δ 12.08 (s, 1H), 10.71 (s, 1H), 8.24 (d, |
| | | J = 3.1 Hz, 1H), 7.60-7.51 (m, 2H), 7.17 (t, J = 7.8 Hz, 1H), 6.87 (dd, J = 7.6, |
| | | 0.9 Hz, 1H), 2.44 (s, 3H). |
| 257 | A | LC-MS (ES) m/z: [M + 1]+: 333 |
| | | 1H NMR (300 MHZ, DMSO-d6, ppm) δ 12.11-12.04 (m, 1H), 10.54 (s, 1H), |
| | | 8.21 (d, J = 3.1 Hz, 1H), 7.53 (s, 1H), 6.89 (d, J = 7.7 Hz, 1H), 6.70 (d, J = 7.7 Hz, |
| | | 1H), 2.62 (s, 3H), 2.44 (s, 3H). |
| 258 | A | LC-MS (ES) m/z: [M − 1]−: 317 |
| | | 1H NMR (300 MHZ, DMSO-d6, ppm) δ 12.03 (s, 1H), 10.33 (s, 1H), 9.39 (s, 1H), |
| | | 8.25 (d, J = 3.1 Hz, 1H), 7.50 (d, J = 8.0 Hz, 1H), 7.11 (t, J = 7.8 Hz, 1H), 6.74 |
| | | (d, J = 7.6 Hz, 1H), 2.41 (s, 3H). |
| 259 | A | LC-MS (ES) m/z: [M + 1]+: 320 |
| | | 1H NMR (300 MHZ, DMSO-d6, ppm) δ 12.12 (d, J = 3.2 Hz, 1H), 10.71 (s, 1H), |
| | | 9.11 (s, 1H), 8.22 (d, J = 3.2 Hz, 1H), 6.84 (d, J = 7.7 Hz, 1H), 6.63 (d, J = 7.7 Hz, |
| | | 1H), 2.60 (s, 3H). |
| 260 | A | LC-MS (ES) m/z: [M − 1]−: 318 |
| | | 1H NMR (300 MHZ, DMSO-d6, ppm) δ 12.17 (d, J = 3.2 Hz, 1H), 10.87 (s, 1H), |
| | | 9.23 (s, 1H), 8.23 (d, J = 3.2 Hz, 1H), 6.90 (dd, J = 7.7, 1.1 Hz, 1H), 6.63 (d, |
| | | J = 7.7 Hz, 1H), 2.64 (s, 3H). |
| 261 | A | LC-MS (ES) m/z: [M − 1]−: 317 |
| | | 1H NMR (300 MHZ, DMSO-d6, ppm) δ 12.15-11.98 (m, 1H), 10.56 (s, 1H), |
| | | 8.65 (d, J = 1.8 Hz, 1H), 8.21 (d, J = 3.1 Hz, 1H), 7.70 (d, J = 1.8 Hz, 1H), 6.89 |
| | | (d, J = 7.7 Hz, 1H), 6.68 (d, J = 7.7 Hz, 1H), 2.62 (s, 3H). |
| 262 | A | LC-MS (ES) m/z: [M + 1]+: 288 |
| | | 1H NMR (300 MHZ, DMSO-d6, ppm) δ 11.93 (s, 1H), 10.08 (s, 1H), 8.20 (dd, |
| | | J = 4.6, 2.3 Hz, 2H), 7.83 (t, J = 1.7 Hz, 1H), 7.47 (d, J = 7.9 Hz, 1H), 7.15 (t, |
| | | J = 7.8 Hz, 1H), 6.95 (d, J = 7.6 Hz, 1H), 6.69 (d, J = 2.0 Hz, 1H). |
| 263 | A | LC-MS (ES) m/z: [M + 1]+: 302 |
| | | 1H NMR (300 MHz, DMSO-d6, ppm) δ 11.93 (s, 1H), 9.90 (s, 1H), 8.21-8.11 |
| | | (m, 2H), 7.82 (t, J = 1.8 Hz, 1H), 6.86 (dd, J = 7.6, 1.0 Hz, 1H), 6.76 (d, |
| | | J = 7.7 Hz, 1H), 6.68 (d, J = 2.0 Hz, 1H), 2.60 (s, 3H). |
| 264 | A | LC-MS (ES) m/z: [M + 1]+: 344 |
| | | 1H NMR (300 MHZ, DMSO-d6, ppm) δ 11.55 (s, 1H), 10.46 (s, 1H), 7.57 (s, 1H), |
| | | 7.52 (d, J = 2.8 Hz, 1H), 6.78 (dd, J = 10.8, 8.3 Hz, 1H), 6.67 (dd, J = 8.4, 4.2 Hz, |
| | | 1H), 2.84 (q, J = 7.6 Hz, 2H), 1.22 (t, J = 7.6 Hz, 3H). |
| 265 | A | LC-MS (ES) m/z: [M − 1]−: 324 |
| | | 1H NMR (300 MHZ, DMSO-d6, ppm) δ 1.21-11.12 (m, 1H), 10.53 (s, 1H), 7.60 |
| | | (s, 1H), 7.51 (d, J = 2.7 Hz, 1H), 7.37 (d, J = 8.0 Hz, 1H), 7.04 (t, J = 7.8 Hz, |
| | | 1H), 6.83 (dd, J = 7.6, 1.0 Hz, 1H), 2.82 (q, J = 7.6 Hz, 2H), 1.20 (t, J = 7.6 Hz, 3H). |
| 266 | A | LC-MS (ES) m/z: [M − 1]−: 302 |
| | | 1H NMR (300 MHZ, DMSO-d6, ppm) δ 11.91 (s, 1H), 10.23 (s, 1H), 8.21 (d, |
| | | J = 3.0 Hz, 1H), 7.91 (dd, J = 5.0, 1.4 Hz, 1H), 7.53-7.43 (m, 2H), 7.18-7.05 |
| | | (m, 2H), 6.87 (dd, J = 7.7, 1.0 Hz, 1H). |
| 267 | A | LC-MS (ES) m/z: [M − 1]−: 302 |
| | | 1H NMR (300 MHz, DMSO-d6, ppm) δ 11.94 (s, 1H), 10.08 (s, 1H), 8.21 (s, 1H), |
| | | 8.08 (dd, J = 3.0, 1.3 Hz, 1H), 7.71 (dd, J = 5.2, 3.0 Hz, 1H), 7.44 (d, J = 8.0 Hz, |
| | | 1H), 7.28 (dd, J = 5.2, 1.4 Hz, 1H), 7.10 (t, J = 7.8 Hz, 1H), 6.83 (d, J = 7.6 Hz, 1H). |
| 268 | A | LC-MS (ES) m/z: [M + 1]+: 318 |
| | | 1H NMR (300 MHz, DMSO-d6, ppm) δ 11.94-11.89 (m, 1H), 10.07 (s, 1H), |
| | | 8.18 (d, J = 3.1 Hz, 1H), 7.90 (dd, J = 5.0, 1.4 Hz, 1H), 7.47 (dd, J = 3.8, 1.4 Hz, 1H), |
| | | 7.12 (dd, J = 5.0, 3.7 Hz, 1H), 6.85 (d, J = 7.7 Hz, 1H), 6.67 (d, J = 7.7 Hz, 1H), |
| | | 2.60 (s, 3H). |
| 269 | A | LC-MS (ES) m/z: [M + 1]+: 318 |
| | | 1H NMR (300 MHZ, DMSO-d6, ppm) δ 11.98-11.91 (m, 1H), 9.89 (s, 1H), 8.19 |
| | | (d, J = 2.9 Hz, 1H), 8.04 (dd, J = 3.0, 1.3 Hz, 1H), 7.72 (dd, J = 5.1, 3.0 Hz, 1H), |
| | | 7.28 (dd, J = 5.1, 1.4 Hz, 1H), 6.82 (dd, J = 7.7, 1.1 Hz, 1H), 6.63 (d, J = 7.7 Hz, |
| | | 1H), 2.58 (s, 3H). |
| 270 | A | LCMS (ES) m/z: [M − 1]−: 288 |
| | | 1H NMR (400 MHZ, DMSO-d6, ppm) δ 12.33 (s, 1H), 11.85-11.44 (m, 1H), |
| | | 9.55 (s, 1H), 8.28 (d, J = 3.1 Hz, 1H), 7.59 (d, J = 7.9 Hz, 1H), 7.19 (t, J = 7.8 Hz, |
| | | 1H), 7.00 (dd, J = 7.6, 1.0 Hz, 1H). |
| 271 | A | LCMS (ES) m/z: [M − 1]−: 302 |
| | | 1H NMR (400 MHZ, DMSO-d6, ppm) δ 12.32 (s, 1H), 11.47 (s, 1H), 9.55 (s, 1H), |
| | | 8.26 (d, J = 3.1 Hz, 1H), 7.03-6.58 (m, 2H), 2.64 (s, 3H). |
| 272 | A | LCMS (ES) m/z: [M − 1]−: 330 |
| | | 1H NMR (400 MHZ, DMSO-d6, ppm) δ 11.78 (s, 1H), 10.73 (s, 1H), 8.71 (s, 1H), |
| | | 7.92 (dd, J = 3.1, 1.5 Hz, 1H), 7.76 (s, 1H), 7.48 (d, J = 8.0 Hz, 1H), 7.09 (t, |
| | | J = 7.8 Hz, 1H), 6.83 (d, J = 7.6 Hz, 1H). |
| 273 | A | LCMS (ES) m/z: [M + 1]+: 319 |
| | | 1H NMR (400 MHZ, DMSO-d6, ppm) δ 12.01 (s, 1H), 10.26 (s, 1H), 8.52 (s, 1H), |
| | | 8.23 (d, J = 2.8 Hz, 1H), 7.50 (d, J = 7.9 Hz, 1H), 7.12 (t, J = 7.8 Hz, 1H), 6.76 |
| | | (dd, J = 7.6, 1.0 Hz, 1H), 2.36 (s, 3H). |
| 274 | A | LCMS (ES) m/z: [M + 1]+: 330 |
| | | 1H NMR (400 MHZ, DMSO-d6, ppm) δ 11.56 (s, 1H), 10.47 (s, 1H), 7.57-7.49 |
| | | (m, 2H), 6.77 (dd, J = 10.7, 8.3 Hz, 1H), 6.68 (dd, J = 8.4, 4.3 Hz, 1H), 2.51 |
| | | (d, J = 4.7 Hz, 3H). |
| 275 | A | LCMS (ES) m/z: [M + 1]+: 312 |
| | | 1H NMR (400 MHZ, DMSO-d6, ppm) δ 11.17 (d, J = 2.8 Hz, 1H), 10.54 (s, 1H), |
| | | 7.58 (s, 1H), 7.51 (d, J = 2.8 Hz, 1H), 7.37 (d, J = 8.0 Hz, 1H), 7.04 (t, J = 7.8 Hz, |
| | | 1H), 6.88-6.81 (m, 1H), 2.51 (d, J = 4.7 Hz, 3H). |
| 276 | A | LCMS (ES) m/z: [M + 1]+: 377 |
| | | 1H NMR (400 MHZ, DMSO-d6, ppm) δ 12.00-11.94 (m, 1H), 10.38 (s, 1H), |
| | | 8.21 (d, J = 2.7 Hz, 1H), 7.93 (s, 1H), 7.54-7.45 (m, 1H), 7.13 (t, J = 7.8 Hz, |
| | | 1H), 6.87 (dd, J = 7.7, 1.0 Hz, 1H), 4.95 (s, 1H), 3.05 (s, 2H), 1.08 (s, 6H). |
| 277 | A | LCMS (ES) m/z: [M − 1]−: 287 |
| | | 1H NMR (400 MHZ, DMSO-d6, ppm) δ 12.08 (s, 1H), 10.36 (s, 1H), 9.57 (s, 1H), |
| | | 8.92 (s, 1H), 8.25 (d, J = 3.1 Hz, 1H), 7.54 (d, J = 8.0 Hz, 1H), 7.18 (t, J = 7.8 Hz, |
| | | 1H), 6.89 (dd, J = 7.7, 0.9 Hz, 1H). |
| 278 | A | LCMS (ES) m/z: [M + 1]+: 391 |
| | | 1H NMR (400 MHZ, DMSO-d6, ppm) δ 11.98 (s, 1H), 10.19 (s, 1H), 8.18 (d, |
| | | J = 3.0 Hz, 1H), 7.89 (s, 1H), 6.90-6.81 (m, 1H), 6.68 (d, J = 7.7 Hz, 1H), 4.95 |
| | | (s, 1H), 3.06 (s, 2H), 2.61 (s, 3H), 1.10 (s, 6H). |
| 279 | A | CMS (ES) m/z: [M − 1]−: 333 |
| | | 1H NMR (400 MHZ, DMSO-d6, ppm) δ 12.43 (s, 1H), 10.62 (s, 1H), 8.28 (s, 1H), |
| | | 7.55 (s, 1H), 6.95 (dd, J = 10.2, 8.4 Hz, 1H), 6.77 (dd, J = 8.5, 4.4 Hz, 1H), 2.84 |
| | | (q, J = 7.6 Hz, 2H), 1.22 (t, J = 7.5 Hz, 3H). |
| 280 | A | LCMS (ES) m/z: [M + 1]+: 288 |
| | | 1H NMR (400 MHZ, DMSO-d6, ppm) δ 12.03 (s, 1H), 10.48 (s, 1H), 8.23 (d, |
| | | J = 3.0 Hz, 1H), 7.99 (d, J = 1.5 Hz, 1H), 7.49 (d, J = 7.9 Hz, 1H), 7.12 (t, |
| | | J = 7.8 Hz, 1H), 7.05 (dd, J = 3.6, 0.9 Hz, 1H), 6.84-6.75 (m, 1H), 6.63 |
| | | (dd, J = 3.5,1.8 Hz, 1H). |
| 281 | A | LCMS (ES) m/z: [M + 1]+: 302 |
| | | 1H NMR (400 MHZ, DMSO-d6, ppm) δ 12.07-12.00 (m, 1H), 10.31 (s, 1H), |
| | | 8.20 (d, J = 3.0 Hz, 1H), 8.00 (dd, J = 1.8, 0.9 Hz, 1H), 6.99 (dd, J = 3.5, 0.9 Hz, |
| | | 1H), 6.84 (dd, J = 7.7, 1.0 Hz, 1H), 6.66-6.54 (m, 2H), 2.63-2.57 (m, 3H). |
| 282 | A | LCMS (ES) m/z: [M − 1]−: 333 |
| | | 1H NMR (400 MHZ, DMSO-d6, ppm) δ 12.03 (s, 1H), 9.96 (s, 1H), 9.37 (s, 1H), |
| | | 8.64 (s, 1H), 8.08 (d, J = 3.0 Hz, 1H), 6.57 (d, J = 2.1 Hz, 2H), 3.86 (s, 3H). |
| 283 | A | LCMS (ES) m/z: [M − 1]−: 301 |
| | | 1H NMR (400 MHz, DMSO-d6, ppm) δ 12.07 (d, J = 3.2 Hz, 1H), 10.20 (s, 1H), |
| | | 9.53 (s, 1H), 8.90 (s, 1H), 8.22 (d, J = 3.1 Hz, 1H), 6.90 (dd, J = 7.6, 1.0 Hz, 1H), |
| | | 6.72 (d, J = 7.7 Hz, 1H), 2.62 (s, 3H). |
| 284 | A | LCMS (ES) m/z: [M + 1]+: 287 |
| | | 1H NMR (400 MHZ, DMSO-d6, ppm) δ 11.86 (s, 2H), 9.87 (s, 1H), 8.20 (s, 1H), |
| | | 7.40 (d, J = 7.9 Hz, 1H), 7.09 (t, J = 7.9 Hz, 1H), 6.93 (s, 1H), 6.85 (d, J = 7.6 Hz, |
| | | 1H), 6.61-6.56 (m, 1H), 6.13 (t, J = 3.1 Hz, 1H). |
| 285 | A | LCMS (ES) m/z: [M + 1]+: 287 |
| | | 1H NMR (400 MHZ, DMSO-d6, ppm) δ 11.77 (s, 1H), 11.47 (s, 1H), 9.62 (s, 1H), |
| | | 8.18 (d, J = 2.8 Hz, 1H), 7.36 (d, J = 7.9 Hz, 1H), 7.19 (s, 1H), 7.10 (t, J = 7.8 Hz, |
| | | 1H), 7.02 (d, J = 7.7 Hz, 1H), 6.81 (d, J = 2.7 Hz, 1H), 6.28 (s, 1H). |
| 286 | A | LCMS (ES) m/z: [M − 1]−: 299 |
| | | 1H NMR (400 MHZ, DMSO-d6, ppm) δ 11.71 (d, 2H), 9.43 (s, 1H), 8.14 (s, 1H), |
| | | 7.13 (t, J = 1.9 Hz, 1H), 6.81 (d, J = 3.7 Hz, 3H), 6.26 (dd, J = 2.9, 1.6 Hz, 1H), |
| | | 2.57 (s, 3H). |
| 287 | A | LCMS (ES) m/z: [M + 1]+: 335 |
| | | 1H NMR (400 MHZ, DMSO-d6, ppm) δ 11.82-11.76 (m, 1H), 10.53 (s, 1H), |
| | | 9.34 (s, 1H), 8.25 (s, 1H), 8.13 (d, J = 3.1 Hz, 1H), 6.96 (d, J = 2.2 Hz, 1H), |
| | | 6.52 (d, J = 2.2 Hz, 1H), 3.76 (s, 3H). |
| 288 | A | LCMS (ES) m/z: [M + 1]+: 335 |
| | | 1H NMR (400 MHZ, DMSO-d6, ppm) δ 12.03 (s, 1H), 10.16 (s, 1H), 9.35 (s, 1H), |
| | | 8.15 (s, 1H), 8.09 (d, J = 2.8 Hz, 1H), 6.68-6.55 (m, 2H), 3.87 (s, 3H). |
| 289 | A | LCMS (ES) m/z: [M + 1]+: 335 |
| | | 1H NMR (400 MHZ, DMSO-d6, ppm) δ 12.14 (s, 1H), 10.56 (s, 1H), 8.13-8.10 |
| | | (m, 3H), 6.61-6.53 (m, 2H), 3.85 (s, 3H). |
| 290 | A | LCMS (ES) m/z: [M − 1]−: 317 |
| | | 1H NMR (400 MHZ, DMSO-d6, ppm) δ 12.18 (d, J = 3.0 Hz, 1H), 10.51 (s, 1H), |
| | | 8.75 (s, 1H), 8.11 (d, J = 3.1 Hz, 1H), 7.70 (s, 1H), 6.59 (d, J = 1.5 Hz, 2H), 3.87 |
| | | (s, 3H). |
| 291 | A | LCMS (ES) m/z: [M + 1]+: 333 |
| | | 1H NMR (400 MHZ, DMSO-d6, ppm) δ 11.95 (s, 1H), 10.19 (s, 1H), 9.42 (s, 1H), |
| | | 8.67 (s, 1H), 8.18 (s, 1H), 7.31 (s, 1H), 6.57 (s, 1H), 2.58 (q, J = 8.4, 7.8 Hz, 2H), |
| | | 1.08 (t, J = 7.5 Hz, 3H). |
| 292 | A | LCMS (ES) m/z: [M + 1]+: 311 |
| | | 1H NMR (400 MHZ, DMSO-d6, ppm) δ 11.08 (s, 1H), 9.93 (s, 1H), 7.80-7.74 |
| | | (m, 2H), 7.50 (d, J = 2.1 Hz, 1H), 7.21 (dd, J = 7.3, 1.7 Hz, 1H), 7.03-6.97 (m, |
| | | 1H), 7.01-6.92 (m, 1H), 3.63 (s, 3H). |
| 293 | A | LCMS (ES) m/z: [[M + 1]+: 297 |
| | | 1H NMR (400 MHZ, DMSO-d6, ppm) δ 13.10-10.10 (s, 2H), 7.82 (d, J = 1.2 Hz, |
| | | 1H), 7.72 (d, J = 1.2 Hz, 1H), 7.49 (s, 1H), 7.19 (dd, J = 6.9, 2.0 Hz, 1H), |
| | | 7.01-6.91 (m, 2H). |
| 294 | A | LCMS (ES) m/z: [M + 1]+: 302 |
| | | 1H NMR (400 MHZ, DMSO-d6, ppm) δ 11.95 (s, 1H), 10.13 (s, 1H), 8.21 (s, 1H), |
| | | 7.85-7.75 (m, 2H), 7.37 (dd, J = 7.9, 1.0 Hz, 1H), 7.09 (t, J = 7.8 Hz, 1H), 7.02 |
| | | (dd, J = 7.7, 1.1 Hz, 1H), 3.63 (s, 3H). |
| 295 | A | LCMS (ES) m/z: [M − 1]−: 288 |
| | | 1H NMR (400 MHZ, DMSO-d6, ppm) δ 12.22 (s, 1H), 11.28 (s, 1H), 8.28 (d, |
| | | J = 3.1 Hz, 1H), 7.58 (d, J = 7.9 Hz, 1H), 7.24 (t, J = 7.8 Hz, 1H), 7.11-7.05 (m, 1H). |
| 296 | A | LCMS (ES) m/z: [M − 1]−: 302 |
| | | 1H NMR (400 MHZ, DMSO-d6, ppm) δ 12.23 (d, J = 3.2 Hz, 1H), 11.11 (s, 1H), |
| | | 8.27 (d, J = 3.1 Hz, 1H), 7.03-6.89 (m, 2H), 2.68-2.61 (m, 3H). |
| 297 | A | LCMS (ES) m/z: [M + 1]+: 301 |
| | | 1H NMR (400 MHZ, DMSO-d6, ppm) δ 12.14-11.51 (s, 2H), 9.61 (s, 1H), 8.16 |
| | | (s, 1H), 6.90 (s, 1H), 6.80 (d, J = 6.9 Hz, 1H), 6.63 (s, 1H), 6.54 (s, 1H), 6.12 |
| | | (s, 1H), 2.58 (s, 3H). |
| 298 | A | LCMS (ES) m/z: [M − 1]−: 333 |
| | | 1H NMR (400 MHZ, DMSO-d6, ppm) δ 11.83 (d, J = 3.1 Hz, 1H), 10.35 (s, 1H), |
| | | 9.49 (s, 1H), 8.72 (s, 1H), 8.15 (d, J = 3.1 Hz, 1H), 6.97-6.92 (m, 1H), 6.45 |
| | | (d, J = 2.7 Hz, 1H), 3.74 (s, 3H). |
| 299 | A | LCMS (ES) m/z: [M + 1]+: 335 |
| | | 1H NMR (400 MHZ, DMSO-d6, ppm) δ 11.87 (s, 1H), 10.92 (s, 1H), 8.18-8.07 |
| | | (m, 3H), 6.94 (d, J = 6.1 Hz, 1H), 6.58-6.49 (m, 1H), 3.75 (s, 3H). |
| 300 | A | LCMS (ES) m/z: [M − 1]−: 337 |
| | | 1H NMR (400 MHZ, DMSO-d6, ppm) δ 12.36 (s, 1H), 10.31 (s, 1H), 9.43 |
| | | (s, 1H), 8.70 (s, 1H), 8.35 (d, J = 2.3 Hz, 1H), 7.18 (d, J = 8.2 Hz, 1H), 6.73 |
| | | (d, J = 8.2 Hz, 1H). |
| 301 | A | LCMS (ES) m/z: [M − 1]−: 337 |
| | | 1H NMR (400 MHZ, DMSO-d6, ppm) δ 12.36 (s, 1H), 10.53 (s, 1H), 9.36 (s, 1H), |
| | | 8.35 (d, J = 3.0 Hz, 1H), 8.22 (s, 1H), 7.20 (d, J = 8.2 Hz, 1H), 6.79 (d, |
| | | J = 8.2 Hz, 1H). |
| 302 | A | LCMS (ES) m/z: [M + 1]+: 339 |
| | | 1H NMR (400 MHZ, DMSO-d6, ppm) δ 12.47 (d, J = 3.3 Hz, 1H), 10.95 (s, 1H), |
| | | 8.38 (d, J = 3.1 Hz, 1H), 8.17-8.09 (m, 2H), 7.17 (d, J = 8.2 Hz, 1H), 6.78 |
| | | (d, J = 8.2 Hz, 1H). |
| 303 | A | LCMS (ES) m/z: [M − 1]−: 321 |
| | | 1H NMR (400 MHZ, DMSO-d6, ppm) δ 12.54-12.49 (m, 1H), 10.88-10.84 (m, |
| | | 1H), 8.74 (s, 1H), 8.38 (d, J = 3.1 Hz, 1H), 7.78 (s, 1H), 7.19 (d, J = 8.2 Hz, 1H), |
| | | 6.77 (d, J = 8.2 Hz, 1H). |
| 304 | A | LCMS (ES) m/z: [M + 1]+: 333 |
| | | 1H NMR (400 MHZ, DMSO-d6, ppm) δ 11.92 (d, J = 3.2 Hz, 1H), 10.42 (s, 1H), |
| | | 9.35 (d, J = 0.9 Hz, 1H), 8.23-8.15 (m, 2H), 7.33 (d, J = 1.4 Hz, 1H), 6.65 |
| | | (d, J = 1.4 Hz, 1H), 2.61 (q, J = 7.5 Hz, 2H), 1.10 (t, J = 7.6 Hz, 3H). |
| 305 | A | LCMS (ES) m/z: [M − 1]−: 331 |
| | | 1H NMR (400 MHZ, DMSO-d6, ppm) δ 12.00 (d, J = 3.2 Hz, 1H), 10.80 (s, 1H), |
| | | 8.15 (d, J = 16.6 Hz, 3H), 7.29 (s, 1H), 6.63 (d, J = 1.4 Hz, 1H), 2.58 (t, J = 7.6 Hz, |
| | | 2H), 1.06 (t, J = 7.5 Hz, 3H). |
| 306 | A | LCMS (ES) m/z: [M − 1]−: 332 |
| | | 1H NMR (400 MHZ, DMSO-d6, ppm) δ 11.97-11.90 (m, 1H), 10.26 (s, 1H), |
| | | 8.22 (d, J = 2.7 Hz, 1H), 7.64 (d, J = 1.6 Hz, 1H), 7.52-7.41 (m, 2H), 7.14 |
| | | (t, J = 7.8 Hz, 1H), 6.90 (dd, J = 7.7, 0.9 Hz, 1H), 5.27 (t, J = 5.8 Hz, 1H), 4.39 |
| | | (d, J = 5.6 Hz, 2H). |
| 307 | A | LCMS (ES) m/z: [M + 1]+: 316 |
| | | 1H NMR (400 MHZ, DMSO-d6, ppm) δ 12.05 (s, 1H), 10.11-9.95 (m, 1H), 8.23 |
| | | (s, 1H), 7.72 (s, 1H), 7.37 (d, J = 7.8 Hz, 1H), 7.10 (t, J = 7.8 Hz, 1H), 7.03 |
| | | (d, J = 7.6 Hz, 1H), 3.54 (s, 3H), 2.29 (s, 3H). |
| 308 | A | LCMS (ES) m/z: [M + 1]+: 305 |
| | | 1H NMR (400 MHZ, DMSO-d6, ppm) δ 1H NMR (400 MHZ, DMSO-d6, ppm) |
| | | δ 12.02 (s, 1H), 10.58 (s, 1H), 9.23 (d, J = 4.6 Hz, 1H), 8.23 (d, J = 2.8 Hz, 1H), |
| | | 7.64 (d, J = 4.7 Hz, 1H), 7.46 (d, J = 8.0 Hz, 1H), 7.09 (t, J = 7.8 Hz, 1H), 6.85 |
| | | (dd, J = 7.6, 0.9 Hz, 1H). |
| 309 | A | LCMS (ES) m/z: [M − 1]−: 317 |
| | | 1H NMR (400 MHZ, DMSO-d6, ppm) δ 11.96-11.88 (m, 1H), 10.81 (s, 1H), |
| | | 8.20-8.06 (m, 3H), 7.29 (s, 1H), 6.66 (d, J = 1.4 Hz, 1H), 2.28 (s, 3H). |
| 310 | A | LCMS (ES) m/z: [M + 1]+: 349 |
| | | 1H NMR (400 MHZ, DMSO-d6, ppm) δ 12.00 (s, 1H), 10.65 (s, 1H), 8.20 (s, 1H), |
| | | 8.04 (s, 1H), 7.46 (d, J = 7.9 Hz, 1H), 7.14 (t, J = 7.8 Hz, 1H), 6.90 (d, J = 7.6 |
| | | Hz, 1H), 4.71 (s, 2H), 3.38 (s, 3H). |
| 311 | A | LCMS (ES) m/z: [M − 1]−: 332 |
| | | 1H NMR (400 MHZ, DMSO-d6, ppm) δ 11.95-11.90 (m, 1H), 10.08 (s, 1H), |
| | | 8.22 (d, J = 3.1 Hz, 1H), 7.98 (d, J = 1.6 Hz, 1H), 7.44 (d, J = 7.9 Hz, 1H), 7.15 |
| | | (q, J = 1.2 Hz, 1H), 7.11 (t, J = 7.8 Hz, 1H), 6.88 (dd, J = 7.6, 1.0 Hz, 1H), 5.68 |
| | | (t, J = 5.8 Hz, 1H), 4.60 (dd, J = 5.8, 1.1 Hz, 2H). |
| 312 | A | LCMS (ES) m/z: [M + 1]+: 349 |
| | | 1H NMR (400 MHZ, DMSO-d6, ppm) δ 12.10 (s, 1H), 10.90 (s, 1H), 8.25 (d, |
| | | J = 3.1 Hz, 1H), 8.03 (d, J = 0.9 Hz, 1H), 7.51 (d, J = 7.9 Hz, 1H), 7.12 (t, J = 7.8 |
| | | Hz, 1H), 6.87 (dd, J = 7.7, 1.0 Hz, 1H), 4.68 (d, J = 0.9 Hz, 2H), 3.27 (s, 3H). |
| 313 | A | LCMS (ES) m/z: [M − 1]−: 317 |
| | | 1H NMR (400 MHZ, DMSO-d6, ppm) δ 11.98 (s, 1H), 10.55 (s, 1H), 8.22 (d, |
| | | J = 2.4 Hz, 1H), 7.48-7.41 (m, 2H), 7.10 (t, J = 7.8 Hz, 1H), 6.90 (d, J = 7.6 Hz, |
| | | 1H), 2.58 (s, 3H). |
| 314 | A | LCMS (ES) m/z: [M − 1]−: 317 |
| | | 1H NMR (400 MHZ, DMSO-d6, ppm) δ 11.86 (d, J = 3.3 Hz, 1H), 10.22 (s, 1H), |
| | | 9.44 (s, 1H), 8.69 (s, 1H), 8.15 (d, J = 3.1 Hz, 1H), 7.29 (s, 1H), 6.63 (d, J = 1.4 Hz, |
| | | 1H), 2.31 (s, 3H). |
| 315 | A | LCMS (ES) m/z: [M − 1]−: 317 |
| | | 1H NMR (400 MHZ, DMSO-d6, ppm) δ 11.85 (d, J = 3.3 Hz, 1H), 10.44 (s, 1H), |
| | | 9.34 (s, 1H), 8.22 (s, 1H), 8.15 (d, J = 3.1 Hz, 1H), 7.32 (s, 1H), 6.69 (d, J = 1.4 Hz, |
| | | 1H), 2.32 (s, 3H). |
| 316 | I | LCMS (ES) m/z: [M + 1]+: 319 |
| | | 1H NMR (400 MHZ, DMSO-d6, ppm) δ 11.79 (s, 1H), 9.76 (s, 1H), 8.19 (d, |
| | | J = 2.3 Hz, 1H), 7.41 (d, J = 7.9 Hz, 1H), 7.14 (t, J = 7.8 Hz, 1H), 7.05-6.95 |
| | | (m, 2H), 6.58 (s, 2H), 5.79-5.69 (m, 1H). |
| 317 | I | LCMS (ES) m/z: [M + 1]+: 320 |
| | | 1H NMR (400 MHZ, DMSO-d6, ppm) δ 11.87 (d, J = 3.1 Hz, 1H), 10.01 (s, 1H), |
| | | 8.22 (d, J = 3.1 Hz, 1H), 7.85 (s, 2H), 7.47 (d, J = 7.9 Hz, 1H), 7.30 (s, 1H), 7.18 |
| | | (t, J = 7.8 Hz, 1H), 7.02 (d, J = 7.7 Hz, 1H). |
| 318 | A | LCMS (ES) m/z: [M + 1]+: 335 |
| | | 1H NMR (400 MHZ, DMSO-d6, ppm) δ 7.82 (s, 1H), 7.07 (d, J = 7.2 Hz, 1H), |
| | | 6.91-6.76 (m, 2H), 6.51 (s, 1H), 6.08 (s, 2H), 3.74 (s, 3H). |
| 319 | A | LCMS (ES) m/z: [M + 1]+: 363 |
| | | 1H NMR (400 MHZ, DMSO-d6, ppm) δ 12.05 (d, J = 3.1 Hz, 1H), 10.81 (s, 1H), |
| | | 8.24 (d, J = 3.1 Hz, 1H), 7.87 (d, J = 1.0 Hz, 1H), 7.50 (d, J = 7.9 Hz, 1H), 7.12 |
| | | (t, J = 7.8 Hz, 1H), 6.90 (dd, J = 7.7, 1.0 Hz, 1H), 3.50 (t, J = 5.9 Hz, 2H), 3.24 |
| | | (s, 3H), 3.10 (td, J = 5.9, 1.0 Hz, 2H). |
| 320 | A | LCMS (ES) m/z: [M + 1]+: 329 |
| | | 1H NMR (400 MHZ, DMSO-d6, ppm) δ 13.26 (s, 1H), 10.81 (s, 1H), 7.58 |
| | | (d, J = 8.2 Hz, 2H), 7.16 (t, J = 7.8 Hz, 1H), 7.07-6.99 (m, 1H), 2.44 (s, 3H). |
| 32 | A | LCMS (ES) m/z: [M − 1]−: 331 |
| | | 1H NMR (400 MHZ, DMSO-d6, ppm) δ 11.96 (s, 1H), 10.38 (s, 1H), 8.15 (s, 1H), |
| | | 7.41 (s, 1H), 6.79 (d, J = 7.8 Hz, 1H), 6.72 (d, J = 7.7 Hz, 1H), 2.57 (s, 6H). |
| 322 | A | LCMS (ES) m/z: [M + 1]+: 348 |
| | | 1H NMR (400 MHZ, DMSO-d6, ppm) δ 11.89 (s, 1H), 10.01 (s, 1H), 8.21 |
| | | (d, J = 2.4 Hz, 1H), 7.87 (d, J = 1.5 Hz, 1H), 7.44 (d, J = 7.9 Hz, 1H), 7.11-7.07 |
| | | (m, 2H), 6.88 (d, J = 7.6 Hz, 1H), 4.88 (t, J = 5.0 Hz, 1H), 3.57 (q, J = 5.9 Hz, 2H), |
| | | 2.89 (t, J = 6.2 Hz, 2H). |
| 323 | A | LCMS (ES) m/z: [M + 1]+: 337 |
| | | 1H NMR (400 MHZ, DMSO-d6, ppm) δ 12.43 (s, 1H), 10.65 (s, 1H), 8.28 |
| | | (d, J = 2.1 Hz, 1H), 7.52 (s, 1H), 6.96 (dd, J = 10.2, 8.4 Hz, 1H), 6.76 (dd, |
| | | J = 8.4, 4.3 Hz, 1H), 2.44 (s, 3H). |
| 324 | A | LCMS (ES) m/z: [M − 1]−: 347 |
| | | 1H NMR (400 MHZ, DMSO-d6, ppm) δ 12.08 (s, 1H), 10.40 (s, 1H), 8.10 (d, |
| | | J = 3.0 Hz, 1H), 7.52 (s, 1H), 6.69 (d, J = 8.3 Hz, 1H), 6.61 (d, J = 8.4 Hz, 1H), |
| | | 3.88 (s, 3H), 2.45 (s, 3H). |
| 325 | A | LCMS (ES) m/z: [M + 1]+: 353 |
| | | 1H NMR (400 MHZ, DMSO-d6, ppm) δ 12.44 (s, 1H), 10.77 (s, 1H), 8.36 |
| | | (d, J = 2.7 Hz, 1H), 7.55 (s, 1H), 7.21 (d, J = 8.2 Hz, 1H), 6.82 (d, |
| | | J = 8.2 Hz, 1H), 2.44 (s, 3H). |
| 326 | A | LCMS (ES) m/z: [M + 1]+: 319 |
| | | 1H NMR (400 MHZ, DMSO-d6, ppm) δ 12.01 (s, 1H), 10.26 (s, 1H), 8.52 (s, 1H), |
| | | 8.23 (d, J = 2.8 Hz, 1H), 7.50 (d, J = 7.9 Hz, 1H), 7.12 (t, J = 7.8 Hz, 1H), 6.76 |
| | | (dd, J = 7.6, 1.0 Hz, 1H), 2.36 (s, 3H). |
| 327 | A | LCMS (ES) m/z: [M + 1]+: 341 |
| | | 1H NMR (400 MHZ, DMSO-d6, ppm) δ 11.13 (s, 1H), 10.1 (s, 1 H), 7.80-7.73 |
| | | (m, 2H), 7.48 (s, 1H), 7.19 (d, J = 7.3 Hz, 1H), 6.98 (s, 2H), 4.98 (s, 1H), 3.98 |
| | | (d, J = 5.5 Hz, 2H), 3.61 (d, J = 6.4 Hz, 2H). |
| 328 | A | LCMS (ES) m/z: [M + 1]+: 341 |
| | | 1H NMR (400 MHZ, DMSO-d6, ppm) δ 11.07 (s, 1H), 10.24 (s, 1H), 7.84 (s, 1H), |
| | | 7.51 (d, J = 2.8 Hz, 1H), 7.37 (s, 1H), 7.30 (d, J = 8.0 Hz, 1H), 6.99 (t, J = 7.8 Hz, |
| | | 1H), 6.82 (d, J = 7.6 Hz, 1H), 5.07 (s, 1H), 4.19 (t, J = 5.3 Hz, 2H), 3.65 (t, |
| | | J = 5.3 Hz, 2H). |
| 329 | A | LCMS (ES) m/z: [M + 1]+: 355 |
| | | 1H NMR (400 MHZ, DMSO-d6, ppm) δ 11.07 (s, 1H), 9.85 (s, 1H), 7.81-7.73 |
| | | (m, 2H), 7.49 (d, J = 2.7 Hz, 1H), 7.27-7.16 (m, 1H), 7.01-6.89 (m, 2H), 4.11 |
| | | (t, J = 5.1 Hz, 2H), 3.53 (t, J = 5.1 Hz, 2H), 3.16 (s, 3H). |
| 330 | A | LCMS (ES) m/z: [M + 1]+: 355 |
| | | 1H NMR (400 MHZ, DMSO-d6, ppm) δ 11.07 (s, 1H), 10.27 (s, 1H), 7.84 (s, 1H), |
| | | 7.55-7.50 (m, 1H), 7.39 (s, 1H), 7.31 (d, J = 8.0 Hz, 1H), 7.00 (td, J = 7.8, 1.5 |
| | | Hz, 1H), 6.80 (d, J = 7.6 Hz, 1H), 4.28 (t, J = 5.2 Hz, 2H), 3.54 (t, J = 5.2 Hz, |
| | | 2H), 3.15 (d, J = 1.4 Hz, 3H). |
| 331 | A | LCMS (ES) m/z: [M + 1]+: 346 |
| | | 1H NMR (400 MHZ, DMSO-d6, ppm) δ 11.95 (s, 1H), 10.03 (s, 1H), 8.20 (d, |
| | | J = 2.9 Hz, 1H), 7.78 (dd, J = 14.4, 1.3 Hz, 2H), 7.38 (d, J = 7.8 Hz, 1H), 7.08 (t, |
| | | J = 7.8 Hz, 1H), 6.99 (d, J = 7.6 Hz, 1H), 4.12 (t, J = 5.1 Hz, 2H), 3.53 (t, J = 5.0 Hz, |
| | | 2H), 3.17 (s, 3H). |
| 332 | A | LCMS (ES) m/z: [M + 1]+: 346 |
| | | 1H NMR (400 MHZ, DMSO-d6, ppm) δ 11.97 (s, 1H), 10.38 (s, 1H), 8.24 (d, |
| | | J = 3.0 Hz, 1H), 7.86 (s, 1H), 7.47 (d, J = 8.0 Hz, 1H), 7.37 (s, 1H), 7.11 (t, J = 7.8 |
| | | Hz, 1H), 6.81 (d, J = 7.7 Hz, 1H), 4.27 (t, J = 5.3 Hz, 2H), 3.55 (t, J = 5.3 Hz, 2H), |
| | | 3.16 (s, 3H). |
| 333 | A | LCMS (ES) m/z: [M + 1]+: 363 |
| | | 1H NMR (400 MHZ, DMSO-d6, ppm) δ 11.98 (s, 1H), 10.43 (s, 1H), 8.23 (d, |
| | | J = 3.1 Hz, 1H), 7.99 (s, 1H), 7.52 (d, J = 8.0 Hz, 1H), 7.16 (t, J = 7.8 Hz, 1H), |
| | | 6.90 (dd, J = 7.7, 1.0 Hz, 1H), 3.62 (t, J = 5.9 Hz, 2H), 3.23 (d, J = 6.6 Hz, 5H). |
| 334 | I | LCMS (ES) m/z: [M + 1]+: 320 |
| | | 1H NMR (400 MHZ, DMSO-d6, ppm) δ 12.01 (s, 1H), 10.67 (s, 1H), 8.23 (s, 1H), |
| | | 7.48 (d, J = 8.2 Hz, 1H), 7.12 (t, J = 7.8 Hz, 1H), 6.91 (dd, J = 7.7, 1.0 Hz, 1H), |
| | | 6.24 (s, 1H), 5.79 (s, 2H). |
| 335 | A | LCMS (ES) m/z: [M + 1]+: 320 |
| | | 1H NMR (400 MHZ, DMSO-d6, ppm) δ 14.35 (s, 1H), 10.38 (s, 1H), 9.44 (s, 1H), |
| | | 8.70 (s, 1H), 7.00 (dd, J = 7.5, 1.1 Hz, 1H), 6.76 (d, J = 7.5 Hz, 1H), 2.64 . |
| | | (d, J = 1.0 Hz, 3H) |
| 336 | A | LCMS (ES) m/z: [M − 1]−: 322 |
| | | 1H NMR (400 MHZ, DMSO-d6, ppm) δ 14.80 (s, 1H), 10.48 (s, 1H), 9.45 (d, |
| | | J = 1.5 Hz, 1H), 8.69 (d, J = 1.6 Hz, 1H), 7.08 (dd, J = 9.8, 8.1 Hz, 1H), 6.81 |
| | | (dd, J = 8.4, 4.2 Hz, 1H). |
| 337 | A | LCMS (ES) m/z: [M + 1]+: 338 |
| | | 1H NMR (400 MHZ, DMSO-d6, ppm) δ 14.83 (s, 1H), 10.93 (s, 1H), 7.55 (s, 1H), |
| | | 7.12 (dd, J = 9.8, 8.2 Hz, 1H), 6.94 (dd, J = 8.2, 4.1 Hz, 1H), 2.46 (s, 3H). |
| 338 | A | LCMS (ES) m/z: [M + 1]+: 319 |
| | | 1H NMR (400 MHZ, DMSO-d6, ppm) δ 12.04-11.99 (m, 1H), 10.41 (s, 1H), |
| | | 9.23 (d, J = 4.7 Hz, 1H), 8.21 (d, J = 3.1 Hz, 1H), 7.62 (d, J = 4.6 Hz, 1H), 6.81 |
| | | (dd, J = 7.7, 1.0 Hz, 1H), 6.65 (d, J = 7.7 Hz, 1H), 2.59 (s, 3H). |
| 339 | A | LCMS (ES) m/z: [M − 1]−: 333 |
| | | 1H NMR (400 MHZ, DMSO-d6, ppm) δ 12.12-12.07 (m, 1H), 10.76 (s, 1H), |
| | | 8.25 (d, J = 3.1 Hz, 1H), 7.56 (d, J = 8.0 Hz, 1H), 7.23-7.14 (m, 2H), 6.94 |
| | | (dd, J = 7.7, 1.0 Hz, 1H), 3.94 (s, 3H). |
| 340 | A | LCMS (ES) m/z: [M − 1]−: 335 |
| | | 1H NMR (400 MHZ, DMSO-d6, ppm) δ 12.13 (d, J = 3.0 Hz, 1H), 10.81 (s, 1H), |
| | | 8.25 (d, J = 3.1 Hz, 1H), 7.79 (s, 1H), 7.57 (d, J = 8.0 Hz, 1H), 7.17 (t, J = 7.8 Hz, |
| | | 1H), 6.89-6.83 (m, 1H), 5.57 (s, 1H), 5.46 (s, 1H). 19F NMR (376 MHZ, |
| | | DMSO) δ −213.61. |
| 341 | A | LCMS (ES) m/z: [M + 1]+: 334 |
| | | 1H NMR (400 MHZ, DMSO-d6, ppm) δ 14.41 (s, 1H), 10.81 (s, 1H), 7.56 (s, 1H), |
| | | 7.04 (dd, J = 7.5, 1.1 Hz, 1H), 6.87 (d, J = 7.5 Hz, 1H), 2.67 (d, J = 1.0 Hz, 3H), |
| | | 2.45 (s, 3H). |
| 342 | A | LCMS (ES) m/z: [M + 1]+: 320 |
| | | 1H NMR (400 MHZ, DMSO-d6, ppm) δ 14.36 (s, 1H), 10.60 (s, 1H), 9.38 (d, |
| | | J = 0.9 Hz, 1H), 8.20 (d, J = 0.9 Hz, 1H), 7.02 (dd, J = 7.5, 1.1 Hz, 1H), 6.81 |
| | | (d, J = 7.5 Hz, 1H), 2.68-2.64 (m, 3H). |
| 343 | A | LCMS (ES) m/z: [M + 1]+: 340 |
| | | 1H NMR (400 MHZ, DMSO-d6, ppm) δ 14.73 (s, 1H), 10.59 (s, 1H), 9.48 (s, 1H), |
| | | 8.73 (s, 1H), 7.35 (d, J = 8.0 Hz, 1H), 6.87 (d, J = 8.0 Hz, 1H). |
| 344 | A | LCMS (ES) m/z: [M + 1]+: 383 |
| | | 1H NMR (400 MHZ, DMSO-d6, ppm) δ 12.05 (d, J = 3.1 Hz, 1H), 10.59 (s, 1H), |
| | | 8.23 (d, J = 3.0 Hz, 1H), 7.98 (s, 2H), 7.55-7.49 (m, 2H), 7.47 (d, J = 4.0 Hz, |
| | | 1H), 7.16 (t, J = 7.8 Hz, 1H), 6.87 (dd, J = 7.7, 0.9 Hz, 1H). |
| 345 | A | LCMS (ES) m/z: [M + 1]+: 373 |
| | | 1H NMR (400 MHZ, DMSO-d6, ppm) δ12.54 (s, 1H), 10.69 (s, 1H), 9.56 (s, 1H), |
| | | 8.79 (s, 1H), 8.51 (d, J = 2.9 Hz, 1H), 7.56 (d, J = 8.1 Hz, 1H), 7.04 (d, J = 8.1 |
| | | Hz, 1H). |
| 346 | A | LCMS (ES) m/z:[M + 1]+: 317 |
| | | 1H NMR (400 MHZ, DMSO-d6, ppm) δ 8.91 (s, 1H), 7.78 (s, 1H), 6.75 (d, |
| | | J = 7.7 Hz, 1H), 6.58-6.51 (m, 1H), 2.45 (s, 3H), 2.25 (s, 3H). |
| 347 | A | LCMS (ES) m/z: [M − 1]−: 371 |
| | | 1H NMR (400 MHZ, DMSO-d6, ppm) δ 12.17 (s, 1H), 10.94 (s, 1H), 8.25 (d, |
| | | J = 3.1 Hz, 1H), 8.18 (s, 1H), 7.60 (d, J = 8.0 Hz, 1H), 7.19 (t, J = 7.8 Hz, 1H), |
| | | 6.89 (d, J = 7.6 Hz, 1H). |
| 348 | A | LCMS (ES) m/z: [M − 1]−: 353 |
| | | 1H NMR (400 MHZ, DMSO-d6, ppm) δ12.16 (d, J = 3.1 Hz, 1H), 10.88 (s, 1H), |
| | | 8.25 (d, J = 3.2 Hz, 1H), 7.92 (s, 1H), 7.57 (d, J = 8.0 Hz, 1H), 6.94-7.33 (m, |
| | | 2H), 6.86 (dd, J = 7.6, 0.9 Hz, 1H). 19F NMR (282 MHZ, DMSO-d6, ppm) |
| | | δ −114.25, −114.59. |
| 349 | A | LCMS (ES) m/z: [M + 1]+: 383 |
| | | 1H NMR (400 MHZ, DMSO-d6, ppm) δ 12.06 (d, J = 3.2 Hz, 1H), 10.26 (s, 1H), |
| | | 8.25 (dd, J = 5.1, 2.3 Hz, 2H), 7.93 (s, 2H), 7.71 (t, J = 1.3 Hz, 1H), 7.50 (d, |
| | | J = 7.9 Hz, 1H), 7.13 (t, J = 7.8 Hz, 1H), 6.79 (dd, J = 7.7, 1.1 Hz, 1H). |
| 350 | A | LCMS (ES) m/z: [M + 1]+: 383 |
| | | 1H NMR (400 MHZ, DMSO-d6, ppm) δ 12.14 (s, 1H), 11.04 (s, 1H), 8.25 (d, |
| | | J = 3.4 Hz, 2H), 7.54 (d, J = 8.0 Hz, 1H), 7.15 (t, J = 7.8 Hz, 1H), 6.88 (dd, |
| | | J = 7.6, 1.0 Hz, 1H). |
| 351 | A | LCMS (ES) m/z: [M − H]−: 371 |
| | | 1H NMR (400 MHZ, DMSO-d6, ppm) δ 12.53 (s, 1H), 10.90 (s, 1H), 9.37 (d, |
| | | J = 0.9 Hz, 1H), 8.51 (d, J = 3.2 Hz, 1H), 8.34 (d, J = 0.9 Hz, 1H), 7.59 (d, |
| | | J = 8.2 Hz, 1H), 7.11-7.06 (m, 1H). |
| 352 | A | LCMS (ES) m/z: [M − 1]−: 371 |
| | | 1H NMR (400 MHZ, DMSO-d6, ppm) δ 12.64-12.55 (m, 1H), 11.25 (s, 1H), |
| | | 8.51 (d, J = 3.1 Hz, 1H), 8.13 (dd, J = 25.4, 3.1 Hz, 2H), 7.56 (d, J = 8.1 Hz, 1H), |
| | | 7.13 (d, J = 8.2 Hz, 1H). |
| 353 | | LCMS (ES) m/z: [M − 1]−: 319 |
| | | 1H NMR (400 MHZ, DMSO-d6, ppm) δ 11.84-12.33 (s, 2H), 10.30 (s, 1H), 8.22 |
| | | (dd, J = 3.1, 1.7 Hz, 1H), 7.56-7.45 (m, 2H), 7.20 (td, J = 7.8, 1.7 Hz, 1H), 7.04 |
| | | (dt, J = 7.6, 1.3 Hz, 1H). |
| 354 | A | LCMS (ES) m/z: [M + 1]+: 321 |
| | | 1H NMR (400 MHZ, DMSO-d6, ppm) δ 12.56 (s, 1H), 11.00 (s, 1H), 8.33 (d, |
| | | J = 2.9 Hz, 1H), 6.97 (d, J = 9.1 Hz, 2H), 6.75 (dd, J = 8.5, 4.3 Hz, 1H), 2.29 |
| | | (s, 3H). 19F NMR (300 MHZ, DMSO-d6, ppm) δ −124.8. |
| 355 | A | LCMS (ES) m/z: [M + 1]+: 345 |
| | | 1H NMR (400 MHZ, DMSO-d6, ppm) δ 12.07-12.01 (m, 1H), 10.65 (s, 1H), |
| | | 8.22 (d, J = 3.0 Hz, 1H), 7.53 (d, J = 8.0 Hz, 1H), 7.46 (s, 1H), 7.17 (t, J = 7.8 Hz, |
| | | 1H), 6.89 (d, J = 7.5 Hz, 1H), 2.23 (tt, J = 8.2, 4.9 Hz, 1H), 1.06-0.90 (m, 2H), |
| | | 0.90-0.79 (m, 2H). |
| 356 | A | LCMS (ES) m/z: [M − 1]−: 301 |
| | | 1H NMR (400 MHZ, DMSO-d6, ppm) δ 12.21-12.16 (m, 1H), 11.03 (s, 1H), |
| | | 8.26 (d, J = 3.1 Hz, 1H), 7.56 (d, J = 8.0 Hz, 1H), 7.17 (t, J = 7.8 Hz, 1H), 6.97 |
| | | (s, 1H), 6.84 (dd, J = 7.6, 1.0 Hz, 1H), 2.27 (s, 3H). |
| 357 | A | LCMS (ES) m/z: [M + 1]+: 318 |
| | | 1H NMR (400 MHZ, DMSO-d6, ppm) δ14.47 (s, 1H), 11.14 (s, 1H), 7.03 (d, |
| | | J = 7.5 Hz, 1H), 6.94 (s, 1H), 6.87 (d, J = 7.5 Hz, 1H), 2.66 (s, 3H), 2.28 (s, 3H). |
| 358 | A | LCMS (ES) m/z: [M + 1]+: 397 |
| | | 1H NMR (400 MHZ, DMSO-d6, ppm) δ 12.30 (d, J = 2.9 Hz, 1H), 10.48 (s, 1H), |
| | | 8.35 (d, J = 2.9 Hz, 1H), 8.00 (s, 1H), 7.22 (d, J = 8.2 Hz, 1H), 6.85 (d, J = 8.2 Hz, |
| | | 1H), 6.33 (s, 1H), 1.47 (s, 6H). |
| 359 | A | LCMS (ES) m/z: [M + 1]+: 371 |
| | | 1H NMR (400 MHZ, DMSO-d6, ppm) δ 12.29 (s, 1H), 11.29 (s, 1H), 9.22 (d, |
| | | J = 1.6 Hz, 1H), 8.24 (d, J = 3.1 Hz, 1H), 6.94-6.87 (m, 1H), 6.75 (d, J = 7.7 Hz, |
| | | 1H), 2.63 (s, 3H). |
| 360 | A | LCMS (ES) m/z: [M + 1]+: 333 |
| | | 1H NMR (400 MHZ, DMSO-d6, ppm) δ 12.08 (m, 1H), 10.66 (s, 1H), 8.24 (d, |
| | | J = 3.1 Hz, 1H), 7.55 (d, J = 9.8 Hz, 2H), 7.17 (t, J = 7.8 Hz, 1H), 6.88 (dd, J = 7.8, |
| | | 1.0 Hz, 1H), 2.78 (q, J = 7.6 Hz, 2H), 1.18 (t, J = 7.6 Hz, 3H). |
| 361 | | LCMS (ES) m/z: [M − H]−: 391 |
| | | 1H NMR (400 MHZ, DMSO-d6, ppm) δ 11.99 (s, 1H), 10.32 (s, 1H), 8.22 (d, |
| | | J = 2.8 Hz, 1H), 7.56-7.44 (m, 2H), 7.18 (t, J = 7.8 Hz, 1H), 7.03 (d, J = 7.5 Hz, |
| | | 1H), 4.67 (s, 1H), 3.57 (s, 2H), 0.96 (s, 6H). |
| 362 | A | LCMS (ES) m/z: [M + 1]+: 317 |
| | | 1H NMR (400 MHZ, DMSO-d6, ppm) δ 11.99-11.93 (m, 1H), 10.32 (s, 1H), |
| | | 8.57 (s, 1H), 8.19 (d, J = 3.1 Hz, 1H), 6.87-6.79 (m, 1H), 6.72 (d, J = 7.7 Hz, |
| | | 1H), 4.05 (s, 3H), 2.59 (s, 3H). |
| 363 | A | LCMS (ES) m/z: [M + 1]+: 372 |
| | | 1H NMR (400 MHZ, DMSO-d6, ppm) δ 11.03 (s, 1H), 10.30 (s, 1H), 8.00 (s, 1H), |
| | | 7.50 (d, J = 2.8 Hz, 1H), 7.35 (d, J = 7.9 Hz, 1H), 7.05 (t, J = 7.8 Hz, 1H), 6.93 |
| | | (dd, J = 7.6, 1.0 Hz, 1H), 6.29 (s, 1H), 1.44 (s, 6H). |
| 364 | A | ES, m/z: [M + 1]+: 313 |
| | | 1H NMR (400 MHZ, DMSO-d6, ppm) δ 13.27 (s, 1H), 10.78 (s, 1H), 7.63 (s, 1H), |
| | | 7.56 (d, J = 8.1 Hz, 1H), 7.16 (t, J = 7.7 Hz, 1H), 7.04 (dd, J = 7.4, 1.0 Hz, 1H), |
| | | 2.51 (s, 3H). |
| 365 | A | LCMS (ES) m/z: [M + 1]+: 357 |
| | | 1H NMR (400 MHZ, DMSO-d6, ppm) δ 12.32 (s, 1H), 11.45 (s, 1H), 9.24 (s, 1H), |
| | | 8.28 (d, J = 3.1 Hz, 1H), 7.60 (d, J = 8.0 Hz, 1H), 7.19 (t, J = 7.8 Hz, 1H), 6.92 |
| | | (d, J = 7.6 Hz, 1H). |
| 366 | A | LCMS (ES) m/z: [M + 1]+: 334 |
| | | 1H NMR (400 MHZ, DMSO-d6, ppm) δ 11.95 (s, 1H), 10.08 (s, 1H), 8.20 (s, 1H), |
| | | 7.84 (dd, J = 13.4, 1.3 Hz, 2H), 7.38 (dd, J = 7.9, 1.1 Hz, 1H), 7.08 (t, J = 7.8 Hz, |
| | | 1H), 6.98 (dd, J = 7.7, 1.1 Hz, 1H), 4.73 (t, J = 4.7 Hz, 1H), 4.58 (t, J = 4.7 Hz, |
| | | 1H), 4.35 (t, J = 4.7 Hz, 1H), 4.26 (t, J = 4.7 Hz, 1H). |
| 367 | A | LCMS (ES) m/z: [M + 1]+: 324 |
| | | 1H NMR (400 MHZ, DMSO-d6, ppm) δ 13.71 (s, 1H), δ 12.23 (s, 1H), 9.20 |
| | | (d, J = 4.7 Hz, 1H), 8.50 (s, 1H), 7.79 (d, J = 4.7 Hz, 1H), 7.70 (d, J = 4.6 Hz, 1H). |
| 368 | A | LCMS (ES) m/z: [M + 1]+: 303 |
| | | 1H NMR (400 MHZ, DMSO-d6, ppm) δ 11.98-11.91 (m, 1H), 10.49 (s, 1H), |
| | | 8.62 (s, 1H), 8.22 (d, J = 3.0 Hz, 1H), 7.46 (d, J = 8.0 Hz, 1H), 7.11 (t, J = 7.8 Hz, |
| | | 1H), 6.92 (d, J = 7.7 Hz, 1H), 4.05 (s, 3H). |
| 369 | A | LCMS (ES) m/z: [M + 1]+: 302 |
| | | 1H NMR (400 MHZ, DMSO-d6, ppm) δ 11.36 (s, 1H), 10.48 (s, 1H), 7.97 (d, |
| | | J = 1.3 Hz, 1H), 7.76 (d, J = 1.3 Hz, 1H), 7.64 (t, J = 2.9 Hz, 1H), 7.43 (d, |
| | | J = 8.1 Hz, 1H), 7.17 (d, J = 8.1 Hz, 1H), 6.55 (dd, J = 3.1, 1.9 Hz, 1H), 3.65 (s, 3H). |
| 370 | A | LCMS (ES) m/z: [M + 1]+: 305 |
| | | 1H NMR (400 MHZ, DMSO-d6, ppm) δ 11.34 (s, 1H), 10.85 (s, 1H), 9.33 (s, 1H), |
| | | 8.36 (s, 1H), 7.63 (t, J = 2.9 Hz, 1H), 7.50 (d, J = 8.0 Hz, 1H), 7.07 (d, J = 8.0 Hz, |
| | | 1H), 6.59 (dd, J = 3.1, 1.9 Hz, 1H). |
| 371 | A | LCMS (ES) m/z: [M + 1]+: 394 |
| | | 1H NMR (400 MHZ, DMSO-d6, ppm) δ 13.32 (s, 1H), 10.92 (s, 1H), 8.43 (s, 2H), |
| | | 8.37 (s, 1H), 7.60 (d, J = 8.1 Hz, 1H), 7.18 (t, J = 7.8 Hz, 1H), 7.05 (dd, J = 7.5, |
| | | 0.9 Hz, 1H). |
| 372 | A | LCMS (ES) m/z: [M + 1]+: 385 |
| | | 1H NMR (400 MHZ, DMSO-d6, ppm) δ 14.48 (s, 1H), 11.09 (s, 1H), 8.45 (s, 2H), |
| | | 8.35 (s, 1H), 7.83 (d, J = 8.2 Hz, 1H), 7.33 (t, J = 7.8 Hz, 1H), 7.10-7.01 (m, 1H) |
| 373 | A | LCMS (ES) m/z: [M + 1]+: 384 |
| | | 1H NMR (400 MHZ, DMSO-d6, ppm) δ 12.14 (d, J = 3.2 Hz, 1H), 10.81 (s, 1H), |
| | | 8.44 (s, 2H), 8.33 (s, 1H), 8.26 (d, J = 3.1 Hz, 1H), 7.57 (d, J = 8.0 Hz, 1H), 7.19 |
| | | (t, J = 7.8 Hz, 1H), 6.88 (dd, J = 7.6, 1.0 Hz, 1H). |
| 374 | A | LCMS (ES) m/z: [M + 1]+: 334 |
| | | 1H NMR (400 MHZ, DMSO-d6, ppm) δ 12.04-11.99 (m, 1H), 10.43 (s, 1H), |
| | | 8.24 (d, J = 2.8 Hz, 1H), 7.95 (s, 1H), 7.47 (d, J = 8.0 Hz, 1H), 7.39 (s, 1H), 7.11 |
| | | (t, J = 7.8 Hz, 1H), 6.79 (d, J = 7.7 Hz, 1H), 4.73 (t, J = 4.7 Hz, 1H), 4.61 (t, |
| | | J = 4.7 Hz, 1H), 4.50 (t, J = 4.8 Hz, 1H), 4.43 (t, J = 4.7 Hz, 1H). |
| 375 | | LCMS (ES) m/z: [M − 1]−: 391 |
| | | 1H NMR (400 MHZ, DMSO-d6, ppm) δ 11.99 (d, J = 3.1 Hz, 1H), 10.38 (s, 1H), |
| | | 8.22 (d, J = 3.1 Hz, 1H), 7.60-7.48 (m, 2H), 7.18 (t, J = 7.8 Hz, 1H), 6.96 (dd, J = 7.7, |
| | | 1.0 Hz, 1H), 4.81 (s, 1H), 4.18 (s, 2H), 1.14 (s, 6H). |
| 376 | A | LCMS (ES) m/z: [M + 1]+: 312 |
| | | 1H NMR (400 MHZ, DMSO-d6, ppm) δ 11.21-11.15 (m, 1H), 10.72 (s, 1H), |
| | | 7.53 (d, J = 2.8 Hz, 1H), 7.37 (dt, J = 7.9, 0.9 Hz, 1H), 7.04 (t, J = 7.7 Hz, 1H), |
| | | 6.94 (dd, J = 7.6, 1.1 Hz, 1H), 6.59 (d, J = 1.0 Hz, 1H), 2.46 (d, J = 0.9 Hz, 3H). |
| 377 | A | |
| 378 | A | LCMS (ES) m/z: [M + 1]+: 397 |
| | | 1H NMR (400 MHZ, DMSO-d6, ppm) δ 11.6 (d, J = 2.7 Hz, 1H), 10.9 (s, 1H), |
| | | 8.17 (s, 1H), 7.78 (d, J = 2.8 Hz, 1H), 7.59 (d, J = 8.1 Hz, 1H), 7.16 (d, |
| | | J = 8.1 Hz, 1H), 6.32 (s, 1H), 1.44 (s, 6H). |
| 379 | A | LCMS (ES) m/z: [M + 1]+: 339 |
| | | 1H NMR (400 MHZ, DMSO-d6, ppm) δ 11.65 (d, J = 2.9 Hz, 1H), 10.90 (s, 1H), |
| | | 9.35 (d, J = 0.9 Hz, 1H), 8.37 (d, J = 0.9 Hz, 1H), 7.79 (d, J = 2.9 Hz, 1H), 7.59 |
| | | (d, J = 8.0 Hz, 1H), 7.11 (d, J = 8.0 Hz, 1H). |
| 380 | A | LCMS (ES) m/z: [M + 1]+: 317 |
| | | 1H NMR (400 MHZ, DMSO-d6, ppm) δ 12.10 (d, J = 3.3 Hz, 1H), 10.72 (s, 1H), |
| | | 8.23 (d, J = 3.1 Hz, 1H), 6.89 (dd, J = 7.7, 1.0 Hz, 1H), 6.80 (d, J = 7.7 Hz, 1H), |
| | | 6.59 (d, J = 1.1 Hz, 1H), 2.62 (s, 3H), 2.48 (d, J = 0.9 Hz, 3H). |
| 381 | A | LCMS (ES) m/z: [M + 1]+: 303 |
| | | 1H NMR (400 MHZ, DMSO-d6, ppm) δ 12.12-12.06 (m, 1H), 10.88 (s, 1H), |
| | | 8.26 (d, J = 3.2 Hz, 1H), 7.54 (d, J = 8.0 Hz, 1H), 7.17 (t, J = 7.8 Hz, 1H), 6.98 |
| | | (dd, J = 7.7, 1.0 Hz, 1H), 6.61 (d, J = 1.0 Hz, 1H), 2.48 (d, J = 0.9 Hz, 3H). |
| 382 | | LCMS (ES) m/z: [M + 1]+: 393 |
| | | 1H NMR (400 MHZ, DMSO-d6, ppm) δ 12.00 (s, 1H), 10.41 (s, 1H), 8.22 (d, |
| | | J = 3.0 Hz, 1H), 7.60-7.48 (m, 2H), 7.17 (t, J = 7.8 Hz, 1H), 6.94 (d, J = 7.6 Hz, |
| | | 1H), 5.12 (s, 2H), 3.69 (s, 3H). |
| 383 | A | LCMS (ES) m/z: [M + 1]+: 337 |
| | | 1H NMR (400 MHZ, DMSO-d6, ppm) δ 12.32 (s, 1H), 10.59 (s, 1H), 8.61 (s, 1H), |
| | | 8.34 (s, 1H), 7.15 (dd, J = 8.2, 1.2 Hz, 1H), 6.85 (dd, J = 8.2, 1.2 Hz, 1H), 4.05 (s, 3H). |
| 384 | A | LCMS (ES) m/z: [M − H]−: 337 |
| | | 1H NMR (400 MHZ, DMSO-d6, ppm) δ 11.70 (s, 1H), 11.25 (s, 1H), 8.16 (d, |
| | | J = 3.1 Hz, 1H), 8.09 (d, J = 3.1 Hz, 1H), 7.80 (d, J = 2.9 Hz, 1H), 7.56 (t, |
| | | J = 7.7 Hz, 1H), 7.13 (d, J = 8.1 Hz, 1H). |
| 385 | A | LCMS (ES) m/z: [M + 1]+: 337 |
| | | 1H NMR (400 MHZ, DMSO-d6, ppm) δ 11.71 (s, 1H), 11.10 (s, 1H), 7.84-7.78 |
| | | (m, 2H), 7.58 (d, J = 8.0 Hz, 1H), 7.08 (d, J = 8.1 Hz, 1H), 2.48 (s, 3H). |
| 386 | A | LCMS (ES) m/z: [M + 1]+: 392 |
| | | 1H NMR (400 MHZ, DMSO-d6, ppm) δ 11.14 (d, J = 2.8 Hz, 1H), 10.45 (s, 1H), |
| | | 7.97 (s, 2H), 7.50 (dd, J = 6.7, 2.1 Hz, 3H), 7.37 (d, J = 7.9 Hz, 1H), 7.05 (t, |
| | | J = 7.8 Hz, 1H), 6.87 (d, J = 7.5 Hz, 1H). |
| 387 | A | LCMS (ES) m/z: [M + 1]+: 392 |
| | | 1H NMR (400 MHZ, DMSO-d6, ppm) δ 11.16-11.09 (m, 1H), 10.13 (s, 1H), |
| | | 8.26 (d, J = 1.7 Hz, 1H), 7.91 (s, 2H), 7.72 (d, J = 1.7 Hz, 1H), 7.50 (d, J = 2.7 |
| | | Hz, 1H), 7.33 (d, J = 7.9 Hz, 1H), 7.01 (t, J = 7.8 Hz, 1H), 6.80 (d, J = 7.5 Hz, 1H). |
| 388 | A | LCMS (ES) m/z: [M + 1]+: 305 |
| | | 1H NMR (400 MHZ, DMSO-d6, ppm) δ 11.41 (s, 1H), 11.22 (s, 1H), 8.16 (d, |
| | | J = 3.1 Hz, 1H), 8.08 (d, J = 3.1 Hz, 1H), 7.65 (t, J = 2.9 Hz, 1H), 7.48 (d, J = 8.0 |
| | | Hz, 1H), 7.08 (d, J = 8.1 Hz, 1H), 6.59 (dd, J = 3.1, 1.9 Hz, 1H). |
| 389 | A | LCMS (ES) m/z: [M + 1]+: 303 |
| | | 1H NMR (400 MHZ, DMSO-d6, ppm) δ 11.41 (s, 1H), 11.06 (s, 1H), 7.79 (d, |
| | | J = 1.1 Hz, 1H), 7.65 (t, J = 2.9 Hz, 1H), 7.49 (dd, J = 8.0, 1.0 Hz, 1H), 7.02 |
| | | (dd, J = 8.0, 1.1 Hz, 1H), 6.60 (dd, J = 3.1, 1.9 Hz, 1H), 2.47 (s, 3H). |
| 390 | A | LCMS (ES) m/z: [M − H]−: 317 |
| | | 1H NMR (400 MHZ, DMSO-d6, ppm) δ 11.01 (s, 1H), 10.78 (s, 1H), 9.33 |
| | | (d, J = 0.9 Hz, 1H), 8.35 (d, J = 0.9 Hz, 1H), 7.45 (d, J = 8.0 Hz, 1H), 7.41-7.35 |
| | | (m, 1H), 7.04 (d, J = 8.0 Hz, 1H), 2.43-2.39 (m, 3H). |
| 391 | A | LCMS (ES) m/z: [M + 1]+: 393 |
| | | 1H NMR (400 MHZ, DMSO-d6, ppm) δ 11.21 (d, J = 2.6 Hz, 1H), 10.66 (s, 1H), |
| | | 8.41 (s, 2H), 8.32 (s, 1H), 7.52 (d, J = 2.7 Hz, 1H), 7.41 (d, J = 8.0 Hz, 1H), 7.07 |
| | | (t, J = 7.8 Hz, 1H), 6.86 (d, J = 7.5 Hz, 1H). |
| 392 | A | LCMS (ES) m/z: [M + 1]+: 393 |
| | | 1H NMR (400 MHZ, DMSO-d6, ppm) δ 13.23 (s, 1H), 10.69 (s, 1H), 7.99 (s, 2H), |
| | | 7.60-7.49 (m, 3H), 7.16 (t, J = 7.8 Hz, 1H), 7.01 (dd, J = 7.4, 0.9 Hz, 1H). |
| 393 | A | LCMS (ES) m/z: [M + 1]+: 319 |
| | | 1H NMR (400 MHZ, DMSO-d6, ppm) δ 11.07-11.02 (m, 1H), 10.60 (s, 1H), |
| | | 9.56 (s, 1H), 8.78 (s, 1H), 7.46-7.36 (m, 2H), 7.00 (d, J = 8.1 Hz, 1H), 2.41 (s, 3H). |
| 394 | H | LCMS (ES) m/z: [M + 1]+: 390 |
| | | 1H NMR (400 MHZ, DMSO-d6, ppm) δ 11.92 (s, 1H), 10.21 (s, 1H), 8.14 (s, 1H), |
| | | 7.32 (s, 1H), 7.16-7.00 (m, 2H), 6.91 (dd, J = 7.7, 1.0 Hz, 1H), 4.71 (s, 1H), |
| | | 4.05 (s, 2H), 3.44 (s, 3H), 1.14 (s, 6H). |
| 395 | A | LCMS (ES) m/z: [M − H]−: 337 |
| | | 1H NMR (400 MHZ, DMSO-d6, ppm) δ 11.67 (s, 1H), 10.70 (s, 1H), 9.58 (s, 1H), |
| | | 8.80 (s, 1H), 7.80 (d, J = 2.9 Hz, 1H), 7.56 (d, J = 8.1 Hz, 1H), 7.07 |
| | | (d, J = 8.1 Hz, 1H). |
| 396 | A | LCMS (ES) m/z: [M + 1]+: 319 |
| | | 1H NMR (400 MHZ, DMSO-d6, ppm) δ 11.12 (s, 1H), 11.06 (s, 1H), 8.14 (d, |
| | | J = 3.0 Hz, 1H), 8.07 (d, J = 3.1 Hz, 1H), 7.46-7.36 (m, 2H), 7.05 (d, J = 8.0 Hz, |
| | | 1H), 2.41 (s, 3H). |
| 397 | A | LCMS (ES) m/z: [M + 1]+: 317 |
| | | 1H NMR (400 MHZ, DMSO-d6, ppm) δ 11.07 (s, 1H), 10.97 (s, 1H), 7.76 |
| | | (s, 1H), 7.48-7.36 (m, 2H), 6.99 (d, J = 8.0 Hz, 1H), 2.50-2.39 (m, 6H). |
| 398 | A | LCMS (ES) m/z: [M + 1]+: 392 |
| | | 1H NMR (400 MHZ, DMSO-d6, ppm) δ 11.13 (s, 1H), 10.36 (s, 1H), 8.32 |
| | | (d, J = 1.7 Hz, 1H), 7.74 (d, J = 1.7 Hz, 1H), 7.58-7.47 (m, 3H), 7.37 (d, |
| | | J = 7.9 Hz, 1H), 7.04 (t, J = 7.8 Hz, 1H), 6.89-6.81 (m, 1H). |
| 403 | A | LCMS (ES) m/z: [M + 1]+: 373 1H NMR (400 MHZ, DMSO-d6, ppm) δ 13.65 |
| | | (s, 1H), 10.77 (s, 1H), 7.62 (s, 1H), 7.20 (d, J = 8.0 Hz, 1H), 6.92 (d, J = 8.0 Hz, |
| | | 1H), 2.19 (td, J = 8.5, 4.3 Hz, 1H), 1.13 (dt, J = 8.3, 3.5 Hz, 2H), 0.99-0.91 (m, 2H) |
| 404 | A | LCMS (ES) m/z: [[M + 1]+: 393 1H NMR (400 MHZ, DMSO-d6, ppm) δ 13.24 |
| | | (s, 1H), 10.58-10.53 (m, 1H), 8.35 (d, J = 1.7 Hz, 1H), 7.73 (d, J = 1.7 Hz, 1H), |
| | | 7.55 (d, J = 7.6 Hz, 3H), 7.15 (t, J = 7.8 Hz, 1H), 7.01-6.95 (m, 1H). |
| 405 | A | LCMS (ES) m/z: [M − H]−-: 346 1H NMR (400 MHZ, DMSO-d6, ppm) δ 11.50 |
| | | (s, 1H), 10.17 (s, 1H), 9.44 (s, 1H), 8.70 (s, 1H), 7.58 (d, J = 2.9 Hz, 1H), 7.01 |
| | | (d, J = 8.2 Hz, 1H), 6.70 (d, J = 8.2 Hz, 1H). |
| 406 | A | LCMS (ES) m/z: [M − H]−: 370 1H NMR (400 MHZ, DMSO-d6, ppm) δ 11.57 |
| | | (d, J = 2.8 Hz, 1H), 10.53 (s, 1H), 7.63-7.55 (m, 2H), 7.05 (d, J = 8.2 Hz, 1H), 6.72 |
| | | (d, J = 8.1 Hz, 1H), 2.18 (tt, J = 8.2, 4.8 Hz, 1H), 1.12 (dt, J = 8.3, 3.4 Hz, 2H), |
| | | 0.98-0.90 (m, 2H). |
| 407 | A | LCMS (ES) m/z: [M + 1]+: 349 1H NMR (400 MHZ, DMSO-d6, ppm) δ 13.58 |
| | | (s, 1H), 10.41 (s, 1H), 9.49 (s, 1H), 8.74 (s, 1H), 7.16 (d, J = 8.0 Hz, 1H), 6.85 |
| | | (d, J = 8.0 Hz, 1H). |
| 408 | A | LCMS (ES) m/z: [M + 1]+: 376 1H NMR (400 MHZ, DMSO-d6, ppm) δ |
| | | 11.97-11.92 (m, 1H), 11.01 (s, 1H), 9.89 (s, 1H), 8.24 (s, 1H), 7.47 |
| | | (d, J = 8.0 Hz, 1H), 7.15 (t, J = 7.8 Hz, 1H), 7.03 (s, 1H), 6.98 |
| | | (d, J = 7.6 Hz, 1H), 4.64 (s, 1H), 3.39 (s, 2H), 0.96 (s, 6H). |
| 409 | I | LCMS (ES) m/z: [M + 1]+: 319 1H NMR (400 MHZ, DMSO-d6, ppm) δ 11.89 |
| | | (s, 1H), 10.13 (s, 1H), 8.21 (d, J = 2.5 Hz, 1H), 7.46 (dd, J = 8.0, 1.0 Hz, 1H), 7.14 |
| | | (t, J = 7.8 Hz, 1H), 6.97-6.88 (m, 2H), 6.29 (d, J = 1.9 Hz, 1H), 5.08 (s, 2H). |
| 410 | I | LCMS (ES) m/z: [M + 1]+: 319 1H NMR (400 MHZ, DMSO-d6, ppm) δ 11.82 |
| | | (s, 1H), 9.90 (s, 1H), 8.21 (d, J = 2.8 Hz, 1H), 7.52 (d, J = 5.4 Hz, 1H), 7.40 (dd, |
| | | J = 8.0, 1.0 Hz, 1H), 7.10 (t, J = 7.8 Hz, 1H), 6.96 (dd, J = 7.7, 1.0 Hz, 1H), 6.49 |
| | | (d, J = 5.3 Hz, 1H), 5.97 (s, 2H). |
| 411 | A | LCMS (ES) m/z: [M+1]+: 362 1H NMR (400 MHZ, DMSO-d6, ppm) δ 11.95 |
| | | (s, 1H), 9.70 (s, 1H), 8.19 (d, J = 2.5 Hz, 1H), 7.98 (s, 1H), 7.48 (d, J = 7.9 Hz, 1H), |
| | | 7.14 (t, J = 7.8 Hz, 1H), 6.91 (d, J = 7.6 Hz, 1H), 3.76 (s, 2H), 2.24 (s, 6H). |
| 412 | A | LCMS (ES) m/z: [M + 1]+: 386 1H NMR (400 MHZ, DMSO-d6, ppm) δ 11.08 |
| | | (d, J = 2.4 Hz, 1H), 10.39 (s, 1H), 8.16 (s, 1H), 7.57 (s, 1H), 7.50 (d, J = 2.8 Hz, 1H), |
| | | 7.35 (dt, J = 7.9, 0.9 Hz, 1H), 7.05 (t, J = 7.8 Hz, 1H), 6.91 (dd, J = 7.6, 1.1 Hz, 1H), |
| | | 4.81 (dd, J = 6.5, 0.8 Hz, 2H), 4.73-4.64 (m, 2H). |
| 413 | A | LCMS (ES) m/z: [M + 1]+: 386 1H NMR (400 MHZ, DMSO-d6, ppm) δ 11.16 |
| | | (s, 1H), 10.76 (s, 1H), 8.22 (s, 1H), 7.51 (d, J = 2.8 Hz, 1H), 7.34 (d, J = 7.9 Hz, |
| | | 1H), 7.18 (s, 1H), 7.01 (t, J = 7.8 Hz, 1H), 6.89 (dd, J = 7.6, 1.1 Hz, 1H), 4.72 |
| | | (q, J = 7.0 Hz, 4H). |
| 414 | A | LCMS (ES) m/z: [M + 1]+: 391 1H NMR (400 MHZ, DMSO-d6, ppm) δ 12.00 |
| | | (d, J = 3.2 Hz, 1H), 10.35 (s, 1H), 8.20 (d, J = 3.1 Hz, 1H), 8.11 (s, 1H), 7.59 |
| | | (s, 1H), 6.88 (dd, J = 7.7, 0.9 Hz, 1H), 6.72 (d, J = 7.7 Hz, 1H), 4.88-4.79 (m, 2H), |
| | | 4.75-4.67 (m, 2H), 2.61 (d, J = 0.9 Hz, 3H). |
| 415 | A | LCMS (ES) m/z: [M + 1]+: 391 1H NMR (400 MHZ, DMSO-d6, ppm) δ 12.08 |
| | | (d, J = 3.2 Hz, 1H), 10.75 (s, 1H), 8.26-8.17 (m, 2H), 7.19 (s, 1H), 6.85 (dd, J = 7.7, |
| | | 1.0 Hz, 1H), 6.72 (d, J = 7.7 Hz, 1H), 4.73 (q, J = 6.9 Hz, 4H), 2.63-2.56 (m, 3H). |
| 416 | A | LCMS (ES) m/z: [M + 1]+: 383 1H NMR (400 MHZ, DMSO-d6, ppm) δ 12.10 |
| | | (d, J = 3.1 Hz, 1H), 10.59 (s, 1H), 8.28-8.19 (m, 2H), 6.91 (d, J = 7.7 Hz, 1H), |
| | | 6.70 (d, J = 7.7 Hz, 1H), 2.63 (s, 3H), 2.17 (t, J = 19.2 Hz, 3H). |
| 417 | A | LCMS (ES) m/z: [M + 1]+: 360 1H NMR (400 MHZ, DMSO-d6, ppm) δ 14.30 |
| | | (s, 1H), 10.50 (s, 1H), 7.87 (s, 1H), 7.04 (dd, J = 7.5, 1.1 Hz, 1H), 6.87 (d, |
| | | J = 7.5 Hz, 1H), 2.66 (d, J = 1.0 Hz, 3H), 2.55-2.43 (m, 1H), 1.25-1.08 (m, 2H), |
| | | 1.04 (dt, J = 4.8, 3.3 Hz, 2H). |
| 418 | A | LCMS (ES) m/z: [M + 1]+: 346 1H NMR (400 MHZ, DMSO-d6, ppm) δ 14.31 |
| | | (s, 1H), 10.67 (s, 1H), 7.91 (s, 1H), 7.77 (dd, J = 8.3, 0.8 Hz, 1H), 7.31 (dd, J = 8.2, |
| | | 7.4 Hz, 1H), 7.06 (dd, J = 7.5, 0.9 Hz, 1H), 2.57-2.42 (m, 1H), 1.26-1.10 (m, 2H), |
| | | 1.13-0.97 (m, 2H). |
| 419 | A | LCMS (ES) m/z: [M + 1]+: 311 1H NMR (400 MHZ, DMSO-d6, ppm) δ 11.89 |
| | | (s, 1H), 10.77 (s, 1H), 9.30 (s, 1H), 8.24 (s, 1H), 7.86 (s, 1H), 6.80 (s, 1H). |
| 420 | A | LCMS (ES) m/z: [M + 1]+: 319 1H NMR (400 MHZ, DMSO-d6, ppm) δ 10.88-10.83 |
| | | (m, 1H), 10.32 (s, 1H), 9.31 (d, J = 0.9 Hz, 1H), 8.20 (d, J = 0.9 Hz, 1H), |
| | | 7.46 (d, J = 7.9 Hz, 1H), 7.34 (d, J = 2.6 Hz, 1H), 6.99 (t, J = 7.8 Hz, 1H), 6.83 |
| | | (dd, J = 7.6, 1.0 Hz, 1H), 4.06-4.01 (m, 2H). |

### Example 3—Targeted RBM39 Degradation Assay

Molecular glues potentiate the productive formation of a complex between RBM39 protein and DCAF15, the substrate recognition subunit of an E3 ubiquitin ligase complex, which is thought to lead to subsequent ubiquitination and proteosomal degradation of RBM39 protein. To confirm ubiquitin and proteosomal dependent degradation of RBM39 by compounds disclosed herein, OVCAR3 cells were cultured for 6 h with compounds at 1 μM and with and without MG132 (a proteasomal degrader) at 5 μM and Pevonedistat (MLN4924) NEDDylation Inhibitor at 0.3 uM. 50 μg of protein per sample was loaded per lane of SIDS PAGE gel and proteins were transferred to PVDF membrane using iBlot2 dry blotting system High MW Protocol: 10 min at 2.5 A, up to 25V. Membranes were blocked in TBST/5% BSA for 1 hr at room temperature with shaking at 100 rpm. Membrane and probed with RBM39 and β-actin:1-in-4000) in TBST/5% milk with shaking at 100 rpm rabbit antibody at 1:1000 dilution and probed with fluorescent secondary antibody conjugated with 800 nm and Anti-B-Actin (CST, #3700 mouse antibody at a dilution of 1:4000) as a loading control. The membranes were washed with 1×TBST for 4×5 mins at R/T, incubated with secondary antibody anti-rabbit IgG antibody HRP linked (1-in-10000 dilution) and IRDye 680 anti-mouse antibody (1-in-10000 dilution), then diluted in TBST/5% milk for 1 h at room temperature. The membranes were washed in TBST for 5 min×4 times. The loading control protein and target protein bands were analyzed after rinsing the membrane once with TBS, and the band signal was quantified.

### Example 4—CellTiter-Glo Assay for Assessing Impact of RBM39 Degraders on Cell Viability & Proliferation In Vitro

A CeIlTiter-Glo assay was used to assess compound cytotoxicity on cell proliferation in OVCAR3, SK-N-SH, SK-N-AS, Kelly, IMR32, SK-N-BE-2C cell lines. Cells were harvested into cell culture medium and counted. The cells were diluted with culture medium to the cell densities listed in the table below, and 50 μL of cell suspension was added into each well of 384-well cell culture plate, except for low control cell. For the low control well, 50 μL of phosphate buffered saline was added.

| No | Cell Line | Cells/well |
|---|---|---|
| 1 | SK-N-SH | 4K |
| 2 | SK-N-AS | 6K |
| 3 | Kelly | 4K |
| 4 | IMR32 | 5k/10K |
| 5 | SKNBE2C | 2K |
| 6 | OVCAR3 | 2K/4K/8K |

The plates were covered and incubated at room temperature for 30 minutes without shaking, then incubated overnight at 37° C. and 5% CO₂ for cell attachment. Test compounds were dissolved to make a 10 mM DMSO stock solution. A10 μL aliquot of stock solution was placed in a 384 LDV-plate, and a 3 fold, 11-point dilution was carried out via transferring 4 μL compound into 8 μL DMSO.

Next, 50 nL of diluted compound was transferred into the cell plate at a final concentration of 10 μM. A high control was prepared with 50 nL DMSO. The plates were spun at room temperature at 1,000 RPM for 1 minute.

After compound treatment for 72 hours, the plate was removed from the incubator and equilibrated at room temperature for 15 minutes. Next, 40 μL of CellTiter-Glo reagent was added to each well (at 1:1 to culture medium), the plates were incubated at room temperature for 30 minutes, and then read.

### Example 5—DCAF15 RBM39 Complex Formation Assay

A TR-FRET assay was used to assess the effect of compounds disclosed herein on the interaction between RBM39 and the DCAF15 complex. Test compounds were dissolved to form a 10 mM DMSO stock solution. A 45 μL aliquot of the stock solution was transferred to a 384 pp-plate, and a threefold, 8-point dilution was performed via transferring 15 μL compound solution into 30 μL of DMSO. The plates were then spun at room temperature at 1,000 RPM for 1 minute.

A 30 nL aliquot of the diluted compound was transferred to a 384 well plate, which was incubated at room temperature for 15 minutes. Next, Solutions 1, 2, and 3 were prepared as described in the below tables. A 5 μL aliquot of Solution 2 was added to each well, followed by 5 μL of Solution 3 to start the reaction. The final volume of each well was 10 μL. The plates were incubated at room temperature for 60 minutes, and then read.

### Solution 1

| No | Component | Final Concentration |
|---|---|---|
| 1 | HEPES,pH7.5 | 25 mM |
| 2 | NaCl | 100 mM |
| 3 | BSA | 0.1 mg/ml |
| 4 | Tween 20 | 0.005% |
| 5 | TCEP | 0.5 mM |

### Solution 2

| No | Component | Final Concentration |
|---|---|---|
| 1 | RBM39-Flag | 50 nM |
| 2 | APC-labeled anti-FLAG antibody | 50 nM |

### Solution 3

| No | Component | Final Concentration |
|---|---|---|
| 1 | His-DCAF15-DDA1-DDB1 | 10 nM |
| 2 | Europium-labeled anti-His antibody | 0.5 nM |

Next, an RBM39 (R150-D331) 3× Flag used in TR-FRET assay was prepared:

| | |
|---|---|
| RBM39(R150-D331) 3xFlag, removed GST tag | 220332-QXJ220623 |

Recombinant RBM39 protein is composed of the R1R2 of RBM39 (aa 150 to 331; UniProt: Q14498). The coding sequence was sub-cloned into pGEX4T-1-RBM39-flag vector, expressed as a GST-fusion protein with N-terminal TEV protease cleavage site. A 3× Flag tag was added to the C-terminal of R1R2 used in a FRET assay. The results of the FRET assay are shown in the table below.

| Compound | EC50 (nM) |
|---|---|
| 2 | 1025.9 |
| 3 | 429.1 |
| 7 | 106.5 |
| 8 | 786.9 |
| 9 | 436.3 |
| 10 | 75.7 |
| 11 | 108.1 |
| 12 | 384.0 |
| 13 | 297.6 |
| 24 | 269.0 |
| 25 | 1087.4 |
| 27 | 2797.4 |
| 32 | 135.6 |
| 34 | 301.4 |
| 36 | 353.8 |
| 37 | 427.4 |
| 38 | 4190.8 |
| 39 | 520.0 |
| 40 | 275.0 |
| 41 | 363.1 |
| 42 | 55.2 |
| 43 | >30000 |
| 44 | 904.2 |
| 45 | 394.2 |
| 47 | 97.0 |
| 48 | 124.9 |
| 49 | 189.7 |
| 50 | 965.7 |
| 51 | 164.4 |
| 55 | 302.0 |
| 57 | >30000 |
| 58 | 1080.7 |
| 59 | 801.9 |
| 60 | 1356.9 |
| 61 | 1790.4 |
| 63 | 1167.5 |
| 64 | 3443.6 |
| 65 | 1082.2 |
| 67 | 520.1 |
| 68 | 708.0 |
| 69 | 420.7 |
| 70 | 337.6 |
| 71 | 532.1 |
| 72 | 431.5 |
| 73 | 738.2 |
| 74 | 1448.7 |
| 75 | 2177.2 |
| 76 | 3315.3 |
| 77 | 827.9 |
| 78 | 3247.1 |
| 79 | 9709.4 |
| 81 | 55.6 |
| 82 | 1441.1 |
| 83 | 104.7 |
| 84 | 174.7 |
| 85 | 95.9 |
| 86 | 2070.6 |
| 87 | 276.7 |
| 89 | 173.7 |
| 90 | 389.1 |
| 91 | 76.6 |
| 93 | 1518.8 |
| 94 | 45.2 |
| 95 | 104.3 |
| 97 | 105.2 |
| 98 | 196.3 |
| 99 | 517.9 |
| 100 | 363.8 |
| 102 | >30000 |
| 103 | 8785.4 |
| 104 | 127.7 |
| 105 | 299.1 |
| 107 | 325.3 |
| 108 | 1972.3 |
| 109 | 2069.1 |
| 110 | 729.2 |
| 111 | 1633.8 |
| 112 | 1364.0 |
| 113 | 1619.6 |
| 114 | 3262.1 |
| 116 | 2133.8 |
| 117 | 99.6 |
| 118 | 168.3 |
| 120 | 359.2 |
| 121 | 916.8 |
| 123 | >30000 |
| 126 | 959.6 |
| 127 | 959.3 |
| 128 | 649.9 |
| 129 | 1816.7 |
| 130 | 1132.5 |
| 131 | 3795.9 |
| 132 | 6651.5 |
| 133 | 2668.0 |
| 134 | 1762.5 |
| 135 | 47.0 |
| 136 | 510.7 |
| 138 | 422.8 |
| 139 | 6067.5 |
| 140 | 6617.6 |
| 141 | 918.7 |
| 142 | 1956.8 |
| 143 | 2150.0 |
| 144 | 1564.4 |
| 146 | 505.2 |
| 147 | 2034.4 |
| 148 | 1311.0 |
| 149 | 896.6 |
| 150 | 1254.5 |
| 151 | 343.9 |
| 152 | 280.9 |
| 153 | 940.5 |
| 157 | 1844.0 |
| 158 | 432.7 |
| 159 | 449.6 |
| 160 | 673.5 |
| 161 | 978.2 |
| 162 | 591.4 |
| 163 | 892.9 |
| 185 | 1014.8 |
| 186 | 257.5 |
| 187 | 228.0 |
| 198 | 47.0 |
| 199 | 963.7 |
| 200 | 207.0 |
| 201 | 391.3 |
| 202 | 241.1 |
| 203 | 583.4 |
| 204 | 130.2 |
| 205 | 631.7 |
| 215 | 419.7 |
| 254 | 3921.2 |
| 255 | 1161.9 |

### Example 6—Western Blotting for Assessing IC50 of Compounds on RBM39 Degradation

A Western blot assay was used to assess the effect of compounds disclosed herein on RBM39 in OVCAR3 cell lines.

Cells were harvested into cell culture medium and counted. The cells were diluted with culture medium for below cell densities and 2 mL of cell suspension was added to each well of 6-well cell culture plate. The plates were covered and incubated at room temperature for 30 minutes without shaking, then incubated at 37° C. and 5% CO₂ overnight for cell attachment.

Test compounds were dissolved to form a 10 mM DMSO stock solution, dilute compounds to 1000× final concentration. A 2 μL aliquot of diluted compound was added to the cell plate. For the Vehicle Control, a 2 μL aliquot of DMSO was used. The plate was gently shaken to mix.

After compound treatment, the media was aspirated and the plates washed with ice cold phosphate buffered saline. Fresh, ice cold 1×RIPA lysis buffer supplemented with protease and phosphatase inhibitors was added to the cells on ice or cold plate, and the cells were pipetted to lyse. The plate was then incubated 5-15 min on ice with shaking, then centrifuged at 4° C. and 15,000 rpm for 10 minutes, and the supernatant was collected.

The cell lysate was mixed with loading dye and reducing agent, heated for 10 min at 95° C., and spun down briefly (10-15 sec) at 13000 rpm at room temperature. Next, a 50 μg sample of protein in was loaded into a gel in 1×MOPS buffer, and the samples were run at 125 V for 120 minutes. The samples were transferred using dry blotting system to a PVDF membrane. High MW Protocol: 10 min at 2.5 A, up to 25V.

Next, the membranes were blocked in TBST/5% BSA for 1 hr at room temperature with shaking at 100 rpm, then hybridized for 16-20 hours at 4° C. with primary antibodies (RBM39:1-in-1000 dilution; β-actin:1-in-4000) in TBST/5% milk with shaking at 100 rpm. The membranes were washed with 1×TBST for 4×5 mins at room temperature, and incubated with secondary antibody anti-rabbit IgG antibody HRP linked (1-in-10000 dilution) and IRDye 680 anti-mouse antibody (1-in-10000 dilution), and diluted in TBST/5% milk for 1 h at room temperature.

The membrane was washed in TBST, 5 min×4 times, the loading control protein and target protein bands were detected after rinsing membrane once with TBS, and the band signal was quantified.

### Example 7—In Vivo Efficacy Studies on Tumor Growth in a Subcutaneous Xenograft Model

Compounds are evaluated for their in vivo efficacy in Cell Line Derived Xenograft model of OVCAR3, (human ovarian cancer cell line, cat #HTB #161 ATCC) to assess in vivo efficacy. Compounds are formulated with 40% PEG400/5% Tween80/55% HP-b-CD (10% w/v). 1×10⁷ OVCAR3 cells in 0.1 ml of PBS mixed with Matrigel (1:1 in volume) are inoculated subcutaneously in the right flank of in Female BALB/c Nude mice at 6-8 weeks of age for tumor development. When the xenografts reach about ˜100-150 mm³ in size, the tumor bearing mice are randomly grouped into study groups (n=10). Randomization is performed based on “Matched distribution” method. The date of randomization is denoted as day 0. Tumor bearing mice are treated by oral gavage with vehicle (40% PEG400/5% Tween80/55% HP-b-CD (10% w/v) or compound at 10, 30 & 100 mg/kg BID for 25 days. Tumor sizes are measured twice weekly in two dimensions using a caliper and the volume are expressed in mm³ using the “V=(L×W×W)/2, where V is tumor volume, L is tumor length (the longest tumor dimension) and W is tumor width (the longest tumor dimension perpendicular to L). Dosing as well as tumor volume and body weight measurements are conducted in a Laminar Flow Cabinet.

1. A compound of Formula (I), or a pharmaceutically acceptable salt thereof: 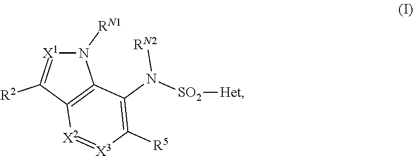
wherein
R^{N1} is H or C₁₋₆alkyl optionally substituted with 1, 2, or 3 R⁷;
R^{N2} is H or C₁₋₆alkyl optionally substituted with 1, 2, or 3 R⁷;
X¹ is CR¹ or N;
X² is CR³ or N;
X³ is CR⁴ or N;
R¹ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R² is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R³ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁴ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
R⁵ is H, C₁₋₆alkyl, C₁₋₆haloalkyl, C₁₋₆alkoxy, halo, OH, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl;
each R^{N} is independently H, C₁₋₆alkyl optionally substituted with 1, 2, or 3 R⁷, or C₃₋₁₀cycloalkyl;
Het is a 5-, 6-, 7-, 8-, 9-, 10-, 11-, or 12-membered heteroaryl comprising 1, 2, or 3 ring heteroatoms selected from O, S, and N and optionally substituted with 1, 2, or 3 R⁶;
each R⁶ is independently halo, CN, C₁₋₆alkyl, C₁₋₆haloalkyl, C₁₋₆alkoxy, NR^{N}R^{N}, COOH, C(O)NR^{N}R^{N}, C₁₋₆alkylene-C(O)OR^{N}, C₁₋₆alkylene-C(O)NR^{N}R^{N}, SO₂NR^{N}R^{N}, P(O)(R^{N})(R^{N}), C(O)-5- or 6-membered heterocycloalkyl comprising 1, 2, or 3 ring heteroatoms selected from O, S, and N, C₁₋₆alkylene-C₃₋₁₀cycloalkyl, C₃₋₁₀cycloalkyl, 4-6-membered heterocycloalkyl comprising 1, 2, or 3 ring heteroatoms selected from O, S, and N, C₆₋₁₀aryl, or 5- or 6-membered heteroaryl comprising 1, 2, or 3 ring heteroatoms selected from O, S, and N, wherein the C₃₋₁₀cycloalkyl, 4-6-membered heterocycloalkyl, C₆₋₁₀aryl, or 5- or 6-membered heteroaryl can optionally be substituted with 1, 2, or 3 R⁷ and each C₁₋₆alkyl, C₁₋₆alkylene, or C₁₋₆alkoxy can be optionally substituted with 1 or 2 substituents independently selected from C₁₋₆alkoxy, OH, CN, CO₂H, NR^{N}R^{N}, and CO₂C₁₋₆alkyl; and
each R⁷ is independently OH, halo, CN, C₁₋₆alkyl, C₁₋₆haloalkyl, C₁₋₆alkoxy, NH₂, NH(C₁₋₆alkyl), or N(C₁₋₆alkyl)₂.

2. The compound or salt of claim 1, wherein Het is a 5-membered heteroaryl comprising 1, 2, or 3 ring heteroatoms selected from O, S, and N and optionally substituted with 1, 2, or 3 R⁶.

3. The compound or salt of claim 2, wherein Het is thiazolyl optionally substituted with 1, 2, or 3 R⁶.

4. The compound or salt of claim 1, wherein Het is 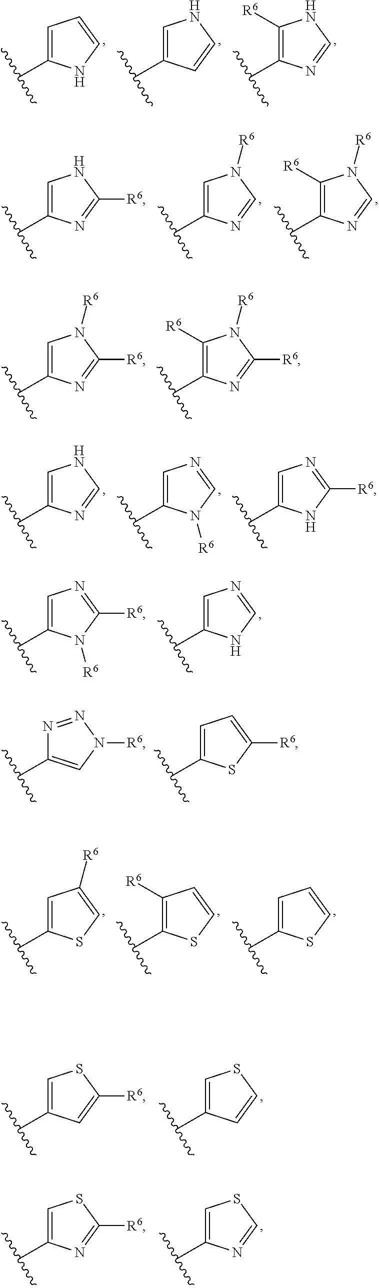 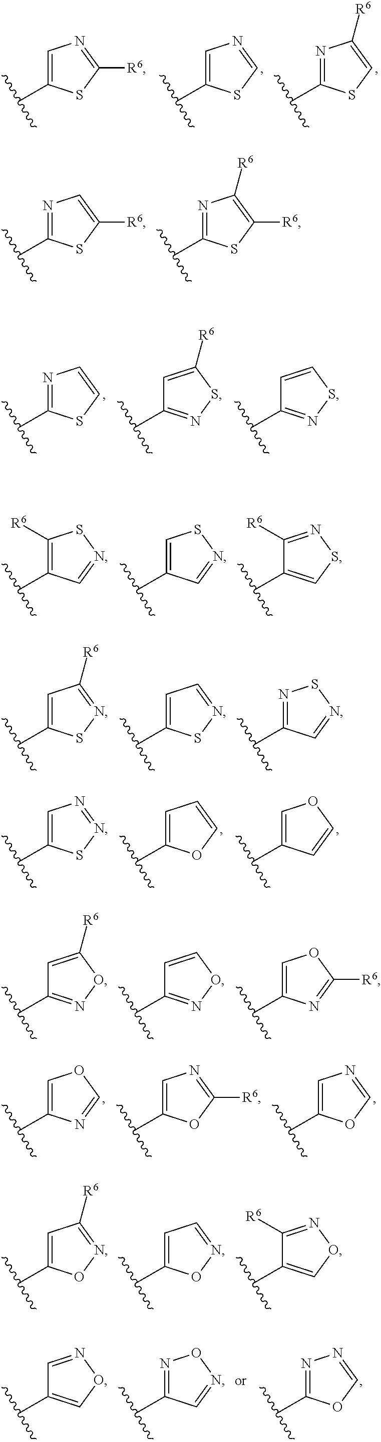
R⁶ is C₁₋₆alkyl, C₁₋₆haloalkyl, C₁₋₆alkoxy, C₃₋₁₀cycloalkyl, P(O)(Me)₂, or C(O)-morpholinyl.

5. The compound or salt of claim 1, wherein X¹ is CR¹, X² is CR³, and X³ is CR⁴.

6. The compound or salt of claim 1, wherein one of X¹, X², and X³ is N.

7. (canceled)

8. (canceled)

9. The compound or salt of claim 1, having the structure of Formula (Ia):

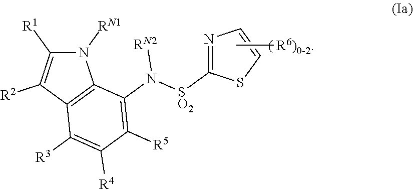

10. The compound or salt of claim 1, wherein R^{N1} is H.

11. The compound or salt of claim 1, wherein R^{N2} is H.

12. The compound or salt of claim 1, wherein R¹ is H or C₁₋₆alkyl, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl.

13. (canceled)

14. The compound or salt of claim 1, wherein R² is H, C₁₋₆alkyl, halo, or CN, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl.

15. (canceled)

16. (canceled)

17. (canceled)

18. The compound or salt of claim 1, wherein R³ is H, C₁₋₆alkyl, or halo, and the C₁₋₆alkyl can optionally be substituted with 1, 2, or 3 substituents independently selected from C₁₋₆alkoxy, OH, CN, CO₂H, NR^{N}R^{N}, and CO₂C₁₋₆alkyl.

19. (canceled)

20. (canceled)

21. (canceled)

22. (canceled)

23. (canceled)

24. The compound or salt of claim 1, wherein R⁴ is H.

25. The compound or salt of claim 1, wherein R⁵ is H.

26. (canceled)

27. (canceled)

28. (canceled)

29. The compound or salt of claim 1, wherein each R⁶ is independently halo, CN, C₁₋₆alkyl, C₁₋₆haloalkyl, C₁₋₆alkoxy, COOH, C(O)NR^{N}R^{N}, C₁₋₆alkylene-C(O)OR^{N}, P(O)(R^{N})(R^{N}), C(O)-5- or 6-membered heterocycloalkyl comprising 1, 2, or 3 ring heteroatoms selected from O, S, and N, C₃₋₅cycloalkyl, 5- or 6-membered heterocycloalkyl comprising 1, 2, or 3 ring heteroatoms selected from O, S, and N, C₆₋₁₀aryl, or 5- or 6-membered heteroaryl comprising 1, 2, or 3 ring heteroatoms selected from O, S, and N, wherein the C₃₋₅cycloalkyl, 5- or 6-membered heterocycloalkyl, C₆₋₁₀aryl, or 5- or 6-membered heteroaryl can optionally be substituted with 1, 2, or 3 R⁷ and each C₁₋₆ alkyl or C₁₋₆alkylene can be optionally substituted with 1 or 2 substituents independently selected from C₁₋₆alkoxy, OH, CN, CO₂H, NR^{N}R^{N}, and CO₂C₁₋₆alkyl.

30. (canceled)

31. (canceled)

32. (canceled)

33. (canceled)

34. (canceled)

35. (canceled)

36. (canceled)

37. (canceled)

38. The compound or salt of claim 1, wherein each R⁷ is independently halo, C₁₋₆alkyl, or C₁₋₆haloalkyl.

39. (canceled)

40. (canceled)

41. A compound listed in Table A, or a pharmaceutically acceptable salt thereof;
TABLE A

| Compound No. | Structure |
|---|---|
| 1 | 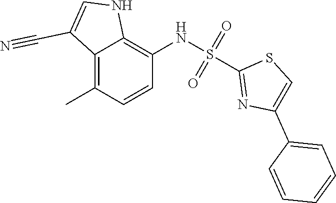 [SMILES: Cc1ccc(NS(=O)(=O)c2nc(-c3ccccc3)cs2)c2[nH]cc(C#N)c12] |
| 2 | 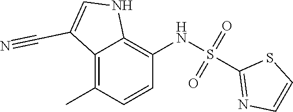 [SMILES: Cc1ccc(NS(=O)(=O)c2nccs2)c2[nH]cc(C#N)c12] |
| 3 | 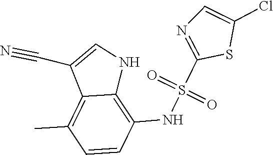 [SMILES: Cc1ccc(NS(=O)(=O)c2ncc(Cl)s2)c2[nH]cc(C#N)c12] |
| 4 | 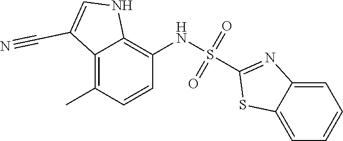 [SMILES: Cc1ccc(NS(=O)(=O)c2nc3ccccc3s2)c2[nH]cc(C#N)c12] |
| 5 | 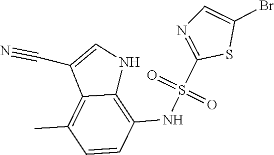 [SMILES: Cc1ccc(NS(=O)(=O)c2ncc(Br)s2)c2[nH]cc(C#N)c12] |
| 6 | 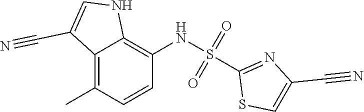 [SMILES: Cc1ccc(NS(=O)(=O)c2nc(C#N)cs2)c2[nH]cc(C#N)c12] |
| 7 | 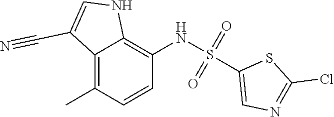 [SMILES: Cc1ccc(NS(=O)(=O)c2cnc(Cl)s2)c2[nH]cc(C#N)c12] |
| 8 | 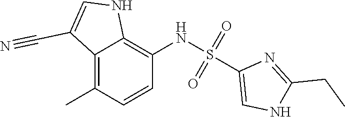 [SMILES: CCc1nc(S(=O)(=O)Nc2ccc(C)c3c(C#N)c[nH]c23)c[nH]1] |
| 9 | 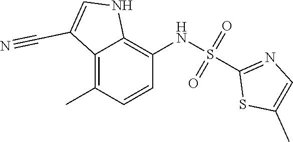 [SMILES: Cc1cnc(S(=O)(=O)Nc2ccc(C)c3c(C#N)c[nH]c23)s1] |
| 10 | 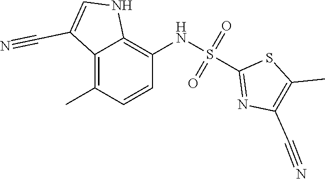 [SMILES: Cc1sc(S(=O)(=O)Nc2ccc(C)c3c(C#N)c[nH]c23)nc1C#N] |
| 11 | 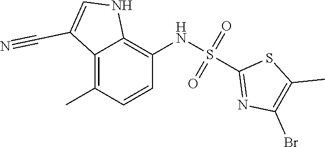 [SMILES: Cc1sc(S(=O)(=O)Nc2ccc(C)c3c(C#N)c[nH]c23)nc1Br] |
| 12 | 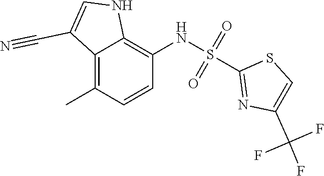 [SMILES: Cc1ccc(NS(=O)(=O)c2nc(C(F)(F)F)cs2)c2[nH]cc(C#N)c12] |
| 13 | 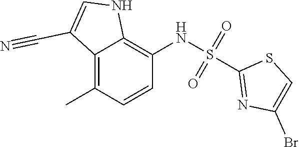 [SMILES: Cc1ccc(NS(=O)(=O)c2nc(Br)cs2)c2[nH]cc(C#N)c12] |
| 14 | 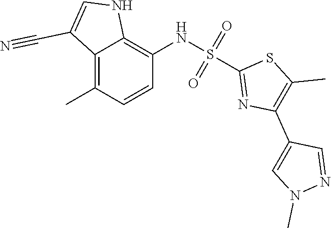 [SMILES: Cc1sc(S(=O)(=O)Nc2ccc(C)c3c(C#N)c[nH]c23)nc1-c1cnn(C)c1] |
| 15 | 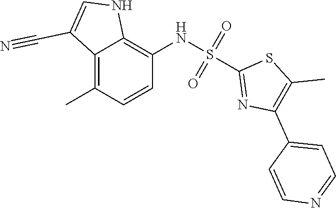 [SMILES: Cc1sc(S(=O)(=O)Nc2ccc(C)c3c(C#N)c[nH]c23)nc1-c1ccncc1] |
| 16 | 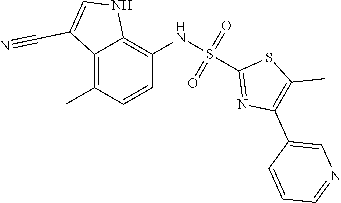 [SMILES: Cc1sc(S(=O)(=O)Nc2ccc(C)c3c(C#N)c[nH]c23)nc1-c1cccnc1] |
| 17 | 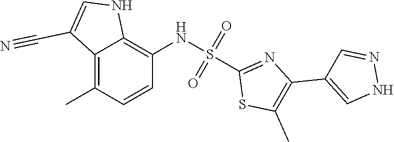 [SMILES: Cc1sc(S(=O)(=O)Nc2ccc(C)c3c(C#N)c[nH]c23)nc1-c1cn[nH]c1] |
| 18 | 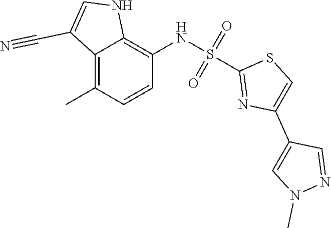 [SMILES: Cc1ccc(NS(=O)(=O)c2nc(-c3cnn(C)c3)cs2)c2[nH]cc(C#N)c12] |
| 19 | 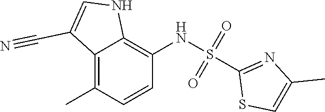 [SMILES: Cc1csc(S(=O)(=O)Nc2ccc(C)c3c(C#N)c[nH]c23)n1] |
| 20 | 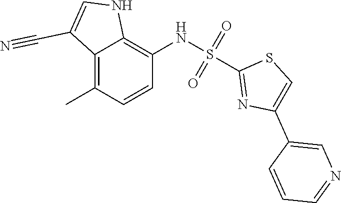 [SMILES: Cc1ccc(NS(=O)(=O)c2nc(-c3cccnc3)cs2)c2[nH]cc(C#N)c12] |
| 21 | 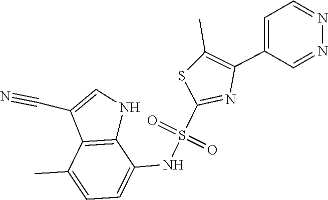 [SMILES: Cc1sc(S(=O)(=O)Nc2ccc(C)c3c(C#N)c[nH]c23)nc1-c1ccnnc1] |
| 22 | 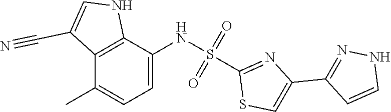 [SMILES: Cc1ccc(NS(=O)(=O)c2nc(-c3cc[nH]n3)cs2)c2[nH]cc(C#N)c12] |
| 23 | 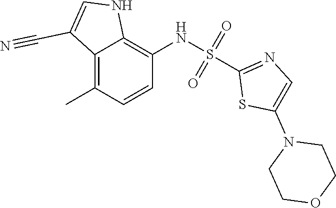 [SMILES: Cc1ccc(NS(=O)(=O)c2ncc(N3CCOCC3)s2)c2[nH]cc(C#N)c12] |
| 24 | 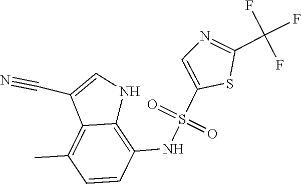 [SMILES: Cc1ccc(NS(=O)(=O)c2cnc(C(F)(F)F)s2)c2[nH]cc(C#N)c12] |
| 25 | 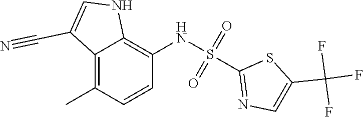 [SMILES: Cc1ccc(NS(=O)(=O)c2ncc(C(F)(F)F)s2)c2[nH]cc(C#N)c12] |
| 26 | 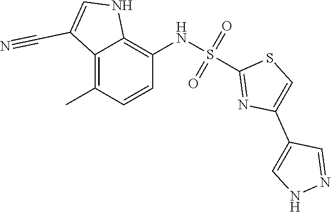 [SMILES: Cc1ccc(NS(=O)(=O)c2nc(-c3cn[nH]c3)cs2)c2[nH]cc(C#N)c12] |
| 27 | 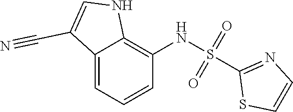 [SMILES: N#Cc1c[nH]c2c(NS(=O)(=O)c3nccs3)cccc12] |
| 28 | 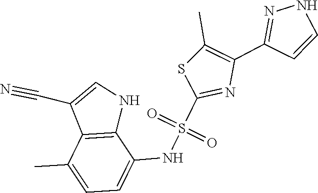 [SMILES: Cc1sc(S(=O)(=O)Nc2ccc(C)c3c(C#N)c[nH]c23)nc1-c1cc[nH]n1] |
| 29 | 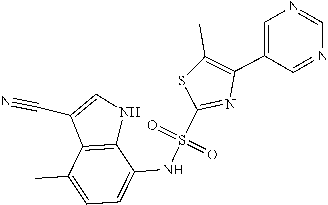 [SMILES: Cc1sc(S(=O)(=O)Nc2ccc(C)c3c(C#N)c[nH]c23)nc1-c1cncnc1] |
| 30 | 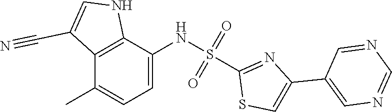 [SMILES: Cc1ccc(NS(=O)(=O)c2nc(-c3cncnc3)cs2)c2[nH]cc(C#N)c12] |
| 31 | 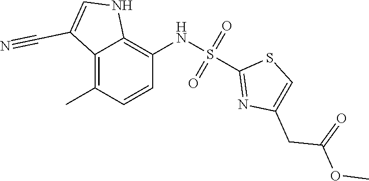 [SMILES: COC(=O)Cc1csc(S(=O)(=O)Nc2ccc(C)c3c(C#N)c[nH]c23)n1] |
| 32 | 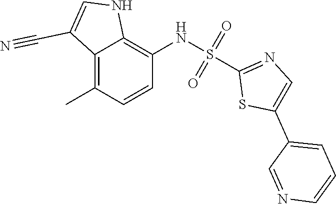 [SMILES: Cc1ccc(NS(=O)(=O)c2ncc(-c3cccnc3)s2)c2[nH]cc(C#N)c12] |
| 33 | 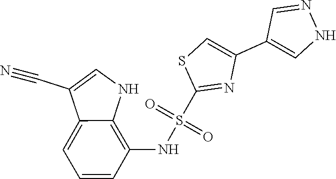 [SMILES: N#Cc1c[nH]c2c(NS(=O)(=O)c3nc(-c4cn[nH]c4)cs3)cccc12] |
| 34 | 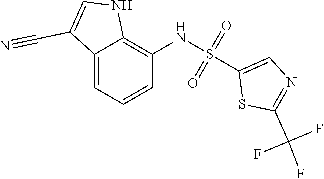 [SMILES: N#Cc1c[nH]c2c(NS(=O)(=O)c3cnc(C(F)(F)F)s3)cccc12] |
| 35 | 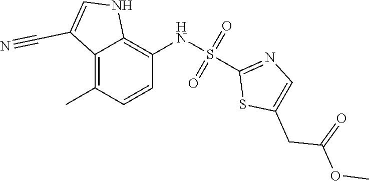 [SMILES: COC(=O)Cc1cnc(S(=O)(=O)Nc2ccc(C)c3c(C#N)c[nH]c23)s1] |
| 36 | 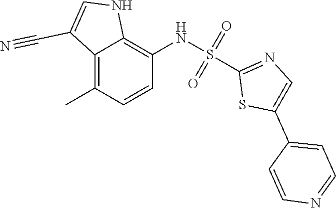 [SMILES: Cc1ccc(NS(=O)(=O)c2ncc(-c3ccncc3)s2)c2[nH]cc(C#N)c12] |
| 37 | 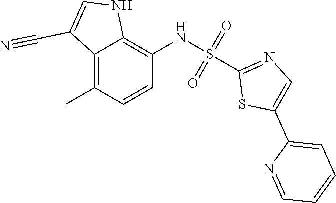 [SMILES: Cc1ccc(NS(=O)(=O)c2ncc(-c3ccccn3)s2)c2[nH]cc(C#N)c12] |
| 38 | 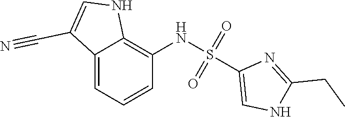 [SMILES: CCc1nc(S(=O)(=O)Nc2cccc3c(C#N)c[nH]c23)c[nH]1] |
| 39 | 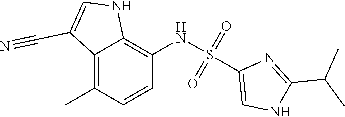 [SMILES: Cc1ccc(NS(=O)(=O)c2c[nH]c(C(C)C)n2)c2[nH]cc(C#N)c12] |
| 40 | 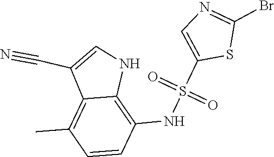 [SMILES: Cc1ccc(NS(=O)(=O)c2cnc(Br)s2)c2[nH]cc(C#N)c12] |
| 41 | 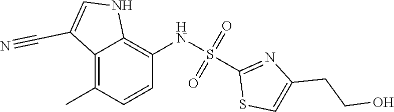 [SMILES: Cc1ccc(NS(=O)(=O)c2nc(CCO)cs2)c2[nH]cc(C#N)c12] |
| 42 | 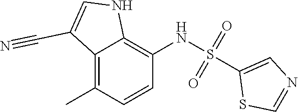 [SMILES: Cc1ccc(NS(=O)(=O)c2cncs2)c2[nH]cc(C#N)c12] |
| 43 | 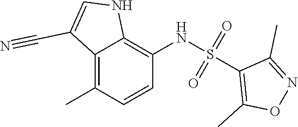 [SMILES: Cc1noc(C)c1S(=O)(=O)Nc1ccc(C)c2c(C#N)c[nH]c12] |
| 44 | 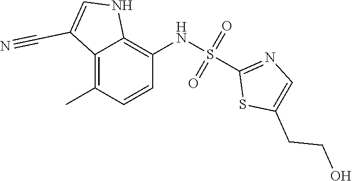 [SMILES: Cc1ccc(NS(=O)(=O)c2ncc(CCO)s2)c2[nH]cc(C#N)c12] |
| 45 | 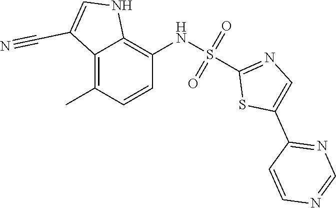 [SMILES: Cc1ccc(NS(=O)(=O)c2ncc(-c3ccncn3)s2)c2[nH]cc(C#N)c12] |
| 46 | 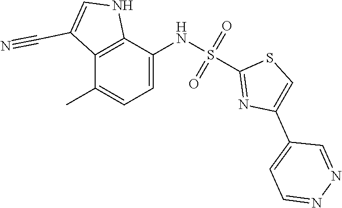 [SMILES: Cc1ccc(NS(=O)(=O)c2nc(-c3ccnnc3)cs2)c2[nH]cc(C#N)c12] |
| 47 | 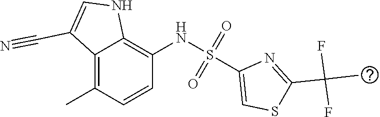 [SMILES: Cc1ccc(NS(=O)(=O)c2csc(C(C)(F)F)n2)c2[nH]cc(C#N)c12] |
| 48 | 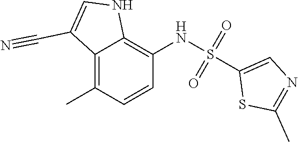 [SMILES: Cc1ncc(S(=O)(=O)Nc2ccc(C)c3c(C#N)c[nH]c23)s1] |
| 49 | 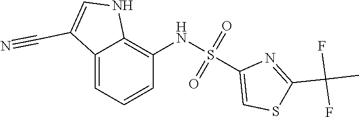 [SMILES: CC(F)(F)c1nc(S(=O)(=O)Nc2cccc3c(C#N)c[nH]c23)cs1] |
| 50 | 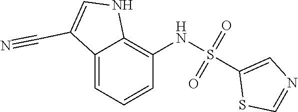 [SMILES: N#Cc1c[nH]c2c(NS(=O)(=O)c3cncs3)cccc12] |
| 51 | 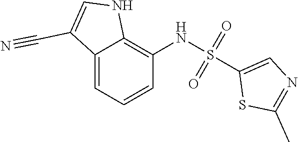 [SMILES: Cc1ncc(S(=O)(=O)Nc2cccc3c(C#N)c[nH]c23)s1] |
| 52 | 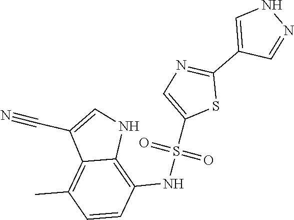 [SMILES: Cc1ccc(NS(=O)(=O)c2cnc(-c3cn[nH]c3)s2)c2[nH]cc(C#N)c12] |
| 53 | 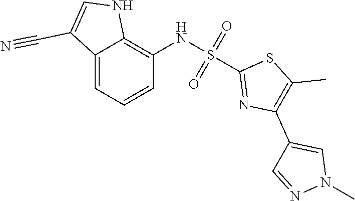 [SMILES: Cc1sc(S(=O)(=O)Nc2cccc3c(C#N)c[nH]c23)nc1-c1cnn(C)c1] |
| 54 | 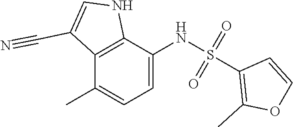 [SMILES: Cc1occc1S(=O)(=O)Nc1ccc(C)c2c(C#N)c[nH]c12] |
| 55 | 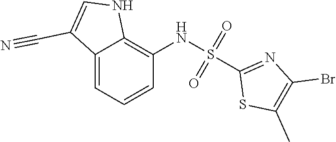 [SMILES: Cc1sc(S(=O)(=O)Nc2cccc3c(C#N)c[nH]c23)nc1Br] |
| 56 | 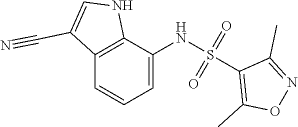 [SMILES: Cc1noc(C)c1S(=O)(=O)Nc1cccc2c(C#N)c[nH]c12] |
| 57 | 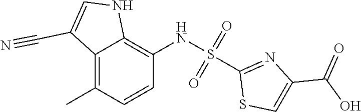 [SMILES: Cc1ccc(NS(=O)(=O)c2nc(C(=O)O)cs2)c2[nH]cc(C#N)c12] |
| 58 | 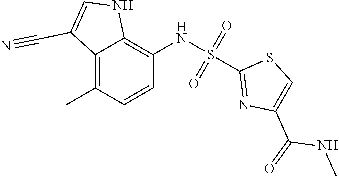 [SMILES: CNC(=O)c1csc(S(=O)(=O)Nc2ccc(C)c3c(C#N)c[nH]c23)n1] |
| 59 | 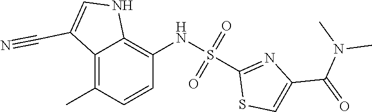 [SMILES: Cc1ccc(NS(=O)(=O)c2nc(C(=O)N(C)C)cs2)c2[nH]cc(C#N)c12] |
| 60 | 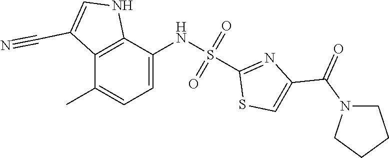 [SMILES: Cc1ccc(NS(=O)(=O)c2nc(C(=O)N3CCCC3)cs2)c2[nH]cc(C#N)c12] |
| 61 | 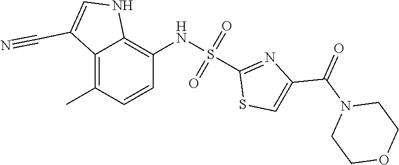 [SMILES: Cc1ccc(NS(=O)(=O)c2nc(C(=O)N3CCOCC3)cs2)c2[nH]cc(C#N)c12] |
| 62 | 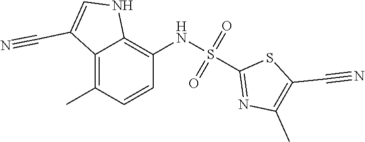 [SMILES: Cc1nc(S(=O)(=O)Nc2ccc(C)c3c(C#N)c[nH]c23)sc1C#N] |
| 63 | 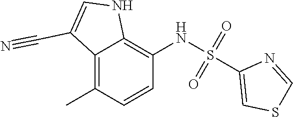 [SMILES: Cc1ccc(NS(=O)(=O)c2cscn2)c2[nH]cc(C#N)c12] |
| 64 | 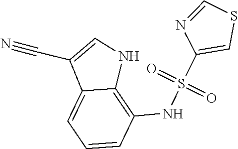 [SMILES: N#Cc1c[nH]c2c(NS(=O)(=O)c3cscn3)cccc12] |
| 65 | 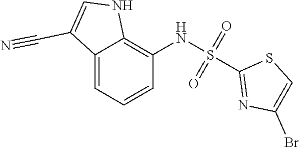 [SMILES: N#Cc1c[nH]c2c(NS(=O)(=O)c3nc(Br)cs3)cccc12] |
| 66 | 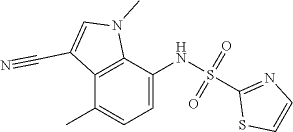 [SMILES: Cc1ccc(NS(=O)(=O)c2nccs2)c2c1c(C#N)cn2C] |
| 67 | 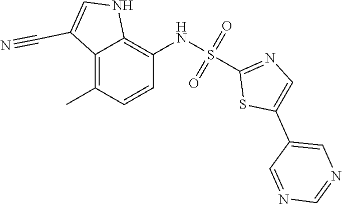 [SMILES: Cc1ccc(NS(=O)(=O)c2ncc(-c3cncnc3)s2)c2[nH]cc(C#N)c12] |
| 68 | 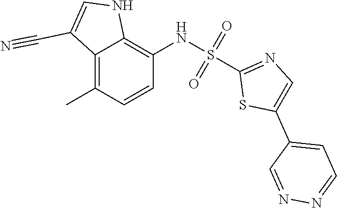 [SMILES: Cc1ccc(NS(=O)(=O)c2ncc(-c3ccnnc3)s2)c2[nH]cc(C#N)c12] |
| 69 | 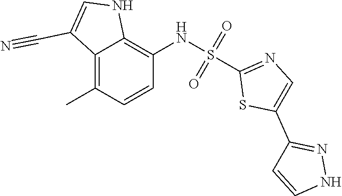 [SMILES: Cc1ccc(NS(=O)(=O)c2ncc(-c3cc[nH]n3)s2)c2[nH]cc(C#N)c12] |
| 70 | 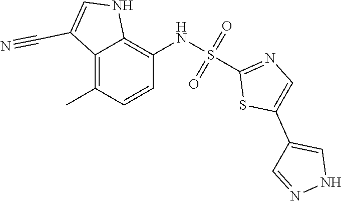 [SMILES: Cc1ccc(NS(=O)(=O)c2ncc(-c3cn[nH]c3)s2)c2[nH]cc(C#N)c12] |
| 71 | 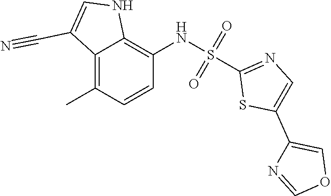 [SMILES: Cc1ccc(NS(=O)(=O)c2ncc(-c3cocn3)s2)c2[nH]cc(C#N)c12] |
| 72 | 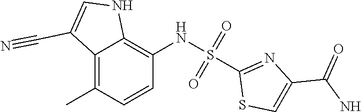 [SMILES: Cc1ccc(NS(=O)(=O)c2nc(C(N)=O)cs2)c2[nH]cc(C#N)c12] |
| 73 | 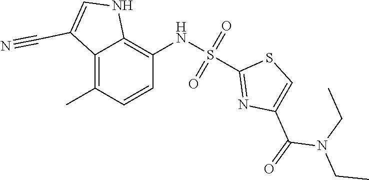 [SMILES: CCN(CC)C(=O)c1csc(S(=O)(=O)Nc2ccc(C)c3c(C#N)c[nH]c23)n1] |
| 74 | 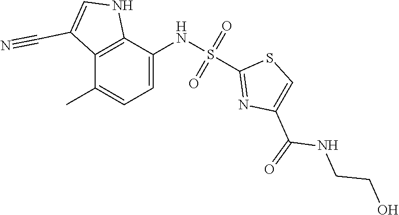 [SMILES: Cc1ccc(NS(=O)(=O)c2nc(C(=O)NCCO)cs2)c2[nH]cc(C#N)c12] |
| 75 | 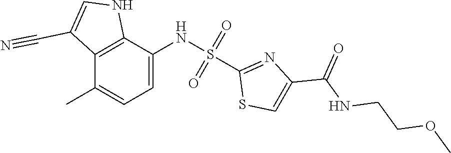 [SMILES: COCCNC(=O)c1csc(S(=O)(=O)Nc2ccc(C)c3c(C#N)c[nH]c23)n1] |
| 76 | 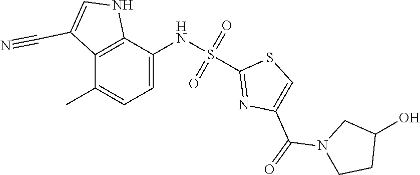 [SMILES: Cc1ccc(NS(=O)(=O)c2nc(C(=O)N3CCC(O)C3)cs2)c2[nH]cc(C#N)c12] |
| 77 | 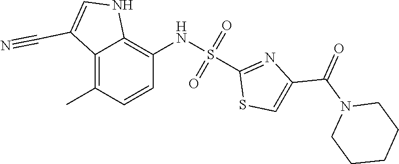 [SMILES: Cc1ccc(NS(=O)(=O)c2nc(C(=O)N3CCCCC3)cs2)c2[nH]cc(C#N)c12] |
| 78 | 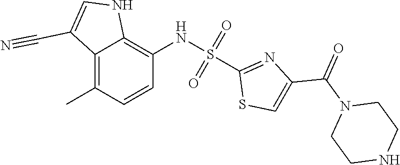 [SMILES: Cc1ccc(NS(=O)(=O)c2nc(C(=O)N3CCNCC3)cs2)c2[nH]cc(C#N)c12] |
| 79 | 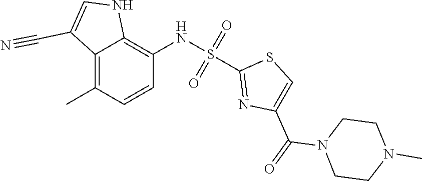 [SMILES: Cc1ccc(NS(=O)(=O)c2nc(C(=O)N3CCN(C)CC3)cs2)c2[nH]cc(C#N)c12] |
| 80 | 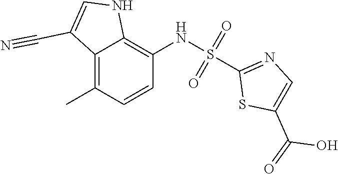 [SMILES: Cc1ccc(NS(=O)(=O)c2ncc(C(=O)O)s2)c2[nH]cc(C#N)c12] |
| 81 | 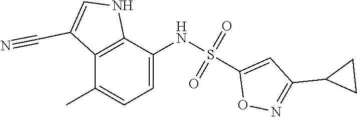 [SMILES: Cc1ccc(NS(=O)(=O)c2cc(C3CC3)no2)c2[nH]cc(C#N)c12] |
| 82 | 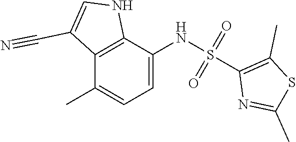 [SMILES: Cc1nc(S(=O)(=O)Nc2ccc(C)c3c(C#N)c[nH]c23)c(C)s1] |
| 83 | 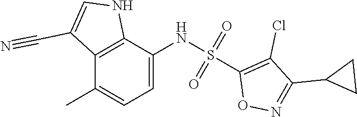 [SMILES: Cc1ccc(NS(=O)(=O)c2onc(C3CC3)c2Cl)c2[nH]cc(C#N)c12] |
| 84 | 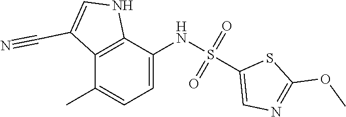 [SMILES: COc1ncc(S(=O)(=O)Nc2ccc(C)c3c(C#N)c[nH]c23)s1] |
| 85 | 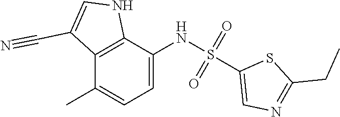 [SMILES: CCc1ncc(S(=O)(=O)Nc2ccc(C)c3c(C#N)c[nH]c23)s1] |
| 86 | 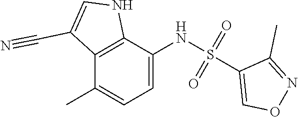 [SMILES: Cc1nocc1S(=O)(=O)Nc1ccc(C)c2c(C#N)c[nH]c12] |
| 87 | 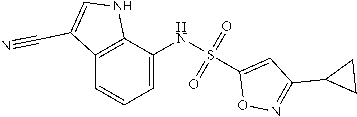 [SMILES: N#Cc1c[nH]c2c(NS(=O)(=O)c3cc(C4CC4)no3)cccc12] |
| 88 | 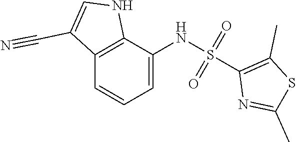 [SMILES: Cc1nc(S(=O)(=O)Nc2cccc3c(C#N)c[nH]c23)c(C)s1] |
| 89 | 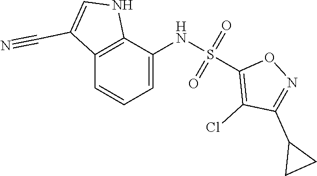 [SMILES: N#Cc1c[nH]c2c(NS(=O)(=O)c3onc(C4CC4)c3Cl)cccc12] |
| 90 | 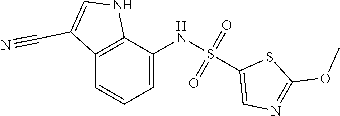 [SMILES: COc1ncc(S(=O)(=O)Nc2cccc3c(C#N)c[nH]c23)s1] |
| 91 | 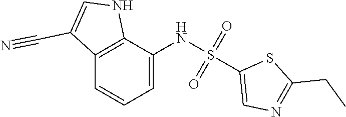 [SMILES: CCc1ncc(S(=O)(=O)Nc2cccc3c(C#N)c[nH]c23)s1] |
| 92 | 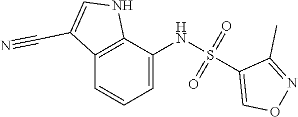 [SMILES: Cc1nocc1S(=O)(=O)Nc1cccc2c(C#N)c[nH]c12] |
| 93 | 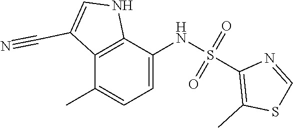 [SMILES: Cc1scnc1S(=O)(=O)Nc1ccc(C)c2c(C#N)c[nH]c12] |
| 94 | 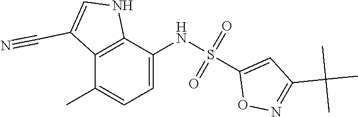 [SMILES: Cc1ccc(NS(=O)(=O)c2cc(C(C)(C)C)no2)c2[nH]cc(C#N)c12] |
| 95 | 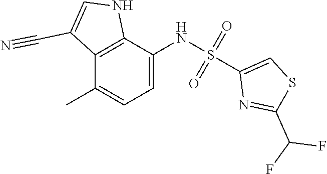 [SMILES: Cc1ccc(NS(=O)(=O)c2csc(C(F)F)n2)c2[nH]cc(C#N)c12] |
| 96 | 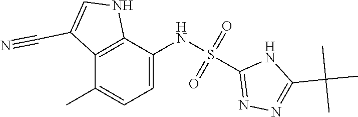 [SMILES: Cc1ccc(NS(=O)(=O)c2nnc(C(C)(C)C)[nH]2)c2[nH]cc(C#N)c12] |
| 97 | 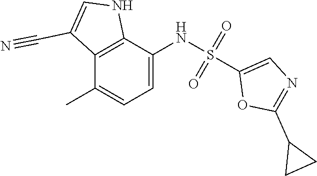 [SMILES: Cc1ccc(NS(=O)(=O)c2cnc(C3CC3)o2)c2[nH]cc(C#N)c12] |
| 98 | 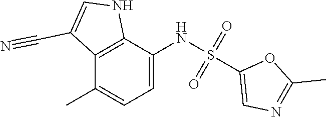 [SMILES: Cc1ncc(S(=O)(=O)Nc2ccc(C)c3c(C#N)c[nH]c23)o1] |
| 99 | 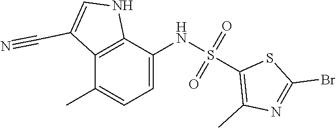 [SMILES: Cc1nc(Br)sc1S(=O)(=O)Nc1ccc(C)c2c(C#N)c[nH]c12] |
| 100 | 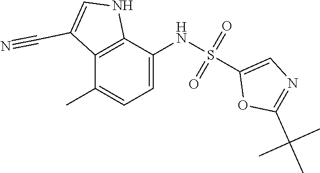 [SMILES: Cc1ccc(NS(=O)(=O)c2cnc(C(C)(C)C)o2)c2[nH]cc(C#N)c12] |
| 101 | 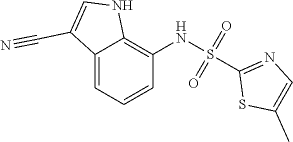 [SMILES: Cc1cnc(S(=O)(=O)Nc2cccc3c(C#N)c[nH]c23)s1] |
| 102 | 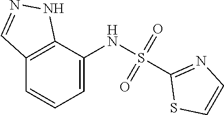 [SMILES: O=S(=O)(Nc1cccc2cn[nH]c12)c1nccs1] |
| 103 | 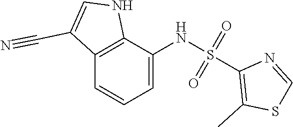 [SMILES: Cc1scnc1S(=O)(=O)Nc1cccc2c(C#N)c[nH]c12] |
| 104 | 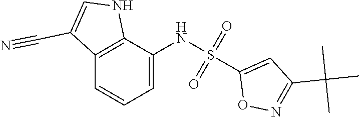 [SMILES: CC(C)(C)c1cc(S(=O)(=O)Nc2cccc3c(C#N)c[nH]c23)on1] |
| 105 | 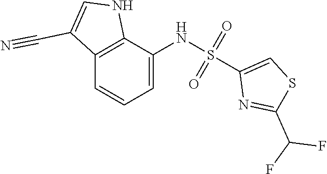 [SMILES: N#Cc1c[nH]c2c(NS(=O)(=O)c3csc(C(F)F)n3)cccc12] |
| 106 | 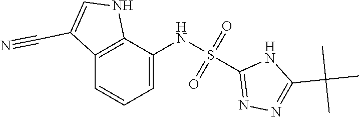 [SMILES: CC(C)(C)c1nnc(S(=O)(=O)Nc2cccc3c(C#N)c[nH]c23)[nH]1] |
| 107 | 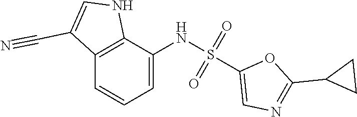 [SMILES: N#Cc1c[nH]c2c(NS(=O)(=O)c3cnc(C4CC4)o3)cccc12] |
| 108 | 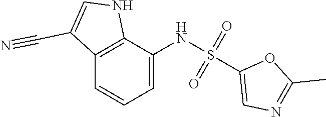 [SMILES: Cc1ncc(S(=O)(=O)Nc2cccc3c(C#N)c[nH]c23)o1] |
| 109 | 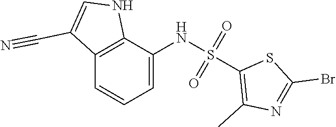 [SMILES: Cc1nc(Br)sc1S(=O)(=O)Nc1cccc2c(C#N)c[nH]c12] |
| 110 | 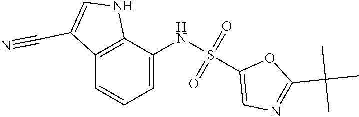 [SMILES: CC(C)(C)c1ncc(S(=O)(=O)Nc2cccc3c(C#N)c[nH]c23)o1] |
| 111 | 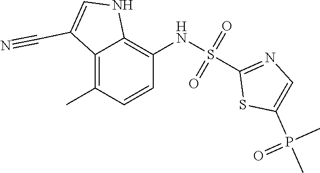 [SMILES: Cc1ccc(NS(=O)(=O)c2ncc(P(C)(C)=O)s2)c2[nH]cc(C#N)c12] |
| 112 | 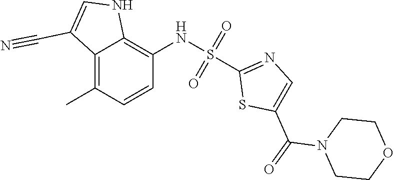 [SMILES: Cc1ccc(NS(=O)(=O)c2ncc(C(=O)N3CCOCC3)s2)c2[nH]cc(C#N)c12] |
| 113 | 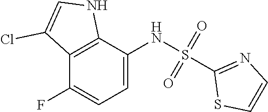 [SMILES: O=S(=O)(Nc1ccc(F)c2c(Cl)c[nH]c12)c1nccs1] |
| 114 | 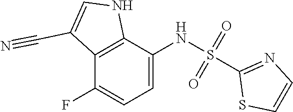 [SMILES: N#Cc1c[nH]c2c(NS(=O)(=O)c3nccs3)ccc(F)c12] |
| 115 | 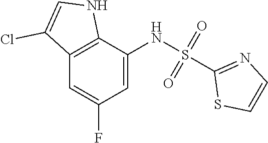 [SMILES: O=S(=O)(Nc1cc(F)cc2c(Cl)c[nH]c12)c1nccs1] |
| 116 | 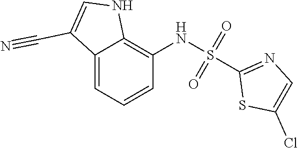 [SMILES: N#Cc1c[nH]c2c(NS(=O)(=O)c3ncc(Cl)s3)cccc12] |
| 117 | 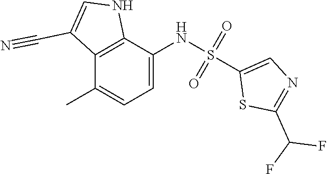 [SMILES: Cc1ccc(NS(=O)(=O)c2cnc(C(F)F)s2)c2[nH]cc(C#N)c12] |
| 118 | 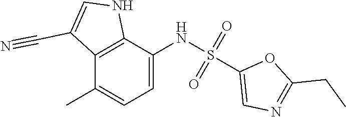 [SMILES: CCc1ncc(S(=O)(=O)Nc2ccc(C)c3c(C#N)c[nH]c23)o1] |
| 119 | 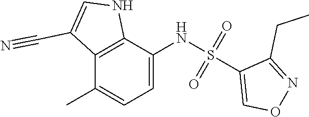 [SMILES: CCc1nocc1S(=O)(=O)Nc1ccc(C)c2c(C#N)c[nH]c12] |
| 120 | 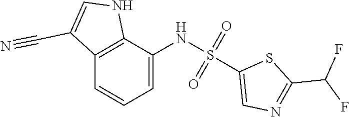 [SMILES: N#Cc1c[nH]c2c(NS(=O)(=O)c3cnc(C(F)F)s3)cccc12] |
| 121 | 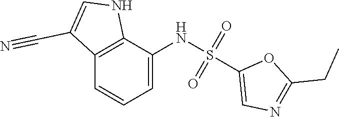 [SMILES: CCc1ncc(S(=O)(=O)Nc2cccc3c(C#N)c[nH]c23)o1] |
| 122 | 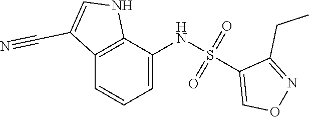 [SMILES: CCc1nocc1S(=O)(=O)Nc1cccc2c(C#N)c[nH]c12] |
| 123 | 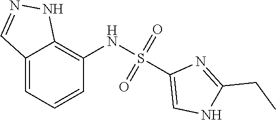 [SMILES: CCc1nc(S(=O)(=O)Nc2cccc3cn[nH]c23)c[nH]1] |
| 124 | 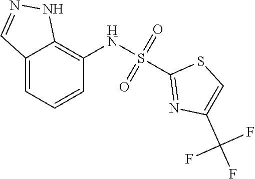 [SMILES: O=S(=O)(Nc1cccc2cn[nH]c12)c1nc(C(F)(F)F)cs1] |
| 125 | 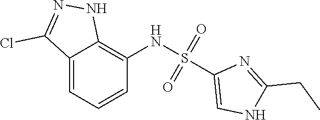 [SMILES: CCc1nc(S(=O)(=O)Nc2cccc3c(Cl)n[nH]c23)c[nH]1] |
| 126 | 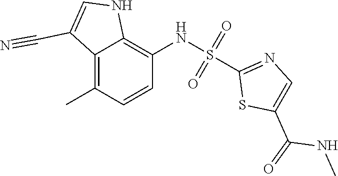 [SMILES: CNC(=O)c1cnc(S(=O)(=O)Nc2ccc(C)c3c(C#N)c[nH]c23)s1] |
| 127 | 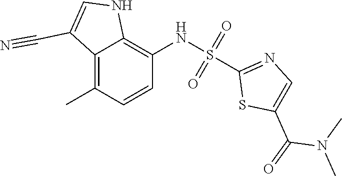 [SMILES: Cc1ccc(NS(=O)(=O)c2ncc(C(=O)N(C)C)s2)c2[nH]cc(C#N)c12] |
| 128 | 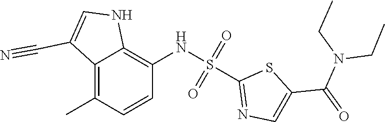 [SMILES: CCN(CC)C(=O)c1cnc(S(=O)(=O)Nc2ccc(C)c3c(C#N)c[nH]c23)s1] |
| 129 | 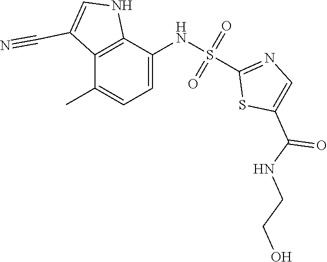 [SMILES: Cc1ccc(NS(=O)(=O)c2ncc(C(=O)NCCO)s2)c2[nH]cc(C#N)c12] |
| 130 | 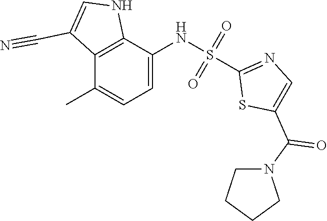 [SMILES: Cc1ccc(NS(=O)(=O)c2ncc(C(=O)N3CCCC3)s2)c2[nH]cc(C#N)c12] |
| 131 | 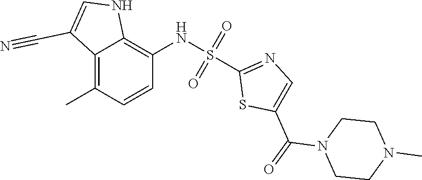 [SMILES: Cc1ccc(NS(=O)(=O)c2ncc(C(=O)N3CCN(C)CC3)s2)c2[nH]cc(C#N)c12] |
| 132 | 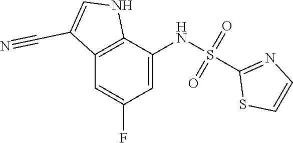 [SMILES: N#Cc1c[nH]c2c(NS(=O)(=O)c3nccs3)cc(F)cc12] |
| 133 | 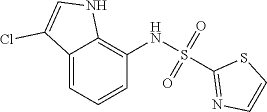 [SMILES: O=S(=O)(Nc1cccc2c(Cl)c[nH]c12)c1nccs1] |
| 134 | 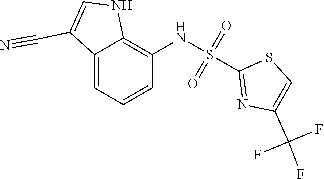 [SMILES: N#Cc1c[nH]c2c(NS(=O)(=O)c3nc(C(F)(F)F)cs3)cccc12] |
| 135 | 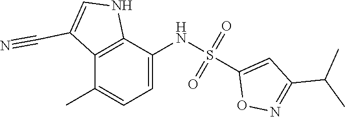 [SMILES: Cc1ccc(NS(=O)(=O)c2cc(C(C)C)no2)c2[nH]cc(C#N)c12] |
| 136 | 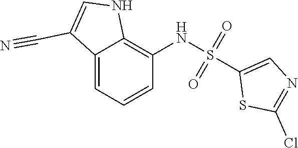 [SMILES: N#Cc1c[nH]c2c(NS(=O)(=O)c3cnc(Cl)s3)cccc12] |
| 137 | 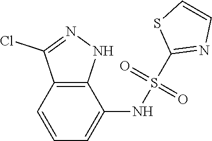 [SMILES: O=S(=O)(Nc1cccc2c(Cl)n[nH]c12)c1nccs1] |
| 138 | 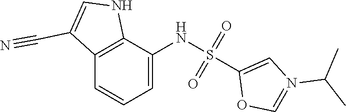 |
| 139 | 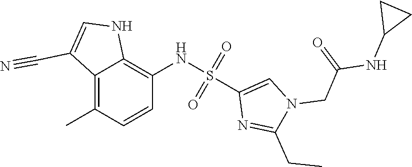 [SMILES: CCc1nc(S(=O)(=O)Nc2ccc(C)c3c(C#N)c[nH]c23)cn1CC(=O)NC1CC1] |
| 140 | 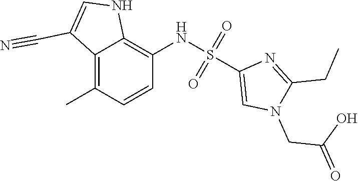 [SMILES: CCc1nc(S(=O)(=O)Nc2ccc(C)c3c(C#N)c[nH]c23)cn1CC(=O)O] |
| 141 | 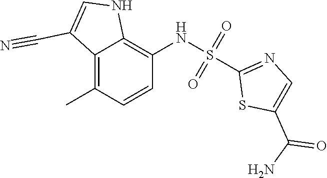 [SMILES: Cc1ccc(NS(=O)(=O)c2ncc(C(N)=O)s2)c2[nH]cc(C#N)c12] |
| 142 | 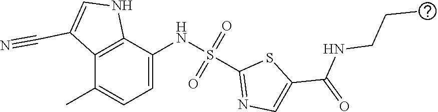 [SMILES: CCCNC(=O)c1cnc(S(=O)(=O)Nc2ccc(C)c3c(C#N)c[nH]c23)s1] |
| 143 | 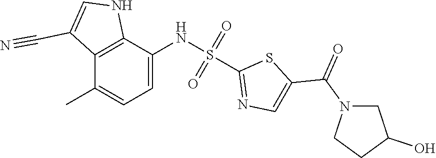 [SMILES: Cc1ccc(NS(=O)(=O)c2ncc(C(=O)N3CCC(O)C3)s2)c2[nH]cc(C#N)c12] |
| 144 | 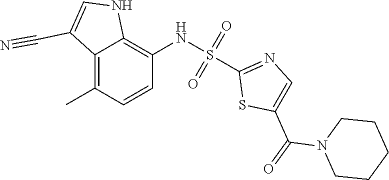 [SMILES: Cc1ccc(NS(=O)(=O)c2ncc(C(=O)N3CCCCC3)s2)c2[nH]cc(C#N)c12] |
| 145 | 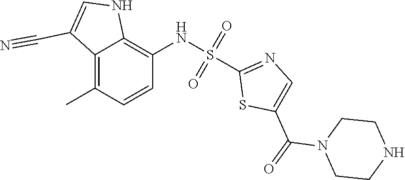 [SMILES: Cc1ccc(NS(=O)(=O)c2ncc(C(=O)N3CCNCC3)s2)c2[nH]cc(C#N)c12] |
| 146 | 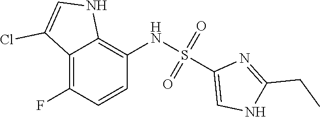 [SMILES: CCc1nc(S(=O)(=O)Nc2ccc(F)c3c(Cl)c[nH]c23)c[nH]1] |
| 147 | 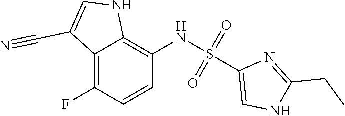 [SMILES: CCc1nc(S(=O)(=O)Nc2ccc(F)c3c(C#N)c[nH]c23)c[nH]1] |
| 148 | 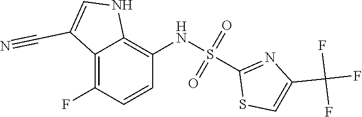 [SMILES: N#Cc1c[nH]c2c(NS(=O)(=O)c3nc(C(F)(F)F)cs3)ccc(F)c12] |
| 149 | 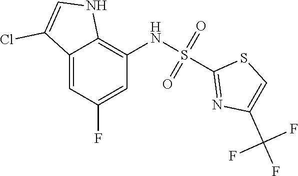 [SMILES: O=S(=O)(Nc1cc(F)cc2c(Cl)c[nH]c12)c1nc(C(F)(F)F)cs1] |
| 150 | 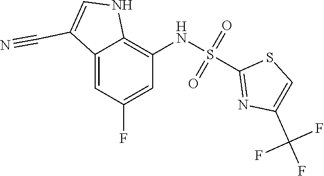 [SMILES: N#Cc1c[nH]c2c(NS(=O)(=O)c3nc(C(F)(F)F)cs3)cc(F)cc12] |
| 151 | 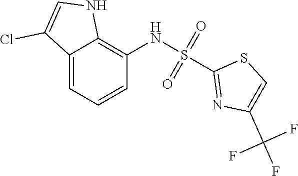 [SMILES: O=S(=O)(Nc1cccc2c(Cl)c[nH]c12)c1nc(C(F)(F)F)cs1] |
| 152 | 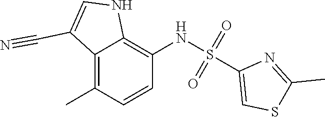 [SMILES: Cc1nc(S(=O)(=O)Nc2ccc(C)c3c(C#N)c[nH]c23)cs1] |
| 153 | 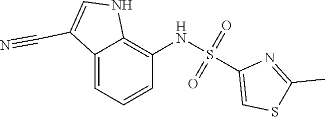 [SMILES: Cc1nc(S(=O)(=O)Nc2cccc3c(C#N)c[nH]c23)cs1] |
| 154 | 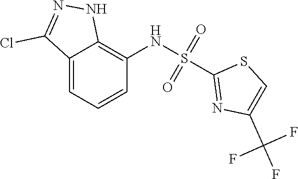 [SMILES: O=S(=O)(Nc1cccc2c(Cl)n[nH]c12)c1nc(C(F)(F)F)cs1] |
| 155 | 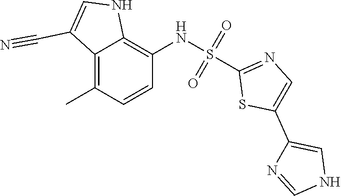 [SMILES: Cc1ccc(NS(=O)(=O)c2ncc(-c3c[nH]cn3)s2)c2[nH]cc(C#N)c12] |
| 156 | 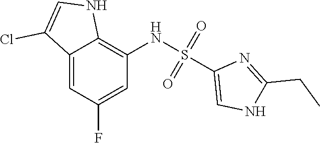 [SMILES: CCc1nc(S(=O)(=O)Nc2cc(F)cc3c(Cl)c[nH]c23)c[nH]1] |
| 157 | 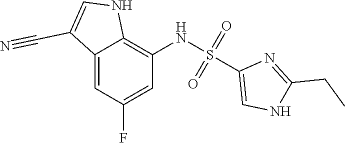 [SMILES: CCc1nc(S(=O)(=O)Nc2cc(F)cc3c(C#N)c[nH]c23)c[nH]1] |
| 158 | 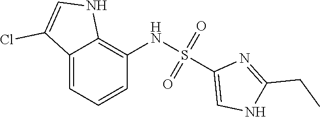 [SMILES: CCc1nc(S(=O)(=O)Nc2cccc3c(Cl)c[nH]c23)c[nH]1] |
| 159 | 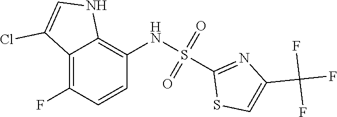 [SMILES: O=S(=O)(Nc1ccc(F)c2c(Cl)c[nH]c12)c1nc(C(F)(F)F)cs1] |
| 160 | 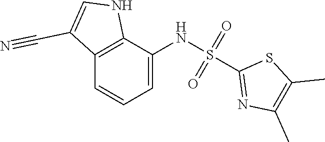 [SMILES: Cc1nc(S(=O)(=O)Nc2cccc3c(C#N)c[nH]c23)sc1C] |
| 161 | 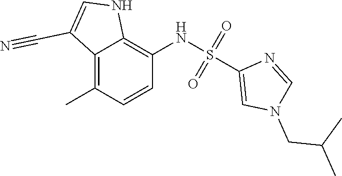 [SMILES: Cc1ccc(NS(=O)(=O)c2cn(CC(C)C)cn2)c2[nH]cc(C#N)c12] |
| 162 | 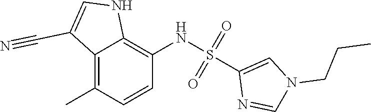 [SMILES: CCCn1cnc(S(=O)(=O)Nc2ccc(C)c3c(C#N)c[nH]c23)c1] |
| 163 | 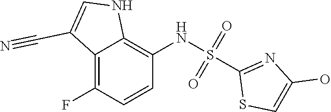 [SMILES: N#Cc1c[nH]c2c(NS(=O)(=O)c3nc(O)cs3)ccc(F)c12] |
| 164 | 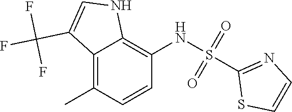 [SMILES: Cc1ccc(NS(=O)(=O)c2nccs2)c2[nH]cc(C(F)(F)F)c12] |
| 165 | 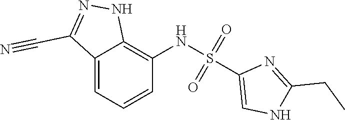 [SMILES: CCc1nc(S(=O)(=O)Nc2cccc3c(C#N)n[nH]c23)c[nH]1] |
| 166 | 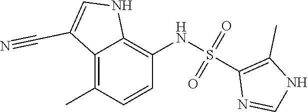 [SMILES: Cc1[nH]cnc1S(=O)(=O)Nc1ccc(C)c2c(C#N)c[nH]c12] |
| 167 | 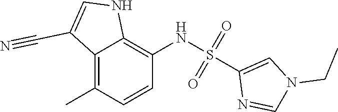 [SMILES: CCn1cnc(S(=O)(=O)Nc2ccc(C)c3c(C#N)c[nH]c23)c1] |
| 168 | 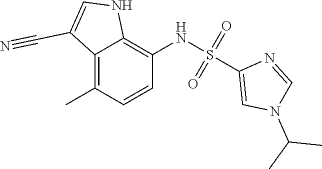 [SMILES: Cc1ccc(NS(=O)(=O)c2cn(C(C)C)cn2)c2[nH]cc(C#N)c12] |
| 169 | 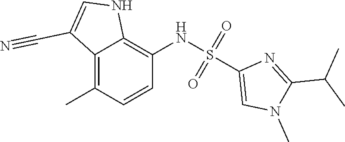 [SMILES: Cc1ccc(NS(=O)(=O)c2cn(C)c(C(C)C)n2)c2[nH]cc(C#N)c12] |
| 170 | 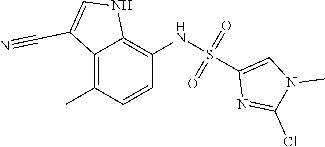 [SMILES: Cc1ccc(NS(=O)(=O)c2cn(C)c(Cl)n2)c2[nH]cc(C#N)c12] |
| 171 | 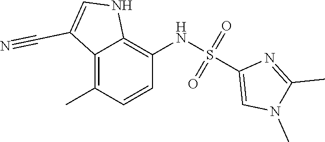 [SMILES: Cc1ccc(NS(=O)(=O)c2cn(C)c(C)n2)c2[nH]cc(C#N)c12] |
| 172 | 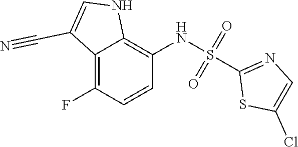 [SMILES: N#Cc1c[nH]c2c(NS(=O)(=O)c3ncc(Cl)s3)ccc(F)c12] |
| 173 | 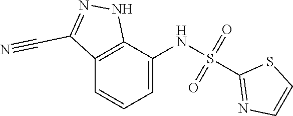 [SMILES: N#Cc1n[nH]c2c(NS(=O)(=O)c3nccs3)cccc12] |
| 174 | 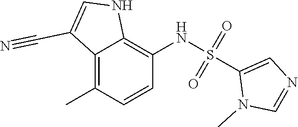 [SMILES: Cc1ccc(NS(=O)(=O)c2cncn2C)c2[nH]cc(C#N)c12] |
| 175 | 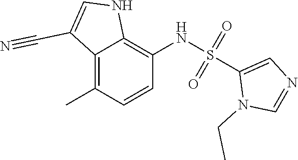 [SMILES: CCn1cncc1S(=O)(=O)Nc1ccc(C)c2c(C#N)c[nH]c12] |
| 176 | 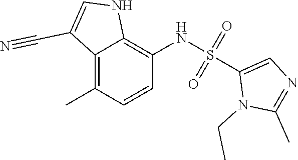 [SMILES: CCn1c(S(=O)(=O)Nc2ccc(C)c3c(C#N)c[nH]c23)cnc1C] |
| 177 | 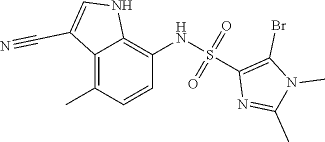 [SMILES: Cc1ccc(NS(=O)(=O)c2nc(C)n(C)c2Br)c2[nH]cc(C#N)c12] |
| 178 | 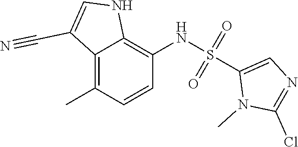 [SMILES: Cc1ccc(NS(=O)(=O)c2cnc(Cl)n2C)c2[nH]cc(C#N)c12] |
| 179 | 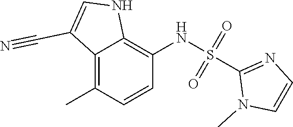 [SMILES: Cc1ccc(NS(=O)(=O)c2nccn2C)c2[nH]cc(C#N)c12] |
| 180 | 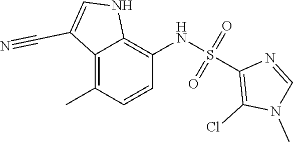 [SMILES: Cc1ccc(NS(=O)(=O)c2ncn(C)c2Cl)c2[nH]cc(C#N)c12] |
| 181 | 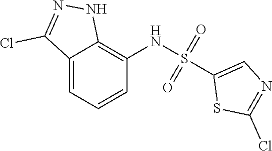 [SMILES: O=S(=O)(Nc1cccc2c(Cl)n[nH]c12)c1cnc(Cl)s1] |
| 182 | 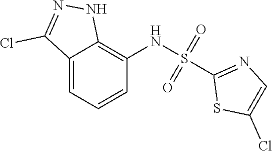 [SMILES: O=S(=O)(Nc1cccc2c(Cl)n[nH]c12)c1ncc(Cl)s1] |
| 183 | 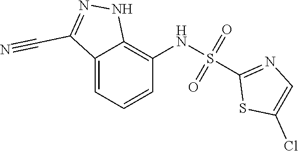 [SMILES: N#Cc1n[nH]c2c(NS(=O)(=O)c3ncc(Cl)s3)cccc12] |
| 184 | 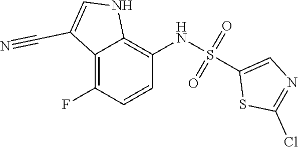 [SMILES: N#Cc1c[nH]c2c(NS(=O)(=O)c3cnc(Cl)s3)ccc(F)c12] |
| 185 | 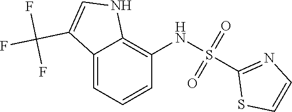 [SMILES: O=S(=O)(Nc1cccc2c(C(F)(F)F)c[nH]c12)c1nccs1] |
| 186 | 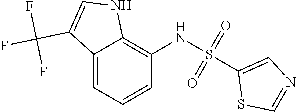 [SMILES: O=S(=O)(Nc1cccc2c(C(F)(F)F)c[nH]c12)c1cncs1] |
| 187 | 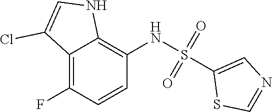 [SMILES: O=S(=O)(Nc1ccc(F)c2c(Cl)c[nH]c12)c1cncs1] |
| 188 | 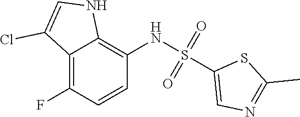 [SMILES: Cc1ncc(S(=O)(=O)Nc2ccc(F)c3c(Cl)c[nH]c23)s1] |
| 189 | 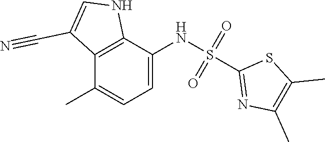 [SMILES: Cc1nc(S(=O)(=O)Nc2ccc(C)c3c(C#N)c[nH]c23)sc1C] |
| 190 | 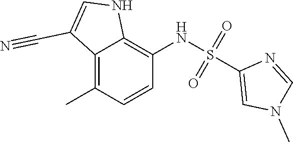 [SMILES: Cc1ccc(NS(=O)(=O)c2cn(C)cn2)c2[nH]cc(C#N)c12] |
| 191 | 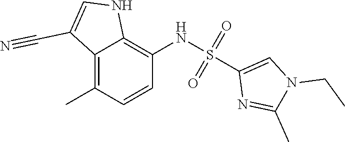 [SMILES: CCn1cc(S(=O)(=O)Nc2ccc(C)c3c(C#N)c[nH]c23)nc1C] |
| 192 | 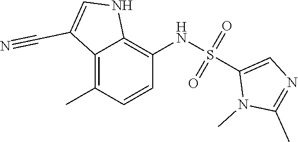 [SMILES: Cc1ccc(NS(=O)(=O)c2cnc(C)n2C)c2[nH]cc(C#N)c12] |
| 193 | 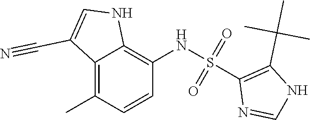 [SMILES: Cc1ccc(NS(=O)(=O)c2nc[nH]c2C(C)(C)C)c2[nH]cc(C#N)c12] |
| 194 | 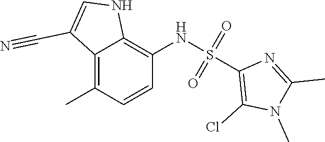 [SMILES: Cc1ccc(NS(=O)(=O)c2nc(C)n(C)c2Cl)c2[nH]cc(C#N)c12] |
| 195 | 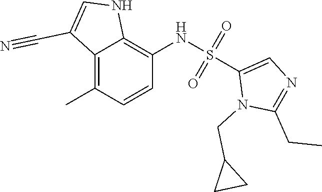 [SMILES: CCc1ncc(S(=O)(=O)Nc2ccc(C)c3c(C#N)c[nH]c23)n1CC1CC1] |
| 196 | 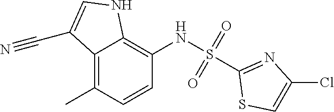 [SMILES: Cc1ccc(NS(=O)(=O)c2nc(Cl)cs2)c2[nH]cc(C#N)c12] |
| 197 | 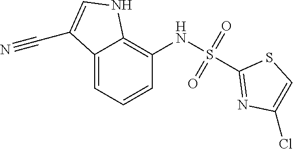 [SMILES: N#Cc1c[nH]c2c(NS(=O)(=O)c3nc(Cl)cs3)cccc12] |
| 198 | 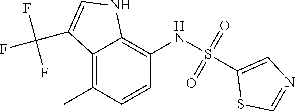 [SMILES: Cc1ccc(NS(=O)(=O)c2cncs2)c2[nH]cc(C(F)(F)F)c12] |
| 199 | 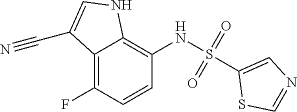 [SMILES: N#Cc1c[nH]c2c(NS(=O)(=O)c3cncs3)ccc(F)c12] |
| 200 | 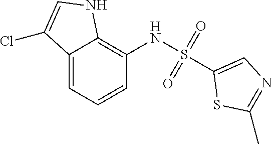 [SMILES: Cc1ncc(S(=O)(=O)Nc2cccc3c(Cl)c[nH]c23)s1] |
| 201 | 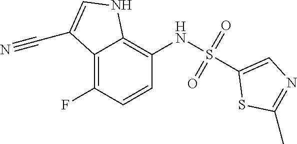 [SMILES: Cc1ncc(S(=O)(=O)Nc2ccc(F)c3c(C#N)c[nH]c23)s1] |
| 202 | 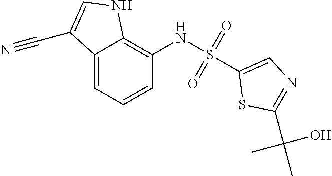 [SMILES: CC(C)(O)c1ncc(S(=O)(=O)Nc2cccc3c(C#N)c[nH]c23)s1] |
| 203 | 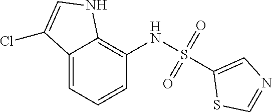 [SMILES: O=S(=O)(Nc1cccc2c(Cl)c[nH]c12)c1cncs1] |
| 204 | 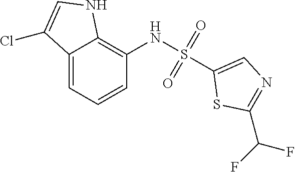 [SMILES: O=S(=O)(Nc1cccc2c(Cl)c[nH]c12)c1cnc(C(F)F)s1] |
| 205 | 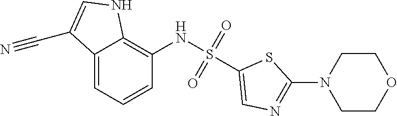 [SMILES: N#Cc1c[nH]c2c(NS(=O)(=O)c3cnc(N4CCOCC4)s3)cccc12] |
| 206 | 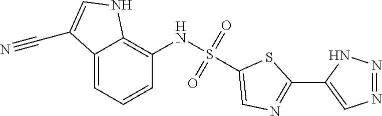 [SMILES: N#Cc1c[nH]c2c(NS(=O)(=O)c3cnc(-c4cnn[nH]4)s3)cccc12] |
| 207 | 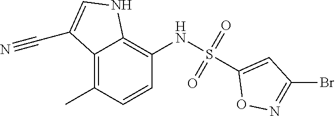 [SMILES: Cc1ccc(NS(=O)(=O)c2cc(Br)no2)c2[nH]cc(C#N)c12] |
| 208 | 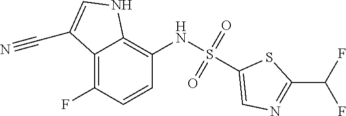 [SMILES: N#Cc1c[nH]c2c(NS(=O)(=O)c3cnc(C(F)F)s3)ccc(F)c12] |
| 209 | 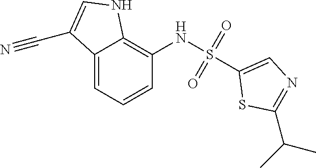 [SMILES: CC(C)c1ncc(S(=O)(=O)Nc2cccc3c(C#N)c[nH]c23)s1] |
| 210 | 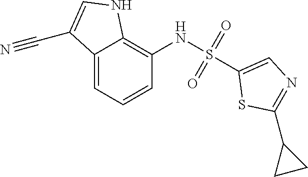 [SMILES: N#Cc1c[nH]c2c(NS(=O)(=O)c3cnc(C4CC4)s3)cccc12] |
| 211 | 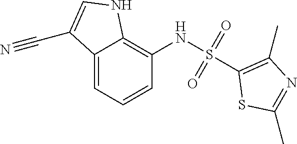 [SMILES: Cc1nc(C)c(S(=O)(=O)Nc2cccc3c(C#N)c[nH]c23)s1] |
| 212 | 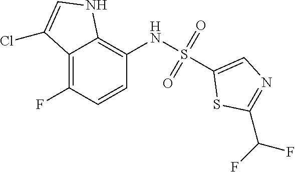 [SMILES: O=S(=O)(Nc1ccc(F)c2c(Cl)c[nH]c12)c1cnc(C(F)F)s1] |
| 213 | 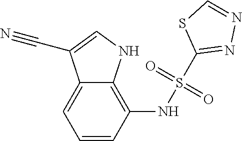 [SMILES: N#Cc1c[nH]c2c(NS(=O)(=O)c3nncs3)cccc12] |
| 214 | 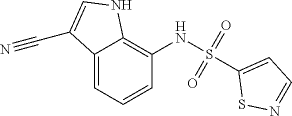 [SMILES: N#Cc1c[nH]c2c(NS(=O)(=O)c3ccns3)cccc12] |
| 215 | 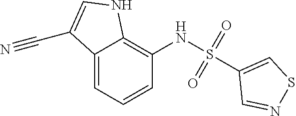 [SMILES: N#Cc1c[nH]c2c(NS(=O)(=O)c3cnsc3)cccc12] |
| 216 | 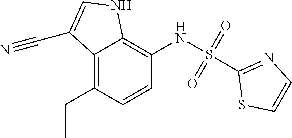 [SMILES: CCc1ccc(NS(=O)(=O)c2nccs2)c2[nH]cc(C#N)c12] |
| 217 | 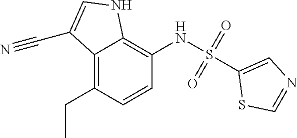 [SMILES: CCc1ccc(NS(=O)(=O)c2cncs2)c2[nH]cc(C#N)c12] |
| 218 | 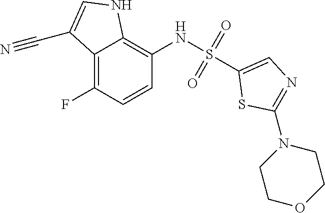 [SMILES: N#Cc1c[nH]c2c(NS(=O)(=O)c3cnc(N4CCOCC4)s3)ccc(F)c12] |
| 219 | 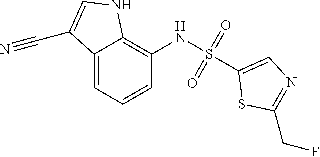 [SMILES: N#Cc1c[nH]c2c(NS(=O)(=O)c3cnc(CF)s3)cccc12] |
| 220 | 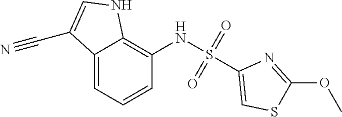 [SMILES: COc1nc(S(=O)(=O)Nc2cccc3c(C#N)c[nH]c23)cs1] |
| 221 | 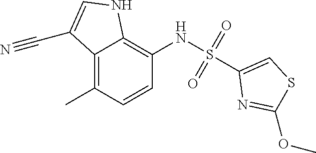 [SMILES: COc1nc(S(=O)(=O)Nc2ccc(C)c3c(C#N)c[nH]c23)cs1] |
| 222 | 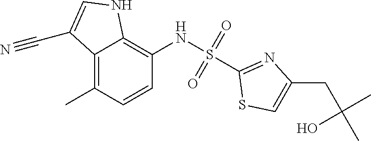 [SMILES: Cc1ccc(NS(=O)(=O)c2nc(CC(C)(C)O)cs2)c2[nH]cc(C#N)c12] |
| 223 | 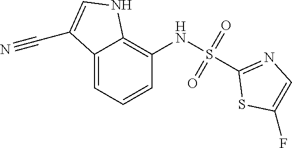 [SMILES: N#Cc1c[nH]c2c(NS(=O)(=O)c3ncc(F)s3)cccc12] |
| 224 | 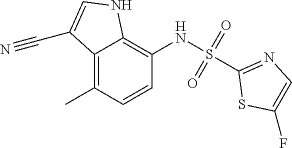 [SMILES: Cc1ccc(NS(=O)(=O)c2ncc(F)s2)c2[nH]cc(C#N)c12] |
| 225 | 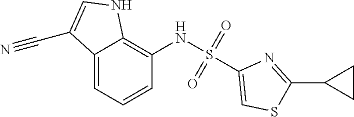 [SMILES: N#Cc1c[nH]c2c(NS(=O)(=O)c3csc(C4CC4)n3)cccc12] |
| 226 | 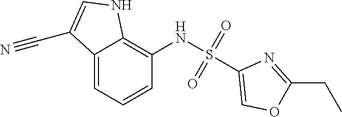 [SMILES: CCc1nc(S(=O)(=O)Nc2cccc3c(C#N)c[nH]c23)co1] |
| 227 | 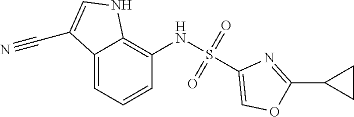 [SMILES: N#Cc1c[nH]c2c(NS(=O)(=O)c3coc(C4CC4)n3)cccc12] |
| 228 | 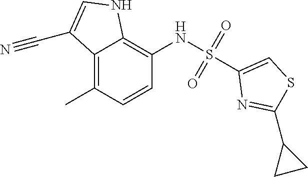 [SMILES: Cc1ccc(NS(=O)(=O)c2csc(C3CC3)n2)c2[nH]cc(C#N)c12] |
| 229 | 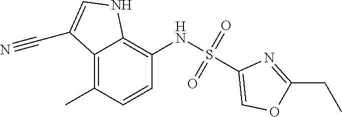 [SMILES: CCc1nc(S(=O)(=O)Nc2ccc(C)c3c(C#N)c[nH]c23)co1] |
| 230 | 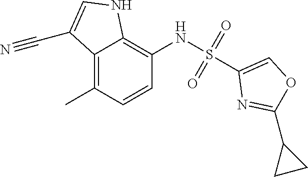 [SMILES: Cc1ccc(NS(=O)(=O)c2coc(C3CC3)n2)c2[nH]cc(C#N)c12] |
| 231 | 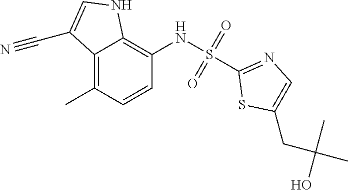 [SMILES: Cc1ccc(NS(=O)(=O)c2ncc(CC(C)(C)O)s2)c2[nH]cc(C#N)c12] |
| 232 | 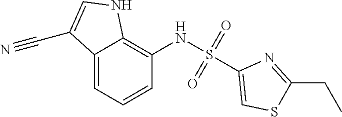 [SMILES: CCc1nc(S(=O)(=O)Nc2cccc3c(C#N)c[nH]c23)cs1] |
| 233 | 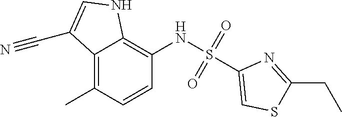 [SMILES: CCc1nc(S(=O)(=O)Nc2ccc(C)c3c(C#N)c[nH]c23)cs1] |
| 234 | 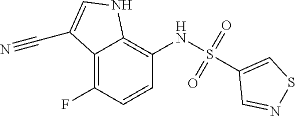 [SMILES: N#Cc1c[nH]c2c(NS(=O)(=O)c3cnsc3)ccc(F)c12] |
| 235 | 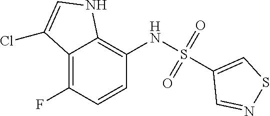 [SMILES: O=S(=O)(Nc1ccc(F)c2c(Cl)c[nH]c12)c1cnsc1] |
| 236 | 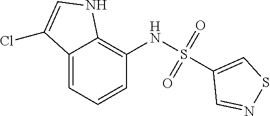 [SMILES: O=S(=O)(Nc1cccc2c(Cl)c[nH]c12)c1cnsc1] |
| 237 | 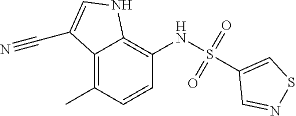 [SMILES: Cc1ccc(NS(=O)(=O)c2cnsc2)c2[nH]cc(C#N)c12] |
| 238 | 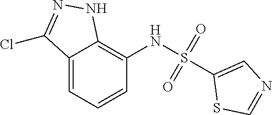 [SMILES: O=S(=O)(Nc1cccc2c(Cl)n[nH]c12)c1cncs1] |
| 239 | 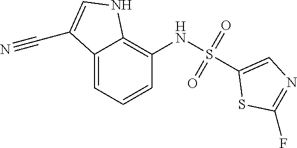 [SMILES: N#Cc1c[nH]c2c(NS(=O)(=O)c3cnc(F)s3)cccc12] |
| 240 | 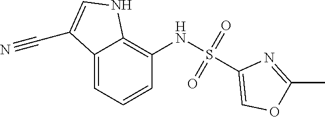 [SMILES: Cc1nc(S(=O)(=O)Nc2cccc3c(C#N)c[nH]c23)co1] |
| 241 | 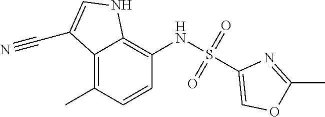 [SMILES: Cc1nc(S(=O)(=O)Nc2ccc(C)c3c(C#N)c[nH]c23)co1] |
| 242 | 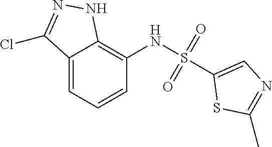 [SMILES: Cc1ncc(S(=O)(=O)Nc2cccc3c(Cl)n[nH]c23)s1] |
| 243 | 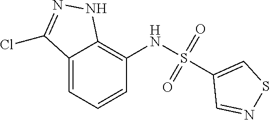 [SMILES: O=S(=O)(Nc1cccc2c(Cl)n[nH]c12)c1cnsc1] |
| 244 | 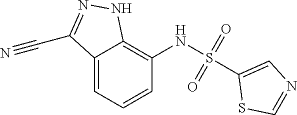 [SMILES: N#Cc1n[nH]c2c(NS(=O)(=O)c3cncs3)cccc12] |
| 245 | 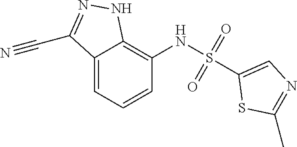 [SMILES: Cc1ncc(S(=O)(=O)Nc2cccc3c(C#N)n[nH]c23)s1] |
| 246 | 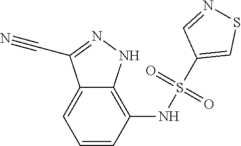 [SMILES: N#Cc1n[nH]c2c(NS(=O)(=O)c3cnsc3)cccc12] |
| 247 | 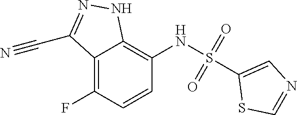 [SMILES: N#Cc1n[nH]c2c(NS(=O)(=O)c3cncs3)ccc(F)c12] |
| 248 | 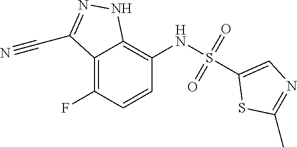 [SMILES: Cc1ncc(S(=O)(=O)Nc2ccc(F)c3c(C#N)n[nH]c23)s1] |
| 249 | 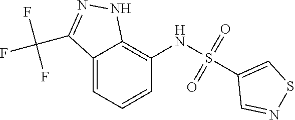 [SMILES: O=S(=O)(Nc1cccc2c(C(F)(F)F)n[nH]c12)c1cnsc1] |
| 250 | 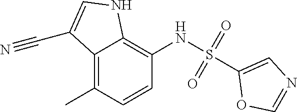 [SMILES: Cc1ccc(NS(=O)(=O)c2cnco2)c2[nH]cc(C#N)c12] |
| 251 | 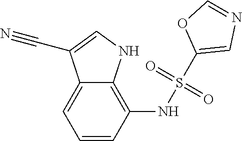 [SMILES: N#Cc1c[nH]c2c(NS(=O)(=O)c3cnco3)cccc12] |
| 252 | 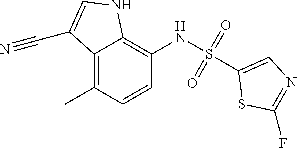 [SMILES: Cc1ccc(NS(=O)(=O)c2cnc(F)s2)c2[nH]cc(C#N)c12] |
| 253 | 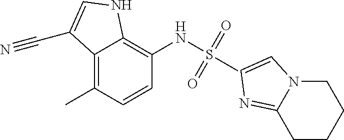 |
| 254 | 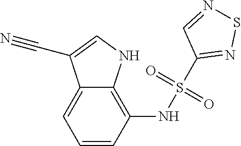 [SMILES: N#Cc1c[nH]c2c(NS(=O)(=O)c3cnsn3)cccc12] |
| 255 | 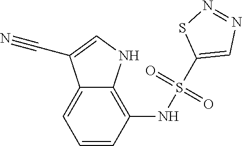 [SMILES: N#Cc1c[nH]c2c(NS(=O)(=O)c3cnns3)cccc12] |
| 256 | 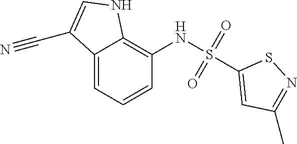 [SMILES: Cc1cc(S(=O)(=O)Nc2cccc3c(C#N)c[nH]c23)sn1] |
| 257 | 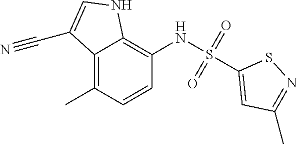 [SMILES: Cc1cc(S(=O)(=O)Nc2ccc(C)c3c(C#N)c[nH]c23)sn1] |
| 258 | 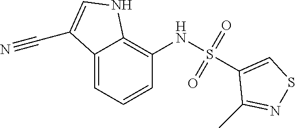 [SMILES: Cc1nscc1S(=O)(=O)Nc1cccc2c(C#N)c[nH]c12] |
| 259 | 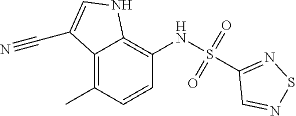 [SMILES: Cc1ccc(NS(=O)(=O)c2cnsn2)c2[nH]cc(C#N)c12] |
| 260 | 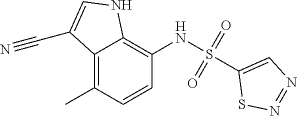 [SMILES: Cc1ccc(NS(=O)(=O)c2cnns2)c2[nH]cc(C#N)c12] |
| 261 | 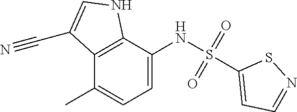 [SMILES: Cc1ccc(NS(=O)(=O)c2ccns2)c2[nH]cc(C#N)c12] |
| 262 | 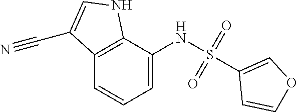 [SMILES: N#Cc1c[nH]c2c(NS(=O)(=O)c3ccoc3)cccc12] |
| 263 | 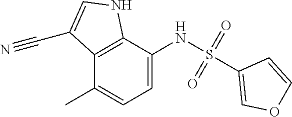 [SMILES: Cc1ccc(NS(=O)(=O)c2ccoc2)c2[nH]cc(C#N)c12] |
| 264 | 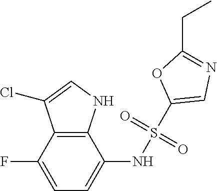 [SMILES: CCc1ncc(S(=O)(=O)Nc2ccc(F)c3c(Cl)c[nH]c23)o1] |
| 265 | 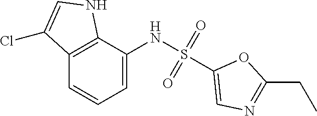 [SMILES: CCc1ncc(S(=O)(=O)Nc2cccc3c(Cl)c[nH]c23)o1] |
| 266 | 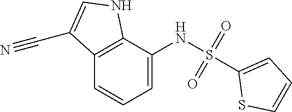 [SMILES: N#Cc1c[nH]c2c(NS(=O)(=O)c3cccs3)cccc12] |
| 267 | 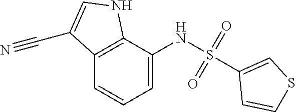 [SMILES: N#Cc1c[nH]c2c(NS(=O)(=O)c3ccsc3)cccc12] |
| 268 | 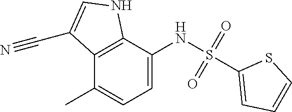 [SMILES: Cc1ccc(NS(=O)(=O)c2cccs2)c2[nH]cc(C#N)c12] |
| 269 | 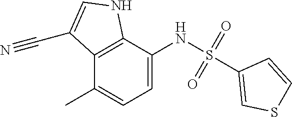 [SMILES: Cc1ccc(NS(=O)(=O)c2ccsc2)c2[nH]cc(C#N)c12] |
| 270 | 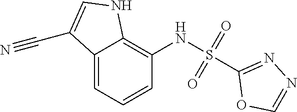 [SMILES: N#Cc1c[nH]c2c(NS(=O)(=O)c3nnco3)cccc12] |
| 271 | 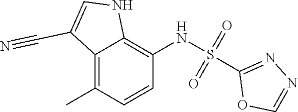 [SMILES: Cc1ccc(NS(=O)(=O)c2nnco2)c2[nH]cc(C#N)c12] |
| 272 | 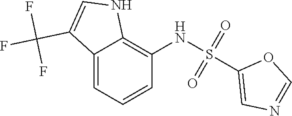 [SMILES: O=S(=O)(Nc1cccc2c(C(F)(F)F)c[nH]c12)c1cnco1] |
| 273 | 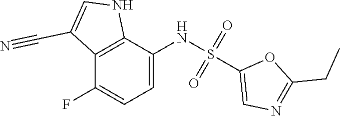 [SMILES: CCc1ncc(S(=O)(=O)Nc2ccc(F)c3c(C#N)c[nH]c23)o1] |
| 274 | 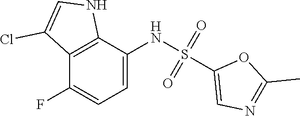 [SMILES: Cc1ncc(S(=O)(=O)Nc2ccc(F)c3c(Cl)c[nH]c23)o1] |
| 275 | 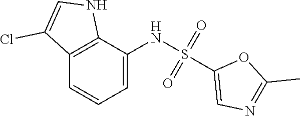 [SMILES: Cc1ncc(S(=O)(=O)Nc2cccc3c(Cl)c[nH]c23)o1] |
| 276 | 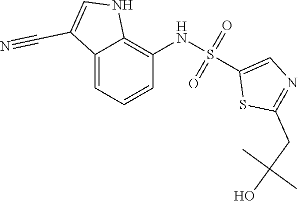 [SMILES: CC(C)(O)Cc1ncc(S(=O)(=O)Nc2cccc3c(C#N)c[nH]c23)s1] |
| 277 | 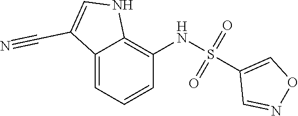 [SMILES: N#Cc1c[nH]c2c(NS(=O)(=O)c3cnoc3)cccc12] |
| 278 | 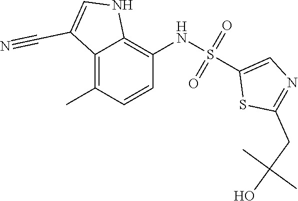 [SMILES: Cc1ccc(NS(=O)(=O)c2cnc(CC(C)(C)O)s2)c2[nH]cc(C#N)c12] |
| 279 | 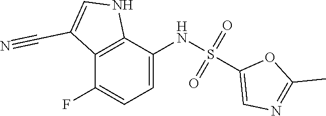 [SMILES: Cc1ncc(S(=O)(=O)Nc2ccc(F)c3c(C#N)c[nH]c23)o1] |
| 280 | 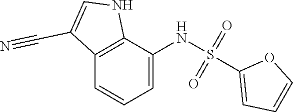 [SMILES: N#Cc1c[nH]c2c(NS(=O)(=O)c3ccco3)cccc12] |
| 281 | 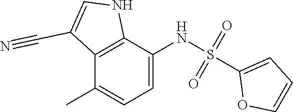 [SMILES: Cc1ccc(NS(=O)(=O)c2ccco2)c2[nH]cc(C#N)c12] |
| 282 | 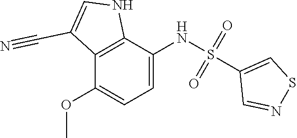 [SMILES: COc1ccc(NS(=O)(=O)c2cnsc2)c2[nH]cc(C#N)c12] |
| 283 | 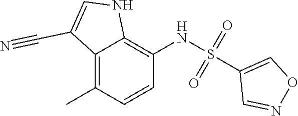 [SMILES: Cc1ccc(NS(=O)(=O)c2cnoc2)c2[nH]cc(C#N)c12] |
| 284 | 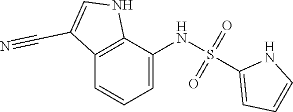 [SMILES: N#Cc1c[nH]c2c(NS(=O)(=O)c3ccc[nH]3)cccc12] |
| 285 | 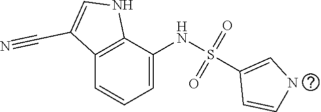 [SMILES: N#Cc1c[nH]c2c(NS(=O)(=O)c3cc[nH]c3)cccc12] |
| 286 | 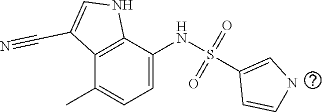 [SMILES: Cc1ccc(NS(=O)(=O)c2cc[nH]c2)c2[nH]cc(C#N)c12] |
| 287 | 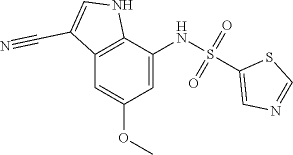 [SMILES: COc1cc(NS(=O)(=O)c2cncs2)c2[nH]cc(C#N)c2c1] |
| 288 | 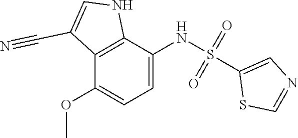 [SMILES: COc1ccc(NS(=O)(=O)c2cncs2)c2[nH]cc(C#N)c12] |
| 289 | 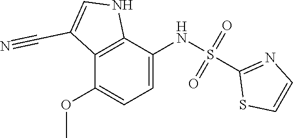 [SMILES: COc1ccc(NS(=O)(=O)c2nccs2)c2[nH]cc(C#N)c12] |
| 290 | 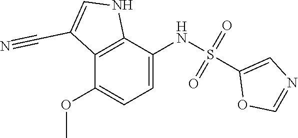 [SMILES: COc1ccc(NS(=O)(=O)c2cnco2)c2[nH]cc(C#N)c12] |
| 291 | 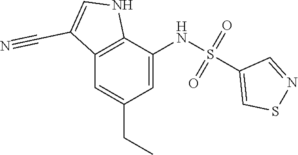 [SMILES: CCc1cc(NS(=O)(=O)c2cnsc2)c2[nH]cc(C#N)c2c1] |
| 292 | 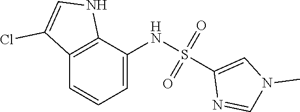 [SMILES: Cn1cnc(S(=O)(=O)Nc2cccc3c(Cl)c[nH]c23)c1] |
| 293 | 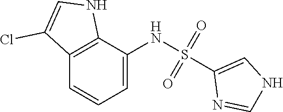 [SMILES: O=S(=O)(Nc1cccc2c(Cl)c[nH]c12)c1c[nH]cn1] |
| 294 | 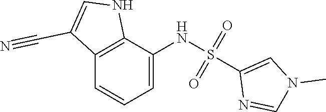 [SMILES: Cn1cnc(S(=O)(=O)Nc2cccc3c(C#N)c[nH]c23)c1] |
| 295 | 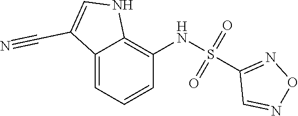 [SMILES: N#Cc1c[nH]c2c(NS(=O)(=O)c3cnon3)cccc12] |
| 296 | 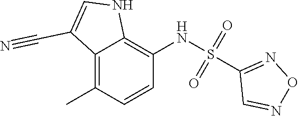 [SMILES: Cc1ccc(NS(=O)(=O)c2cnon2)c2[nH]cc(C#N)c12] |
| 297 | 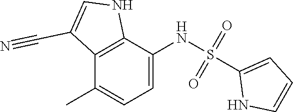 [SMILES: Cc1ccc(NS(=O)(=O)c2ccc[nH]2)c2[nH]cc(C#N)c12] |
| 298 | 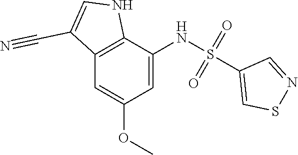 [SMILES: COc1cc(NS(=O)(=O)c2cnsc2)c2[nH]cc(C#N)c2c1] |
| 299 | 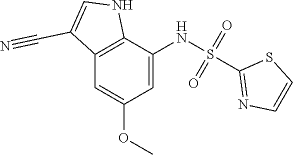 [SMILES: COc1cc(NS(=O)(=O)c2nccs2)c2[nH]cc(C#N)c2c1] |
| 300 | 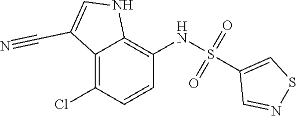 [SMILES: N#Cc1c[nH]c2c(NS(=O)(=O)c3cnsc3)ccc(Cl)c12] |
| 301 | 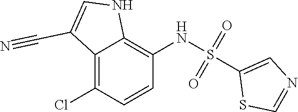 [SMILES: N#Cc1c[nH]c2c(NS(=O)(=O)c3cncs3)ccc(Cl)c12] |
| 302 | 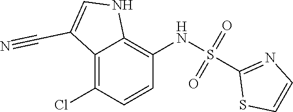 [SMILES: N#Cc1c[nH]c2c(NS(=O)(=O)c3nccs3)ccc(Cl)c12] |
| 303 | 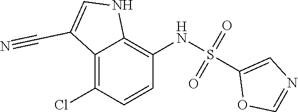 [SMILES: N#Cc1c[nH]c2c(NS(=O)(=O)c3cnco3)ccc(Cl)c12] |
| 304 | 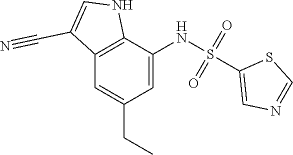 [SMILES: CCc1cc(NS(=O)(=O)c2cncs2)c2[nH]cc(C#N)c2c1] |
| 305 | 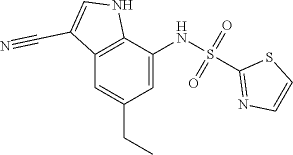 [SMILES: CCc1cc(NS(=O)(=O)c2nccs2)c2[nH]cc(C#N)c2c1] |
| 306 | 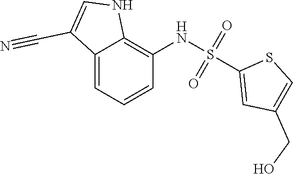 [SMILES: N#Cc1c[nH]c2c(NS(=O)(=O)c3cc(CO)cs3)cccc12] |
| 307 | 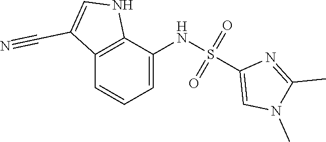 [SMILES: Cc1nc(S(=O)(=O)Nc2cccc3c(C#N)c[nH]c23)cn1C] |
| 308 | 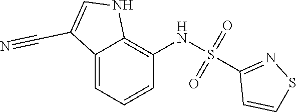 [SMILES: N#Cc1c[nH]c2c(NS(=O)(=O)c3ccsn3)cccc12] |
| 309 | 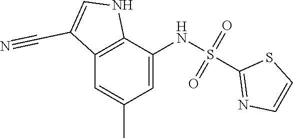 [SMILES: Cc1cc(NS(=O)(=O)c2nccs2)c2[nH]cc(C#N)c2c1] |
| 310 | 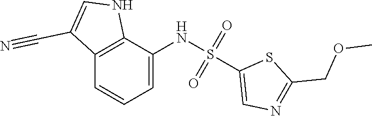 [SMILES: COCc1ncc(S(=O)(=O)Nc2cccc3c(C#N)c[nH]c23)s1] |
| 311 | 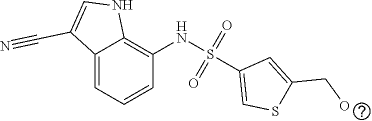 [SMILES: N#Cc1c[nH]c2c(NS(=O)(=O)c3csc(CO)c3)cccc12] |
| 312 | 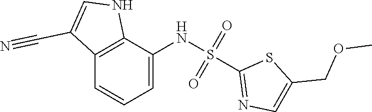 [SMILES: COCc1cnc(S(=O)(=O)Nc2cccc3c(C#N)c[nH]c23)s1] |
| 313 | 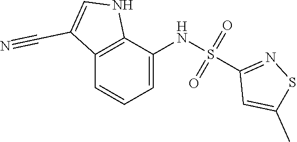 [SMILES: Cc1cc(S(=O)(=O)Nc2cccc3c(C#N)c[nH]c23)ns1] |
| 314 | 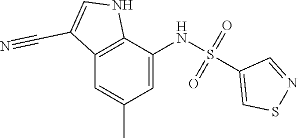 [SMILES: Cc1cc(NS(=O)(=O)c2cnsc2)c2[nH]cc(C#N)c2c1] |
| 315 | 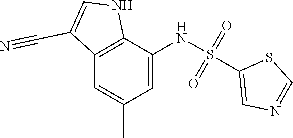 [SMILES: Cc1cc(NS(=O)(=O)c2cncs2)c2[nH]cc(C#N)c2c1] |
| 316 | 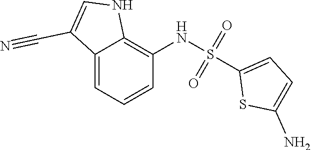 [SMILES: N#Cc1c[nH]c2c(NS(=O)(=O)c3ccc(N)s3)cccc12] |
| 317 | 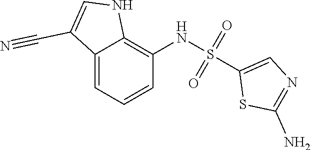 [SMILES: N#Cc1c[nH]c2c(NS(=O)(=O)c3cnc(N)s3)cccc12] |
| 318 | 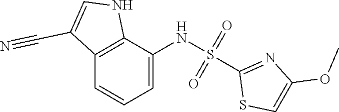 [SMILES: COc1csc(S(=O)(=O)Nc2cccc3c(C#N)c[nH]c23)n1] |
| 319 | 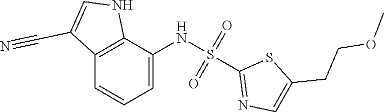 [SMILES: COCCc1cnc(S(=O)(=O)Nc2cccc3c(C#N)c[nH]c23)s1] |
| 320 | 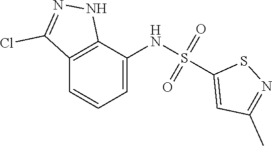 [SMILES: Cc1cc(S(=O)(=O)Nc2cccc3c(Cl)n[nH]c23)sn1] |
| 321 | 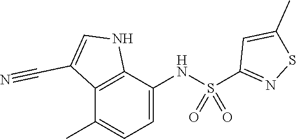 [SMILES: Cc1cc(S(=O)(=O)Nc2ccc(C)c3c(C#N)c[nH]c23)ns1] |
| 322 | 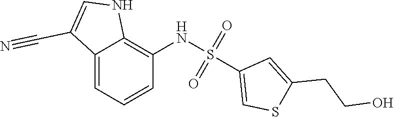 [SMILES: N#Cc1c[nH]c2c(NS(=O)(=O)c3csc(CCO)c3)cccc12] |
| 323 | 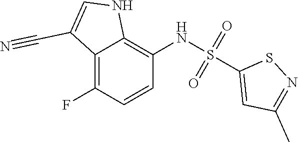 [SMILES: Cc1cc(S(=O)(=O)Nc2ccc(F)c3c(C#N)c[nH]c23)sn1] |
| 324 | 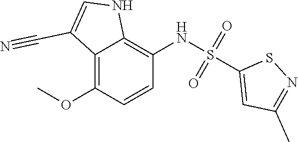 [SMILES: COc1ccc(NS(=O)(=O)c2cc(C)ns2)c2[nH]cc(C#N)c12] |
| 325 | 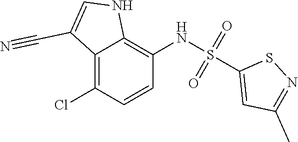 [SMILES: Cc1cc(S(=O)(=O)Nc2ccc(Cl)c3c(C#N)c[nH]c23)sn1] |
| 326 | 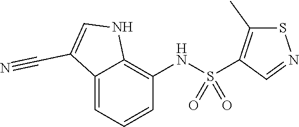 [SMILES: Cc1sncc1S(=O)(=O)Nc1cccc2c(C#N)c[nH]c12] |
| 327 | 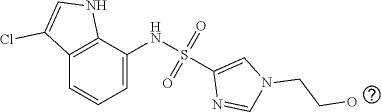 [SMILES: O=S(=O)(Nc1cccc2c(Cl)c[nH]c12)c1cn(CCO)cn1] |
| 328 | 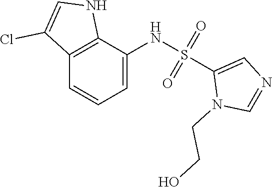 [SMILES: O=S(=O)(Nc1cccc2c(Cl)c[nH]c12)c1cncn1CCO] |
| 329 | 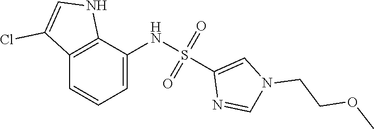 [SMILES: COCCn1cnc(S(=O)(=O)Nc2cccc3c(Cl)c[nH]c23)c1] |
| 330 | 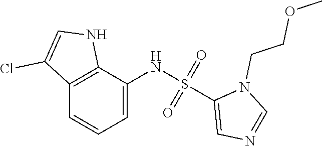 [SMILES: COCCn1cncc1S(=O)(=O)Nc1cccc2c(Cl)c[nH]c12] |
| 331 | 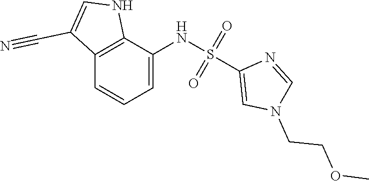 [SMILES: COCCn1cnc(S(=O)(=O)Nc2cccc3c(C#N)c[nH]c23)c1] |
| 332 | 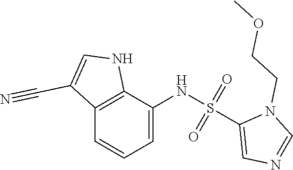 [SMILES: COCCn1cncc1S(=O)(=O)Nc1cccc2c(C#N)c[nH]c12] |
| 333 | 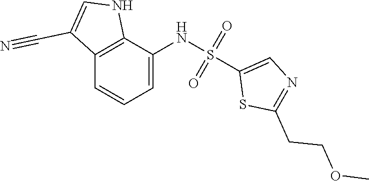 [SMILES: COCCc1ncc(S(=O)(=O)Nc2cccc3c(C#N)c[nH]c23)s1] |
| 334 | 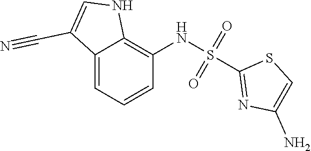 [SMILES: N#Cc1c[nH]c2c(NS(=O)(=O)c3nc(N)cs3)cccc12] |
| 335 | 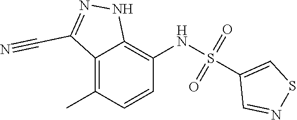 [SMILES: Cc1ccc(NS(=O)(=O)c2cnsc2)c2[nH]nc(C#N)c12] |
| 336 | 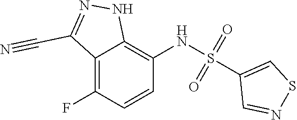 [SMILES: N#Cc1n[nH]c2c(NS(=O)(=O)c3cnsc3)ccc(F)c12] |
| 337 | 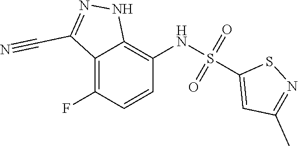 [SMILES: Cc1cc(S(=O)(=O)Nc2ccc(F)c3c(C#N)n[nH]c23)sn1] |
| 338 | 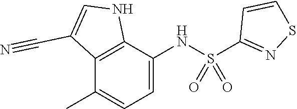 [SMILES: Cc1ccc(NS(=O)(=O)c2ccsn2)c2[nH]cc(C#N)c12] |
| 339 | 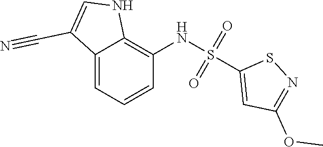 [SMILES: COc1cc(S(=O)(=O)Nc2cccc3c(C#N)c[nH]c23)sn1] |
| 340 | 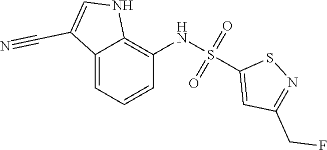 [SMILES: N#Cc1c[nH]c2c(NS(=O)(=O)c3cc(CF)ns3)cccc12] |
| 341 | 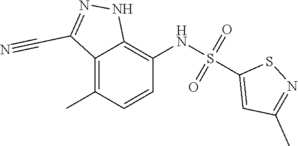 [SMILES: Cc1cc(S(=O)(=O)Nc2ccc(C)c3c(C#N)n[nH]c23)sn1] |
| 342 | 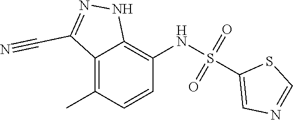 [SMILES: Cc1ccc(NS(=O)(=O)c2cncs2)c2[nH]nc(C#N)c12] |
| 343 | 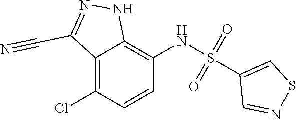 [SMILES: N#Cc1n[nH]c2c(NS(=O)(=O)c3cnsc3)ccc(Cl)c12] |
| 344 | 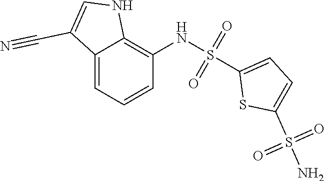 [SMILES: N#Cc1c[nH]c2c(NS(=O)(=O)c3ccc(S(N)(=O)=O)s3)cccc12] |
| 345 | 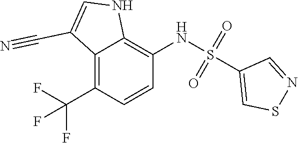 [SMILES: N#Cc1c[nH]c2c(NS(=O)(=O)c3cnsc3)ccc(C(F)(F)F)c12] |
| 346 | 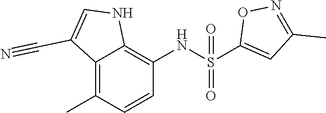 [SMILES: Cc1cc(S(=O)(=O)Nc2ccc(C)c3c(C#N)c[nH]c23)on1] |
| 347 | 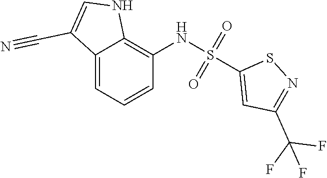 [SMILES: N#Cc1c[nH]c2c(NS(=O)(=O)c3cc(C(F)(F)F)ns3)cccc12] |
| 348 | 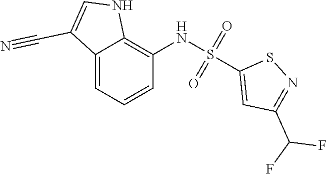 [SMILES: N#Cc1c[nH]c2c(NS(=O)(=O)c3cc(C(F)F)ns3)cccc12] |
| 349 | 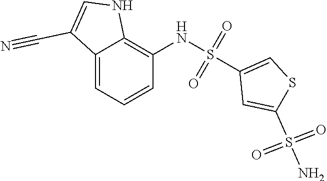 [SMILES: N#Cc1c[nH]c2c(NS(=O)(=O)c3csc(S(N)(=O)=O)c3)cccc12] |
| 350 | 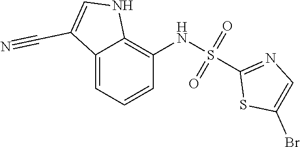 [SMILES: N#Cc1c[nH]c2c(NS(=O)(=O)c3ncc(Br)s3)cccc12] |
| 351 | 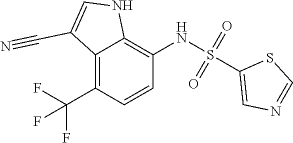 [SMILES: N#Cc1c[nH]c2c(NS(=O)(=O)c3cncs3)ccc(C(F)(F)F)c12] |
| 352 | 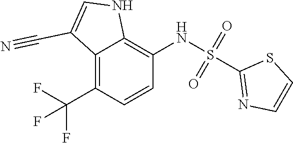 [SMILES: N#Cc1c[nH]c2c(NS(=O)(=O)c3nccs3)ccc(C(F)(F)F)c12] |
| 353 | 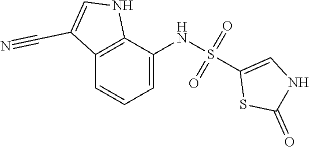 [SMILES: N#Cc1c[nH]c2c(NS(=O)(=O)c3c[nH]c(=O)s3)cccc12] |
| 354 | 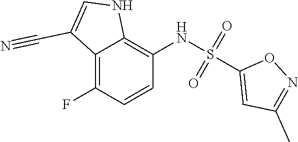 [SMILES: Cc1cc(S(=O)(=O)Nc2ccc(F)c3c(C#N)c[nH]c23)on1] |
| 355 | 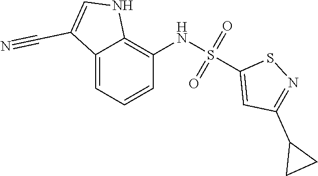 [SMILES: N#Cc1c[nH]c2c(NS(=O)(=O)c3cc(C4CC4)ns3)cccc12] |
| 356 | 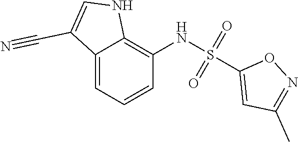 [SMILES: Cc1cc(S(=O)(=O)Nc2cccc3c(C#N)c[nH]c23)on1] |
| 357 | 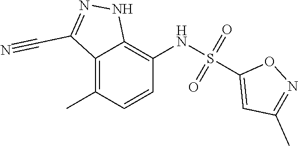 [SMILES: Cc1cc(S(=O)(=O)Nc2ccc(C)c3c(C#N)n[nH]c23)on1] |
| 358 | 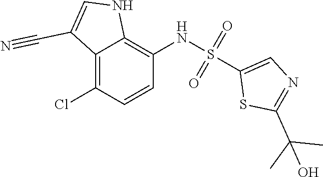 [SMILES: CC(C)(O)c1ncc(S(=O)(=O)Nc2ccc(Cl)c3c(C#N)c[nH]c23)s1] |
| 359 | 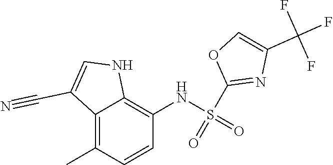 [SMILES: Cc1ccc(NS(=O)(=O)c2nc(C(F)(F)F)co2)c2[nH]cc(C#N)c12] |
| 360 | 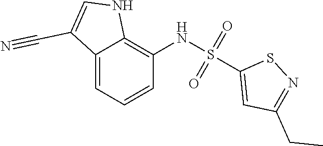 [SMILES: CCc1cc(S(=O)(=O)Nc2cccc3c(C#N)c[nH]c23)sn1] |
| 361 | 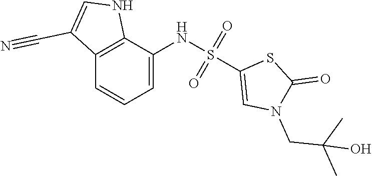 [SMILES: CC(C)(O)Cn1cc(S(=O)(=O)Nc2cccc3c(C#N)c[nH]c23)sc1=O] |
| 362 | 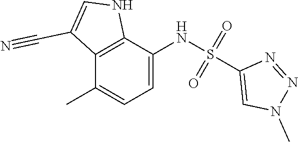 [SMILES: Cc1ccc(NS(=O)(=O)c2cn(C)nn2)c2[nH]cc(C#N)c12] |
| 363 | 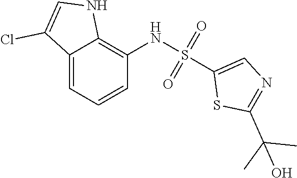 [SMILES: CC(C)(O)c1ncc(S(=O)(=O)Nc2cccc3c(Cl)c[nH]c23)s1] |
| 364 | 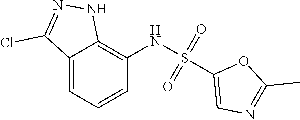 [SMILES: Cc1ncc(S(=O)(=O)Nc2cccc3c(Cl)n[nH]c23)o1] |
| 365 | 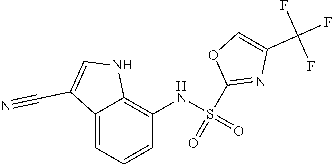 [SMILES: N#Cc1c[nH]c2c(NS(=O)(=O)c3nc(C(F)(F)F)co3)cccc12] |
| 366 | 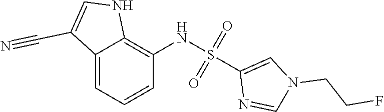 [SMILES: N#Cc1c[nH]c2c(NS(=O)(=O)c3cn(CCF)cn3)cccc12] |
| 367 | 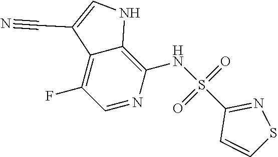 [SMILES: N#Cc1c[nH]c2c(NS(=O)(=O)c3ccsn3)ncc(F)c12] |
| 368 | 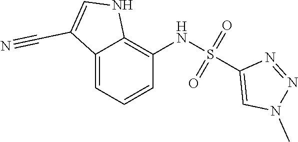 [SMILES: Cn1cc(S(=O)(=O)Nc2cccc3c(C#N)c[nH]c23)nn1] |
| 369 | 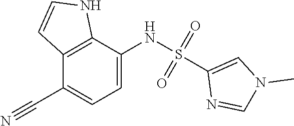 [SMILES: Cn1cnc(S(=O)(=O)Nc2ccc(C#N)c3cc[nH]c23)c1] |
| 370 | 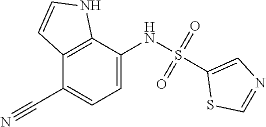 [SMILES: N#Cc1ccc(NS(=O)(=O)c2cncs2)c2[nH]ccc12] |
| 371 | 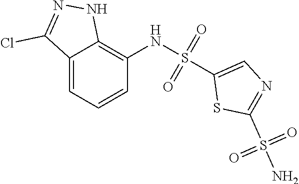 [SMILES: NS(=O)(=O)c1ncc(S(=O)(=O)Nc2cccc3c(Cl)n[nH]c23)s1] |
| 372 | 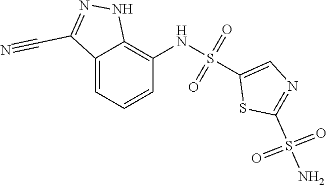 [SMILES: N#Cc1n[nH]c2c(NS(=O)(=O)c3cnc(S(N)(=O)=O)s3)cccc12] |
| 373 | 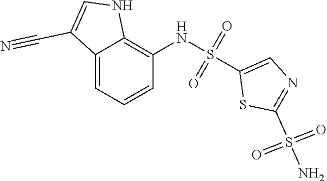 [SMILES: N#Cc1c[nH]c2c(NS(=O)(=O)c3cnc(S(N)(=O)=O)s3)cccc12] |
| 374 | 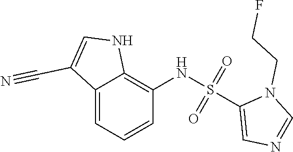 [SMILES: N#Cc1c[nH]c2c(NS(=O)(=O)c3cncn3CCF)cccc12] |
| 375 | 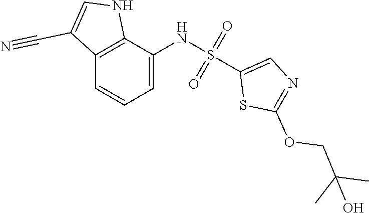 [SMILES: CC(C)(O)COc1ncc(S(=O)(=O)Nc2cccc3c(C#N)c[nH]c23)s1] |
| 376 | 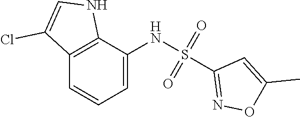 [SMILES: Cc1cc(S(=O)(=O)Nc2cccc3c(Cl)c[nH]c23)no1] |
| 377 | 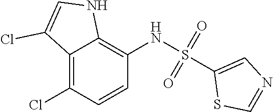 [SMILES: O=S(=O)(Nc1ccc(Cl)c2c(Cl)c[nH]c12)c1cncs1] |
| 378 | 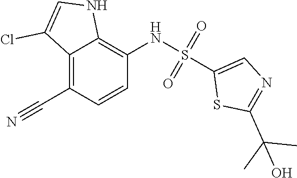 [SMILES: CC(C)(O)c1ncc(S(=O)(=O)Nc2ccc(C#N)c3c(Cl)c[nH]c23)s1] |
| 379 | 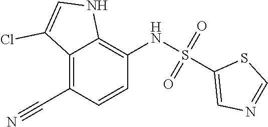 [SMILES: N#Cc1ccc(NS(=O)(=O)c2cncs2)c2[nH]cc(Cl)c12] |
| 380 | 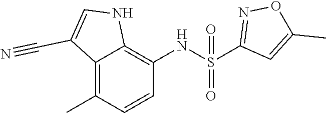 [SMILES: Cc1cc(S(=O)(=O)Nc2ccc(C)c3c(C#N)c[nH]c23)no1] |
| 381 | 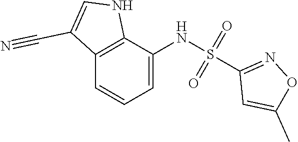 [SMILES: Cc1cc(S(=O)(=O)Nc2cccc3c(C#N)c[nH]c23)no1] |
| 382 | 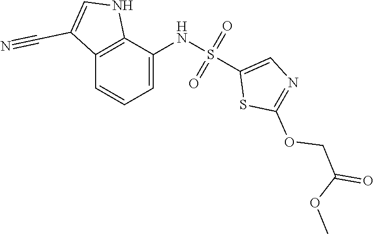 [SMILES: COC(=O)COc1ncc(S(=O)(=O)Nc2cccc3c(C#N)c[nH]c23)s1] |
| 383 | 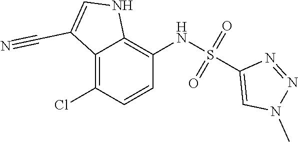 [SMILES: Cn1cc(S(=O)(=O)Nc2ccc(Cl)c3c(C#N)c[nH]c23)nn1] |
| 384 | 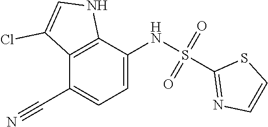 [SMILES: N#Cc1ccc(NS(=O)(=O)c2nccs2)c2[nH]cc(Cl)c12] |
| 385 | 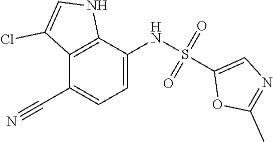 [SMILES: Cc1ncc(S(=O)(=O)Nc2ccc(C#N)c3c(Cl)c[nH]c23)o1] |
| 386 | 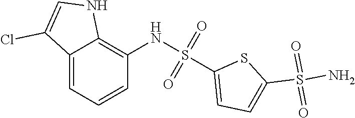 [SMILES: NS(=O)(=O)c1ccc(S(=O)(=O)Nc2cccc3c(Cl)c[nH]c23)s1] |
| 387 | 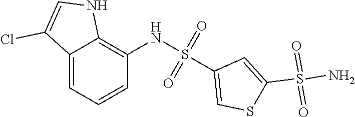 [SMILES: NS(=O)(=O)c1cc(S(=O)(=O)Nc2cccc3c(Cl)c[nH]c23)cs1] |
| 388 | 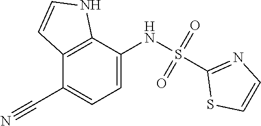 [SMILES: N#Cc1ccc(NS(=O)(=O)c2nccs2)c2[nH]ccc12] |
| 389 | 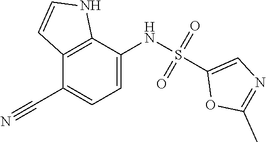 [SMILES: Cc1ncc(S(=O)(=O)Nc2ccc(C#N)c3cc[nH]c23)o1] |
| 390 | 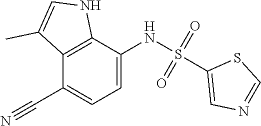 [SMILES: Cc1c[nH]c2c(NS(=O)(=O)c3cncs3)ccc(C#N)c12] |
| 391 | 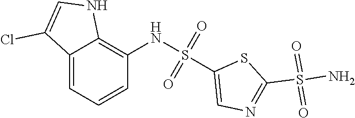 [SMILES: NS(=O)(=O)c1ncc(S(=O)(=O)Nc2cccc3c(Cl)c[nH]c23)s1] |
| 392 | 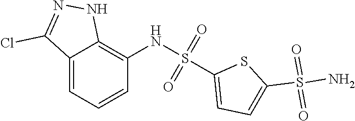 [SMILES: NS(=O)(=O)c1ccc(S(=O)(=O)Nc2cccc3c(Cl)n[nH]c23)s1] |
| 393 | 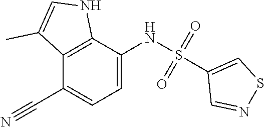 [SMILES: Cc1c[nH]c2c(NS(=O)(=O)c3cnsc3)ccc(C#N)c12] |
| 394 | 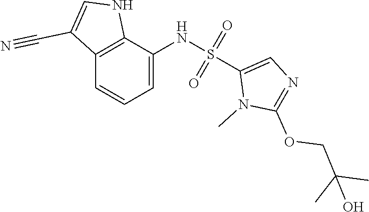 [SMILES: Cn1c(S(=O)(=O)Nc2cccc3c(C#N)c[nH]c23)cnc1OCC(C)(C)O] |
| 395 | 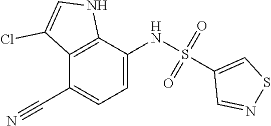 [SMILES: N#Cc1ccc(NS(=O)(=O)c2cnsc2)c2[nH]cc(Cl)c12] |
| 396 | 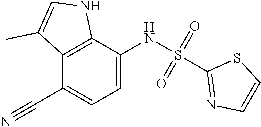 [SMILES: Cc1c[nH]c2c(NS(=O)(=O)c3nccs3)ccc(C#N)c12] |
| 397 | 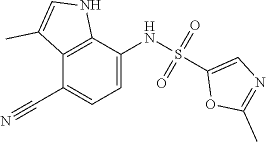 [SMILES: Cc1ncc(S(=O)(=O)Nc2ccc(C#N)c3c(C)c[nH]c23)o1] |
| 398 | 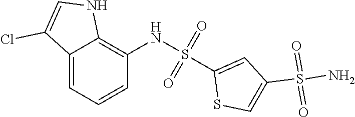 [SMILES: NS(=O)(=O)c1csc(S(=O)(=O)Nc2cccc3c(Cl)c[nH]c23)c1] |
| 399 | 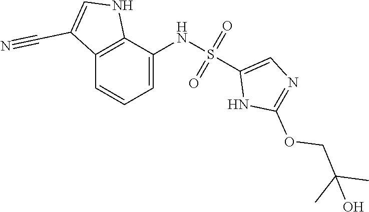 [SMILES: CC(C)(O)COc1ncc(S(=O)(=O)Nc2cccc3c(C#N)c[nH]c23)[nH]1] |
| 400 | 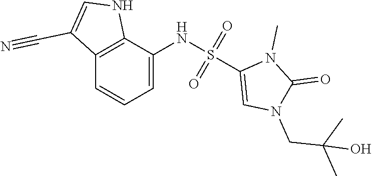 [SMILES: Cn1c(S(=O)(=O)Nc2cccc3c(C#N)c[nH]c23)cn(CC(C)(C)O)c1=O] |
| 401 | 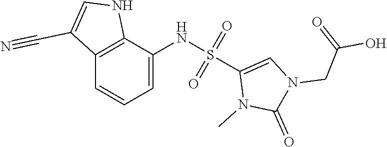 [SMILES: Cn1c(S(=O)(=O)Nc2cccc3c(C#N)c[nH]c23)cn(CC(=O)O)c1=O] |
| 402 | 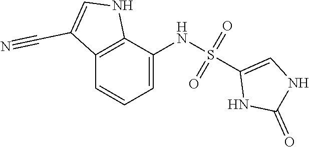 [SMILES: N#Cc1c[nH]c2c(NS(=O)(=O)c3c[nH]c(=O)[nH]3)cccc12] |
| 403 | 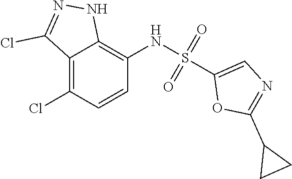 [SMILES: O=S(=O)(Nc1ccc(Cl)c2c(Cl)n[nH]c12)c1cnc(C2CC2)o1] |
| 404 | 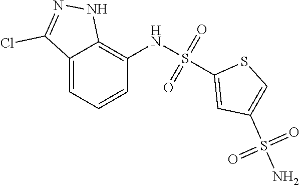 [SMILES: NS(=O)(=O)c1csc(S(=O)(=O)Nc2cccc3c(Cl)n[nH]c23)c1] |
| 405 | 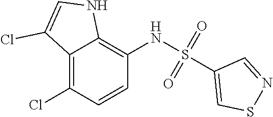 [SMILES: O=S(=O)(Nc1ccc(Cl)c2c(Cl)c[nH]c12)c1cnsc1] |
| 406 | 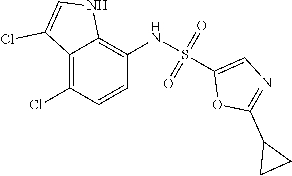 [SMILES: O=S(=O)(Nc1ccc(Cl)c2c(Cl)c[nH]c12)c1cnc(C2CC2)o1] |
| 407 | 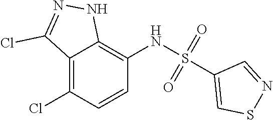 [SMILES: O=S(=O)(Nc1ccc(Cl)c2c(Cl)n[nH]c12)c1cnsc1] |
| 408 | 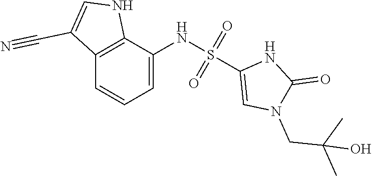 [SMILES: CC(C)(O)Cn1cc(S(=O)(=O)Nc2cccc3c(C#N)c[nH]c23)[nH]c1=O] |
| 409 | 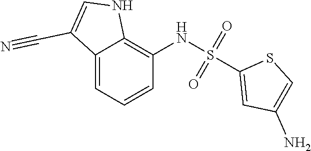 [SMILES: N#Cc1c[nH]c2c(NS(=O)(=O)c3cc(N)cs3)cccc12] |
| 410 | 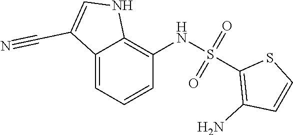 [SMILES: N#Cc1c[nH]c2c(NS(=O)(=O)c3sccc3N)cccc12] |
| 411 | 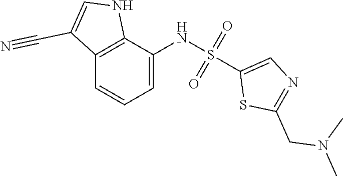 [SMILES: CN(C)Cc1ncc(S(=O)(=O)Nc2cccc3c(C#N)c[nH]c23)s1] |
| 412 | 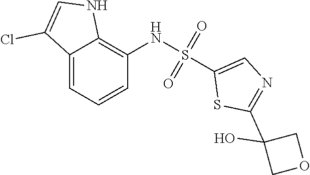 [SMILES: O=S(=O)(Nc1cccc2c(Cl)c[nH]c12)c1cnc(C2(O)COC2)s1] |
| 413 | 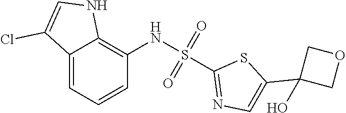 [SMILES: O=S(=O)(Nc1cccc2c(Cl)c[nH]c12)c1ncc(C2(O)COC2)s1] |
| 414 | 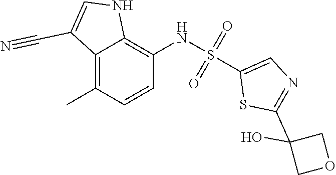 [SMILES: Cc1ccc(NS(=O)(=O)c2cnc(C3(O)COC3)s2)c2[nH]cc(C#N)c12] |
| 415 | 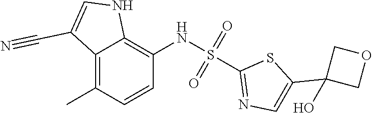 [SMILES: Cc1ccc(NS(=O)(=O)c2ncc(C3(O)COC3)s2)c2[nH]cc(C#N)c12] |
| 416 | 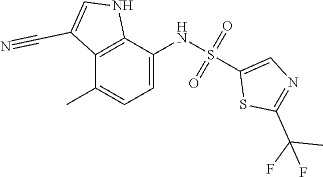 [SMILES: Cc1ccc(NS(=O)(=O)c2cnc(C(C)(F)F)s2)c2[nH]cc(C#N)c12] |
| 417 | 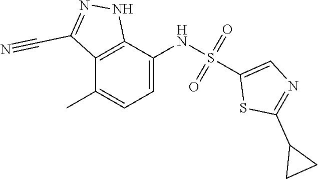 [SMILES: Cc1ccc(NS(=O)(=O)c2cnc(C3CC3)s2)c2[nH]nc(C#N)c12] |
| 418 | 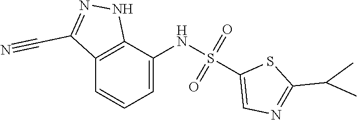 [SMILES: CC(C)c1ncc(S(=O)(=O)Nc2cccc3c(C#N)n[nH]c23)s1] |
| 419 | 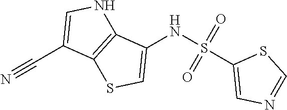 [SMILES: N#Cc1c[nH]c2c(NS(=O)(=O)c3cncs3)csc12] |
| 420 | 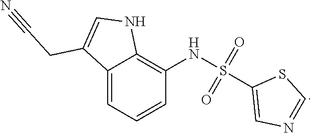 [SMILES: N#CCc1c[nH]c2c(NS(=O)(=O)c3cncs3)cccc12 ; "."] |

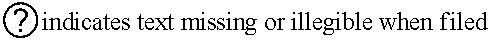

42. A pharmaceutical composition comprising the compound or salt of claim 1 and a pharmaceutically acceptable excipient.

43. A method of modulating an RBM39 protein, comprising contacting the RBM39 protein with the compound or salt of claim 1.

44. (canceled)

45. (canceled)

46. (canceled)

47. A method of treating a disease associated with aberrant RBM39 activity in a subject, comprising administering to the subject a therapeutically effective amount of the compound or salt of claim 1.

48. (canceled)

49. (canceled)
